(19) United States
(12) Patent Application Publication
(10) Pub. No.: US 2025/0290040 A1
(43) Pub. Date: Sep. 18, 2025

(54) GAMMA DELTA T CELL COMPOSITIONS AND METHODS OF USE

(71) Applicant: BEIGENE SWITZERLAND GMBH, Basel (CH)

(72) Inventors: Jhang-Sian YU, Taipei (TW); Shih-Min HUANG, Taipei (TW); Lien-Szu WU, Taipei (TW); Jin-Yi LU, Taipei (TW); Ao HU, Shanghai (CN); Yi-Han DAI, Taipei (TW); Chia-Hung LIN, Taipei (TW); Minhong YAN, Shanghai (CN); Yo-Chuen LIN, Taipei (TW); De-Kuan CHANG, Taipei (TW); Shiou-Ling JIAN, Taipei (TW)

(73) Assignee: BEIGENE SWITZERLAND GMBH, Basel (CH)

(21) Appl. No.: 19/080,477

(22) Filed: Mar. 14, 2025

Related U.S. Application Data
(60) Provisional application No. 63/565,614, filed on Mar. 15, 2024.
(60) Provisional application No. 63/675,543, filed on Jul. 25, 2024.

(30) Foreign Application Priority Data
Oct. 18, 2024 (WO) PCT/CN2024/125782

(51) Int. Cl. C12N 5/0783; A61K 40/00; C07K 14/57; C07K 14/725; C07K 16/28; C12N 9/22; C12N 15/11; C12N 15/90

(52) U.S. Cl. CPC C12N 5/0636; C07K 14/57; C07K 14/7051; C07K 16/2803; C12N 9/22; C12N 15/11; C12N 15/907; A61K 40/00; C12N 2310/20; C12N 2506/45

(57) ABSTRACT

The present disclosure provides genetically modified iPSC-derived γδT cells and their precursors. A double genomic disruption in the suppressor of cytokine signaling 1 (SOCS1) gene and the cytokine-inducible sh2-containing protein (CISH) gene are provided, as is a triple genomic disruption in genes for SOCS1, CISH, and Bcl-2 interacting mediator of cell death (BIM), as is a quadruple genomic disruption in genes for SOCS1, CISH, BIM, and cell surface death receptor (FAS), as is a quintuple genomic disruption in genes for SOCS1, CISH, BIM, β-2-Microglobulin (B2M), and class II transactivator (CITTA), as is a sextuple genomic disruption in genes for SOCS1, CISH, BIM, B2M, CITTA, and FAS. Also provided is genetically modified iPSC-derived γδT cells and their precursors with improved proliferation and tumor killing activity. Also provided are genetically modified iPSC-derived γδT cells and their precursors further comprising CD19 CAR. The present disclosure further provides methods making and using such cells, as well as gene editing systems.

### CROSS-REFERENCE TO RELATED APPLICATIONS

This application claims priority to and the benefit of U.S. Patent Application No. 63/565,614 filed on Mar. 15, 2024, U.S. Patent Application No. 63/675,543 filed on Jul. 25, 2024, and PCT Application No. PCT/CN2024/125782 filed on Oct. 18, 2024, the contents of which are hereby incorporated herein by reference in their entirety.

### SEQUENCE LISTING

The instant application contains a sequence listing which has been submitted electronically in xml format, and is hereby incorporated by reference in its entirety. Said xml file was created on Mar. 10, 2025, is 126 bytes in size, and named 138881_1259_Sequence_Listing.xml.

### FIELD OF THE DISCLOSURE AND BACKGROUND

This disclosure relates to genetically modified γδT cells and their precursors. A gamma-delta T cell (γδT-cell) is a type of T cell that has a T-cell receptor (TCR) composed of a γ glycoprotein chain and a δ glycoprotein chain on the surface of the cell. Unlike an alpha-beta T cell (αβT-cell), which has an α glycoprotein chain and a β glycoprotein chain and is activated in a major histocompatibility complex-dependent manner, a γδT-cell has no such activation restriction. Furthermore, a γδT-cell can recognize whole proteins and can distinguish between healthy and abnormal cells (e.g., infected, or cancerous). γδT-cells can be genetically modified to tailor the γδT-cell for specific roles in diagnostic and therapeutic settings.

Human BIM gene (Gene ID: 10018), also called BCL2L11, or “Bcl-2 Interacting Mediator of cell death” is transcribed as three splice variants: BIM_{EL}, BIM_{L}, and BIM_{S}. BIM is a member of BH3 (Bcl-2 homology 3)-only proteins, which can directly activate the proapoptotic effector proteins BAX and BAK. On the other hand, BIM also can indirectly activate BAX and BAK by binding to antiapoptotic BCL-2 family members (BCL-2, BCL-xL, MCL-1 and A1).

Cytokine-inducible SH2-containing protein (CISH)/(CIS1) (Gene ID: 1154, NM_013324.7, NP_037456.5) is a member of the suppressor of cytokine signaling (SOCS) protein family. The SOCS family members are negative regulators of cytokine signal transduction that inhibit the JAK/STAT pathway. Each SOCS family member contains a central SH2 domain and a conserved carboxy-terminal motif designated as the SOCS box. These proteins are important regulators of cytokine signaling, proliferation, differentiation, and immune responses.

The SOCS family consists of at least eight members including the originally identified CIS1, as well as SOCS1, SOCS2, SOCS3, SOCS4, SOCS5, SOCS6, and SOCS7. SOCS1 (Suppressor of cytokine signaling 1) also known as JAB (Janus kinase binding protein), SSI-1 (Stat-induced Stat inhibitor-1), Gene ID: 8651, NM_003745.2, NP_003736.1, and TIP3 (Tec-interacting protein 3), is a cytokine-regulated SOCS family member that directly inhibits JAK family members through interaction within their kinase activation loop. In addition to inhibiting JAK/STAT signaling, SOCS1 can also negatively regulate Toll-like receptors that contribute to innate immunity.

Human FAS cell surface death receptor protein (Gene ID: 355, NM_000043.6, NP_000034.1), also known as “FAS” or “CD95,” is one of five tumor necrosis factor (TNF) superfamily death receptors. Human FAS can induce caspase-dependent apoptosis following engagement with its extracellular ligand (FAS ligand (FASL)). The FASL-FAS signaling-induced apoptotic pathway plays an important role in immune regulation, development of cancer, and progression of cancer. In addition, FASL-FAS signaling also has a role in the auto-regulatory circuit for CAR-engineered lymphocyte persistence.

β-2-Microglobulin (Gene ID: 567, NM 004048, NP_004039), also known as “β2M” or “B2M”, is a component of the human leukocyte antigen (HLA) class I molecules found on the surface of nearly all nucleated cells. The B2M gene, which encodes β2M, is located on chromosome 15 (15q21.1) and consists of 4 exons. It encodes a 12 kDa protein. β2M associates non-covalently with the HLA class I heavy chain, enabling proper folding and stabilization of HLA class I. β2M-free HLA class I molecules are eventually degraded by proteasomes in the cytosol. Genetic ablation of the 02M gene eliminates surface HLA class I expression, reducing CD8+ T cell-mediated allogeneic responses.

The Class II transactivator gene (Gene ID: 4261, NM_001286402.1, NP_001273331.1), encodes the Class II transactivator protein, which is also called “CIITA”. The CIITA gene is located on chromosome 16 (16p13.13) and has 19 exons. It encodes a 130 kDa protein. CIITA serves as a master regulator of HLA class II expression by recruiting and facilitating the assembly of DNA-binding proteins including the regulatory factor X (RFX) complex, cAMP response element-binding protein (CREB), and nuclear factor Y (NF-Y). Together, this complex enables HLA class II expression. Genetic ablation of the CIITA gene eliminates surface HLA class II expression, reducing CD4+ T cell-mediated allogeneic responses.

Allogeneic immune recognition and rejection remain obstacles in cellular transplantation and cell therapy development. A major cause of transplant rejection is HLA mismatch. To suppress these HLA mismatch-mediated responses, genetic ablation of HLA in iPSC-derived γδ T cells can be conducted.

Interferon-gamma receptor (IFNGR) is a cytokine receptor of interferon gamma (IFN-7). IFNGR comprises a heterodimer of interferon-gamma receptor 1 (IFNGR1) and interferon-gamma receptor 2 (IFNGR2). IFN-γ can signal IFNGR-expressing cells, enabling the immunomodulatory effects of IFN-γ, including augmenting antigen presentation through both the MHC class I and class II pathways. CD132, also called “the common γ chain (γc) of interleukin-2 receptor” or “IL2RG,” is a cytokine receptor subunit that is common to the cytokine receptor complexes of at least IL-2, IL-4, IL-7, IL-9, IL-15, and IL-21. Interleukin-2 receptor beta subunit, also known as “CD122” or “IL2RB,” is a cytokine receptor subunit of the cytokine receptor complex of IL-2 (IL2R). IL2R is a protein complex found on the surface of cells, including T cells, NK cells and other immune cells. IL-2 mediated IL2R signaling promotes the differentiation of T cells into effector T cells and memory T cells. IL-2 mediated IL2R signaling enables downstream signaling pathways through the Janus kinase (JAK)/signal transducer and activator of transcription (STAT) pathway, e.g., STAT1, STAT5. Modulating these signaling pathways may, for example, be useful in treating diseases such as cancer by helping to promote killing of tumor cells by immune cells.

CD19 is a protein that is primarily expressed on the surface of B cells, which are a type of white blood cell involved in the immune response. It plays an important role in the regulation and activation of B cells. CD19 is a co-receptor for the B-cell receptor (BCR), and it helps enhance the signaling pathways that promote B cell activation, survival, and differentiation. Because of its specific expression on B cells, CD19 has been targeted in certain therapeutic approaches.

There remains an unmet need for therapeutic γδT-cells. Such modified cells would be useful in a variety of diagnostic and therapeutic settings, such as in the treatment of human disease and disorders.

### SUMMARY

The present inventors have herein demonstrated that genomic disruptions in 1) an endogenous suppressor of cytokine signaling 1 (SOCS1) gene; and 2) an endogenous cytokine-inducible SH2-containing protein (CISH) gene, and optionally 3) an endogenous Bcl-2 Interacting Mediator of cell death (BIM) gene, and optionally 4) an endogenous FAS cell surface death receptor (FAS) gene, and optionally, 5) an endogenous β-2-Microglobulin (B2M) gene, and optionally 6) an endogenous class II transactivator (CIITA) gene in γδT (e.g., iPSC-derived γδT) cells confer enhanced cytotoxicity and persistence and/or increased survival in the γδT cells. The modified cells can be used for a variety of therapeutic and diagnostic purposes.

In some embodiments, the present disclosure provides a modified γδT cell (e.g., from primary γδT cell or differentiated from modified iPSC), comprising a genomic disruption in an endogenous suppressor of cytokine signaling 1 (SOCS1) gene that suppresses or eliminates expression of a functional SOCS1 protein and a genomic disruption in an endogenous cytokine-inducible SH2-containing protein (CISH) gene that suppresses or eliminates expression of a functional CISH protein, which shows synergistic effects in promoting gdT persistence and/or enhanced gdT cytotoxicity.

In some embodiments, the present disclosure provides a modified γδT (e.g., from primary γδT cell or differentiated from modified iPSC), comprising a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell, showing better survival ability of gdT.

In some embodiments, the present disclosure provides a modified γδT (e.g., from primary γδT cell or differentiated from modified iPSC), comprising (1) a genomic disruption in an endogenous suppressor of cytokine signaling 1 (SOCS1) gene that suppresses or eliminates expression of a functional SOCS1 protein, (2) a genomic disruption in an endogenous cytokine-inducible SH2-containing protein (CISH) gene that suppresses or eliminates expression of a functional CISH protein, and (3) a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell, showing enhanced gdT persistence, enhanced gdT cytotoxicity and better survival ability of gdT cells.

In some embodiments, the present disclosure provides a modified γδT cell (e.g., from primary γδT cell or differentiated from modified iPSC), comprising (1) a genomic disruption in an endogenous suppressor of cytokine signaling 1 (SOCS1) gene that suppresses or eliminates expression of a functional SOCS1 protein and a genomic disruption in an endogenous cytokine-inducible SH2-containing protein (CISH) gene that suppresses or eliminates expression of a functional CISH protein, and/or (2) a genomic disruption in an exon 2C of an endogenous BIM gene, wherein the expression and/or function of BIM_{EL} and BIM_{L} splice variants are reduced in the modified cell relative to that in an unmodified control and the expression and/or function of the BIM_{S} splice variant is retained in the modified cell, optionally further comprising (a) a genomic disruption in an endogenous FAS cell surface death receptor (FAS) gene, optionally further comprising (b) a genomic disruption in an endogenous β-2-Microglobulin (B2M) gene, optionally further comprising (c) a genomic disruption in an endogenous Class II transactivator (CIITA) gene, and/or optionally further comprising (d) one or more exogenous polynucleotides encoding an IFNγ signal converter, showing any one or more of the following features:
(1) enhanced gdT persistence and/or enhanced gdT cytotoxicity;
(2) enhanced survival ability of gdT;
(3) enhanced durable killing (e.g., around 20 rounds of killing in an embodiment);
(4) ability to secrete higher amounts of effector molecules; and
(5) improved cell expansion.

The present disclosure also provides a modified iPSC or an intermediate cell differentiated from iPSC having 1) a genomic disruption in an endogenous suppressor of cytokine signaling 1 (SOCS1) gene that suppresses or eliminates expression of SOCS1 and a genomic disruption in an endogenous cytokine-inducible SH2-containing protein (CISH) gene that suppresses or eliminates expression of CISH, and/or (2) a genomic disruption in an exon 2C of an endogenous BIM gene, wherein the expression and/or function of BIM_{EL} and BIM_{L} splice variants are reduced in the modified cell relative to that in an unmodified control and the expression and/or function of the BIM_{S} splice variant is retained in the modified cell, optionally further comprising (a) a genomic disruption in an endogenous FAS cell surface death receptor (FAS) gene, optionally further comprising (b) a genomic disruption in an endogenous β-2-Microglobulin (B2M) gene, optionally further comprising (c) a genomic disruption in an endogenous Class II transactivator (CIITA) gene, and/or optionally further comprising (d) one or more exogenous polynucleotides encoding an IFNγ signal converter, which can be used for differentiation into the modified gdT cells.

The above-mentioned modified iPSC, γδT cell, or intermediate cell differentiated from an iPSC can further comprises an exogenous polynucleotide encoding a CAR which specifically binds human CD19 (anti-CD19 CAR).

In some embodiments, the present disclosure provides a modified γδT cell (e.g., from primary γδT cell or differentiated from modified iPSC) comprising an exogenous polynucleotide encoding a CAR which specifically binds human CD19, and further comprising (1) a genomic disruption in an endogenous suppressor of cytokine signaling 1 (SOCS1) gene that suppresses or eliminates expression of a functional SOCS1 protein and a genomic disruption in an endogenous cytokine-inducible SH2-containing protein (CISH) gene that suppresses or eliminates expression of a functional CISH protein, and/or (2) a genomic disruption in an exon 2C of an endogenous BIM gene, wherein the expression and/or function of BIM_{EL} and BIM_{L} splice variants are reduced in the modified cell relative to that in an unmodified control and the expression and/or function of the BIM_{S} splice variant is retained in the modified cell, optionally further comprising (a) a genomic disruption in an endogenous FAS cell surface death receptor (FAS) gene, optionally further comprising (b) a genomic disruption in an endogenous β-2-Microglobulin (B2M) gene, optionally further comprising (c) a genomic disruption in an endogenous Class II transactivator (CIITA) gene, and/or optionally further comprising (d) one or more exogenous polynucleotides encoding an IFNγ signal converter, showing any one or more of the following features:
(1) specific cytotoxicity against CD19 positive B cells (especially CD19 positive abnormal B cells);
(2) excellent cytotoxicity against CD19 positive B cells (especially CD19 positive abnormal B cells), comparable or better than that of CD19-CAR αβT cells;
(3) ability to secrete higher amounts of effector molecules and/or lower amount of inflammatory or immune-modulatory cytokines, e.g., compared with CD19-CAR αβT cells;
(4) improved cell expansion; and
(5) enhanced durable killing against CD19 positive B cells (especially CD19 positive abnormal B cells) (e.g., more than 35 rounds of killing in an embodiment).

The present disclosure also provides a modified iPSC, γδT cell, or intermediate cell differentiated from an iPSC, comprising one or more exogenous polynucleotides encoding an IFNγ signal converter, wherein the IFNγ signal converter comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB); further comprising an exogenous polynucleotide encoding a chimeric antigen receptor (CAR), showing excellent durable killing and excellent cell proliferation (e.g., synergistic effect).

The present disclosure encompasses, inter alia, the following embodiments, among others disclosed herein.
1. A modified iPSC, γδT cell, or intermediate cell differentiated from an iPSC, comprising a genomic disruption in an endogenous suppressor of cytokine signaling 1 (SOCS1) gene that suppresses or eliminates expression of a functional SOCS1 protein and a genomic disruption in an endogenous cytokine-inducible SH2-containing protein (CISH) gene that suppresses or eliminates expression of a functional CISH protein.
2. The modified cell of embodiment 1, wherein the genomic disruption is in exon 2 of the SOCS1 gene; optionally wherein the genomic disruption is an inactivating nucleic acid mutation, optionally wherein the mutation is an insertion, deletion, or substitution.
3. The modified cell of embodiment 1 or embodiment 2, wherein the genomic disruption is in exon 3 or exon 4 of the CISH gene; optionally wherein the genomic disruption is an inactivating nucleic acid mutation, optionally wherein the mutation is an insertion, deletion, or substitution.
4. The modified cell of any one of embodiments 1-3, wherein the genomic disruption is within a target sequence in SOCS1 that comprises SEQ ID NO: 17 or SEQ ID NO: 23 and/or the genomic disruption is within a target sequence in CISH that comprises SEQ ID NO: 15, SEQ ID NO: 16 or SEQ ID NO: 24.
5. The modified cell of any one of embodiments 1-4, wherein the genomic disruption within SOCS1 and CISH is made by administering guide RNAs to said cell, optionally wherein the guide RNAs are single-guide RNAs (sgRNAs) or crRNAs, and wherein an endonuclease or a nucleic acid encoding the endonuclease is also administered to said cell.
6. The modified cell of embodiment 5, wherein the guide RNAs comprise the guide sequence of any one or more of SEQ ID NOs: 5, 11, 3, 4, and 12, or comprises the sgRNA or crRNA sequence of any one or more of SEQ ID NOs: 29, 37, 27, 28, and 38.
7. The modified cell of any one of embodiments 1-6, further comprising a genomic disruption in an endogenous Bcl-2 Interacting Mediator of cell death (BIM) gene.
8. The modified cell of embodiment 7, wherein the genomic disruption in the BIM gene is in exon 2C of the BIM gene.
9. The modified cell of embodiment 7 or embodiment 8, wherein the expression and/or function of BIM_{EL} and BIM_{L} splice variants are reduced in the modified cell relative to that in an unmodified control and the expression and/or function of BIM_{S} splice variant is retained in the modified cell.
10. The modified cell of any one of embodiments 7-9, wherein the genomic disruption is an inactivating nucleic acid mutation in an exon 2C of an endogenous BIM gene, optionally wherein the mutation is an insertion, deletion, or substitution.
11. The modified cell of any one of embodiments 7-10, wherein the genomic disruption is within a target sequence in BIM that comprises any one of SEQ ID NOs: 14, 18, 19, 20, 21, and 22.
12. The modified cell of any one of embodiments 7-11, wherein the genomic disruption within BIM is made by administering a guide RNA to said cell, optionally wherein the guide RNA is a single-guide RNA (sgRNA) or crRNAs, and optionally wherein an endonuclease or a nucleic acid encoding the endonuclease is also administered to said cell.
13. The modified cell of embodiment 12, wherein the guide RNA comprises the guide sequence of any one of SEQ ID NOs: 2, 6, 7, 8, 9, and 10, or comprises the sgRNA or crRNA sequence of any one or more of SEQ ID NOs: 26, 32, 33, 34, 35, and 36.
14. The modified cell of any one of embodiments 1-13, further comprising a genomic disruption in an endogenous FAS cell surface death receptor (FAS) gene that suppresses or eliminates expression of a functional FAS protein.
15. The modified cell of embodiment 14, wherein the genomic disruption in the FAS gene is in exon 1 of the FAS gene.
16. The modified cell of embodiment 14 or embodiment 15, wherein the genomic disruption is an inactivating nucleic acid mutation in exon 1 of an endogenous FAS gene, optionally wherein the mutation is an insertion, deletion, or substitution.
17. The modified cell of any one of embodiments 14-16, wherein the genomic disruption is within a target sequence in FAS that comprises SEQ ID NO: 62.
18. The modified cell of any one of embodiments 14-17, wherein the genomic disruption within FAS is made by administering a guide RNA to said cell, optionally wherein the guide RNA is a single-guide RNA (sgRNA) or crRNAs, and optionally wherein an endonuclease or a nucleic acid encoding the endonuclease is also administered to said cell.
19. The modified cell of embodiment 18, wherein the guide RNA comprises the guide sequence of SEQ ID NO: 61, or comprises the sgRNA sequence of SEQ ID NO: 60.
20. The modified cell of any one of embodiments 1-19, further comprising a genomic disruption in an endogenous β-2-Microglobulin (B2M) gene that suppresses or eliminates expression of a functional B2M protein.
21. The modified cell of embodiment 20, wherein the genomic disruption in the B2M gene is in exon 2 of the B2M gene.
22. The modified cell of embodiment 20 or embodiment 21, wherein the genomic disruption is an inactivating nucleic acid mutation in exon 2 of an endogenous B2M gene, optionally wherein the mutation is an insertion, deletion, or substitution.
23. The modified cell of any one of embodiments 20-22, wherein the genomic disruption is within a target sequence in B2M that comprises SEQ ID NO: 56.
24. The modified cell of any one of embodiments 20-23, wherein the genomic disruption within B2M is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
25. The modified cell of embodiment 24, wherein the guide RNA comprises the guide sequence of SEQ ID NO: 55, or comprises the sgRNA sequence of SEQ ID NO: 54.
26. The modified cell of any one of embodiments 1-25, further comprising a genomic disruption in an endogenous Class II transactivator (CIITA) gene that suppresses or eliminates expression of a functional CIITA protein.
27. The modified cell of embodiment 26, wherein the genomic disruption in the CIITA gene is in exon 3 of the CIITA gene.
28. The modified cell of embodiment 26 or embodiment 27, wherein the genomic disruption is an inactivating nucleic acid mutation in exon 3 of an endogenous CIITA gene, optionally wherein the mutation is an insertion, deletion, or substitution.
29. The modified cell of any one of embodiments 26-28, wherein the genomic disruption is within a target sequence in CIITA that comprises SEQ ID NO: 59.
30. The modified cell of any one of embodiments 26-29, wherein the genomic disruption within CIITA is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
31. The modified cell of embodiment 30, wherein the guide RNA comprises the guide sequence of SEQ ID NO: 58, or comprises the sgRNA sequence of SEQ ID NO: 57.
32. The modified cell of any one of embodiments 1-31, further comprising a genomic disruption in an endogenous T-cell receptor alpha constant region (TRAC) gene that suppresses or eliminates expression of a functional TRAC protein.
33. The modified cell of any one of embodiments 1-32, wherein the genomic disruption is an inactivating nucleic acid mutation, optionally wherein the mutation is an insertion, deletion, or substitution.
34. The modified cell of any one of embodiments 1-33, further comprising one or more exogenous polynucleotides encoding an IFNγ signal converter, wherein the IFNγ signal converter comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB).
35. The modified cell of embodiment 34, wherein the amino acid sequence of ECD of the first IFNγ fusion protein subunit is SEQ ID NO: 63, and the amino acid sequence of ECD of the second IFNγ fusion protein subunit is SEQ ID NO: 64.
36. The modified cell of embodiment 34 or embodiment 35, wherein the amino acid sequence of intracellular domain of the first IFNγ fusion protein subunit is SEQ ID NO: 69, and the amino acid sequence of intracellular domain of the second IFNγ fusion protein subunit is SEQ ID NO: 70.
37. The modified cell of any one of embodiments 34-36, wherein the first IFNγ fusion protein subunit further comprises a transmembrane domain between the ECD and the intracellular domain thereof, and the second IFNγ fusion protein subunit further comprises a transmembrane domain between the ECD and the intracellular domain thereof.
38. The modified cell of embodiment 37, wherein the transmembrane domain of the first IFNγ fusion protein subunit is of the interleukin-2 receptor subunit gamma, and the transmembrane domain of the second IFNγ fusion protein subunit is of the interleukin-2 receptor subunit beta.
39. The modified cell of embodiment 38, wherein the amino acid sequence of transmembrane domain of the first IFNγ fusion protein subunit is SEQ ID NO: 67, and the amino acid sequence of transmembrane domain of the second IFNγ fusion protein subunit is SEQ ID NO: 68.
40. The modified cell of any one of embodiments 34-39, wherein the first IFNγ fusion protein subunit further comprises a membrane-proximal region between the ECD and the intracellular domain thereof, and the second IFNγ fusion protein subunit further comprises a membrane-proximal region between the ECD and the intracellular domain thereof.
41. The modified cell of embodiment 40, wherein the membrane-proximal region of the first IFNγ fusion protein subunit is of the interleukin-2 receptor subunit gamma, and the membrane-proximal region of the second IFNγ fusion protein subunit is of the interleukin-2 receptor subunit beta.
42. The modified cell of embodiment 41, wherein the amino acid sequence of membrane-proximal region of the first IFNγ fusion protein subunit is SEQ ID NO: 65, and the amino acid sequence of membrane-proximal region of the second IFNγ fusion protein subunit is SEQ ID NO: 66.
43. The modified cell of any one of embodiment 34-42, wherein a first exogenous polynucleotide encoding the first IFNγ fusion protein subunit of the IFNγ signal converter is inserted into a SOCS1 locus.
44. The modified cell of any one of embodiments 34-43, wherein a second exogenous polynucleotide encoding the second IFNγ fusion protein subunit of the IFNγ signal converter is inserted into a ROSA26 locus.
45. The modified cell of any one of embodiments 34-44, the first IFNγ fusion protein subunit of the IFNγ signal converter comprises an amino acid sequence of SEQ ID NO: 71, and the second IFNγ fusion protein subunit of the IFNγ signal converter comprises an amino acid sequence of SEQ ID NO: 72; optionally the first exogenous polynucleotide encodes an amino acid sequence comprising SEQ ID NO: 81, and the second exogenous polynucleotide encodes an amino acid sequence comprising SEQ ID NO: 82.
46. The modified cell of any one of embodiments 34-45, wherein the amino acid sequence of the first IFNγ fusion protein subunit is SEQ ID NO: 71, and the amino acid sequence of the second IFNγ fusion protein subunit is SEQ ID NO: 72.
47. The modified cell of any one of embodiments 1-46, further comprising an exogenous polynucleotide encoding a B2M-HLA-E fusion protein (B2M-HLA-E) comprising at least a portion of B2M fused with at least of a portion of HLA-E.
48. The modified cell of embodiment 47, wherein the exogenous polynucleotide encoding the B2M-HLA-E is inserted into a B2M locus.
49. The modified cell of embodiment 47 or embodiment 48, wherein the B2M-HLA-E comprises an amino acid sequence of SEQ ID NO: 85.
50. The modified cell of any one of embodiments 1-49, further comprising an exogenous polynucleotide encoding a chimeric antigen receptor (CAR).
51. The modified cell of embodiment 50, wherein the exogenous polynucleotide encoding the CAR is inserted into an AAVS1 locus.
52. The modified cell of embodiment 50 or embodiment 51, wherein the exogenous polynucleotide encodes the CAR which specifically binds human CD19.
53. The modified cell of embodiment 52, wherein the CAR which specifically binds human CD19 comprises the amino acid of SEQ ID NO: 83 or SEQ ID NO: 84.
54. The modified cell of any one of embodiments 1-53, wherein the iPSC is derived from a primary T cell (T-iPSC).
55. The modified cell of any one of embodiments 1-54, wherein the iPSC is derived from a primary γδT cell (γδT-iPSC).
56. The modified cell of any one of embodiments 1-55, wherein the γδT cell is Vd2 γδT cell.
57. The modified cell of any one of embodiments 1-56, wherein the γδT cell is an iPSC-derived γδT (iγδT) cell, optionally the γδT cell is a γδT cell derived from a T-iPSC (T-iγδT).
58. The modified cell of any one of embodiments 1-57, wherein the γδT cell is a γδT cell differentiated from a γδT-iPSC (γδT-iγδT).
59. The modified cell of any one of embodiments 1-58, wherein the intermediate cell differentiated from the iPSC is a γδT-iHSC, a γδT-iCLP, and/or an immature T-iγδT cell.
60. A population of cells comprising the modified cell of any one of embodiments 1-59.
61. The population of cells of embodiment 60, frozen in storage at about −196 degrees Celsius.
62. A method of generating a modified iPSC, γδT cell, or intermediate cell differentiated from an iPSC comprising contacting the cell with guide RNAs and an endonuclease or a nucleic acid encoding the endonuclease, wherein the contacting results in a genomic disruption in an endogenous SOCS1 gene that suppresses or eliminates expression of a functional SOCS1 protein and a genomic disruption in an endogenous CISH gene that suppresses or eliminates expression of a functional CISH protein.
63. The method of embodiment 62, wherein the iPSC is derived from a primary T cell (T-iPSC).
64. The method of embodiment 62 or embodiment 63, wherein the iPSC is derived from a primary γδT cell (γδT-iPSC).
65. The method of any one of embodiments 62-64, wherein the γδT cell is Vd2 γδT cell.
66. The method of any one of embodiments 62-65, wherein the γδT cell is an iPSC-derived γδT (iγδT) cell, optionally the γδT cell is a γδT cell derived from a T-iPSC (T-iγδT).
67. The method of any one of embodiments 62-66, wherein the γδT cell is a γδT cell differentiated from a γδT-iPSC (γδT-iγδT).
68. The method of any one of embodiments 62-67, wherein the intermediate cell differentiated from the iPSC is a γδT-iHSC, a γδT-iCLP, and/or an immature T-iγδT cell.
69. The method of any one of embodiments 62-68, wherein the genomic disruption is in exon 2 of the SOCS1 gene; optionally wherein the genomic disruption is an inactivating nucleic acid mutation, optionally wherein the mutation is an insertion, deletion, or substitution.
70. The method of any one of embodiments 62-69, wherein the genomic disruption is in exon 3 or exon 4 of the CISH gene; optionally wherein the genomic disruption is an inactivating nucleic acid mutation, optionally wherein the mutation is an insertion, deletion, or substitution.
71. The method of any one of embodiments 62-70, wherein the genomic disruption is within a target sequence in SOCS1 that comprises SEQ ID NO: 17 or SEQ ID NO: 23 and/or the genomic disruption is within a target sequence in CISH that comprises SEQ ID NO: 15, SEQ ID NO: 16 or SEQ ID NO: 24.
72. The method of any one of embodiments 62-71, wherein the genomic disruption within SOCS1 and CISH is made by administering guide RNAs to said cell, optionally wherein the guide RNAs are single-guide RNAs (sgRNAs) or crRNAs, and optionally wherein a Cas endonuclease or a nucleic acid encoding the Cas endonuclease is also administered to said cell.
73. The method of embodiment 72, wherein the guide RNAs comprise the guide sequence of any one or more of SEQ ID NOs: 5, 11, 3, 4, and 12, or comprises the sgRNA or crRNA sequence of any one or more of SEQ ID NOs: 29, 37, 27, 28, and 38.
74. The method of any one of embodiments 62-73, further comprising inhibiting expression of an endogenous BIM gene by causing a genomic disruption in an endogenous BIM gene.
75. The method of embodiment 74, wherein the genomic disruption in the BIM gene is in exon 2C of the BIM gene.
76. The method of embodiment 74 or embodiment 75, wherein the expression and/or function of BIM_{EL} and BIM_{L} splice variants are reduced in the cell relative to that in an unmodified control and the expression and/or function of BIM_{S} splice variant is retained in the cell.
77. The method of any one of embodiments 74-76, wherein the genomic disruption is an inactivating nucleic acid mutation in an exon 2C of an endogenous BIM gene, optionally wherein the mutation is an insertion, deletion, or substitution.
78. The method of any one of embodiments 74-77, wherein the genomic disruption is within a target sequence in BIM that comprises any one of SEQ ID NOs: 14, 18, 19, 20, 21, and 22.
79. The method of any one of embodiments 74-78, wherein the genomic disruption within BIM is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
80. The method of embodiment 79, wherein the guide RNA comprises the guide sequence of any one of SEQ ID NOs: 2, 6, 7, 8, 9, and 10, or comprises the sgRNA or crRNA sequence of any one or more of SEQ ID NOs: 26, 32, 33, 34, 35, and 36.
81. The method of any one of embodiments 62-80, further comprising inhibiting expression of an endogenous FAS gene by causing a genomic disruption in an endogenous FAS gene that suppresses or eliminates expression of a functional FAS protein.
82. The method of embodiment 81, wherein the genomic disruption in the FAS gene is in exon 1 of the FAS gene.
83. The method of embodiment 81 or embodiment 82, wherein the genomic disruption is an inactivating nucleic acid mutation in exon 1 of an endogenous FAS gene, optionally wherein the mutation is an insertion, deletion, or substitution.
84. The method of any one of embodiments 81-83, wherein the genomic disruption is within a target sequence in FAS that comprises SEQ ID NO: 62.
85. The method of any one of embodiments 81-84, wherein the genomic disruption within FAS is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
86. The method of embodiment 85, wherein the guide RNA comprises the guide sequence of SEQ ID NO: 61, or comprises the sgRNA sequence of SEQ ID NO: 60.
87. The method of any one of embodiments 62-86, further comprising inhibiting expression of an endogenous B2M gene by causing a genomic disruption in an endogenous B2M gene that suppresses or eliminates expression of a functional B2M protein.
88. The method of embodiment 87, wherein the genomic disruption in the B2M gene is in exon 2 of the B2M gene.
89. The method of embodiment 87 or embodiment 88, wherein the genomic disruption is an inactivating nucleic acid mutation in exon 2 of an endogenous B2M gene, optionally wherein the mutation is an insertion, deletion, or substitution.
90. The method of any one of embodiments 87-89, wherein the genomic disruption is within a target sequence in B2M that comprises SEQ ID NO: 56.
91. The method of any one of embodiments 87-90, wherein the genomic disruption within B2M is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
92. The method of embodiment 91, wherein the guide RNA comprises the guide sequence of SEQ ID NO: 55, or comprises the sgRNA sequence of SEQ ID NO: 54.
93. The method of any one of embodiments 62-92, further comprising inhibiting expression of an endogenous CIITA gene by causing a genomic disruption in an endogenous CIITA gene that suppresses or eliminates expression of a functional CIITA protein.
94. The method of embodiment 93, wherein the genomic disruption in the CIITA gene is in exon 3 of the CIITA gene.
95. The method of embodiment 93 or embodiment 94, wherein the genomic disruption is an inactivating nucleic acid mutation in exon 3 of an endogenous CIITA gene, optionally wherein the mutation is an insertion, deletion, or substitution.
96. The method of any one of embodiments 93-95, wherein the genomic disruption is within a target sequence in CIITA that comprises SEQ ID NO: 59.
97. The method of any one of embodiments 93-96, wherein the genomic disruption within CIITA is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
98. The method of embodiment 97, wherein the guide RNA comprises the guide sequence of SEQ ID NO: 58, or comprises the sgRNA sequence of SEQ ID NO: 57.
99. The method of any one of embodiments 62-98, further comprising inhibiting expression of an endogenous TRAC gene by causing a genomic disruption in an endogenous TRAC gene that suppresses or eliminates expression of a functional TRAC protein.
100. The method of any one of embodiments 62-99, wherein the genomic disruption is an inactivating nucleic acid mutation, optionally wherein the mutation is an insertion, deletion, or substitution.
101. The method of any one of embodiments 62-100, further comprising introducing one or more exogenous polynucleotides encoding an IFNγ signal converter into the cell, wherein the IFNγ signal converter comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB).
102. The method of embodiment 101, wherein a first exogenous polynucleotide encoding the first IFNγ fusion protein subunit of the IFNγ signal converter is inserted into a SOCS1 locus.
103. The method of embodiment 101 or 102, wherein a second exogenous polynucleotide encoding the second IFNγ fusion protein subunit of the IFNγ signal converter is inserted into a ROSA26 locus.
104. The method of any one of embodiments 101-103, the first IFNγ fusion protein subunit of the IFNγ signal converter comprises an amino acid sequence of SEQ ID NO: 71, and the second IFNγ fusion protein subunit of the IFNγ signal converter comprises an amino acid sequence of SEQ ID NO: 72; optionally the first exogenous polynucleotide encodes an amino acid sequence comprising SEQ ID NO: 81, and the second exogenous polynucleotide encodes an amino acid sequence comprising SEQ ID NO: 82.
105. The method of any one of embodiments 62-104, further comprising introducing an exogenous polynucleotide encoding a B2M-HLA-E fusion protein (B2M-HLA-E) comprising at least a portion of B2M fused with at least of a portion of HLA-E into the cell.
106. The method of embodiment 105, wherein the exogenous polynucleotide encoding the B2M-HLA-E is inserted into a B2M locus.
107. The method of any one of embodiments 105-106, wherein the B2M-HLA-E comprises an amino acid sequence of SEQ ID NO: 85.
108. The method of any one of embodiments 62-107, further comprising introducing an exogenous polynucleotide encoding a chimeric antigen receptor (CAR) into the cell.
109. The method of embodiment 108, wherein the exogenous polynucleotide encoding the CAR is inserted into an AAVS1 locus.
110. The method of embodiment 108 or embodiment 109, wherein the exogenous polynucleotide encodes the CAR which specifically binds human CD19.
111. The method of embodiment 110, wherein the CAR which specifically binds human CD19 comprises the amino acid of SEQ ID NO: 83 or SEQ ID NO: 84.
112. A modified iPSC, γδT cell, or intermediate cell differentiated from an iPSC, comprising a genomic disruption in an exon 2C of an endogenous BIM gene, wherein the expression and/or function of BIM_{EL} and BIM_{L} splice variants are reduced in the modified cell relative to that in an unmodified control, and the expression and/or function of BIM_{S} splice variant is retained in the modified cell.
113. The modified cell of embodiment 112, wherein the genomic disruption is an inactivating nucleic acid mutation in the exon 2C, optionally wherein the mutation is an insertion, deletion, or substitution.
114. The modified cell of embodiment 112 or embodiment 113, wherein the genomic disruption is within a target sequence in BIM that comprises any one of SEQ ID NOs: 14, 20, 21, and 22.
115. The modified cell of any one of embodiments 112-114, wherein the genomic disruption within BIM is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
116. The modified cell of embodiment 115, wherein the guide RNA comprises any one of SEQ ID NOs: 26, 34, 35 and 36, or a guide sequence differing by no more than 3 nucleotides from any one of SEQ ID Nos: 2, 8, 9 and 10.
117. The modified cell of embodiment 115 or embodiment 116, wherein the guide RNA comprises SEQ ID NO: 36 or a guide sequence differing by no more than 3 nucleotides from SEQ ID NO: 10, and the endonuclease is a MAD7 endonuclease.
118. The modified cell of embodiment 115 or embodiment 116, wherein the guide RNA comprises any one of SEQ ID NOs: 26, 34, and 35, or a guide sequence differing by no more than 3 nucleotides from any one of SEQ ID Nos: 2, 8, and 9, and the endonuclease is a Cas9 endonuclease.
119. A method of inhibiting expression of an endogenous BIM gene in a iPSC, γδT cell, or intermediate cell differentiated from an iPSC, comprising contacting the cell with a guide RNA and an endonuclease or a nucleic acid encoding the endonuclease, wherein the contacting results in a genomic disruption in an exon 2C of an endogenous BIM gene, wherein the expression and/or function of BIM_{EL} and BIM_{L} splice variants are reduced in the cell relative to that in an unmodified control and the expression and/or function of BIM_{S} splice variant is retained in the cell.
120. The method of embodiment 119, wherein the genomic disruption is an inactivating nucleic acid mutation in the exon 2C, optionally wherein the mutation is an insertion, deletion, or substitution.
121. The method of embodiment 119 or embodiment 120, wherein the genomic disruption is within a target sequence in BIM that comprises any one of SEQ ID NOs: 14, 20, 21, and 22.
122. The method of any one of embodiments 119-121, wherein the guide RNA comprises the guide sequence of any one of SEQ ID NOs: 2, 8, 9, and 10, or comprises the sgRNA or crRNA sequence of any one of SEQ ID NOs 26, 34, 35, and 36; optionally wherein the guide RNA is a single-guide RNA (sgRNA) or crRNA.
123. The method of any one of embodiments 119-122, wherein the γδT cell is a γδT cell differentiated from a γδT-iPSC (γδT-iγδT).
124. The method of any one of embodiments 119-123, wherein the cell is generated from the method of any one of 62-111.
125. A modified cell generated from the method of any one of embodiments 62-111 and embodiments 119-124.
126. A modified iPSC, γδT cell, or intermediate cell differentiated from an iPSC, comprising one or more exogenous polynucleotides encoding an IFNγ signal converter, wherein the IFNγ signal converter comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB); optionally further comprising an exogenous polynucleotide encoding a chimeric antigen receptor (CAR), optionally wherein the exogenous polynucleotide encoding the CAR is inserted into an AAVS1 locus, optionally wherein the exogenous polynucleotide encodes the CAR which specifically binds human CD19, optionally wherein the CAR which specifically binds human CD19 comprises the amino acid of SEQ ID NO: 83 or SEQ ID NO: 84.
127. The modified cell of embodiment 126, wherein the amino acid sequence of ECD of the first IFNγ fusion protein subunit is SEQ ID NO: 63, and the amino acid sequence of ECD of the second IFNγ fusion protein subunit is SEQ ID NO: 64.
128. The modified cell of embodiment 126 or embodiment 127, wherein the amino acid sequence of intracellular domain of the first IFNγ fusion protein subunit is SEQ ID NO: 69, and the amino acid sequence of intracellular domain of the second IFNγ fusion protein subunit is SEQ ID NO: 70.
129. The modified cell of any one of embodiments 126-128, wherein the first IFNγ fusion protein subunit further comprises a transmembrane domain between the ECD and the intracellular domain thereof, and the second IFNγ fusion protein subunit further comprises a transmembrane domain between the ECD and the intracellular domain thereof.
130. The modified cell of embodiment 129, wherein the transmembrane domain of the first IFNγ fusion protein subunit is of the interleukin-2 receptor subunit gamma, and the transmembrane domain of the second IFNγ fusion protein subunit is of the interleukin-2 receptor subunit beta.
131. The modified cell of embodiment 130, wherein the amino acid sequence of transmembrane domain of the first IFNγ fusion protein subunit is SEQ ID NO: 67, and the amino acid sequence of transmembrane domain of the second IFNγ fusion protein subunit is SEQ ID NO: 68.
132. The modified cell of any one of embodiments 126-131, wherein the first IFNγ fusion protein subunit further comprises a membrane-proximal region between the ECD and the intracellular domain thereof, and the second IFNγ fusion protein subunit further comprises a membrane-proximal region between the ECD and the intracellular domain thereof.
133. The modified cell of embodiment 132, wherein the membrane-proximal region of the first IFNγ fusion protein subunit is of the interleukin-2 receptor subunit gamma, and the membrane-proximal region of the second IFNγ fusion protein subunit is of the interleukin-2 receptor subunit beta.
134. The modified cell of embodiment 133, wherein the amino acid sequence of membrane-proximal region of the first IFNγ fusion protein subunit is SEQ ID NO: 65, and the amino acid sequence of membrane-proximal region of the second IFNγ fusion protein subunit is SEQ ID NO: 66.
135. The modified cell of any one of embodiment 126-134, wherein a first exogenous polynucleotide encoding the first IFNγ fusion protein subunit of the IFNγ signal converter is inserted into a SOCS1 locus.
136. The modified cell of any one of embodiments 126-135, wherein a second exogenous polynucleotide encoding the second IFNγ fusion protein subunit of the IFNγ signal converter is inserted into a ROSA26 locus.
137. The modified cell of any one of embodiments 126-136, the first IFNγ fusion protein subunit of the IFNγ signal converter comprises an amino acid sequence of SEQ ID NO: 71, and the second IFNγ fusion protein subunit of the IFNγ signal converter comprises an amino acid sequence of SEQ ID NO: 72; optionally the first exogenous polynucleotide encodes an amino acid sequence comprising SEQ ID NO: 81, and the second exogenous polynucleotide encodes an amino acid sequence comprising SEQ ID NO: 82.
138. The modified cell of any one of embodiments 126-137, wherein the amino acid sequence of the first IFNγ fusion protein subunit is SEQ ID NO: 71, and the amino acid sequence of the second IFNγ fusion protein subunit is SEQ ID NO: 72.
139. A method of enhancing the proliferation and killing potency of an iPSC, a γδT cell, or an intermediate cell differentiated from an iPSC comprising introducing one or more exogenous polynucleotides encoding an IFNγ signal converter into said iPSC, a γδT cell, or an intermediate cell differentiated from an iPSC, wherein the IFNγ signal converter comprising a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB); optionally further comprising introducing an exogenous polynucleotide encoding a chimeric antigen receptor (CAR) into the cell, optionally wherein the exogenous polynucleotide encoding the CAR is inserted into an AAVS1 locus, optionally wherein the exogenous polynucleotide encodes the CAR which specifically binds human CD19, optionally wherein the CAR which specifically binds human CD19 comprises the amino acid of SEQ ID NO: 83 or SEQ ID NO: 84.
140. The method of embodiment 139, wherein a first exogenous polynucleotide encoding the first IFNγ fusion protein subunit of the IFNγ signal converter is inserted into a SOCS1 locus.
141. The method of embodiment 139 or 140, wherein a second exogenous polynucleotide encoding the second IFNγ fusion protein subunit of the IFNγ signal converter is inserted into a ROSA26 locus.
142. The method of any one of embodiments 139-141, the first IFNγ fusion protein subunit of the IFNγ signal converter comprises an amino acid sequence of SEQ ID NO: 71, and the second IFNγ fusion protein subunit of the IFNγ signal converter comprises an amino acid sequence of SEQ ID NO: 72; optionally the first exogenous polynucleotide encodes an amino acid sequence comprising SEQ ID NO: 81, and the second exogenous polynucleotide encodes an amino acid sequence comprising SEQ ID NO: 82.
143. The method of any one of embodiments 139-142, wherein the cell is generated from the method of any one of embodiments 62-111.
144. A modified cell generated from the method of any one of embodiments 139-143.
145. The modified cell or method of any one of the preceding embodiments, wherein the cell is a human cell.
146. The modified cell or method of any one of the preceding embodiments, wherein the cell is in vivo, in vitro, ex vivo, or in a human subject.
147. A gene editing system comprising one or more guide RNA and an endonuclease or a nucleic acid encoding the endonuclease, wherein the one or more guide RNAs comprises one or more single guide RNA (sgRNA) or CRISPR RNA (crRNA) that comprises:
   a. a guide sequence that binds to a target sequence comprising any one of SEQ ID NOs: 17, 23, 15, 16, and 24;
   b. a guide sequence that binds to a target sequence comprising any one of SEQ ID NOs: 14, 18, 19, 20, 21, and 22;
   c. a first guide sequence that binds to a target sequence comprising any one of SEQ ID NOs: 17 and 23; and a second guide sequence that binds to a target sequence comprising any one of SEQ ID NOs: 15, 16, and 24;
   d. a first guide sequence that binds to a target sequence comprising any one of SEQ ID NOs: 17 and 23; a second guide sequence that binds to a target sequence comprising any one of SEQ ID NOs: 15, 16, and 24; and a third guide sequence that binds to a target sequence comprising any one of SEQ ID NOs: 14, 18, 19, 20, 21, and 22;
   e. a first guide sequence that binds to a target sequence comprising any one of SEQ ID NOs: 17 and 23; a second guide sequence that binds to a target sequence comprising any one of SEQ ID NOs: 15, 16, and 24;; a third guide sequence that binds to a target sequence comprising any one of SEQ ID NOs: 14, 18, 19, 20, 21, and 22; a fourth guide sequence that binds to a target sequence comprising SEQ ID NO: 56; and, a fifth guide sequence that binds to a target sequence comprising SEQ ID NO: 59;
   f. a first guide sequence that binds to a target sequence comprising any one of SEQ ID NOs: 17 and 23; a second guide sequence that binds to a target sequence comprising any one of SEQ ID NOs: 15, 16, and 24; a third guide sequence that binds to a target sequence comprising any one of SEQ ID NOs: 14, 18, 19, 20, 21, and 22; a sixth guide sequence that binds to a target sequence comprising SEQ ID NO: 62; a fourth guide sequence that binds to a target sequence comprising SEQ ID NO: 56; and a fifth guide sequence that binds to a target sequence comprising SEQ ID NO: 59;
   g. a guide sequence that comprises any one of SEQ ID NOs: 5 and 11;
   h. a sgRNA or crRNA sequence that comprises SEQ ID NO: 29 and 37;
   i. a guide sequence that comprises any one of SEQ ID NOs: 3, 4, and 12;
   j. a sgRNA or crRNA sequence that comprises any one of SEQ ID NOs: 27, 28, and 38;
   k. a guide sequence that comprises any one of SEQ ID NOs: 2, 6, 7, 8, 9, 10;
   l. a sgRNA or crRNA sequence that comprises any one of SEQ ID NOs: 26, 32, 33, 34, 35, and 36;
   m. a first guide sequence that comprises any one of SEQ ID NOs: 5 and 11, and a second guide sequence that comprises any one of SEQ ID NOs: 3, 4, and 12;
   n. a first sgRNA or crRNA sequence that comprises any one of SEQ ID NOs: 29 and 37, and a second sgRNA or crRNA sequence that comprises any one of SEQ ID NOs: 27, 28, and 38;
   o. a first guide sequence that comprises any one of SEQ ID NOs: 5 and 11; a second guide sequence that comprises any one of SEQ ID NOs: 3, 4, and 12; and a third guide sequence that comprises any one of SEQ ID NOs: 2, 6, 7, 8, 9, and 10;
   p. a first sgRNA or crRNA sequence that comprises any one of SEQ ID NOs: 29 and 37; a second sgRNA or crRNA sequence that comprises any one of SEQ ID NOs: 27, 28, and 38; and a third sgRNA or crRNA sequence that comprises any one of SEQ ID NOs: 26, 32, 33, 34, 35, and 36;
   q. a first guide sequence that comprises any one of SEQ ID NOs: 5, and 11; a second guide sequence that comprises any one of SEQ ID NOs: 3, 4, and 12; a third guide sequence that comprises any one of SEQ ID NOs: 2, 6, 7, 8, 9, and 10; a fourth guide sequence that comprises SEQ ID NO: 55; and, a fifth guide sequence that comprises SEQ ID NO: 58;
   r. a first sgRNA or crRNA sequence that comprises any one of SEQ ID NOs: 29 and 37; a second sgRNA or crRNA sequence that comprises any one of SEQ ID NOs: 27, 28, and 38; a third sgRNA or crRNA sequence that comprises any one of SEQ ID NOs: 26, 32, 33, 34, 35, and 36; a fourth sgRNA sequence that comprises SEQ ID NO: 54; and, a fifth sgRNA sequence that comprises SEQ ID NO: 57;
   s. a first guide sequence that comprises any one of SEQ ID NOs: 5, and 11; a second guide sequence that comprises any one of SEQ ID NOs: 3, 4, and 12; a third guide sequence that comprises any one of SEQ ID NOs: 2, 6, 7, 8, 9, and 10; a sixth guide sequence that comprises SEQ ID NO: 61; a fourth guide sequence that comprises SEQ ID NO: 55; and, a fifth guide sequence that comprises SEQ ID NO: 58; or
   t. a first sgRNA or crRNA sequence that comprises any one of SEQ ID NOs: 29 and 37; a second sgRNA or crRNA sequence that comprises any one of SEQ ID NOs: 27, 28, and 38; a third sgRNA or crRNA sequence that comprises any one of SEQ ID NOs: 26, 32, 33, 34, 35, and 36; a sixth sgRNA sequence that comprises SEQ ID NO: 60; a fourth sgRNA sequence comprises SEQ ID NO: 54; and, a fifth sgRNA sequence that comprises SEQ ID NO: 57.
148. The gene editing system of embodiment 147, wherein the endonuclease is a Cas endonuclease.
149. The gene editing system of embodiment 148, wherein the Cas endonuclease is a MAD7 endonuclease.
150. The gene editing system of embodiment 149, wherein:
   a. the target sequence comprises SEQ ID NO: 22; and/or
   b. the crRNA comprises a guide sequence differing by no more than 3 nucleotides from SEQ ID NO: 10.
151. The gene editing system of embodiment 149, wherein:
   a. the target sequence comprises SEQ ID NO: 23; and/or
   b. the crRNA comprises a guide sequence differing by no more than 3 nucleotides from SEQ ID NO: 11.
152. The gene editing system of embodiment 149, wherein:
   a. the target sequence comprises SEQ ID NO: 24; and/or
   b. the crRNA comprises a guide sequence differing by no more than 3 nucleotides from SEQ ID NO: 12.
153. The gene editing system of embodiment 148, wherein the Cas endonuclease is a Cas9 endonuclease.
154. The gene editing system of embodiment 153, wherein:
   a. the target sequence comprises any one of SEQ ID NOs: 14, 18, 19, 20, and 21; and/or
   b. the sgRNA comprises a guide sequence differing by no more than 3 nucleotides from any one of SEQ ID NOs: 2, 6, 7, 8, and 9.
155. The gene editing system of embodiment 153, wherein:
   a. the target sequence comprises SEQ ID NO: 17; and/or
   b. the sgRNA comprises a guide sequence differing by no more than 3 nucleotides from SEQ ID NO: 5.
156. The gene editing system of embodiment 153, wherein:
   a. the target sequence comprises SEQ ID NOs: 15 or 16; and/or
   b. the sgRNA comprises a guide sequence differing by no more than 3 nucleotides from SEQ ID NOs: 3 or 4.
157. The gene editing system of embodiment 153, wherein:
   a. the target sequence comprises SEQ ID NO: 62; and/or
   b. the sgRNA comprises a guide sequence differing by no more than 3 nucleotides from SEQ ID NO: 61.
158. The gene editing system of embodiment 153, wherein:
   a. the target sequence comprises SEQ ID NO: 56; and/or
   b. the sgRNA comprises a guide sequence differing by no more than 3 nucleotides from SEQ ID NO: 55.
159. The gene editing system of embodiment 153, wherein:
   a. the target sequence comprises SEQ ID NO: 59; and/or
   b. the sgRNA comprises a guide sequence differing by no more than 3 nucleotides from SEQ ID NO: 58.
160. A guide RNA comprising SEQ ID NO: 5 (SOCS1), SEQ ID NO: 29 (SOCS1), SEQ ID NO: 11 (SOCS1), SEQ ID NO: 37 (SOCS1), SEQ ID NO: 27 (CISH), SEQ ID NO: 28 (CISH), SEQ ID NO: 12 (CISH), SEQ ID NO: 38 (CISH), SEQ ID NO: 6 (BIM), SEQ ID NO: 32 (BIM), SEQ ID NO: 7 (BIM), SEQ ID NO: 33 (BIM), SEQ ID NO: 8 (BIM), SEQ ID NO: 34 (BIM), SEQ ID NO: 9 (BIM), SEQ ID NO: 35 (BIM), SEQ ID NO: 10 (BIM), or SEQ ID NO: 36 (BIM).
161. A guide RNA comprising a CRISPR RNA (crRNA) sequence, wherein the crRNA sequence comprises a 2′-O-Methyl modification at each of the first three positions on the 5′ end and at each of the fourth to last, third to last, and second to last positions of the crRNA sequence on the 3′ end, and a phosphorothioate linkage between each base in the first three positions on the 5′ end and a phosphorothioate linkage between each base of the last three positions of the crRNA sequence at the 3′ end.
162. The guide RNA of embodiment 161, wherein the guide RNA functions with a MAD7 Cas endonuclease.
163. The guide RNA of embodiment 162, wherein the crRNA is about 56 bases long.
164. The guide RNA of embodiment 163, wherein the crRNA sequence comprises a 2′-O-Methyl modification at each of positions 1, 2, 3, 53, 54, and 55 of the crRNA sequence on the 3′ end.
165. The guide RNA of any one of embodiments 161-164, wherein the crRNA comprises SEQ ID NO: 36, SEQ ID NO: 37, or SEQ ID NO: 38, including its modifications.
166. The guide RNA of any one of embodiments 161-165, wherein the last base of the crRNA is modified, but not with 2′-O-Methyl modification.
167. The guide RNA of embodiment 166, wherein the modification on the last base of the crRNA is a pseudoknot.
168. The guide RNA of embodiment 166, wherein the modification on the last base of the crRNA is a modification that facilitates use with MAD7.
169. A modified iPSC, γδT cell, or intermediate cell differentiated from an iPSC, generated by any of the following:
i) contacting the cell with the gene editing system of any one of embodiments 147-159, wherein the contacting results in a genomic disruption in a target sequence that binds to the single guide RNA (sgRNA) of the gene editing system; or
ii) contacting the cell with the guide RNA of any one of embodiments 160-168 and an endonuclease or a nucleic acid encoding the endonuclease, wherein the contacting results in a genomic disruption in a target sequence that binds to the guide RNA of embodiment 160; and wherein the genomic disruption is an inactivating nucleic acid mutation, optionally wherein the mutation is an insertion, deletion, or substitution.
170. A modified iPSC, γδT cell, or intermediate cell differentiated from an iPSC, comprising a genomic disruption in an exon 2C of an endogenous BIM gene, wherein the expression and/or function of BIM_{EL} and BIM_{L} splice variants are reduced in the modified cell relative to that in an unmodified control, and the expression and/or function of BIM_{S} splice variant is retained in the modified cell, wherein the genomic disruption is within a target sequence in BIM that comprises any one of SEQ ID NOs: 14, 20, 21, and 22.
171. The modified cell of embodiment 170, wherein the genomic disruption is an inactivating nucleic acid mutation in the exon 2C, optionally wherein the mutation is an insertion, deletion, or substitution.
172. The modified cell of any one of embodiments 170-171, wherein the genomic disruption within BIM is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
173. The modified cell of embodiment 172, wherein the guide RNA comprises any one of SEQ ID NOs: 26, 34, 35 and 36, or comprises a guide sequence differing by no more than 3 nucleotides from any one of SEQ ID Nos: 2, 8, 9 and 10.
174. The modified cell of embodiment 172 or embodiment 173, wherein the guide RNA comprises SEQ ID NO: 36 or comprises a guide sequence differing by no more than 3 nucleotides from SEQ ID NO: 10, and the endonuclease is a MAD7 endonuclease.
175. The modified cell of embodiment 172 or embodiment 173, wherein the guide RNA comprises any one of SEQ ID NOs: 26, 34, and 35, or comprises a guide sequence differing by no more than 3 nucleotides from any one of SEQ ID Nos: 2, 8, and 9, and the endonuclease is a Cas9 endonuclease.
176. A modified iPSC, γδT cell, or intermediate cell differentiated from an iPSC, comprising a genomic disruption in a target sequence of an endogenous SOCS1 gene that suppresses or eliminates expression of a functional SOCS1 protein, wherein the target sequence comprises SEQ ID NO: 17 or SEQ ID NO: 23.
177. The modified cell of embodiment 176, wherein the genomic disruption is an inactivating nucleic acid mutation, optionally wherein the mutation is an insertion, deletion, or substitution.
178. The modified cell of embodiment 176 or embodiment 177, wherein the genomic disruption within SOCS1 is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
179. The modified cell of embodiment 178, wherein the guide RNA comprises any one of SEQ ID NOs: 29 and 37, or comprises a guide sequence differing by no more than 3 nucleotides from SEQ ID NO: 5 or 11.
180. The modified cell of embodiment 178 or embodiment 179, wherein the guide RNA comprises SEQ ID NO: 37 or comprises a guide sequence differing by no more than 3 nucleotides from SEQ ID NO: 11, and the endonuclease is a MAD7 endonuclease.
181. The modified cell of embodiment 178 or embodiment 179, wherein the guide RNA comprises SEQ ID NO: 29 or comprises a guide sequence differing by no more than 3 nucleotides from SEQ ID NO: 5, and the endonuclease is a Cas9 endonuclease.
182. A modified iPSC, γδT cell, or intermediate cell differentiated from an iPSC, comprising a genomic disruption in a target sequence of an endogenous CISH gene that suppresses or eliminates expression of CISH, wherein the target sequence comprises SEQ ID NO: 24.
183. The modified cell of embodiment 182, wherein the genomic disruption is an inactivating nucleic acid mutation, optionally wherein the mutation is an insertion, deletion, or substitution.
184. The modified cell of embodiment 182 or embodiment 183, wherein the genomic disruption within CISH is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
185. The modified cell of embodiment 184, wherein the guide RNA comprises SEQ ID NO: 38 or comprises a guide sequence differing by no more than 3 nucleotides from SEQ ID NO: 12.
186. The modified cell of embodiment 184 or embodiment 185, wherein the guide RNA comprises SEQ ID NO: 38 or comprises a guide sequence differing by no more than 3 nucleotides from SEQ ID NO: 12, and the endonuclease is a MAD7 endonuclease.
187. The modified cell of any one of embodiments 169-186, wherein the iPSC is derived from a primary T cell (T-iPSC).
188. The modified cell of any one of embodiments 169-187, wherein the iPSC is derived from a primary γδT cell (γδT-iPSC).
189. The modified cell of any one of embodiments 169-188, wherein the γδT cell is Vd2 γδT cell.
190. The modified cell of any one of embodiments 169-189, wherein the γδT cell is an iPSC-derived γδT (iγδT) cell, optionally the γδT cell is a γδT cell differentiated from a T-iPSC (T-iγδT).
191. The modified cell of any one of embodiments 169-190, wherein the γδT cell is an γδT cell differentiated from a γδT-iPSC (γδT-iγδT).
192. The modified cell of any one of embodiments 169-191, wherein the intermediate cell differentiated from the iPSC is a γδT-iHSC, a γδT-iCLP, and/or an immature T-iγδT cell.
193. The modified cell of any one of embodiments 169-192, wherein the cell is a human cell.
194. The modified cell any one of embodiments 169-193, wherein the cell is in vivo, in vitro, ex vivo, or in a human subject.
195. A modified iPSC, γδT cell, or intermediate cell differentiated from an iPSC, comprising 1) an exogenous polynucleotide encodes a CAR which specifically binds human CD19, 2) a genomic disruption in an endogenous suppressor of cytokine signaling 1 (SOCS1) gene that suppresses or eliminates expression of a functional SOCS1 protein, and 3) a genomic disruption in an endogenous cytokine-inducible SH2-containing protein (CISH) gene that suppresses or eliminates expression of a functional CISH protein.
196. The modified cell of any one of embodiments 195, further comprising a genomic disruption in an endogenous Bcl-2 Interacting Mediator of cell death (BIM) gene.
197. The modified cell of embodiment 196, wherein the genomic disruption in the BIM gene is in exon 2C of the BIM gene.
198. The modified cell of embodiment 197, wherein the expression and/or function of BIM_{EL} and BIM_{L} splice variants are reduced in the modified cell relative to that in an unmodified control and the expression and/or function of BIM_{S} splice variant is retained in the modified cell.
199. The modified cell of any one of embodiments 196-198, wherein the genomic disruption is an inactivating nucleic acid mutation in an exon 2C of an endogenous BIM gene, optionally wherein the mutation is an insertion, deletion, or substitution.
200. The modified cell of any one of embodiments 196-199, wherein the genomic disruption is within a target sequence in BIM that comprises any one of SEQ ID NOs: 14, 18, 19, 20, 21, and 22.
201. The modified cell of any one of embodiments 196-200, wherein the genomic disruption within BIM is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
202. The modified cell of embodiment 201, wherein the guide RNA comprises the guide sequence of any one of SEQ ID NOs: 2, 6, 7, 8, 9, and 10, or comprises the sgRNA or crRNA sequence of any one or more of SEQ ID NOs: 26, 32, 33, 34, 35, and 36.
203. A modified iPSC, γδT cell, or intermediate cell differentiated from an iPSC, comprising 1) an exogenous polynucleotide encodes a CAR which specifically binds human CD19, 2) a genomic disruption in an endogenous suppressor of cytokine signaling 1 (SOCS1) gene that suppresses or eliminates expression of a functional SOCS1 protein, 3) a genomic disruption in an endogenous cytokine-inducible SH2-containing protein (CISH) gene that suppresses or eliminates expression of a functional CISH protein, and 4) a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell.
204. The modified cell of embodiment 203, wherein the genomic disruption is an inactivating nucleic acid mutation in the exon 2C, optionally wherein the mutation is an insertion, deletion, or substitution.
205. The modified cell of embodiment 203 or embodiment 204, wherein the genomic disruption is within a target sequence in BIM that comprises any one of SEQ ID NOs: 14, 20, 21, and 22.
206. The modified cell of any one of embodiments 203-205, wherein the genomic disruption within BIM is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
207. The modified cell of embodiment 206, wherein the guide RNA comprises any one of SEQ ID NOs: 26, 34, 35, and 36, or comprises the guide sequence of any one of SEQ ID NOs: 2, 8, 9 and 10.
208. The modified cell of embodiment 206 or embodiment 207, wherein the guide RNA comprises SEQ ID NO: 36 or comprises the guide sequence of SEQ ID NO: 10, and the endonuclease is a MAD7 endonuclease.
209. The modified cell of embodiment 206 or embodiment 207, wherein the guide RNA comprises any one of SEQ ID NOs: 26, 34, and 35, or comprises the guide sequence of any one of SEQ ID NOs: 2, 8, and 9, and the endonuclease is a Cas9 endonuclease.
210. The modified cell of any one of embodiments 195-209, wherein the exogenous polynucleotide encoding the CAR is inserted into an AAVS1 locus.
211. The modified cell of any one of embodiments 195-210, wherein the CAR which specifically binds human CD19 comprises the amino acid of SEQ ID NO: 83 or SEQ ID NO: 84.
212. The modified cell of any one of embodiments 195-211, wherein the genomic disruption is in exon 2 of the SOCS1 gene; optionally wherein the genomic disruption is an inactivating nucleic acid mutation, optionally wherein the mutation is an insertion, deletion, or substitution.
213. The modified cell of any one of embodiments 195-212, wherein the genomic disruption is in exon 3 or exon 4 of the CISH gene; optionally wherein the genomic disruption is an inactivating nucleic acid mutation, optionally wherein the mutation is an insertion, deletion, or substitution.
214. The modified cell of any one of embodiments 195-213, wherein the genomic disruption is within a target sequence in SOCS1 that comprises SEQ ID NO: 17 or SEQ ID NO: 23 and/or the genomic disruption is within a target sequence in CISH that comprises SEQ ID NO: 15, SEQ ID NO: 16 or SEQ ID NO: 24.
215. The modified cell of any one of embodiments 195-214, wherein the genomic disruption within SOCS1 and CISH is made by administering guide RNAs to said cell, optionally wherein the guide RNAs are single-guide RNAs (sgRNAs), and wherein an endonuclease or a nucleic acid encoding the endonuclease is also administered to said cell.
216. The modified cell of embodiment 215, wherein the guide RNAs comprise any one or more of SEQ ID NOs: 29, 37, 27, 28, and 38, or comprises the guide sequence of any one or more of SEQ ID NOs: 5, 11, 3, 4, and 12.
217. The modified cell of any one of embodiments 195-216, further comprising a genomic disruption in an endogenous FAS cell surface death receptor (FAS) gene that suppresses or eliminates expression of a functional FAS protein.
218. The modified cell of embodiment 217, wherein the genomic disruption in the FAS gene is in exon 1 of the FAS gene.
219. The modified cell of embodiment 217 or embodiment 218, wherein the genomic disruption is an inactivating nucleic acid mutation in exon 1 of an endogenous FAS gene, optionally wherein the mutation is an insertion, deletion, or substitution.
220. The modified cell of any one of embodiments 217-219, wherein the genomic disruption is within a target sequence in FAS that comprises SEQ ID NO: 62.
221. The modified cell of any one of embodiments 217-220, wherein the genomic disruption within FAS is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
222. The modified cell of embodiment 221, wherein the guide RNA comprises SEQ ID NO: 60 or comprises the guide sequence of SEQ ID NO: 61.
223. The modified cell of any one of embodiments 195-222, further comprising a genomic disruption in an endogenous β-2-Microglobulin (B2M) gene that suppresses or eliminates expression of a functional B2M protein.
224. The modified cell of embodiment 223, wherein the genomic disruption in the B2M gene is in exon 2 of the B2M gene.
225. The modified cell of embodiment 223 or embodiment 224, wherein the genomic disruption is an inactivating nucleic acid mutation in exon 2 of an endogenous B2M gene, optionally wherein the mutation is an insertion, deletion, or substitution.
226. The modified cell of any one of embodiments 223-225, wherein the genomic disruption is within a target sequence in B2M that comprises SEQ ID NO: 56.
227. The modified cell of any one of embodiments 223-226, wherein the genomic disruption within B2M is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
228. The modified cell of embodiment 227, wherein the guide RNA comprises SEQ ID NO: 54 or comprises the guide sequence of SEQ ID NO: 55.
229. The modified cell of any one of embodiments 195-228, further comprising a genomic disruption in an endogenous Class II transactivator (CIITA) gene that suppresses or eliminates expression of a functional CIITA protein.
230. The modified cell of embodiment 229, wherein the genomic disruption in the CIITA gene is in exon 3 of the CIITA gene.
231. The modified cell of embodiment 229 or embodiment 230, wherein the genomic disruption is an inactivating nucleic acid mutation in exon 3 of an endogenous CIITA gene, optionally wherein the mutation is an insertion, deletion, or substitution.
232. The modified cell of any one of embodiments 229-231, wherein the genomic disruption is within a target sequence in CIITA that comprises SEQ ID NO: 59.
233. The modified cell of any one of embodiments 229-232, wherein the genomic disruption within CIITA is made by administering a guide RNA to said cell, optionally wherein the guide RNA is a single-guide RNA (sgRNA) or crRNA, and optionally wherein an endonuclease or a nucleic acid encoding the endonuclease is also administered to said cell.
234. The modified cell of embodiment 233, wherein the guide RNA comprises SEQ ID NO: 57 or comprises the guide sequence of SEQ ID NO: 58.
235. The modified cell of any one of embodiments 195-234, further comprising a genomic disruption in an endogenous T-cell receptor alpha constant region (TRAC) gene that suppresses or eliminates expression of a functional TRAC protein.
236. The modified cell of any one of embodiments 195-235, wherein the genomic disruption is an inactivating nucleic acid mutation, optionally wherein the mutation is an insertion, deletion, or substitution.
237. The modified cell of any one of embodiments 195-236, further comprising one or more exogenous polynucleotides encoding an IFNγ signal converter, wherein the IFNγ signal converter comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB).
238. The modified cell of embodiment 237, wherein a first exogenous polynucleotide encoding the first IFNγ fusion protein subunit of the IFNγ signal converter is inserted into a SOCS1 locus.
239. The modified cell of embodiment 237 or embodiment 238, wherein a second exogenous polynucleotide encoding the second IFNγ fusion protein subunit of the IFNγ signal converter is inserted into a ROSA26 locus.
240. The modified cell of any one of embodiments 237-239, the first IFNγ fusion protein subunit of the IFNγ signal converter comprises an amino acid sequence of SEQ ID NO: 71, and the second IFNγ fusion protein subunit of the IFNγ signal converter comprises an amino acid sequence of SEQ ID NO: 72; optionally the first exogenous polynucleotide encodes an amino acid sequence comprising SEQ ID NO: 81, and the second exogenous polynucleotide encodes an amino acid sequence comprising SEQ ID NO: 82.
241. The modified cell of any one of embodiments 237-240, wherein the amino acid sequence of the first IFNγ fusion protein subunit is SEQ ID NO: 71, and the amino acid sequence of the second IFNγ fusion protein subunit is SEQ ID NO: 72.
242. The modified cell of any one of embodiments 195-241, further comprising an exogenous polynucleotide encoding a B2M-HLA-E fusion protein (B2M-HLA-E) comprising at least a portion of B2M fused with at least of a portion of HLA-E.
243. The modified cell of embodiment 242, wherein the exogenous polynucleotide encoding the B2M-HLA-E is inserted into a B2M locus.
244. The modified cell of embodiment 242 or embodiment 243, wherein the B2M-HLA-E comprises an amino acid sequence of SEQ ID NO: 85.
245. The modified cell of any one of embodiments 195-244, wherein the iPSC is derived from a primary T cell (T-iPSC).
246. The modified cell of any one of embodiments 195-245, wherein the iPSC is derived from a primary γδT cell (γδT-iPSC).
247. The modified cell of any one of embodiments 195-246, wherein the γδT cell is Vd2 γδT cell.
248. The modified cell of any one of embodiments 195-247, wherein the γδT cell is an iPSC-derived γδT (iγδT) cell, optionally the γδT cell is a γδT cell derived from a T-iPSC (T-iγδT).
249. The modified cell of any one of embodiments 195-248, wherein the γδT cell is a γδT cell differentiated from a γδT-iPSC (γδT-iγδT).
250. The modified cell of any one of embodiments 195-249, wherein the intermediate cell differentiated from the iPSC is a γδT-iHSC, a γδT-iCLP, and/or an immature T-iγδT cell.
251. A pharmaceutical composition comprising the modified γδT cell of any one of embodiments 195-250 and a pharmaceutically acceptable carrier.
252. A method of killing B cells which are CD19 positive and have abnormal B cell function, comprising administering to a subject in need thereof a therapeutically effective amount of the modified γδT cell of any one of embodiments 195-250 or the pharmaceutical composition of embodiment 251.
253. The method of embodiment 252, wherein the B cells are the CD19 positive B cells in diffuse large B-cell lymphoma (DLBCL) or systemic lupus erythematosus (SLE) patients.
254. A method of treating DLBCL or SLE, comprising administering to a subject in need thereof a therapeutically effective amount of the modified γδT cell of any one of embodiments 195-250 or the pharmaceutical composition of embodiment 251.
255. The modified γδT cell of any one of embodiments 195-250 or the pharmaceutical composition of embodiment 251 for use in therapy.
256. The modified γδT cell of any one of embodiments 195-250 or the pharmaceutical composition of embodiment 251 for use in treatment of DLBCL or SLE.
257. Use of the modified cell of any one of embodiments 195-250 or the pharmaceutical composition of embodiment 251 in the manufacturing of a medicament for treatment of DLBCL or SLE.
258. A method of generating the modified γδT cell of any one of embodiments 195-250, comprising (i) generating the modified iPSC cell of any one of embodiments 195-250, optionally, according to the method of any one of embodiments 62-111, and (ii) differentiating the modified iPSC cell into the modified γδT cell.
259. A method of generating the modified γδT cell of any one of embodiments 1-59, comprising (i) generating the modified iPSC cell of any one of embodiments 1-59, optionally, according to the method of any one of embodiments 62-111, and (ii) differentiating the modified iPSC cell into the modified γδT cell.
260. The method of any one of embodiments 62-111, embodiments 119-124, and embodiments 139-143, further comprising differentiating the modified iPSC generated from the method of any one of embodiments 62-111, embodiments 119-124, and embodiments 139-143 into the modified γδT cell.

Also, the present disclosure encompasses, inter alia, the following embodiments, among others disclosed herein.

Embodiment 1102. A modified iPSC, γδT cell, or intermediate cell differentiated from an iPSC comprising 1) a genomic disruption in an endogenous SOCS1 gene that suppresses or eliminates expression of SOCS1; 2) a genomic disruption in an endogenous CISH gene that suppresses or eliminates expression of CISH; and 3) a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell.

Embodiment 1103. A modified iPSC, γδT cell, or intermediate cell differentiated from an iPSC comprising 1) a genomic disruption in an endogenous SOCS1 gene that suppresses or eliminates expression of SOCS1; 2) a genomic disruption in an endogenous CISH gene that suppresses or eliminates expression of CISH; 3) a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell; 4) a genomic disruption in an endogenous B2M gene that suppresses or eliminates expression of B2M; and, 5) a genomic disruption in an endogenous CIITA gene that suppresses or eliminates expression of CIITA.

Embodiment 1104. A modified iPSC, γδT cell, or intermediate cell differentiated from an iPSC comprising 1) a genomic disruption in an endogenous SOCS1 gene that suppresses or eliminates expression of SOCS1; 2) a genomic disruption in an endogenous CISH gene that suppresses or eliminates expression of CISH; 3) an a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell; 4) a genomic disruption in an endogenous B2M gene that suppresses or eliminates expression of B2M; 5) a genomic disruption in an endogenous CIITA gene that suppresses or eliminates expression of CIITA, and, 6) a genomic disruption in an endogenous FAS gene that suppresses or eliminates expression of FAS.

Embodiment 1105. The modified cell of any one of embodiments 1102-1104, wherein the iPSC is derived from a primary T cell (T-iPSC).

Embodiment 1106. The modified cell of any one of embodiments 1102-1105, wherein the iPSC is derived from a primary γδT cell (γδT-iPSC).

Embodiment 1107. The modified cell of any one of embodiments 1102-1106, wherein the γδT cell is Vd2 γδT cell.

Embodiment 1108. The modified cell of any one of embodiments 1102-1107, wherein the γδT cell is an iPSC-derived γδT (iγδT) cell, optionally the γδT cell is a γδT cell derived from a T-iPSC (T-iγδT).

Embodiment 1109. The modified cell of any one of embodiments 1102-1108, wherein the γδT cell is a γδT cell differentiated from a γδT-iPSC (γδT-iγδT).

Embodiment 1110. The modified cell of any one of embodiments 1102-1109, wherein the intermediate cell differentiated from the iPSC is a γδT-iHSC, a γδT-iCLP, and/or an immature T-iγδT cell.

Embodiment 1111. The modified cell of any one of embodiments 1102-1110, wherein the genomic disruption in SOCS1 is in exon 2 of the SOCS1 gene, and wherein the genomic disruption in CISH is in exon 3 or exon 4 of the CISH gene.

Embodiment 1112. The modified cell of any one of embodiments 1102-1111, wherein the genomic disruption is within a target sequence in SOCS1 that comprises SEQ ID NO: 17 or SEQ ID NO: 23 and the genomic disruption is within a target sequence in CISH that comprises SEQ ID NO: 15, SEQ ID NO: 16 or SEQ ID NO: 24.

Embodiment 1113. The modified cell of any one of embodiments 1102-1112, wherein the genomic disruption within SOCS1 and CISH is made by administering guide RNAs to said cell, optionally wherein the guide RNAs are single-guide RNAs (sgRNAs), and optionally wherein an endonuclease or a nucleic acid encoding an endonuclease is also administered to said cell.

Embodiment 1114. The modified cell of embodiment 1113, wherein the guide RNAs comprise any one or more of SEQ ID NOs: 5, 29, 11, 37, 3, 27, 4, 28, 12, and 38.

Embodiment 1115. The modified cell of any one of embodiments 1102-1114, wherein the genomic disruption is within a target sequence in BIM that comprises any one of SEQ ID NOs: 14, 20, 21, and 22.

Embodiment 1116. The modified cell of any one of embodiments 1102-1115, wherein the genomic disruption within BIM is made by administering a guide RNA to said cell, optionally wherein the guide RNA is a single-guide RNA (sgRNA) or crRNA, and optionally wherein an endonuclease or a nucleic acid encoding an endonuclease is also administered to said cell.

Embodiment 1117. The modified cell of embodiment 1116, wherein the guide RNA comprises any one of SEQ ID NOs: 2, 26, 8, 34, 9, 35, 10 and 36.

Embodiment 1118. The modified cell of any one of embodiments 1102-1117, wherein the genomic disruption in FAS is in exon 1 of the FAS gene.

Embodiment 1119. The modified cell of any one of embodiments 1102-1118, wherein the genomic disruption is within a target sequence in FAS that comprises SEQ ID NO: 62.

Embodiment 1120. The modified cell of any one of embodiments 1102-1119, wherein the genomic disruption within FAS is made by administering a guide RNA to said cell, optionally wherein the guide RNA is a single-guide RNA (sgRNA) or crRNA, and optionally wherein an endonuclease or a nucleic acid encoding an endonuclease is also administered to said cell.

Embodiment 1121. The modified cell of embodiment 1120, wherein the guide RNA comprises any one of SEQ ID NOs: 60 and 61.

Embodiment 1122. The modified cell of any one of embodiments 1102-1121, wherein the genomic disruption in B2M is in exon 2 of the B2M gene.

Embodiment 1123. The modified cell of any one of embodiments 1102-1122, wherein the genomic disruption is within a target sequence in B2M that comprises SEQ ID NO: 56.

Embodiment 1124. The modified cell of any one of embodiments 1102-1123, wherein the genomic disruption within B2M is made by administering a guide RNA to said cell, optionally wherein the guide RNA is a single-guide RNA (sgRNA) or crRNA, and optionally wherein an endonuclease or a nucleic acid encoding an endonuclease is also administered to said cell.

Embodiment 1125. The modified cell of embodiment 1124, wherein the guide RNA comprises any one of SEQ ID NOs: 54 and 55.

Embodiment 1126. The modified cell of any one of embodiments 1102-1125, wherein the genomic disruption in CIITA is in exon 3 of the CIITA gene.

Embodiment 1127. The modified cell of any one of embodiments 1102-1126, wherein the genomic disruption is within a target sequence in CIITA that comprises SEQ ID NO: 59.

Embodiment 1128. The modified cell of any one of embodiments 1102-1127, wherein the genomic disruption within CIITA is made by administering a guide RNA to said cell, optionally wherein the guide RNA is a single-guide RNA (sgRNA) or crRNA, and optionally wherein an endonuclease or a nucleic acid encoding an endonuclease is also administered to said cell.

Embodiment 1129. The modified cell of embodiment 1128, wherein the guide RNA comprises any one of SEQ ID NOs: 57 and 58.

Embodiment 1130. The modified cell of any one of embodiments 1102-1129, further comprising one or more exogenous polynucleotides encoding an IFNγ signal converter, wherein the IFNγ signal converter comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB).

Embodiment 1131. The modified cell of embodiment 1130, wherein the amino acid sequence of the first IFNγ fusion protein subunit is SEQ ID NO: 71, and the amino acid sequence of the second IFNγ fusion protein subunit is SEQ ID NO: 72.

Also, the present disclosure encompasses, inter alia, the following embodiments, among others disclosed herein.

Embodiment 2001: A IFNγ signal converter comprising a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (TL2RB).

Embodiment 2002: The IFNγ signal converter of embodiment 2001, wherein the amino acid sequence of ECD of the first IFNγ fusion protein subunit is SEQ ID NO: 63, and the amino acid sequence of ECD of the second IFNγ fusion protein subunit is SEQ ID NO: 64.

Embodiment 2003: The IFNγ signal converter of embodiment 2001 or embodiment 2002, wherein the amino acid sequence of intracellular domain of the first IFNγ fusion protein subunit is SEQ ID NO: 69, and the amino acid sequence of intracellular domain of the second IFNγ fusion protein subunit is SEQ ID NO: 70.

Embodiment 2004: The IFNγ signal converter of any one of embodiments 2001-2003, wherein the first IFNγ fusion protein subunit further comprises a transmembrane domain between the ECD and the intracellular domain thereof, and the second IFNγ fusion protein subunit further comprises a transmembrane domain between the ECD and the intracellular domain thereof.

Embodiment 2005: The IFNγ signal converter of embodiment 2004, wherein the transmembrane domain of the first IFNγ fusion protein subunit is of the interleukin-2 receptor subunit gamma, and the transmembrane domain of the second IFNγ fusion protein subunit is of the interleukin-2 receptor subunit beta.

Embodiment 2006: The IFNγ signal converter of embodiment 2004, wherein the amino acid sequence of transmembrane domain of the first IFNγ fusion protein subunit is SEQ ID NO: 67, and the amino acid sequence of transmembrane domain of the second IFNγ fusion protein subunit is SEQ ID NO: 68.

Embodiment 2007: The IFNγ signal converter of any one of embodiments 2001-2006, wherein the first IFNγ fusion protein subunit further comprises a membrane-proximal region between the ECD and the intracellular domain thereof, and the second IFNγ fusion protein subunit further comprises a membrane-proximal region between the ECD and the intracellular domain thereof.

Embodiment 2008: The IFNγ signal converter of embodiment 2007, wherein the membrane-proximal region of the first IFNγ fusion protein subunit is of the interleukin-2 receptor subunit gamma, and the membrane-proximal region of the second IFNγ fusion protein subunit is of the interleukin-2 receptor subunit beta.

Embodiment 2009: The IFNγ signal converter of embodiment 2008, wherein the amino acid sequence of membrane-proximal region of the first IFNγ fusion protein subunit is SEQ ID NO: 65, and the amino acid sequence of membrane-proximal region of the second IFNγ fusion protein subunit is SEQ ID NO: 66.

Embodiment 2010: The IFNγ signal converter of any one of embodiments 2001-2009, the first IFNγ fusion protein subunit of the IFNγ signal converter comprises an amino acid sequence of SEQ ID NO: 71, and the second IFNγ fusion protein subunit of the IFNγ signal converter comprises an amino acid sequence of SEQ ID NO: 72; optionally the first exogenous polynucleotide encodes an amino acid sequence comprising SEQ ID NO: 81, and the second exogenous polynucleotide encodes an amino acid sequence comprising SEQ ID NO: 82.

Embodiment 2011: The IFNγ signal converter of any one of embodiments 2001-2010, wherein the amino acid sequence of the first IFNγ fusion protein subunit is SEQ ID NO: 71, and the amino acid sequence of the second IFNγ fusion protein subunit is SEQ ID NO: 72.

### BRIEF DESCRIPTION OF THE DRAWINGS

FIG. 1 shows the knockout efficiency of candidate genes (CISH, SOCS1, and BIM) in T-igdT (iPSC-derived γδT) cells examined by Western blotting.

FIGS. 2A-2F show the synergistic effects of CISH^{-/-}SOCS1^{-/-} double knockout (DKO) and CISH^{-/-}SOCS1^{-/-}BIM^{-/-} triple knockout (TKO) T-igdT cells in enhanced cytotoxicity and/or persistence against HepG2 cells. FIGS. 2A-2D show the serial killing ability of CISH^{-/-} single KO, SOCS1^{-/-}single KO, BIM^{-/-} single KO, CISH^{-/-} SOCS1^{-/-} DKO, and CISH^{-/-}SOCS1^{-/-}BIM^{-/-} TKO T-igdT against HepG2 cells at various effector:target (E:T) ratios with (FIGS. 2C-2D) or without zoledronic acid (FIGS. 2A-2B) monitored by the Incucyte® SX5 live-cell analysis system. FIG. 2E shows the cytotoxicity of CISH % single KO, SOCS1^{-/-} single KO, BIM^{-/-} single KO, CISH^{-/-}SOCS1^{-/-}DKO, and CISH^{-/-}SOCS1^{-/-}BIM^{-/-} TKO T-igdT against HepG2 3D spheroid at a 10:1 E:T ratio without zoledronic acid monitored by the Incucyte® SX5 live-cell analysis system. FIG. 2F shows HepG2 3D spheroid images post coculture with CISH^{-/-} single KO, SOCS1^{-/-} single KO, BIM^{-/-} single KO, CISH % SOCS1^{-/-} DKO and CISH^{-/-}SOCS1^{-/-}BIM^{-/-} TKO T-igdT cells. EGFP positive HepG2 cells were indicated with black arrow (bright area) and surrounded T-igdT cells were indicated with white arrow.

FIG. 3 shows prolonged survival of T-igdT cells with a BIM_{EL} and BIM_{L} deficiency (knockout by BIM sgRNA #5 (SEQ ID NO: 26)) or of CISH^{-/-}SOCS1^{-/-}BIM^{-/-}TKO T-igdT cells, in which the BIM^{-/-} modification has a BIM_{EL} and BIM_{L} deficiency, post IL-15 deprivation for 2-12 days, in which the experimental IL-15 levels were 0 ng/mL, 0.05 ng/ml, 0.3 ng/ml, and 10 ng/ml.

FIGS. 4A-4C show that the BIM short splice variant (BIM_{S}) is necessary for T-igdT cell survival post IL-15 deprivation. FIG. 4A is the schematic representation of BIM gene structure, and there are three main splice variants expressed in T-igdT cells. FIG. 4B shows a knockout of BIM splice variants with different BIM sgRNAs (BIM #5 (SEQ ID NO: 26), #9 (SEQ ID NO: 32), #19 (SEQ ID NO: 33), #24 (SEQ ID NO: 34) or #25 (SEQ ID NO: 35)), as demonstrated by Western blotting. FIG. 4C shows knockout of BIM_{EL} and BIM_{L} splice variants with BIM sgRNA #5 (SEQ ID NO: 26) and #25 (SEQ ID NO: 35) (retaining BIM_{S}) exhibited prolonged survival when T-igdT cells were cultured at 0 and 0.05 ng/mL IL-15 concentration condition.

FIGS. 5A-5C show the editing efficiency of CISH crRNA (CRISPR RNA) using CRISPR/MAD7-mediated gene editing. FIG. 5A shows the insertion-deletion (indel) percentages (ICEd) of the control and CISH crRNA samples calculated by ICE analysis. Data presented as mean±SD. FIG. 5B shows a representative screenshot of the Sanger sequencing chromatogram demonstrating the indel formation after CRISPR/MAD7-mediated gene editing at the CISH locus. The shaded area indicates the targeting site after indel formation by CISH crRNA. FIG. 5C shows the summary chart of single clone selection and analysis for CISH edited cells.

FIGS. 6A-6D show the editing efficiency of SOCS1 crRNA using CRISPR/MAD7-mediated gene editing. FIG. 6A shows the indel percentages (ICEd) of the control and SOCS1 crRNA samples calculated by ICE analysis. Data are presented as mean±SD. FIG. 6C shows a representative screenshot of the Sanger sequencing result demonstrating the indel formation after CRISPR/MAD7-mediated gene editing at the SOCS1 locus. The shaded area indicates the targeting site after indel formation by SOCS1 crRNA. FIG. 6D shows the summary chart of single clone selection and analysis for SOCS1 edited cells. FIG. 6B shows the western blot analysis of SOCS1 protein depletion in two SOCS1 homozygous knockout clones (#13 and #36).

FIGS. 7A-7D show the editing efficiency of BIM crRNA using CRISPR/MAD7-mediated gene editing. FIG. 7A shows the indel percentages (ICEd) of the control and BIM crRNA samples calculated by ICE analysis. Data are presented as mean±SD. FIG. 7C shows a representative screenshot of the Sanger sequencing result demonstrating the indel formation after CRISPR/MAD7-mediated gene editing at the BIM locus. The shaded area indicates the targeting site after indel formation by BIM crRNA. FIG. 7D shows the summary chart of single clone selection and analysis for BIM edited cells. FIG. 7B shows the western blot analysis of BIM protein depletion in one heterozygous (#21) and one homozygous (#39) knockout clone. The BIM designed crRNA was intended to deplete BIM_{EL} and BIM_{L} splice forms but to retain BIM_{S}.

FIG. 8 shows the prolonged survival of FAS-deficient (knockout of the FAS gene by FAS sgRNA #1 (SEQ ID NO: 60)) and functionally enhanced 6KO (B2M^{-/-}/CIITA^{-/-}/CISH^{-/-}/SOCS1^{-/-}/BIM^{-/-}/FAS^{-/-}) T-iγδT cells in the presence of the FAS ligand (FASL; an inducer of cell apoptosis) and in the absence of human recombinant IL-15, compared with 5KO (B2M^{-/-}/CIITA^{-/-}/CISH^{-/-}/SOCS1^{-/-}/BIM^{-/-}) T-iγδT cells.

FIGS. 9A-9D show the enhanced cytotoxicity and persistence against tumor cells in FAS-deficient and functionally enhanced 6KO T-iγδT cells, compared with 5KO T-iγδT cells. FIGS. 9A-9C show the serial killing ability of the 5KO (B2M^{-/-}/CIITA^{-/-}/CISH^{-/-}/SOCS1^{-/-}/BIM^{-/-}) and 6KO (B2M^{-/-}/CIITA^{-/-}/CISH^{-/-}/SOCS1^{-/-}/BIM^{-/-}/FAS^{-/-}) T-iγδT after a first round (FIG. 9A) and after a second round of activation (FIG. 9B-9C) against HepG2 cells at various effector:target (E:T) ratios monitored using live-cell imaging and analysis. FIG. 9D shows the serial killing ability of 5KO (B2M^{-/-}/CIITA^{-/-}/CISH^{-/-}/SOCS1^{-/-}/BIM^{-/-}) and 6KO (B2M^{-/-}/CIITA^{-/-}/CISH^{-/-}/SOCS1^{-/-}/BIM^{-/-}/FAS^{-/-}) T-iγδT against U937 cells at a 5:1 E:T ratio monitored using live-cell imaging and analysis.

FIG. 10 shows the increased effector molecules in FAS-deficient 6KO T-iγδT cells post serial killing against HepG2 cells compared with 5KO T-iγδT cells. The concentration of effector molecules in the culture supernatants post co-culturing with HepG2 cells was quantified using a bead-based multiplex assay.

FIGS. 11A-11C show the increased IFN-g production percentage in FAS-deficient 6KO T-iγδT cells and an enhanced IFN-g-producing ability of individual FAS deficient 6KO T-iγδT cells relative to 5KO T-iγδT cells. FIG. 11A shows the percentage of TCR Vd2⁺ and FAS⁺ cells in 5KO (B2M^{-/-}/CIITA^{-/-}/CISH^{-/-}/SOCS1^{-/-}/BIM^{-/-}/) and 6KO (B2M^{-/-}/CIITA^{-/-}/CISH^{-/-}/SOCS1^{-/-}/BIM^{-/-}/FAS^{-/-}) T-igdT cells examined using a flow cytometer. FIG. 11B shows percentage of IFN-g-producing TCR Vd2⁺ T-igdT cells in 5KO (B2M^{-/-}/CIITA^{-/-}/CISH^{-/-}/SOCS1^{-/-}/BIM^{-/-}) and 6KO (B2M^{-/-}/CIITA^{-/-}/CISH^{-/-}/SOCS1^{-/-}/BIM^{-/-}/FAS^{-/-}) T-igdT cells in the absence of HepG2 cells and post co-culturing with HepG2 cells for 24 hours examined using a flow cytometer. FIG. 1IC shows the IFN-g mean fluorescence intensity (MFI) of individual 5KO (B2M^{-/-}/CIITA^{-/-}/CISH^{-/-}/SOCS1^{-/-}/BIM^{-/-}) and 6KO (B2M^{-/-}/CIITA^{-/-}/CISH^{-/-}/SOCS1^{-/-}/BIM^{-/-}/FAS^{-/-}) T-igdT cells post co-culturing with HepG2 cells for 24 hours examined using a flow cytometer.

FIG. 12 shows the extracellular domains (ECD) of an interferon gamma (IFN-γ) receptor fused with intracellular domains (ICD) of a receptor of a distinct cytokine with a transmembrane domain in between.

FIGS. 13A-13B show that the IFNγR-IL2/IL15R signal converter can induce STAT1 and STAT5 phosphorylation in mature iγδT 5KO (B2M^{-/-}/CIITA^{-/-}/CISH^{-/-}/SOCS1^{-/-}/BIM^{-/-}) cells. FIG. 13A shows the surface expression of IFNγR-IL2/IL15R on mature iγδT 5KO cells transduced with lentivirus encoding the IFNγR-IL2/IL15R signal converter, in which about 19% of iγδT 5KO cells express IFNγR-IL2/IL15R. In contrast, there was no expression (0.099%) of IFNγR-IL2/IL15R in control cells that were not transduced with lentivirus encoding the IFNγR-IL2/IL15R signal converter. FIG. 13B shows that mature iγδT 5KO cells expressing the IFNγR-IL2/IL15R signal converter were treated with 5 ng/mL IFN-γ in a time-dependent manner (15, 60, and 120 minutes). The phosphorylation of STAT1, STAT3, and STAT5 were examined by immunoblotting. The results show that the phosphorylation of STAT1 and STAT5 were greatly induced in mature iγδT 5KO cells expressing the IFNγR-IL2/IL15R signal converter when treated with IFN-γ at 15 minutes. The increase in the phosphorylation of STAT1 and STAT5 was sustained to 120 minutes. The phosphorylation of STAT3 was also induced after IFN-γ treatment.

FIG. 14 shows the increased proliferation of mature iγδT 5KO cells expressing the IFNγR-IL2/IL15R signal converter under each of the following conditions for 6 days: IL-7 and IL-15 starvation; IL-7 and IL-15 starvation plus the addition of 5 ng/mL IFN-γ; or the addition of 1 ng/mL IL-7 and 1 ng/mL IL-15. The percentage of proliferation was analyzed by flow cytometry and normalized to day 0. The results show that IFNγR-IL2/IL15R signal converter could increase cell proliferation of mature iγδT 5KO cells to 42.3% for 6 days under IFN-γ treatment compared to control cells that lack the IFNγR-IL2/IL15R signal converter, which showed an increased cell proliferation of 11.3%.

FIG. 15 shows the enhanced serial killing ability of mature iγδT 5KO cells expressing the IFNγR-IL2/IL15R signal converter against HepG2 cells (a hepatoblastoma cell line) at an effector:target (E:T) ratio of 5:1 without cytokines, as monitored by a live cell imaging instrument the Incucyte SX5 live-cell analysis system (Sartorius). The results show that the IFNγR-IL2/IL15R signal converter greatly enhanced the serial tumor killing effect of mature iγδT 5KO cells against HepG2 cells and maintained the effect for 24 rounds compared to 5 rounds of killing demonstrated by the control cells. The control cells are iγδT 5KO cells without the IFNγR-IL2/IL15R signal converter.

FIGS. 16A-16C show increased Granzyme A (FIG. 16A), Granzyme B (FIG. 16B), and Perforin (FIG. 16C) secretion by mature iγδT 5KO cells expressing the IFNγR-IL2/IL15R signal converter, when co-cultured with HepG2 cells, in a time-dependent manner (24, 48, 72, and 96 hours).

FIGS. 17A-17B demonstrate dose-dependent and antigen-specific cytotoxicity of the modified CD19-CAR iγδT cells. FIG. 17A shows cytotoxic activity of wild-type (WT, unedited) iγδT, modified CD19-CAR iγδT, donor-derived unedited aPT, and donor-derived CD19-CAR aPT cells using a bioluminescence-based cytotoxicity assay. Cytotoxicity against CD19-positive tumor cell lines, Nalm-6-Luc and Raji-Luc, was tested after 24 hours at the indicated effector to target ratios (E:T) and analyzed by the relative bioluminescence activity. FIG. 17B shows significant killing activity of modified CD19-CAR iγδ T cells against Nalm-6, while there were no significant killing activity of modified CD19-CAR iγδ T cells against Nalm-6-CD19KO (Nalm6 cells that are deficient in CD19 expression), as well as several types of human normal primary cells using a FACS-based cytotoxicity assay, indicating CD19-specific killing activity of the modified CD19-CAR iγδT cells.

FIGS. 18A-18B reveal the secretion profile of cytokines and cytolytic granules from WT iγδT, modified CD19-CAR iγδT, donor-derived unedited αβT and donor-derived CD19-CAR αβT cells against Nalm-6-Luc at E:T ratio=2 for 24 hours using bead-based multiplex method. FIG. 18A shows increased production of cytolytic granules including Granzyme A, Granzyme B, Perforin, and Granulysin from the modified CD19-CAR iγδT. FIG. 18B shows the relatively lower secretion of inflammatory (i.e., IL-4, IL-6, and IL-17a) and immune-modulatory cytokines (i.e., GM-CSF, and IL-10) from the modified CD19-CAR iγδT by the comparison with donor-derived CD19-CAR αβT cells.

FIGS. 19A-19B illustrate dose-dependent and antigen-specific cytotoxicity of modified CD19-CAR iγδT cells against healthy donor- (FIG. 19A) and patient-derived PBMCs (FIG. 19B). FIG. 19A shows cytotoxic activity of WT iγδT, modified CD19-CAR iγδT, donor-derived unedited αβT and donor-derived CD19-CAR αβT cells using a FACS-based cytotoxicity assay. Cytotoxicity against healthy donor PBMCs was tested at the indicated effector to PBMC ratios (E:PBMC) and analyzed by the relative viable CD19-positive or CD19-negative cells of the testing samples versus PBMCs alone, showing specific killing activity of modified CD19-CAR iγδT cells on CD19-positive PBMCs. FIG. 19B shows the cytotoxicity of modified CD19-CAR iγδT and donor-derived CD19-CAR αβT cells against PBMCs from DLBCL or SLE patient at the indicated effector to PBMC ratios (E:PBMC).

FIGS. 20A-20B show the remarkable durable cytotoxic response and proliferation dynamics of the modified CD19-CAR iγδT cells. FIG. 20A unveils the prolonged tumor killing capacity of modified CD19-CAR iγδT cells, as observed through microwell-based live-cell imaging over multiple subsequent tumor challenges by Nalm-6-iRFP713 at E:T ratio=2. FIG. 20B presents the cell proliferation trends of modified CD19-CAR iγδT cells over time. The viable CD3-positive T cells were quantified by FACS analysis.

FIG. 21A shows the durable cytotoxic response of the CD19-CAR 5KO iγδT and IFNγSC+CD19-CAR 5KO iγδT cells, which unveils the prolonged tumor killing capacity of IFNγSC+CD19-CAR 5KO iγδT cells, as observed through microwell-based live-cell imaging over multiple subsequent tumor challenges by Nalm-6-iRFP713 at E:T ratio=4.

FIG. 21B shows the proliferation ability of the CD19-CAR 5KO iγδT and IFNγSC+CD19-CAR 5KO iγδT cells, co-culture with CD19-KO or CD19 wild-type (WT) Nalm-6 tumor at ET2 for 24 hours. It unveils the synergistic proliferation activity of IFNγSC and CD19-CAR on iγδT cells. The proliferation-detection assay was based on the incorporation of nucleoside analogues (EdU) and measurement of EdU-positive cells by FACS analysis.

### DETAILED DESCRIPTION

### I. Definitions

Unless stated otherwise, the following terms and phrases have the meanings described below. The definitions are not meant to be limiting in nature and serve to provide a clearer understanding of certain aspects of the present disclosure.

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

Induced pluripotent stem cell (iPSC): As used herein, the terms “induced pluripotent stem cell” or “iPSC” refer to an induced stem cell that is generated from a somatic cell (e.g., a fibroblast or a T cell) using induced pluripotent reprogramming factors, e.g., Yamanaka factors: Oct3/4, Sox2, Klf4 and c-Myc (see Takahashi K, Yamanaka S. Induction of pluripotent stem cells from mouse embryonic and adult fibroblast cultures by defined factors. Cell. 2006 Aug. 25; 126(4):663-76. Further, see Takahashi K, Tanabe K, Ohnuki M, Narita M, Ichisaka T, Tomoda K, Yamanaka S. Induction of pluripotent stem cells from adult human fibroblasts by defined factors. Cell. 2007 Nov. 30; 131(5):861-72).

T-iPSC: As used herein, the terms “T-iPSC”, “T cell-derived iPSC”, “T derived iPSC”, “iPSC derived from T,” “iPSC derived from a T cell” and the like refer to an iPSC that is generated from a T cell using induced pluripotent reprogramming factors, wherein the T cell could be a primary T cell or a γδT cell.

γδT-iPSC: As used herein, the terms “γδT-iPSC,” “γδT derived iPSC”, “iPSC derived from γδT” and the like refer to an iPSC that is generated from a γδ (gamma/delta) T cell (γδT cell) using induced pluripotent reprogramming factors. The γδT cell can be a Vd2 (or Vδ2) γδT cell or a Vd1 (or Vδ1) γδT cell.

γδTcell: As used herein, a “γδT cell,” “γδT,” “gamma/delta T,” and the like refers to a type of T cell that has a T-cell receptor (TCR) composed of a γ glycoprotein chain and a δ glycoprotein chain on the surface of the cell, regardless of how the γδT cell is generated/obtained. The γδT cell can be a primary γδT cell or a γδT cell differentiated from iPSC (iγδT). The γδT cell can be Vd2 (or Vδ2) γδT or Vd1 (or Vδ1) γδT.

iγδT: The terms “iPSC-derived γδTcell,” “iPSC-γδT,” “iγδT,” and the like, as used herein, refers to a γδT cell differentiated from an iPSC.

T-iγδT: The terms “T-iγδT,” “T-igdT,” “T derived iγδT”, and the like, as used herein, refers to a γδT cell differentiated from an iPSC derived from a T cell (T-iPSC).

γδT-iγδT: The terms “γδT-iγδT”, “γδT-iPSC derived γδT” and the like, as used herein, refers to a γδT differentiated from an iPSC derived from a γδ T cell (γδT-iPSC).

The terms “T-iγδT”, “T derived iγδT”, “γδT-iγδT”, and “γδT-iPSC derived γδT” can be used interchangeably herein. During the reprogramming of a T cell to an iPSC, a γδT cell is usually selected for reprogramming into iPSC, if the iPSC is used to differentiation into a T-iγδT.

“An intermediate cell differentiated from an iPSC” and the like, as used herein, refers to a cell differentiated from an iPSC that possesses the ability to be differentiated into a γδT cell.

The intermediate cell differentiated from the iPSC includes but is not limited to a γδT-iHSC, a γδT-iCLP, and/or an immature T-iγδT cell.

iPSC-derivedHematopoietic stem cell (iHSC): As used herein, the terms “iPSC-derived hematopoietic stem cell, or “iHSC” refer to a hematopoietic stem cell generated from an iPSC.

γδT-iPSC Hematopoietic stem cell (γδT-iHSC): As used herein, the terms “γδT-iPSC hematopoietic stem cell” or “γδT-iHSC” refer to a hematopoietic stem cell generated from a γδT-iPSC.

iPSC-derived Common lymphoidprogenitor (iCLP cell): As used herein, the terms “iPSC-derived common lymphoid progenitor,” “iCLP cell,” or “iCLP” refer to a common lymphoid progenitor cell that is differentiated from an iHSC, in which the iHSC is generated from an iPSC.

γδT-iPSC Common lymphoid precursor (γδT-iCLP): As used herein, the terms “γδT-iPSC common lymphoid precursor,” “γδT-iCLP cell,” or “γδT-iCLP” refer to a common lymphoid precursor cell that is differentiated from a T-iHSC, in which the T-iHSC is generated from a γδT-iPSC.

iPSC-derived immature T cell (immature iT cell): As used herein, the terms “iPSC-derived immature T cell” or “immature iT cell” refer to an immature T cell differentiated from an iCLP cell, in which the iCLP cell is differentiated from an iHSC, in which the iHSC is generated from an iPSC.

γδ-TiPSC immature gamma delta T cell (immature T-iγδT cell): As used herein, the terms “γδT-iPSC immature gamma/delta T cell” or “immature T-iγδT cell” refer to an immature γδT cell differentiated from a T-iCLP, in which the T-iCLP is differentiated from a T-iHSC, in which the T-iHSC is generated from a γδT-iPSC.

Genomic Disruption: As used herein, the term “genomic disruption” refers to any genetic alteration (e.g., substitution, deletion, and/or insertion) in the sequence of a gene or epigenetic modification (e.g., DNA methylation, histone modification) without changes to the sequence of the gene. The genomic disruption can result in a change of the transcription of a gene, translation of mRNA, and/or function of a protein. A genomic disruption can include alterations to a coding sequence, a non-coding sequence (e.g., promoter, enhancer, insulator, operon, or silencer), or a combination thereof.

Splice Variant: As used herein, the term “splice variant” refers to mature mRNA and its encoded protein produced by alternatively splicing a gene, e.g., the BIM gene, in which exons of pre-mRNA are reconnected in at least two distinct forms during the process of RNA splicing. The BIM gene is transcribed as three splice variants: BIM_{EL}, which contains coding exons E1, E2A, E2B, E2C, E4 and E5; BIM_{L}, which contains coding exons E1, E2A, E2C, E4 and E5; and BIM_{S}, which contains coding exons E1, E2A, E4 and E5.

Identity: As used herein, the term “identity” in the context of sequence comparisons refers to the number of exact matches between two different sequences in a sequence alignment. Sequence alignment techniques and software include Basic Local Alignment Search Tool (BLAST, which includes e.g., BLASTP for protein sequences and BLASTN for nucleic acid sequences), ClustalOmega, MUSCLE, and MAFFT.

Bcl-2 Interacting Mediator of cell death (BIM): As used herein, the terms “Bcl-2 Interacting Mediator of cell death,” “BIM,” “Human BIM gene,” “Gene ID: 10018,” or “BCL211” refer to a member of the BH3 (Bcl-2 homology 3)-only proteins, which can directly activate the proapoptotic effector proteins BAX and BAK. BIM can also indirectly activate BAX and BAK by binding to antiapoptotic BCL-2 family members (BCL-2, BCL-xL, MCL-1 and A1). BIM is transcribed as three splice variants, BIM_{EL} (NM_138621.5; NP_619527.1, containing coding exons E1, E2A, E2B, E2C, E4 and E5), BIM_{L} (NM 006538.5; NP_006529.1, containing coding exons E1, E2A, E2C, E4 and E5) and BIM_{S} (NM_001204106.1; NP_001191035.1, containing coding exons E1, E2A, E4 and E5).

Cytokine-inducible SH2-containingprotein (CISH): As used herein, the terms “CISH,” “Cytokine-inducible SH2-containing protein,” “CIS1,” “Gene ID: 1154,” “NM_013324.7,” or “NP_037456.5” refer to a member of the suppressor of cytokine signaling (SOCS) protein family. The SOCS family members are negative regulators of cytokine signal transduction that inhibit the JAK/STAT pathway. Each SOCS family member contains a central SH2 domain and a conserved carboxy-terminal motif designated as the SOCS box. These proteins are important regulators of cytokine signaling, proliferation, differentiation, and immune responses.

Suppressor of cytokine signaling 1 (SOCS1): As used herein, the terms “Suppressor of cytokine signaling 1,” “SOCS1,” “Gene ID: 8651,” “NM_003745.2,” “NP_003736.1” “Janus kinase binding protein,” “JAB,” “Stat-induced Stat inhibitor-1,” “SSI-1,” “Tec-interacting protein 3,” or “TIP3” refer to a cytokine-regulated SOCS family member that directly inhibits JAK family members through interaction within its kinase activation loop. In addition to inhibiting JAK/STAT signaling, SOCS1 can also negatively regulate Toll-like receptors that contribute to innate immunity.

FAS cell surface death receptor: As used herein, the terms “FAS cell surface death receptor,” “FAS,” “Gene ID: 355,” “NM_000043.6,” and “NP_000034.1” refer to a tumor necrosis factor (TNF) superfamily death receptor that induce caspase-dependent apoptosis following engagement with its extracellular ligand (FAS ligand (FASL)).

β-2-Microglobulin: As used herein, the terms “β-2-Microglobulin,” “Beta-2-Microglobulin,” “B2M,” “Gene ID: 567,” “NM_004048,” and “NP_004039” refer to a component of the human leukocyte antigen (HLA) class I molecules found on the surface of nearly all nucleated cells. In addition, β2M associates non-covalently with the HLA class I heavy chain, enabling proper folding and stabilization of HLA class I.

Class II transactivator: As used herein, the terms “Class II transactivator,” “CIITA,” “Gene ID: 4261,” “NM_001286402.1,” and “NP_001273331.1” refer to a master regulator of HLA class II expression by recruiting and facilitating the assembly of DNA-binding proteins, including the regulatory factor X (RFX) complex, cAMP response element-binding protein (CREB), and nuclear factor Y (NF-Y).

Clustered Regularly Interspace Short Palindromic Repeats (CRISPR): As used herein, the terms “Clustered Regularly Interspace Short Palindromic Repeats” or “CRISPR” refer to a gene editing system that utilizes a guide sequence and a nuclease to modify a cell's genome at a specific location. The nuclease cuts the cell's genome at the specific location targeted by the guide sequence, allowing a target sequence to be removed, a sequence to be added, or a combination thereof.

Programmable Addition Via Site-Specific Target Elements (PASTE): As used herein, the terms “Programmable Addition Via Site-Specific Target Elements” or “PASTE” refer to a gene editing system that utilizes a guide sequence and a CRISPR-Cas9 nickase fused to a reverse transcriptase and a serine integrase to modify a cell's genome at a specific location. The nickase cuts one strand of double stranded DNA of the cell's genome at the specific location targeted by the guide sequence, allowing a target sequence to be removed, a sequence to be added using the serine integrase, or a combination thereof.

Prime Editing: As used herein, the term “Prime editing” refers to a gene editing system that utilizes a guide sequence and a catalytically impaired Cas nickase (e.g., Cas9 nickase or Cas12 nickase) fused to a reverse transcriptase to modify a cell's genome at a specific location. The nickase cuts one strand of double stranded DNA of the cell's genome at the specific location targeted by the guide sequence, allowing a target sequence to be removed, a sequence to be added using the reverse transcriptase, or a combination thereof.

Base Editing: As used herein, the term “Base editing” refers to a gene editing system that utilizes a guide sequence and a base editor, which is a Cas nickase (e.g., Cas9 nickase or Cas12 nickase) and a nucleoside deaminase, including e.g., a cytosine deaminase or an adenine deaminase, to modify a cell's genome at a specific location. The nickase cuts one strand of double stranded DNA of the cell's genome at the specific location targeted by the guide sequence, allowing the nucleoside deaminase to remove an amino group from a specific type of nucleoside (e.g., cytosine or adenine) at a specific location.

Transcription Activator-like Effector Nuclease (TALEN): As used herein, the terms “Transcription Activator-like Effector Nuclease” or “TALEN” refer to a gene editing system that utilizes an endonuclease, including e.g., Fokl, fused to a transcription activator-like effector (TALE) that is engineered to bind to a desired DNA sequence to promote DNA cleavage by the endonuclease at the specific location designated by the TALE.

Zinc-finger nuclease (ZFN): As used herein, the terms “Zinc-finger nuclease” or “ZFN” refer to a gene editing system that utilizes an endonuclease, including e.g., Fokl, fused to a plurality of zinc fingers. Each zinc finger targets a specific nucleotide sequence at which the endonuclease generates a double-stranded DNA break.

Endonuclease: As used herein, the term “endonuclease” refers to an enzyme that cleaves a phosphodiester bond of a polynucleotide molecule. An endonuclease that targets a specific sequence of nucleotides is a restriction endonuclease. As used herein, the terms “homing endonuclease” and “meganuclease” refer to an endonuclease that targets specific asymmetric sequences of about 12 to and 45 base pairs in length, usually about 14 to about 25 base pairs in length.

Transfection: As used herein, the term “transfection” refers to a variety of techniques for introducing a foreign nucleic acid molecule (e.g., DNA) into a host cell, including electroporation, physical transfection (e.g., microinjection, particle bombardment, or phototransfection), lipid-mediated transfection, diethylaminoethyl (DEAE)-dextran transfection, calcium phosphate precipitation, cationic polymer transfection, or viral transfection.

Interferon-gamma receptor: As used herein, the terms “interferon-gamma receptor,” “IFNGR,” and “IFN-γ receptor” refer to a cytokine receptor comprising a heterodimer. The heterodimer of IFNGR comprises interferon-gamma receptor 1 (IFNGR1) and interferon-gamma receptor 2 (IFNGR2). The interferon-gamma cytokine binds to the IFNGR.

CD132: As used herein, the terms “CD132,” “the common γ chain (γc) of interleukin-2 receptor,” and “IL2RG” refer to a cytokine receptor subunit that is common to the cytokine receptor complexes of at least IL-2, IL-15, IL-4, IL-7, IL9, and IL-21. CD132 comprises an intracellular domain, a transmembrane domain, and a membrane-proximal region.

Interleukin-2 receptor beta subunit: As used herein, the terms “interleukin-2 receptor beta subunit,” “interleukin-2 receptor subunit beta,” “CD122,” and “IL2RB” refer to a cytokine receptor subunit of the cytokine receptor complex of IL-2.

As used herein, the terms “IFNγR-IL2/IL15R signal converter”, “IFNγR signal converter”, “IFNγ signal converter”, “IFN-γ signal converter”, “IFNg signal converter”, and “IFNγSC” refer to a chimeric signaling converter comprising a heterodimer. It comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (TL2RB).

As used herein, “unmodified control” and “unmodified cell” refer to a control cell relative to which a comparison is performed. In some embodiments, the unmodified control is the same as the modified cell herein, except that the unmodified control expresses endogenous levels of BIM splice variants as that of a wild-type cell (e.g., γδT cell). In some embodiments, the unmodified control is the same as the modified cell herein, except that the unmodified control expresses endogenous level of a specific protein or protein combination (under comparison) as that of a wild-type cell (e.g., γδT cell). The unmodified control can also have further modifications (other than the protein(s) under comparison) as those in the modified cell to be compared with.

As used herein, “suppresses or eliminates” means any reduction in the level of a functional protein expressed from a gene as compared to a non-modified control.

As used herein, “inactivating nucleic acid mutation” means any nucleic acid mutation causing any reduction in the level of a functional protein expressed from a gene as compared to a non-modified control.

B2M-HLA-E: As used herein, the terms “B2M-HLA-E”, “B2M-HLAE”, “B2M-HLA-E fusion protein” refer to a fusion protein comprising at least a portion of B2M and a portion of HLA-E, wherein at least a portion of B2M and a portion of HLA-E is connected directly or indirectly via a linker. In some embodiments, B2M-HLA-E is able to engage the inhibitory receptors on the surface of NK cells. In some embodiments, B2M-HLA-E comprises an amino acid sequence of SEQ ID NO: 85.

TRAC: As used herein, the term “TRAC” refers to the T-cell receptor alpha constant region gene. It is part of the T-cell receptor (TCR) complex, which is responsible for recognizing antigens in the context of the major histocompatibility complex (MHC) on antigen-presenting cells.

The term “CAR” and “chimeric antigen receptor” refers to a chimeric receptor engineered to endow them with antigen specificity while retaining or enhancing their ability to recognize, trigger signaling pathway and kill a target cell. Chimeric antigen receptor may comprise, for example, (i) an extracellular binding domain, comprising an antigen-specific component, (e.g., a scFv binding to a specific antigen) and optionally a hinge region; (ii) a transmembrane domain; and (iii) intracellular signaling domain, e.g., one or more costimulatory domains and one or more activation domains. Each domain may be heterogeneous, i.e., comprised of sequences derived from (or corresponding to) different protein chains.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient comprising, or at risk of having, a disorder described herein).

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

As used herein, the term “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion).

The term “therapeutically effective amount” as herein used, refers to the amount of a therapeutic agent that, when administered to a subject for treating a disease, or at least one of the clinical symptoms of a disease or disorder, is sufficient to effect such treatment for the disease, disorder, or symptom. The “therapeutically effective amount” can vary with the therapeutic agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

### II. Modified Cells

Disclosed herein are modified γδT-cells, e.g., iPSC-derived γδT-cells, and their precursors (e.g., an iPSC (e.g., γδT-iPSC)), or an intermediate cell differentiated from an iPSC (e.g., iHSC, iCLP, and immature iγδT cells)).

Modified Cells with One or More Genomic Disruptions

In one aspect, such modified cells comprise one or more genomic disruptions selected from the following: 1) a genomic disruption in the SOCS1 gene, 2) a genomic disruption in the CISH gene, 3) a genomic disruption in the BIM gene, 4) a genomic disruption in the B2M gene, 5) a genomic disruption in the CIITA gene, 6) a genomic disruption in the FAS gene, and 7) a genomic disruption in the TRAC gene.

In another aspect, such modified cells (e.g., iPSC or γδT cell) comprise at least two genomic disruptions—1) in the suppressor of cytokine signaling 1 (SOCS1) gene; and 2) in the cytokine-inducible sh2-containing protein (CISH) gene. In some embodiments, the modified cells comprise at least three genomic disruptions—1) in the SOCS1 gene, 2) in the CISH gene, and 3) in the Bcl-2 interacting mediator of cell death (BIM) gene. In some embodiments, the modified cells comprise at least four genomic disruptions—1) in the SOCS1 gene, 2) in the CISH gene, 3) in the BIM gene, and, for example, 4) in the FAS cell surface death receptor (FAS) gene. In some embodiments, the modified cells comprise at least five genomic disruptions—1) in the SOCS1 gene, 2) in the CISH gene, 3) in the BIM gene, and, for example, 4) in the β-2-Microglobulin (B2M) gene, and 5) in the class II transactivator (CIITA) gene. In some embodiments, the modified cells comprise at least six genomic disruptions—1) in the SOCS1 gene, 2) in the CISH gene, 3) in the BIM gene, and, for example, 4) in the B2M gene, 5) in the CIITA gene, and 6) in the FAS gene. In some embodiments, the modified cells comprise at least seven genomic disruptions—1) in the SOCS1 gene, 2) in the CISH gene, 3) in the BIM gene, and, for example, 4) in the B2M gene, 5) in the CIITA gene, 6) in the FAS gene, and 7) in the TRAC gene.

In another aspect, the modified cell (e.g., iPSC or γδT cell) comprises at least two genomic disruptions—1) a genomic disruption in the SOCS1 gene (e.g., in exon 2 of the SOCS1 gene), and 2) a genomic disruption in the CISH gene (in exon 3 of the CISH gene). In some embodiments, the modified cell (e.g., iPSC or γδT cell) comprises at least three genomic disruptions—1) a genomic disruption in the SOCS1 gene (e.g., in exon 2 of the SOCS1 gene), 2) a genomic disruption in the CISH gene (in exon 3 of the CISH gene), and 3) a genomic disruption in the BIM gene (e.g., in exon 2C of the BIM gene). In some embodiments, the modified cell (e.g., iPSC or γδT cell) comprises at least four genomic disruptions—1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), and, for example, 4) a genomic disruption in the FAS gene. In some embodiments, the modified cell (e.g., iPSC or γδT cell) comprises at least five genomic disruptions—1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), 4) a genomic disruption in the B2M gene, and 5) a genomic disruption in the CIITA gene. In some embodiments, the modified cell (e.g., iPSC or γδT cell) comprises at least six genomic disruptions—1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), 4) a genomic disruption in the FAS gene, 5) a genomic disruption in the B2M gene, and 6) a genomic disruption in the CIITA gene. In some embodiments, the modified cell (e.g., iPSC or γδT cell) further comprises at least seven genomic disruptions—1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), 4) a genomic disruption in the FAS gene, 5) a genomic disruption in the B2M gene, 6) a genomic disruption in the CIITA gene, and 7) a genomic disruption in the TRAC gene.

Modified Cells with One or More Genomic Disruptions and One or More Polynucleotide Incorporations

In one aspect, the modified cell (e.g., iPSC or γδT cell) comprises one or more modifications selected from the following: 1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), 4) a genomic disruption in the FAS gene, 5) a genomic disruption in the B2M gene, 6) a genomic disruption in the CIITA gene, 7) a genomic disruption in the TRAC gene, 8) an exogenous polynucleotide encoding B2M-HLA-E (e.g., inserted into a B2M locus), and 9) one or more exogenous polynucleotides encoding a signal converter (e.g., IFNγ signal converter) comprising a first fusion protein subunit and a second fusion protein subunit (e.g., the first fusion protein subunit of the signal converter is inserted into a SOCS1 locus and the second fusion protein subunit of the signal converter is inserted into a ROSA26 locus, or vice versa).

In another aspects, in addition to the genomic disruption combination described above, the modified cell (e.g., iPSC or γδT cell) further comprises at least one or two of the following: 1) an exogenous polynucleotide encoding B2M-HLA-E (e.g., inserted into a B2M locus), and 2) one or more exogenous polynucleotides encoding an IFNγ signal converter.

In another aspects, in addition to the genomic disruption combination described above, the modified cell (e.g., iPSC or γδT cell) further comprises 1) an exogenous polynucleotide encoding B2M-HLA-E (e.g., inserted into a B2M locus), and 2) one or more exogenous polynucleotides encoding an IFNγ signal converter.

In one embodiment, the modified cell (e.g., iPSC or γδT cell) further comprises all the modifications selected from the following: 1) a genomic disruption in the SOCS1 gene (e.g., in exon 2 of the SOCS1 gene), 2) a genomic disruption in the CISH gene (in exon 3 of the CISH gene), 3) a genomic disruption in the BIM gene (e.g., in exon 2C of the BIM gene), 4) a genomic disruption in the FAS gene, 5) a genomic disruption in the B2M gene, 6) a genomic disruption in the CIITA gene, 7) a genomic disruption in the TRAC gene, 8) an exogenous polynucleotide encoding B2M-HLA-E (e.g., inserted into a B2M locus), and 9) one or more exogenous polynucleotides encoding a signal converter (e.g., IFNγ signal converter) comprising a first fusion protein subunit and a second fusion protein subunit (e.g., the first fusion protein subunit of the signal converter is inserted into a SOCS1 locus and the second fusion protein subunit of the signal converter is inserted into a ROSA26 locus, or vice versa).

In some embodiments, the modified cell is a γδT cell. The γδT cell can be a Vd2 (or Vδ2) γδT or a Vd1 (or Vδ1) γδT. In some embodiments, the γδT cell can be modified before reprogramming to an iPSC. In some embodiments, the modified cell is an iPSC-derived γδT cell (iγδT). In some embodiments, the γδT cell can be a primary γδT cell. In some embodiments, the modified γδT cell is differentiated from the modified iPSC.

In some embodiments, the modified cell is an iPSC. In some embodiments, a somatic cell (e.g., T cell or PBMC) can be modified before reprogramming to an iPSC. An iPSC is a PSC that is generated from a somatic cell using induced pluripotent reprogramming factors. In some embodiments, the somatic cell is a T cell, including a T6 (gamma/delta) T cell, a peripheral blood mononuclear cell (PBMC), a fibroblast, or a cord blood cell.

In some embodiments, the modified cell is an iPSC derived from a primary T cell (T-iPSC).

In some embodiments, the modified cell is a γδT-iPSC. A γδT-iPSC is an iPSC that is generated from a γδ (gamma/delta) T cell (γδT cell) using induced pluripotent reprogramming factors. In some embodiments, the γδT cell used for generating an iPSC using reprograming factors is a Vd2 γδT cell.

In some embodiments, the modified cell is a Vd2 γδT cell. In some embodiments, the modified cell is a primary γδT cell. In some embodiments, the modified cell is an iPSC-derived γδT (iγδT) cell. In some embodiments, the modified cell is an iγδT cell from a T-iPSC (T-iγδT). In some embodiments, the modified cell is a γδT cell from γδT-iPSC (γδT-iγδT).

In some embodiments, the modified cell is an iHSC. An iHSC refers to a hematopoietic stem cell generated from an iPSC. In some embodiments, the modified cell is a γδT-iHSC. A γδT-iHSC refers to a hematopoietic stem cell generated from a γδT-iPSC.

In some embodiments, the modified cell is an iCLP cell, which is a common lymphoid progenitor cell that is differentiated from an iHSC. In some embodiments, the modified cell is a γδT-iCLP, which is a common lymphoid precursor cell that is differentiated from a γδT-iHSC.

In some embodiments, the modified cell is an immature iT cell, which is an immature T cell that is differentiated from an iHSC and/or an iCLP cell. In some embodiments, the modified cell is an immature T-iγδT cell, which is an immature γδT cell that is differentiated from a γδT-iCLP.

In some embodiments, one or more of the SOCS1, CISH, BIM, FAS, B2M, CIITA and TRAC gene are genomically disrupted in a γδT-iPSC, γδT-iHSC, γδT-iCLP, or immature T-iγδT cell before differentiation into the T-iγδT cell.

In some embodiments, the SOCS1 and CISH genes are genomically disrupted in a γδT-iPSC, γδT-iHSC, γδT-iCLP, or immature T-iγδT cell before differentiation into the T-iγδT cell. In some embodiments, the SOCS1, CISH, and BIM genes are genomically disrupted in a γδT-iPSC, γδT-iHSC, γδT-iCLP, or immature T-iγδT cell before differentiation into the T-iγδT cell. In some embodiments, the genomic disruptions of the SOCS1 and CISH genes each occur in distinct cell differentiation stages, e.g., the SOCS1 gene is genomically disrupted in the T-iCLP cell and the CISH gene is genomically disrupted in the immature T-iγδT cell that is differentiated from the T-iCLP containing the genomically disrupted SOCS1 gene. A cell differentiation stage is any one of the following cell types: a stem cell having self-renewal and pluripotency properties (e.g., an induced pluripotent stem cell), an adult stem cell (e.g., hematopoietic stem cell) that is differentiated from the stem cell having self-renewal and pluripotency properties; a progenitor cell (e.g., a common lymphoid progenitor) that is differentiated from the adult stem cell; a specific type of cell (e.g., an immature T cell) that is differentiated from the progenitor cell; and a mature specific type of cell (e.g., a γδT cell) that is differentiated from the specific type of cell. A cell differentiation stage is any one of γδT-iPSC, T-iHSC, T-iCLP, immature T-iγδT cell, or T-iγδT cell.

In some embodiments, the genomic disruptions of the SOCS1, CISH, and BIM genes each occur in distinct cell differentiation stages, e.g., the SOCS1 gene is genomically disrupted in the T-iHSC, the CISH gene is genomically disrupted in the T-iCLP differentiated from the T-iHSC containing the genomically disrupted SOCS1 gene, and the BIM gene is genomically disrupted in the immature T-iγδT cell that is differentiated from the T-iCLP containing the SOCS1 and CISH genomic disruptions. In some embodiments, genomic disruptions of two of the genes selected from SOCS1, CISH, and BIM occur in a single cell differentiation stage and a third genomic disruption of the remaining undisrupted gene occurs in a second cell differentiation stage, e.g., the SOCS1 and CISH genes are genomically disrupted in the T-iHSC and the BIM gene is genomically disrupted in the T-iCLP differentiated from the T-iHSC containing the genomically disrupted SOCS1 and CISH genes. In some embodiments, genomic disruptions of two of the genes selected from SOCS1, CISH, and BIM occur in a single cell differentiation stage. In some embodiments, genomic disruptions in each of the three genes selected from SOCS1, CISH, and BIM occur in a single cell differentiation stage.

In some embodiments, the cells comprise genomic disruptions of the SOCS1, CISH, BIM, and FAS genes. In some embodiments, the genomic disruptions of the SOCS1, CISH, BIM, and FAS genes each occur in a distinct cell differentiation stage. In some embodiments, genomic disruptions of three of the genes selected from FAS, SOCS1, CISH, and BIM occur in a single cell differentiation stage and a fourth genomic disruption of the remaining undisrupted gene occurs in a second cell differentiation stage. In some embodiments, genomic disruptions of two of the genes selected from FAS, SOCS1, CISH, and BIM occur in a single cell differentiation stage. In some embodiments, a genomic disruption of one of the genes selected from FAS, SOCS1, CISH, and BIM occurs in a single cell differentiation stage. In some embodiments, genomic disruptions in each of the four genes selected from FAS, SOCS1, CISH, and BIM occur in a single cell differentiation stage.

In some embodiments, the cells comprise genomic disruptions of the SOCS1, CISH, BIM, B2M and CIITA genes. In some embodiments, the genomic disruptions of the SOCS1, CISH, BIM, B2M and CIITA genes each occur in a distinct cell differentiation stage. In some embodiments, genomic disruptions of four of the genes selected from SOCS1, CISH, BIM, B2M and CIITA occur in a single cell differentiation stage. In some embodiments, genomic disruptions of three of the genes selected from SOCS1, CISH, BIM, B2M and CIITA occur in a single cell differentiation stage. In some embodiments, genomic disruptions of two of the genes selected from SOCS1, CISH, BIM, B2M and CIITA occur in a single cell differentiation stage. In some embodiments, a genomic disruption of one of the genes selected from SOCS1, CISH, BIM, B2M and CIITA occurs in a single cell differentiation stage. In some embodiments, genomic disruptions in each of the genes selected from SOCS1, CISH, BIM, B2M and CIITA occur in a single cell differentiation stage.

In some embodiments, the cells comprise genomic disruptions of the SOCS1, CISH, BIM, B2M, CIITA, and FAS genes. In some embodiments, the genomic disruptions of the SOCS1, CISH, BIM, B2M, CIITA, and FAS genes each occur in a distinct cell differentiation stage. In some embodiments, genomic disruptions of five of the genes selected from SOCS1, CISH, BIM, B2M, CIITA, and FAS occur in a single cell differentiation stage. In some embodiments, genomic disruptions of four of the genes selected from SOCS1, CISH, BIM, B2M, CIITA, and FAS occur in a single cell differentiation stage. In some embodiments, genomic disruptions of three of the genes selected from SOCS1, CISH, BIM, B2M, CIITA, and FAS occur in a single cell differentiation stage. In some embodiments, genomic disruptions of two of the genes selected from SOCS1, CISH, BIM, B2M, CIITA, and FAS occur in a single cell differentiation stage. In some embodiments, a genomic disruption of one of the genes selected from SOCS1, CISH, BIM, B2M, CIITA, and FAS occur in a single cell differentiation stage. In some embodiments, genomic disruptions in each of the genes selected from SOCS1, CISH, BIM, B2M, CIITA, and FAS occur in a single cell differentiation stage.

In some embodiments, one or more of the SOCS1, CISH, BIM, FAS, B2M, and CIITA genes are genomically disrupted in an iPSC cell. In some embodiments, the SOCS1 and CISH genes are genomically disrupted in the iPSC cell. In some embodiments, the SOCS1, CISH, and BIM genes are genomically disrupted in the iPSC cell. In some embodiments, the FAS, SOCS1, CISH, and BIM genes are genomically disrupted in the iPSC cell. In some embodiments, the B2M, CIITA, SOCS1, CISH, and BIM genes are genomically disrupted in the iPSC cell. In some embodiments, the FAS, B2M, CIITA, SOCS1, CISH, and BIM genes are genomically disrupted in the iPSC cell.

In some embodiments, one or more of the SOCS1, CISH, BIM, FAS, B2M, and CIITA genes are genomically disrupted in a γδT-iPSC cell. In some embodiments, the SOCS1 and CISH genes are genomically disrupted in the γδT-iPSC cell. In some embodiments, the SOCS1, CISH, and BIM genes are genomically disrupted in the γδT-iPSC cell. In some embodiments, the FAS, SOCS1, CISH, and BIM genes are genomically disrupted in the γδT-iPSC cell. In some embodiments, the B2M, CIITA, SOCS1, CISH, and BIM genes are genomically disrupted in the γδT-iPSC cell. In some embodiments, the FAS, B2M, CIITA, SOCS1, CISH, and BIM genes are genomically disrupted in the γδT-iPSC cell.

In some embodiments, one or more of the SOCS1, CISH, BIM, FAS, B2M, and CIITA genes are genomically disrupted in an iPSC-derived γδT cell (iTST cell). In some embodiments, the SOCS1 and CISH genes are genomically disrupted the iPSC-derived γδT cell (iγδT cell). In some embodiments, the SOCS1, CISH, and BIM genes are genomically disrupted in the iγδT cell. In some embodiments, the FAS, SOCS1, CISH, and BIM genes are genomically disrupted in the iγδT cell. In some embodiments, the B2M, CIITA, SOCS1, CISH, and BIM genes are genomically disrupted in the iγδT cell. In some embodiments, the FAS, B2M, CIITA, SOCS1, CISH, and BIM genes are genomically disrupted in the iγδT cell.

In some embodiments, one or more of the SOCS1, CISH, BIM, FAS, B2M, and CIITA genes are genomically disrupted in a T-iPSC-derived γδT cell (T-iγδT cell). In some embodiments, the SOCS1 and CISH genes are genomically disrupted the T-iPSC-derived γδT cell (T-iγδT cell). In some embodiments, the SOCS1, CISH, and BIM genes are genomically disrupted in the T-iPSC-derived γδT cell (T-iγδT cell). In some embodiments, the FAS, SOCS1, CISH, and BIM genes are genomically disrupted in the T-iPSC-derived γδT cell (T-iγδT cell). In some embodiments, the B2M, CIITA, SOCS1, CISH, and BIM genes are genomically disrupted in the T-iPSC-derived γδT cell (T-iγδT cell). In some embodiments, the FAS, B2M, CIITA, SOCS1, CISH, and BIM genes are genomically disrupted in the T-iPSC-derived γδT cell (T-iγδT cell).

In some embodiments, one or more of the SOCS1, CISH, BIM, FAS, B2M, and CIITA genes are genomically disrupted in a γδT-iPSC-derived γδT cell (γδT-iγδT cell). In some embodiments, the SOCS1 and CISH genes are genomically disrupted in the γδT-iPSC-derived γδT cell (γδT-iγδT cell). In some embodiments, the SOCS1, CISH, and BIM genes are genomically disrupted in the γδT-iPSC-derived γδT cell (γδT-iγδT cell). In some embodiments, the FAS, SOCS1, CISH, and BIM genes are genomically disrupted in the γδT-iPSC-derived γδT cell (γδT-iγδT cell). In some embodiments, the B2M, CIITA, SOCS1, CISH, and BIM genes are genomically disrupted in the γδT-iPSC-derived γδT cell (γδT-iγδT cell). In some embodiments, the FAS, B2M, CIITA, SOCS1, CISH, and BIM genes are genomically disrupted in the γδT-iPSC-derived γδT cell (γδT-iγδT cell).

In some embodiments, one or more of the SOCS1, CISH, BIM, FAS, B2M, and CIITA genes are genomically disrupted in an iHSC. In some embodiments, the SOCS1 and CISH genes are genomically disrupted the iHSC. In some embodiments, the SOCS1, CISH, and BIM genes are genomically disrupted in the iHSC. In some embodiments, the FAS, SOCS1, CISH, and BIM genes are genomically disrupted in the iHSC. In some embodiments, the B2M, CIITA, SOCS1, CISH, and BIM genes are genomically disrupted in the iHSC. In some embodiments, the FAS, B2M, CIITA, SOCS1, CISH, and BIM genes are genomically disrupted in the iHSC.

In some embodiments, one or more of the SOCS1, CISH, BIM, FAS, B2M, and CIITA genes are genomically disrupted in a γδT-iHSC. In some embodiments, the SOCS1 and CISH genes are genomically disrupted the γδT-iHSC. In some embodiments, the SOCS1, CISH, and BIM genes are genomically disrupted in the γδT-iHSC. In some embodiments, the FAS, SOCS1, CISH, and BIM genes are genomically disrupted in the γδT-iHSC. In some embodiments, the B2M, CIITA, SOCS1, CISH, and BIM genes are genomically disrupted in the γδT-iHSC. In some embodiments, the FAS, B2M, CIITA, SOCS1, CISH, and BIM genes are genomically disrupted in the γδT-iHSC.

In some embodiments, one or more of the SOCS1, CISH, BIM, FAS, B2M, and CIITA genes are genomically disrupted in an iCLP cell. In some embodiments, the SOCS1 and CISH genes are genomically disrupted the iCLP cell. In some embodiments, the SOCS1, CISH, and BIM genes are genomically disrupted in the iCLP cell. In some embodiments, the FAS, SOCS1, CISH, and BIM genes are genomically disrupted in the iCLP cell. In some embodiments, the B2M, CIITA, SOCS1, CISH, and BIM genes are genomically disrupted in the iCLP cell. In some embodiments, the FAS, B2M, CIITA, SOCS1, CISH, and BIM genes are genomically disrupted in the iCLP cell.

In some embodiments, one or more of the SOCS1, CISH, BIM, FAS, B2M, and CIITA genes are genomically disrupted in a γδT-iCLP cell. In some embodiments, the SOCS1 and CISH genes are genomically disrupted the γδT-iCLP cell. In some embodiments, the SOCS1, CISH, and BIM genes are genomically disrupted in the γδT-iCLP cell. In some embodiments, the FAS, SOCS1, CISH, and BIM genes are genomically disrupted in the γδT-iCLP cell. In some embodiments, the B2M, CIITA, SOCS1, CISH, and BIM genes are genomically disrupted in the γδT-iCLP cell. In some embodiments, the FAS, B2M, CIITA, SOCS1, CISH, and BIM genes are genomically disrupted in the γδT-iCLP cell.

In some embodiments, the SOCS1 and CISH genes are genomically disrupted in the γδT-iPSC cell, and the FAS, B2M, CIITA, and/or BIM gene is genomically disrupted in any one of T-iγδT, immature T-iγδT cell, γδT-iHSC, or γδT-iCLP cell.

In some embodiments, the SOCS1 and CISH genes are genomically disrupted in the immature T-iγδT cell, and the FAS, B2M, CIITA, and/or BIM gene is genomically disrupted in any one of γδT-iPSC, T-iγδT, γδT-iHSC, or γδT-iCLP cell.

In some embodiments, the SOCS1 and CISH genes are genomically disrupted in the T-iγδT cell, and the FAS, B2M, CIITA, and/or BIM gene is genomically disrupted in any one of γδT-iPSC, immature T-iγδT cell, γδT-iHSC, or γδT-iCLP cell.

In some embodiments, the SOCS1 and CISH genes are genomically disrupted in the γδT-iHSC, and the FAS, B2M, CIITA, and/or BIM gene is genomically disrupted in any one of γδT-iPSC, immature T-iγδT cell, T-iγδT, or γδT-iCLP cell.

In some embodiments, the SOCS1 and CISH genes are genomically disrupted in the γδT-iCLP cell, and the FAS, B2M, CIITA, and/or BIM gene is genomically disrupted in any one of γδT-iPSC, immature T-iγδT cell, T-iγδT, or γδT-iHSC.

In some embodiments, the exogenous polynucleotide encoding B2M-HLA-E is incorporated into a B2M locus of an iPSC (e.g., γδT-iPSC) and/or one or more exogenous polynucleotides encoding a signal converter (e.g., IFNγ signal converter) are incorporated into an iPSC (e.g., γδT-iPSC).

In some embodiments, the cell is mammalian. In some embodiments, the cell is human.

In some embodiments, the cell is in vivo, in vitro, ex vivo, or in a human subject.

Also encompassed are populations of cells comprising one or more modified cells described herein.

In some embodiments, the cells are stored for a length of time before use. In some embodiments, the time is weeks or months. In some embodiments, the time is months or years.

In some embodiments, the cells are stored frozen in liquid nitrogen (−196 degrees Celsius).

A. Suppressor of Cytokine Signaling 1 (Socs1) Genomic disruption

The present disclosure provides modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising one, two, three, four, five, six, or seven genomic disruptions and/or one, two, three, or four of exogenous polynucleotide incorporations, one of which is a genomic disruption of the endogenous suppressor of cytokine signaling 1 (SOCS1) gene. In some embodiments, the modified cell is a modified T-iγδT cell comprising a genomic disruption of the endogenous SOCS1 gene. In some embodiments, the modified cell is a γδT-cell that is differentiated from a modified (e.g., alone or with any one or more genomic disruptions in CISH, BIM, FAS, B2M, CIITA, and TRAC, and/or any one or more incorporations of B2M-HLA-E and signal converter) or unmodified γδT-iPSC, modified or unmodified T-iHSC, modified or unmodified T-iCLP, or modified or unmodified immature T-iγδT cell. In some embodiments, the modified cell is from a primary γδT-cell.

In some embodiments, the modified cell comprises a genomic disruption in an endogenous SOCS1 gene that suppresses or eliminates expression of the SOCS1 gene relative to an unmodified control. In some embodiments, the genomic disruption in the SOCS1 gene is an inactivating nucleic acid mutation (e.g., knock out). In some embodiments, the inactivating nucleic acid mutation in the SOCS1 gene is an insertion, deletion, substitution, or any combination thereof. As used herein, “suppresses or eliminates” means any reduction in the level of a functional protein expressed from a gene as compared to a non-modified control. As used herein, “inactivating nucleic acid mutation” means any nucleic acid mutation causing any reduction in the level of a functional protein expressed from a gene as compared to a non-modified control. In some embodiments, the genomic disruption in the SOCS1 gene is an endonuclease-mediated insertion-deletion (indel). In some embodiments, the inactivating nucleic acid mutation in the SOCS1 gene is an endonuclease-mediated indel, such as a targeted knockout obtainable by a CRISPR-Cas system (e.g., Cas9, Cas12 or MAD7) designed to target SOCS1.

In some embodiments, the genomic disruption of the endogenous SOCS1 gene is in exon 2. In some embodiments, the genomic disruption is an inactivating nucleic acid mutation in exon 2. In some embodiments, the genomic disruption is within a target sequence in SOCS1 that comprises the nucleotide sequence of any one of SEQ ID NO: 17, SEQ ID NO: 23, or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

Assays for determining the relative expression of a gene or function of a protein encoded by the gene in a modified cell (e.g., T-iγδT-cell) can be performed using various methods. For example, the relative expression of the SOCS1 gene in a modified γδT-cell (e.g., T-iγδT-cell) relative to an unmodified γδT-cell (e.g., an unmodified T-iγδT-cell) or relative to the γδT-cell (e.g., T-iγδT-cell) before modification can be determined using quantitative polymerase chain reaction (qPCR), digital PCR (dPCR), droplet digital PCR (ddPCR), or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### B. Cytokine-Inducible Sh2-Containing Protein (Cish) Genomic Disruption

The present disclosure provides modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising one, two, three, four, five, six, or seven genomic disruptions and/or one, two, three, or four of exogenous polynucleotide incorporations, one of which is a genomic disruption of the endogenous cytokine-inducible sh2-containing protein (CISH) gene. In some embodiments, the modified cell is a modified T-iγδT cell comprising a genomic disruption of the endogenous CISH gene. In some embodiments, the modified cell is a γδT-cell that is differentiated from a modified (e.g., alone or with any one or more genomic disruptions in SOCS1, BIM, FAS, B2M, CIITA, and TRAC, and/or any one or more incorporations of B2M-HLA-E and signal converter) or unmodified γδT-iPSC, a modified or unmodified T-iHSC, a modified or unmodified T-iCLP, or a modified or unmodified immature T-iγδT cell. In some embodiments, the modified cell is from a primary γδT-cell.

In some embodiments, the cell comprises a genomic disruption in an endogenous CISH gene that suppresses or eliminates expression of the CISH gene relative to an unmodified control. In some embodiments, the genomic disruption in the CISH gene is an inactivating nucleic acid mutation (e.g., knock out). In some embodiments, the inactivating nucleic acid mutation in the CISH gene is an insertion, deletion, substitution, or any combination thereof. In some embodiments, the genomic disruption in the CISH gene is an endonuclease-mediated indel. In some embodiments, the inactivating nucleic acid mutation in the CISH gene is an endonuclease-mediated indel, such as a targeted knockout obtainable by a CRISPR-Cas system (e.g., Cas9, Cas12 or MAD7) designed to target CISH.

In some embodiments, the genomic disruption of the endogenous CISH gene is in exon 3, exon 4, or a combination thereof. In some embodiments, the genomic disruption is an inactivating nucleic acid mutation in exon 3, exon 4, or any combination thereof. In some embodiments, the genomic disruption is within a target sequence in the CISH gene that comprises the nucleotide sequence of any one of SEQ ID NO: 15, SEQ ID NO: 16 or SEQ ID NO: 24 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

Assays for determining the relative expression of a gene or function of a protein encoded by the gene in modified cell (e.g., T-iγδT-cell) can be performed using various methods. For example, the relative expression of a CISH gene in a modified γδT-cell (e.g., T-iγδT-cell) relative to an unmodified γδT-cell (e.g., an unmodified T-iγδT-cell) or relative to the γδT-cell (e.g., T-iγδT-cell) before modification can be determined using qPCR, dPCR, ddPCR, or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### C. Bcl-2 Interacting Mediator of Cell Death (Bim) Genomic Disruption

The present disclosure provides modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising one, two, three, four, five, six, or seven genomic disruptions and/or one, two, three, or four of exogenous polynucleotide incorporations, one of which is a genomic disruption of the endogenous Bcl-2 interacting mediator of cell death (BIM) gene. In some embodiments, the modified cell is a modified T-iγδT cell comprising a genomic disruption of the endogenous BIM gene. In some embodiments, the modified cell is a γδT-cell that is differentiated from a modified (e.g., alone or with any one or more genomic disruptions in SOCS1, CISH, FAS, B2M, CIITA, and TRAC, and/or any one or more incorporations of B2M-HLA-E and signal converter) or unmodified γδT-iPSC, modified or unmodified T-iHSC, modified or unmodified T-iCLP, or modified or unmodified immature T-iγδT cell. In some embodiments, the modified cell is from a primary γδT-cell.

In some embodiments, the genomic disruption in the BIM gene is an inactivating nucleic acid mutation (e.g., knock out). In some embodiments, the inactivating nucleic acid mutation in the BIM gene is an insertion, deletion, substitution, or any combination thereof. In some embodiments, the genomic disruption in the BIM gene is an endonuclease-mediated indel.

In some embodiments, the inactivating nucleic acid mutation in the BIM gene is an endonuclease-mediated indel such as a targeted knockout obtainable by a CRISPR-Cas system (e.g., Cas9, Cas12 or MAD7) designed to target BIM.

In some embodiments, the genomic disruption of the endogenous BIM gene is in exon 2A. In some embodiments, the genomic disruption is an inactivating nucleic acid mutation in exon 2A. In some embodiments, the genomic disruption of the endogenous BIM gene in the modified cell reduces the expression and/or function of all splice variant of BIM, BIM_{EL}, BIM_{L} and BIM_{S}, relative to an unmodified control.

In some embodiments, the genomic disruption is within a target sequence in BIM that comprises the nucleotide sequence of any one of SEQ ID NO: 18 and SEQ ID NO: 19, or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

In some embodiments, the genomic disruption of the endogenous BIM gene is in exon 2C. In some embodiments, the genomic disruption is an inactivating nucleic acid mutation in exon 2C.

In some embodiments, the genomic disruption of the endogenous BIM gene in the modified cell reduces the expression and/or function of a splice variant of BIM, BIM_{EL}, relative to an unmodified control. In some embodiments, the genomic disruption of the endogenous BIM gene reduces the expression and/or function of a splice variant of BIM, BIM_{L}, relative to an unmodified control. In some embodiments, the genomic disruption of the endogenous BIM gene reduces the expression and/or function of a splice variants BIM_{EL} and BIM_{L} relative to an unmodified control.

In some embodiments, the genomic disruption of the endogenous BIM gene in the modified cell retains the expression and/or function of a splice variant of BIM, BIMₛ, relative to an unmodified control.

In some embodiments, the genomic disruption of the endogenous BIM gene reduces the expression and/or function of a splice variants BIM_{EL} and BIM_{L}, while retaining the expression and/or function of a splice variant of BIM, BIMₛ, relative to an unmodified control.

In some embodiments, the genomic disruption is within a target sequence in BIM that comprises the nucleotide sequence of any one of SEQ ID NO: 14, SEQ ID NO: 20, SEQ ID NO: 21, or SEQ ID NO: 22 or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

Assays for determining the relative expression of a gene or function of a protein in a modified cell can be performed using various methods. Similarly, assays for determining the relative expression or function of a splice variant can be performed using various methods. For example, the relative expression of BIM_{S}, BIM_{EL} and BIM_{L} in a modified cell (e.g., T-iγδT-cell) relative to an unmodified control (e.g., an unmodified T-iγδT-cell) can be determined using qPCR, dPCR, ddPCR, or nucleotide sequencing (e.g., next generation sequencing). Additionally, the relative expression of BIM_{S}, BIM_{EL} and BIM_{L} in a modified γδT-cell (e.g., T-iγδT-cell) relative to each other in the modified γδT-cell (e.g., T-iγδT-cell) can be determined using qPCR, dPCR, ddPCR, or nucleotide sequencing (e.g., next generation sequencing).

### D. Fas Cell Surface Death Receptor (Fas) Genomic Disruption

The present disclosure provides modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising one, two, three, four, five, six, or seven genomic disruptions and/or one, two, three, or four of exogenous polynucleotide incorporations, one of which is a genomic disruption of the endogenous FAS cell surface death receptor (FAS) gene. In some embodiments, the modified cell is a modified T-iγδT cell comprising a genomic disruption of the endogenous FAS gene. In some embodiments, the modified cell is a γδT-cell that is differentiated from a modified (e.g., with any one or more genomic disruptions in SOCS1, CISH, BIM, B2M, CIITA, and TRAC, and/or any one or more incorporations of B2M-HLA-E and signal converter) or unmodified γδT-iPSC, modified or unmodified T-iHSC, modified or unmodified T-iCLP, or modified or unmodified immature T-iγδT cell. In some embodiments, the genomic disruptions are of at least two or all three of SOCS1, CISH, and BIM, and further in FAS, and optionally further in CIITA and/or B2M. In some embodiments, the modified cell is from a primary γδT-cell.

In some embodiments, the modified cell comprises a genomic disruption in an endogenous FAS gene that suppresses or eliminates expression of the FAS gene relative to an unmodified control. In some embodiments, the genomic disruption in the FAS gene is an inactivating nucleic acid mutation (e.g., knock out). In some embodiments, the inactivating nucleic acid mutation in the FAS gene is an insertion, deletion, substitution, or any combination thereof. In some embodiments, the genomic disruption in the FAS gene is an endonuclease-mediated indel. In some embodiments, the inactivating nucleic acid mutation in the FAS gene is an endonuclease-mediated indel, such as a targeted knockout obtainable by a CRISPR-Cas system (e.g., Cas9, Cas12 or MAD7) designed to target FAS.

In some embodiments, the genomic disruption of the endogenous FAS gene is in exon 1. In some embodiments, the genomic disruption is an inactivating nucleic acid mutation in exon 1. In some embodiments, the genomic disruption is within a target sequence in FAS that comprises the nucleotide sequence of SEQ ID NO: 62 or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

Assays for determining the relative expression of a gene or function of a protein encoded by the gene in a modified cell (e.g., T-iγδT-cell) can be performed using various methods. For example, the relative expression of the FAS gene in a modified γδT-cell (e.g., T-iγδT-cell) relative to an unmodified γδT-cell (e.g., an unmodified T-iγδT-cell) or relative to the γδT-cell (e.g., T-iγδT-cell) before modification can be determined using quantitative polymerase chain reaction (qPCR), digital PCR (dPCR), droplet digital PCR (ddPCR), or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### E. β-2-MICROGLOBULIN (B2M) GENOMIC DISRUPTION

The present disclosure provides modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising one, two, three, four, five, six, or seven genomic disruptions and/or one, two, three, or four of exogenous polynucleotide incorporations, one of which is a genomic disruption of the endogenous β-2-Microglobulin (B2M) gene. In some embodiments, the modified cell is a modified T-iγδT cell comprising a genomic disruption of the endogenous B2M gene. In some embodiments, the modified cell is a γδT-cell that is differentiated from a modified (e.g., with any one or more genomic disruptions in SOCS1, CISH, BIM, FAS, CIITA, and TRAC, and/or any one or more incorporations of B2M-HLA-E and signal converter) or unmodified γδT-iPSC, modified or unmodified T-iHSC, modified or unmodified T-iCLP, or modified or unmodified immature T-iγδT cell. In some embodiments, the genomic disruptions are of at least two or all three of SOCS1, CISH, and BIM, and further in CIITA and/or B2M, and optionally further in FAS. In some embodiments, the modified cell is from a primary γδT-cell.

In some embodiments, the modified cell comprises a genomic disruption in an endogenous B2M gene that suppresses or eliminates expression of the B2M gene relative to an unmodified control. In some embodiments, the genomic disruption in the B2M gene is an inactivating nucleic acid mutation (e.g., knock out). In some embodiments, the inactivating nucleic acid mutation in the B2M gene is an insertion, deletion, substitution, or any combination thereof. In some embodiments, the genomic disruption in the B2M gene is an endonuclease-mediated indel. In some embodiments, the inactivating nucleic acid mutation in the B2M gene is an endonuclease-mediated indel, such as a targeted knockout obtainable by a CRISPR-Cas system (e.g., Cas9, Cas12 or MAD7) designed to target B2M.

In some embodiments, the genomic disruption of the endogenous B2M gene is in exon 2. In some embodiments, the genomic disruption is an inactivating nucleic acid mutation in exon 2. In some embodiments, the genomic disruption is within a target sequence in B2M that comprises the nucleotide sequence of SEQ ID NO: 56 or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

Assays for determining the relative expression of a gene or function of a protein encoded by the gene in a modified cell (e.g., T-iγδT-cell) can be performed using various methods. For example, the relative expression of the B2M gene in a modified γδT-cell (e.g., T-iγδT-cell) relative to an unmodified γδT-cell (e.g., an unmodified T-iγδT-cell) or relative to the γδT-cell (e.g., T-iγδT-cell) before modification can be determined using quantitative polymerase chain reaction (qPCR), digital PCR (dPCR), droplet digital PCR (ddPCR), or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### F. Class Ii Transactivator (Ciita) Genomic Disruption

The present disclosure provides modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising one, two, three, four, five, six, or seven genomic disruptions and/or one, two, three, or four of exogenous polynucleotide incorporations, one of which is a genomic disruption of the endogenous Class II transactivator (CIITA) gene. In some embodiments, the modified cell is a modified T-iγδT cell comprising a genomic disruption of the endogenous CIITA gene. In some embodiments, the modified cell is a γδT-cell that is differentiated from a modified (e.g., with any one or more genomic disruptions in SOCIS1, CISH, BIM, FAS, B2M, and TRAC, and/or any one or more incorporations of B2M-HLA-E and signal converter) or unmodified γδT-iPSC, modified or unmodified T-iHSC, modified or unmodified T-iCLP, or modified or unmodified immature T-iγδT cell. In some embodiments, the genomic disruptions are of at least two or all three of SOCS1, CISH, and BIM, and further in CIITA and/or B2M, and optionally further in FAS. In some embodiments, the modified cell is from a primary γδT-cell.

In some embodiments, the modified cell comprises a genomic disruption in an endogenous CIITA gene that suppresses or eliminates expression of the CIITA gene relative to an unmodified control. In some embodiments, the genomic disruption in the CIITA gene is an inactivating nucleic acid mutation (e.g., knock out). In some embodiments, the inactivating nucleic acid mutation in the CIITA gene is an insertion, deletion, substitution, or any combination thereof. In some embodiments, the genomic disruption in the CIITA gene is an endonuclease-mediated indel. In some embodiments, the inactivating nucleic acid mutation in the CIITA gene is an endonuclease-mediated indel, such as a targeted knockout obtainable by a CRISPR-Cas system (e.g., Cas9, Cas12 or MAD7) designed to target CIITA.

In some embodiments, the genomic disruption of the endogenous CIITA gene is in exon 3. In some embodiments, the genomic disruption is an inactivating nucleic acid mutation in exon 3. In some embodiments, the genomic disruption is within a target sequence in CIITA that comprises the nucleotide sequence of SEQ ID NO: 59 or a nucleotide sequence having at least 70% (e.g., at least 70%, 75%, 80%, 85%, 90%, 91%, 92%, 93%, 94%, 95%, 96%, 97%, 98%, 99% or 100%) identity thereto.

Assays for determining the relative expression of a gene or function of a protein encoded by the gene in a modified cell (e.g., T-iγδT-cell) can be performed using various methods. For example, the relative expression of the CIITA gene in a modified γδT-cell (e.g., T-iγδT-cell) relative to an unmodified γδT-cell (e.g., an unmodified T-iγδT-cell) or relative to the γδT-cell (e.g., T-iγδT-cell) before modification can be determined using quantitative polymerase chain reaction (qPCR), digital PCR (dPCR), droplet digital PCR (ddPCR), or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### G. Trac Genomic Disruption

The present disclosure provides modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising one, two, three, four, five, six or seven genomic disruptions and/or one, two or three of exogenous polynucleotide incorporations, one of which is a genomic disruption in TRAC gene. In some embodiments, the modified cell is a modified T-iTST cell comprising a genomic disruption in the endogenous TRAC gene. In some embodiments, the modified cell is a γδT-cell that is differentiated from a modified γδT-iPSC (e.g., with any one or more genomic disruptions in SOCIS1, CISH, BIM, FAS, B2M, and CIITA, and/or any one or more incorporations of B2M-HLA-E and signal converter) or unmodified γδT-iPSC, modified or unmodified T-iHSC, modified or unmodified T-iCLP, or modified or unmodified immature T-iγδT cell. In some embodiments, the modified cell is from a primary γδT-cell.

In some embodiments, the modified cell comprises a genomic disruption in an endogenous TRAC gene that suppresses or eliminates expression of the TRAC gene relative to an unmodified control. In some embodiments, the genomic disruption in the TRAC gene is an inactivating nucleic acid mutation (e.g., knock out). In some embodiments, the inactivating nucleic acid mutation in the TRAC gene is an insertion, deletion, substitution, or any combination thereof. In some embodiments, the genomic disruption in the TRAC gene is an endonuclease-mediated indel. In some embodiments, the inactivating nucleic acid mutation in the TRAC gene is an endonuclease-mediated indel, such as a targeted knockout obtainable by a CRISPR-Cas system (e.g., Cas9, Cas12 or MAD7) designed to target TRAC.

Assays for determining the relative expression of a gene or function of a protein encoded by the gene in a modified cell (e.g., T-iγδT-cell) can be performed using various methods. For example, the relative expression of the SOCIS1, CISH, BIM, FAS, B2M, CIITA or TRAC gene in a modified γδT-cell (e.g., T-iγδT-cell) relative to an unmodified γδT-cell (e.g., an unmodified T-iγδT-cell) or relative to the γδT-cell (e.g., T-iγδT-cell) before modification can be determined using quantitative polymerase chain reaction (qPCR), digital PCR (dPCR), droplet digital PCR (ddPCR), or nucleotide sequencing (e.g., next generation sequencing). ddPCR can be performed using suitable nucleic acid quantification kits or instruments, including e.g., Qubit BR dsDNA assay (Thermo Fisher Scientific), Qubit HS dsDNA assay (Thermo Fisher Scientific), Nanodrop spectrophotometer (Thermo Fischer Scientific), Stunner spectrophotometer (Unchained Labs), and Lunatic spectrophotometer (Unchained Labs). Nucleotide sequencing can be performed using NextSeq (Ilumina), GridION (Oxford Nanopore Technologies), Revio System (Pacific Biosciences), and the like.

### H. B2M-Hla-E Incorporation

The present disclosure provides modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising one, two, three, four, five, six or seven genomic disruptions and/or one, two, three or four of exogenous polynucleotide incorporations, one of which is an incorporation of B2M-HLA-E. In some embodiments, the modified cell is a modified T-iγδT cell comprising an exogenous polynucleotide encoding B2M-HLA-E. In some embodiments, the modified cell is a γδT-cell that is differentiated from a modified iPSC (e.g., alone or with any one or more genomic disruptions in SOCS1, CISH, BIM, FAS, B2M, CIITA, TRAC, and/or any one or more incorporations of signal converter). In some embodiments, the modified cell is from a primary γδT-cell.

In some embodiments, the modified cell comprises an exogenous polynucleotide encoding B2M-HLA-E which results in the expression of B2M-HLA-E relative to an unmodified control. In some embodiments, the exogenous polynucleotide encoding B2M-HLA-E is incorporated to the modified cell by knock-in. In some embodiments, the knock-in is a targeted knock-in obtainable by a CRISPR-Cas system (e.g., Cas9, Cas12 or MAD7) targeting a genomic locus of interest, in combination with a donor template comprising the exogenous polynucleotide for integration into the genomic locus of interest by homologous-directed repair. In some embodiments, the exogenous polynucleotide encoding B2M-HLA-E is inserted into a B2M locus. In some embodiments, the exogenous polynucleotide encoding B2M-HLA-E is incorporated to the modified cell by a lentiviral vector.

In some embodiments, B2M-HLA-E is a fusion protein comprising at least a portion of B2M fused with at least of a portion of HLA-E. In some embodiments, the B2M-HLA-E comprises an amino acid sequence of SEQ ID NO: 85. In some embodiments, the B2M-HLA-E consists essentially of or consists of an amino acid sequence of SEQ ID NO: 85.

In some embodiments, the CAG promoter was used to drive the expression from the exogenous polynucleotide encoding B2M-HLA-E.

### I. Signal Converter

The present disclosure provides modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iTST cells))) comprising one, two, three, four, five, six or seven genomic disruptions and/or one, two, three or four of exogenous polynucleotide incorporations, one of which is an incorporation of signal converter (e.g., IFNγ signal converter). In some embodiments, the modified cell is a modified T-iγδT cell comprising one or more exogenous polynucleotides encoding a signal converter (e.g., IFNγ signal converter). In some embodiments, the modified cell is a γδT-cell that is differentiated from a modified iPSC (e.g., alone or with any one or more genomic disruptions in SOCS1, CISH, BIM, FAS, B2M, CIITA, TRAC, and/or an incorporation of B2M-HLA-E). In some embodiments, the modified cell is from a primary γδT-cell.

In some embodiments, the signal converter is a chimeric structure of two or more different cytokine receptors, wherein the extracellular structure of the signal converter is from one cytokine receptor A and the intracellular structure of the signal converter is from another cytokine receptor B, the binding of cytokine A with the extracellular structure of the cytokine receptor A will transfer, transit or convert the intracellular signaling via the intracellular structure of the cytokine receptor B, which activates or improves the signal transducer and activator of transcription (STAT) signaling in the cell which is not, little or less activated by the natural cytokine receptor A when binding with cytokine A. In some embodiments, the extracellular structure of the signal converter comprises one or more extracellular domains, from same or different cytokine receptor, or from one or more cytokine receptors. In some embodiments, the intracellular structure of the signal converter comprises one or more intracellular domains, from same or different cytokine receptor, or from one or more cytokine receptors.

In some embodiment, the extracellular structure of the signal converter is from interferon gamma receptor. In some embodiment, the intracellular structure of the signal converter is from interleukin-2 receptor. In some embodiments, the extracellular structure of the signal converter and the intracellular structure is linked via transmembrane structure, wherein the transmembrane structure comprises transmembrane domain from same or different cytokine receptors where the extracellular structure of the signal converter and the intracellular structure are from. In some embodiments, the transmembrane structure comprises transmembrane domain from same cytokine receptors where the extracellular structure of the signal converter or the intracellular structure is from. In some embodiments, the signal converter comprises membrane-proximal region between the ECD and the intracellular domain, said membrane-proximal region is from the same or different cytokine receptors where the extracellular structure of the signal converter and the intracellular structure is from. In some embodiments, the membrane-proximal region is from the same cytokine receptor where the extracellular domain of the signal converter is from, or and the same cytokine receptor where the intracellular domain of the signal converter is from. In some embodiments, the signal converter further comprises a signal peptide that enables trafficking of the signal converter to the cell membrane. In some embodiments, the signal peptide is removed from the signal converter after localization to the membrane.

### Ifnγ Signal Converter

In some embodiment, the extracellular structure of the IFNγ signal converter comprises both the extracellular domain (ECD) of interferon gamma receptor 1 (IFNGR1) and the extracellular domain (ECD) of interferon gamma receptor 2 (IFNGR2). In some embodiments, the intracellular structure of the IFNγ signal converter comprises the intracellular domain of interleukin-2 receptor subunit gamma (IL2RG) and the intracellular domain of interleukin-2 receptor subunit beta (IL2RB), wherein one ECD of IFNGR1 and IFNGR2 is linked or fused with one ICD of IL2RG and TL2RB to form a first IFNγ fusion protein subunit, and the other ECD is linked or fused with the other ICD to form a second IFNγ fusion protein subunit. In some embodiments, the IFNγ signal converter further comprises transmembrane domains of IFNGR1 and IFNGR2, or the IFNγ signal converter further comprises transmembrane domains of IL2RG and IL2RB. In some embodiments, the IFNγ signal converter further comprises membrane-proximal regions of IFNGR1 and IFNGR2, or the signal converter further comprises membrane-proximal regions of IL2RG and IL2RB. In some embodiments, one of the IFNGR1 or IFNGR2 extracellular domain is fused sequentially with transmembrane domain, and intracellular domain of IL2RG to form the first IFNγ fusion protein subunit, the other IFNGR2 or IFNGR1 extracellular domain is fused sequentially with transmembrane domain, and intracellular domain of IL2RB to form the second IFNγ fusion protein subunit. In some embodiment, one of the IFNGR1 or IFNGR2 extracellular domain is fused sequentially with membrane-proximal region, transmembrane domain, and intracellular domain of IL2RG to form the first IFNγ fusion protein subunit, the other IFNGR2 or IFNGR1 extracellular domain is fused sequentially with membrane-proximal region, transmembrane domain, and intracellular domain of IL2RB to form the second IFNγ fusion protein subunit. In some embodiments, the first IFNγ fusion protein subunit and the second IFNγ fusion protein subunit of the IFNγ signal converter further comprise a signal peptide.

In some embodiments, the signal peptide of the first IFNγ fusion protein subunit comprises the amino acid of SEQ ID NO: 79 or SEQ ID NO: 80, and the signal peptide of the second IFNγ fusion protein subunit comprises the amino acid of SEQ ID NO: 79 or SEQ ID NO: 80.

In some embodiments, the amino acid sequence of the signal peptide of the first IFNγ fusion protein subunit is SEQ ID NO: 79, and the amino acid of the signal peptide of the second IFNγ fusion protein subunit is SEQ ID NO: 80. In some embodiments, the amino acid sequence of extracellular domain of the first IFNγ fusion protein subunit is SEQ ID NO: 63, and the amino acid sequence of extracellular domain of the second IFNγ fusion protein subunit is SEQ ID NO: 64. In some embodiments, the amino acid sequence of intracellular domain of the first IFNγ fusion protein subunit is SEQ ID NO: 69, and the amino acid sequence of intracellular domain of the second IFNγ fusion protein subunit is SEQ ID NO: 70. In some embodiments, the amino acid sequence of transmembrane domain of the first IFNγ fusion protein subunit is SEQ ID NO: 67, and the amino acid sequence of transmembrane domain of the second IFNγ fusion protein subunit is SEQ ID NO: 68. The amino acid sequence of membrane-proximal region of the first IFNγ fusion protein subunit is SEQ ID NO: 65, and the amino acid sequence of membrane-proximal region of the second IFNγ fusion protein subunit is SEQ ID NO: 66.

In some embodiments, the present disclosure provides an IFNγ signal converter, wherein the IFNγ signal converter comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB). In some embodiments, the first IFNγ fusion protein subunit of the IFNγ signal converter comprises an amino acid sequence SEQ ID NO: 71, and the second IFNγ fusion protein subunit of the IFNγ signal converter comprises an amino acid sequence of SEQ ID NO: 72. In some embodiments, the first IFNγ fusion protein subunit of the IFNγ signal converter consists essentially of or consists of an amino acid sequence SEQ ID NO: 71, and the second IFNγ fusion protein subunit of the IFNγ signal converter consists essentially of or consists of an amino acid sequence of SEQ ID NO: 72. In some embodiments, the first exogenous polynucleotide encoding an amino acid sequence comprising SEQ ID NO: 81 (the first IFNγ fusion protein subunit with signal peptide), and the second exogenous polynucleotide encoding an amino acid sequence comprising SEQ ID NO: 82 ((the second IFNγ fusion protein subunit with signal peptide)).

In some embodiments, the modified cell comprises one or more exogenous polynucleotides encoding an IFNγ signal converter, which results in the expression of IFNγ signal converter relative to an unmodified control. In some embodiments, the one or more exogenous polynucleotides encoding the IFNγ signal converter are incorporated to the modified cell by knock-in. In some embodiments, the knock-in is a targeted knock-in obtainable by a CRISPR-Cas system (e.g., Cas9, Cas12 or MAD7) targeting a genomic locus of interest, in combination with a donor template comprising the exogenous polynucleotide for integration into the genomic locus of interest by homologous-directed repair. In some embodiments, one or more exogenous polynucleotides encoding the IFNγ signal converter are incorporated to the modified cell by a lentiviral vector.

In some embodiments, the first exogenous polynucleotide encoding the first IFNγ fusion protein subunit of the IFNγ signal converter is inserted into a SOCS1 locus. In some embodiments, the second exogenous polynucleotide encoding the second IFNγ fusion protein subunit of the IFNγ signal converter is inserted into a ROSA26 locus.

In some embodiments, the first exogenous polynucleotide encoding the first IFNγ fusion protein subunit of the IFNγ signal converter is inserted into a ROSA26 locus. In some embodiments, the second exogenous polynucleotide encoding the second IFNγ fusion protein subunit of the IFNγ signal converter is inserted into a SOCS1 locus.

In some embodiments, the CAG promoter was used to drive the expression from the first exogenous polynucleotide encoding the first IFNγ fusion protein subunit and the second exogenous polynucleotide encoding the second IFNγ fusion protein subunit.

### J. Single, Double, Triple, Quadruple, Quintuple, and Sextuple Genomic Disruptions

The present disclosure provides single-gene, double-gene, triple-gene, quadruple-gene, quintuple-gene, or sextuple-gene modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising one or more genomic disruptions in the following genes: the endogenous SOCS1 gene, CISH gene, BIM gene, FAS gene, B2M gene, and CIITA gene. The present disclosure provides double-gene, triple-gene, quadruple-gene, quintuple-gene, or sextuple-gene modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising a genomic disruption of the endogenous SOCS1 and CISH genes, optionally a BIM gene, optionally a FAS gene, optionally a B2M gene, and, optionally a CIITA gene. In some embodiments, the modified cell is a T-iγδT cell comprising a genomic disruption of the endogenous SOCS1 and CISH genes. In some embodiments, the modified cell is a T-iγδT cell comprising a genomic disruption of the endogenous SOCS1, CISH, and BIM genes. In some embodiments, the modified cell is a T-iγδT cell comprising a genomic disruption of the endogenous SOCS1, CISH, BIM, and FAS genes. In some embodiments, the modified cell is a T-iγδT cell comprising a genomic disruption of the endogenous SOCS1, CISH, BIM, B2M, and CIITA genes. In some embodiments, the modified cell is a T-iγδT cell comprising a genomic disruption of the endogenous SOCS1, CISH, BIM, FAS, B2M, and CIITA genes. In some embodiments, the modified cell is differentiated from a γδT-iPSC, T-iHSC, T-iCLP, or immature T-iγδT cell, where the modifications can occur at any of these cell development stages and be carried through to γδT maturity, or wherein the modifications can occur at γδT maturity, i.e., at the T-iγδT stage.

In some embodiments, the modified cell comprises a genomic disruption in an endogenous SOCS1 and CISH genes that suppresses or eliminates expression of the SOCS1 and CISH genes.

The present disclosure provides modified iPSC-derived γδT-cells (and their precursors (e.g., γδT-iPSC, γδT-iHSC, γδT-iCLP)) comprising a genomic disruption in an endogenous SOCS1 gene that suppresses or eliminates expression of SOCS1, a genomic disruption in an endogenous CISH gene that suppresses or eliminates expression of CISH, and, optionally, a genomic disruption in an endogenous BIM gene that suppresses or eliminates expression of BIM.

The present disclosure provides modified iPSC-derived γδT-cells (and their precursors (e.g., γδT-iPSC, γδT-iHSC, γδT-iCLP)) comprising a genomic disruption in an endogenous SOCS1 gene that suppresses or eliminates expression of SOCS1, a genomic disruption in an endogenous CISH gene that suppresses or eliminates expression of CISH, optionally a genomic disruption in an endogenous BIM gene that suppresses or eliminates expression of BIM, and, optionally a genomic disruption in an endogenous FAS gene that suppresses or eliminates expression of FAS.

The present disclosure provides modified iPSC-derived γδT-cells (and their precursors (e.g., γδT-iPSC, γδT-iHSC, γδT-iCLP)) comprising a genomic disruption in an endogenous SOCS1 gene that suppresses or eliminates expression of SOCS1, a genomic disruption in an endogenous CISH gene that suppresses or eliminates expression of CISH, optionally a genomic disruption in an endogenous BIM gene that suppresses or eliminates expression of BIM, optionally a genomic disruption in an endogenous B2M gene that suppresses or eliminates expression of B2M, and, optionally a genomic disruption in an endogenous CIITA gene that suppresses or eliminates expression of CIITA.

The present disclosure provides modified iPSC-derived γδT-cells (and their precursors (e.g., γδT-iPSC, γδT-iHSC, γδT-iCLP)) comprising a genomic disruption in an endogenous SOCS1 gene that suppresses or eliminates expression of SOCS1, a genomic disruption in an endogenous CISH gene that suppresses or eliminates expression of CISH, optionally a genomic disruption in an endogenous BIM gene that suppresses or eliminates expression of BIM, optionally a genomic disruption in an endogenous FAS gene that suppresses or eliminates expression of FAS, optionally a genomic disruption in an endogenous B2M gene that suppresses or eliminates expression of B2M, and, optionally a genomic disruption in an endogenous CIITA gene that suppresses or eliminates expression of CIITA.

In some embodiments, the expression and/or function of BIM_{EL} and BIM_{L} splice variants are reduced in the modified cell relative to that in an unmodified control. In some embodiments, the expression and/or function of BIM_{S} splice variant is retained in the modified cell.

In some embodiments, the genomic disruption is made by administering guide RNAs to the cell, optionally wherein the guide RNAs are single-guide RNAs (sgRNAs), and wherein an endonuclease or a nucleic acid encoding an endonuclease is also administered to the cell. Further information on the guide RNA and endonuclease could be found in other sections of the present disclosure, e.g., Section V: Gene editing systems.

Also encompassed are populations of cells comprising the single-, double-, triple-, quadruple-, quintuple-, or sextuple-genomic disruptions described herein.

In some embodiments, the cells are stored for a length of time before use. In some embodiments, the time is weeks or months. In some embodiments, the time is months or years.

In some embodiments, the cells are stored frozen in liquid nitrogen at −196 degrees Celsius.

### III. Modified Cells Comprising CAR, e.g., Anti-CD19 CAR

The modified cells herein, e.g., the modified cells described in Section II, further comprise an exogenous polynucleotide encoding a chimeric antigen receptor (CAR), e.g., a CAR which specifically binds human CD19 (anti-CD19 CAR).

The present disclosure provides modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising one, two, three, four, five, six or seven genomic disruptions and/or one, two, three or four of exogenous polynucleotide incorporations, one of which is an incorporation of anti-CD19 CAR. In some embodiments, the modified cell is a modified T-iγδT cell comprising an exogenous polynucleotide encoding anti-CD19 CAR. In some embodiments, the modified cell is a γδT-cell that is differentiated from a modified iPSC (e.g., with any one or more genomic disruptions in SOCS1, CISH, BIM, FAS, B2M, CIITA, TRAC, and/or any one or more incorporations of B2M-HLA-E and signal converter). In some embodiments, the modified cell is from a primary γδT-cell.

In some embodiments, the modified cell comprises an exogenous polynucleotide encoding anti-CD19 CAR which results in the expression of anti-CD19 CAR relative to an unmodified control. In some embodiments, the exogenous polynucleotide encoding anti-CD19 CAR is incorporated to the modified cell by knock-in. In some embodiments, the knock-in is a targeted knock-in obtainable by a CRISPR-Cas system (e.g., Cas9, Cas12 or MAD7) targeting a genomic locus of interest, in combination with a donor template comprising the exogenous polynucleotide for integration into the genomic locus of interest by homologous-directed repair. In some embodiments, the exogenous polynucleotide encoding anti-CD19 CAR is inserted into an AAVS1 locus, which is a safe harbor. In some embodiments, the exogenous polynucleotide encoding anti-CD19 CAR is incorporated to the modified cell by a lentiviral vector.

In some embodiments, anti-CD19 CAR comprising an amino acid sequence of SED ID NO: 83. In some embodiments, anti-human CLL1 CAR comprises an amino acid sequence of SED ID NO: 84. In some embodiments, anti-CD19 CAR consists essentially of or consists of an amino acid sequence of SEQ ID NO: 83 or SEQ ID NO: 84.

In some embodiments, the anti-CD19 CAR further comprises a signal peptide (e.g., comprising an amino acid sequence of SEQ ID NO: 73) that enables trafficking of the CAR to the cell membrane. In some embodiments, the signal peptide is removed from the signal converter after localization to the membrane.

In some embodiments, the CAG promoter was used to drive the expression from the exogenous polynucleotide encoding anti-CD19 CAR.

### IV. Functional Enhancement of Proliferation and Killing Activity

In some embodiments, the present disclosure presents modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) with functional enhancement of proliferation and/or tumor killing activity. More specifically, the functional enhancement is achieved by activating or improved activation of signal transducer and activator of transcription (STAT) signaling in the cell. Also disclosed is the method of improving the proliferation and/or tumor killing activity of γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))), by activating or improved activation of signal transducer and activator of transcription (STAT) signaling in the cell.

In some embodiments, the method of functional enhancement of proliferation and/or tumor killing activity is achieved by introducing one or more exogenous polynucleotides encoding a signal converter (IFNγ signal converter) into the iPSC or γδT-cells.

Further information on the signal converter, e.g., IFNγ signal converter could be found in other sections of the present disclosure, e.g., Section II (I): Signal converter.

In some embodiments, the amino acid sequence of ECD of the first IFNγ fusion protein subunit is SEQ ID NO: 63, and the amino acid sequence of ECD of the second IFNγ fusion protein subunit is SEQ ID NO: 64. In some embodiments, the amino acid sequence of intracellular domain of the first IFNγ fusion protein subunit is SEQ ID NO: 69, and the amino acid sequence of intracellular domain of the second IFNγ fusion protein subunit is SEQ ID NO: 70. In some embodiments, the amino acid sequence of transmembrane domain of the first IFNγ fusion protein subunit is SEQ ID NO: 67, and the amino acid sequence of transmembrane domain of the second fusion protein subunit is SEQ ID NO: 68. The amino acid sequence of membrane-proximal region of the first IFNγ fusion protein subunit is SEQ ID NO: 65, and the amino acid sequence of membrane-proximal region of the second IFNγ fusion protein subunit is SEQ ID NO: 66.

In some embodiments, the present disclosure provides the modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) comprising an IFNγ signal converter, wherein the IFNγ signal converter comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB), optionally further comprising an exogenous polynucleotide encoding a chimeric antigen receptor (CAR), optionally wherein the exogenous polynucleotide encoding the CAR is inserted into an AAVS1 locus, optionally wherein the exogenous polynucleotide encodes the CAR which specifically binds human CD19, optionally wherein the CAR which specifically binds human CD19 comprises the amino acid of SEQ ID NO: 83 or SEQ ID NO: 84. In some embodiments, the first IFNγ fusion protein subunit of the IFNγ signal converter comprises an amino acid sequence SEQ ID NO: 71, and the second IFNγ fusion protein subunit of the IFNγ signal converter comprises an amino acid sequence of SEQ ID NO: 72. In some embodiments, the first IFNγ fusion protein subunit of the IFNγ signal converter consists essentially of or consists of an amino acid sequence SEQ ID NO: 71, and the second IFNγ fusion protein subunit of the IFNγ signal converter consists essentially of or consists of an amino acid sequence of SEQ ID NO: 72.

In some embodiments, the first exogenous polynucleotide encoding an amino acid sequence comprising SEQ ID NO: 81 (the first IFNγ fusion protein subunit with signal peptide), and the second exogenous polynucleotide encoding an amino acid sequence comprising SEQ ID NO: 82 ((the second IFNγ fusion protein subunit with signal peptide)).

In some embodiments, the amino acid sequence of the first IFNγ fusion protein subunit is SEQ ID NO: 71, and the amino acid sequence of the second IFNγ fusion protein subunit is SEQ ID NO: 72.

The modified γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) with functional enhancement of proliferation and/or tumor killing activity disclosed herein can be previously or further subject to gene editing for suppressing cytokine signaling disclosed in the other sections of this disclosure.

### V. Gene Editing Systems

Disclosed herein are gene editing systems that are optimized to disrupt the genomes of the cells herein (e.g., the γδT cell, iγδT cell, iPSC, iHSC, iCLP cell, or immature iT cell), specifically within the genes of SOCS1; CISH; BIM; SOCS1 and CISH; SOCS1, CISH, and BIM; SOCS1, CISH, and FAS; SOCS1, CISH, BIM, and FAS; B2M, CIITA, CISH, SOCS1, and BIM; B2M, CIITA, CISH, SOCS1, BIM, and FAS.

### A. Clustered Regularly Interspace Short Palindromic Repeats (CRISPR)

In some embodiments, the gene editing system used to modify γδT-cells, e.g., iPSC-derived γδT-cells (and their precursors (e.g., iPSC (e.g., γδT-iPSC), or an intermediate cell differentiated from an iPSC (e.g., γδT-iHSC, γδT-iCLP, and immature T-iγδT cells))) is clustered regularly interspace short palindromic repeats (CRISPR) system comprising one or more guide RNAs and an endonuclease or a nucleic acid encoding a nuclease. The CRISPR gene editing system comprises a guide RNA, including e.g., a single guide RNA (sgRNA) or crRNA, and a nuclease, including e.g., an endonuclease, or a nucleic acid encoding a nuclease, including e.g., a nucleic acid encoding an endonuclease.

In some embodiments, the guide RNA comprises a guide sequence that binds to a target sequence in SOCS1, CISH, BIM, B2M, CIITA, and/or FAS of the cells used herein (e.g., the T-iγδT cell, the γδT-iPSC, T-iHSC, T-iCLP, or immature T-iγδT cell). In some embodiments, the genomic disruption within SOCS1, CISH, BIM, B2M, CIITA, or FAS gene is made by administering a nuclease (e.g., endonuclease), a plurality of nucleases (e.g., endonucleases), or a nucleic acid encoding an endonuclease(s) and a guide RNA or a plurality of guide RNAs to the cells used herein (e.g., the T-iγδT cell, the γδT-iPSC, T-iHSC, T-iCLP, or immature T-iγδT cell).

In some embodiments, the administration of the nuclease and the guide RNA to the cells used herein (e.g., the T-iγδT cell, the γδT-iPSC, T-iHSC, T-iCLP, or immature T-iγδT cell) is performed sequentially in which a guide RNA targeting a first gene (e.g., SOCS1) is administered initially, followed by administration of a guide RNA targeting a second gene (e.g., CISH). In some embodiments, an administration of a guide RNA targeting a third gene (e.g., BIM) can be performed. In some embodiments, an administration of a guide RNA targeting a fourth gene (e.g., FAS) can be performed. In some embodiments, an administration of a guide RNA targeting a fifth gene (e.g., B2M) can be performed. In some embodiments, an administration of a guide RNA targeting a sixth gene (e.g., CIITA) can be performed. The sequential administration of guide RNA targeting a gene can be performed in any order.

Disclosed herein are single guide RNA (sgRNA) or crRNA nucleotide sequences that bind to a target sequence in the SOCS1 gene in which the sgRNA or crRNA nucleotide sequence comprises any one or more of SEQ ID NOs: 5, 29, 11, 37, and a nucleotide sequence having at least 70% (e.g., at least 70%, 75%, 80%, 85%, 90%, 91%, 92%, 93%, 94%, 95%, 96%, 97%, 98%, 99%, or 100%) identity thereto. Disclosed herein are sgRNA or crRNA nucleotide sequences that bind to a target sequence in the CISH gene in which the sgRNA or crRNA nucleotide sequence comprises any one or more of SEQ ID NOs: 3, 27, 4, 28, 12, 38, and a nucleotide sequence having at least 70% (e.g., at least 70%, 75%, 80%, 85%, 90%, 91%, 92%, 93%, 94%, 95%, 96%, 97%, 98%, 99%, or 100%) identity thereto. Disclosed herein are sgRNA or crRNA nucleotide sequences that bind to a target sequence in the BIM gene in which the sgRNA or crRNA nucleotide sequence comprises any one or more of SEQ ID NOs: 2, 26, 6, 32, 7, 33, 8, 34, 9, 35, 10, 36, or a nucleotide sequence having at least 70% (e.g., at least 70%, 75%, 80%, 85%, 90%, 91%, 92%, 93%, 94%, 95%, 96%, 97%, 98%, 99%, or 100%) identity thereto. Disclosed herein are sgRNA nucleotide sequences that bind to a target sequence in the FAS gene in which the sgRNA nucleotide sequence comprises any one or more of SEQ ID NOs: 60, 61, or a nucleotide sequence having at least 70% (e.g., at least 70%, 75%, 80%, 85%, 90%, 91%, 92%, 93%, 94%, 95%, 96%, 97%, 98%, 99%, or 100%) identity thereto. Disclosed herein are sgRNA nucleotide sequences that bind to a target sequence in the B2M gene in which the sgRNA nucleotide sequence comprises any one or more of SEQ ID NOs: 54, 55, or a nucleotide sequence having at least 70% (e.g., at least 70%, 75%, 80%, 85%, 90%, 91%, 92%, 93%, 94%, 95%, 96%, 97%, 98%, 99%, or 100%) identity thereto. Disclosed herein are sgRNA nucleotide sequences that bind to a target sequence in the CIITA gene in which the sgRNA nucleotide sequence comprises any one or more of SEQ ID NOs: 57, 58, or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

Disclosed herein is a gene editing system comprising one or more guide RNA (e.g., sgRNA or crRNA) and an endonuclease or a nucleic acid encoding an endonuclease, in which the one or more guide RNA comprises a guide sequence or a sgRNA/crRNA sequence comprising:
(1) any one of SEQ ID NOs: 5, 29, 11, and 37;
(2) any one of SEQ ID NOs: 3, 27, 4, 28, 12, and 38;
(3) any one of SEQ ID NOs: 2, 26, 6, 32, 7, 33, 8, 34, 9, 35, 10 and 36;
(4) any one of SEQ ID NOs: 60 and 61;
(5) any one of SEQ ID NOs: 54 and 55;
(6) any one of SEQ ID NOs: 57 and 58;
(7) any one of SEQ ID NOs: 5, 29, 11, and 37 and any one of SEQ ID NOs: 3, 27, 4, 28, 12, and 38;
(8) any one of SEQ ID NOs: 5, 29, 11, and 37, any one of SEQ ID NOs: 3, 27, 4, 28, 12, and 38, and any one of SEQ ID NOs: 2, 26, 6, 32, 7, 33, 8, 34, 9, 35, 10 and 36;
(9) any one of SEQ ID NOs: 5, 29, 11, and 37, any one of SEQ ID NOs: 3, 27, 4, 28, 12, and 38, any one of SEQ ID NOs: 2, 26, 6, 32, 7, 33, 8, 34, 9, 35, 10 and 36, and any one of SEQ ID NOs: 60 and 61;
(10) any one of SEQ ID NOs: 5, 29, 11, and 37, any one of SEQ ID NOs: 3, 27, 4, 28, 12, and 38, any one of SEQ ID NOs: 2, 26, 6, 32, 7, 33, 8, 34, 9, 35, 10 and 36, any one of SEQ ID NOs: 54 and 55, and any one of SEQ ID NOs: 57 and 58; or
(11) any one of SEQ ID NOs: 5, 29, 11, and 37, any one of SEQ ID NOs: 3, 27, 4, 28, 12, and 38, any one of SEQ ID NOs: 2, 26, 6, 32, 7, 33, 8, 34, 9, 35, 10 and 36, any one of SEQ ID NOs: 54 and 55, any one of SEQ ID NOs: 57 and 58, and any one of SEQ ID NOs: 60 and 61.

Disclosed herein is a gene editing system comprising one or more guide RNA (e.g., sgRNA or crRNA) and an endonuclease or a nucleic acid encoding an endonuclease, in which the one or more guide RNA comprises a guide sequence or a sgRNA/crRNA sequence comprising:
(1) any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37;
(2) any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38;
(3) any one or more of SEQ ID NOs: 2, 26, 6, 32, 7, 33, 8, 34, 9, 35, 10, and 36, and a sequence differing by no more than 5, 4, 3, 2, or 1 nucleotide(s) from any one or more of SEQ ID NOs: 2, 26, 6, 32, 7, 33, 8, 34, 9, 35, 10 and 36;
(4) any one of SEQ ID NOs: 60, 61, and a sequence differing by no more than 5, 4, 3, 2, or 1 nucleotide(s) from any one or more of SEQ ID NOs: 60 and 61;
(5) any one of SEQ ID NOs: 54, 55, and a sequence differing by no more than 5, 4, 3, 2, or 1 nucleotide(s) from any one or more of SEQ ID NOs: 54 and 55;
(6) any one of SEQ ID NOs: 57, 58, and a sequence differing by no more than 5, 4, 3, 2, or 1 nucleotide(s) from any one or more of SEQ ID NOs: 57 and 58;
(7) any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37 and any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38;
(8) any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37, any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38, and any one or more of SEQ ID NOs: 2, 26, 6, 32, 7, 33, 8, 34, 9, 35, 10, 36, and a sequence differing by no more than 5, 4, 3, 2, or 1 nucleotide(s) from any one or more of SEQ ID NOs: 2, 26, 6, 32, 7, 33, 8, 34, 9, 35, 10, and 36;
(9) any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37, any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38, any one or more of SEQ ID NOs: 2, 26, 6, 32, 7, 33, 8, 34, 9, 35, 10, 36, and a sequence differing by no more than 5, 4, 3, 2, or 1 nucleotide(s) from any one or more of SEQ ID NOs: 2, 26, 6, 32, 7, 33, 8, 34, 9, 35, 10, and 36, and any one of SEQ ID NOs: 60, 61 and a sequence differing by no more than 5, 4, 3, 2, or 1 nucleotide(s) from any one or more of SEQ ID NOs: 60 and 61;
(10) any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37, any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38, any one or more of SEQ ID NOs: 2, 26, 6, 32, 7, 33, 8, 34, 9, 35, 10, 36, and a sequence differing by no more than 5, 4, 3, 2, or 1 nucleotide(s) from any one or more of SEQ ID NOs: 2, 26, 6, 32, 7, 33, 8, 34, 9, 35, 10, and 36, any one of SEQ ID NOs: 54, 55, and a sequence differing by no more than 5, 4, 3, 2, or 1 nucleotide(s) from any one or more of SEQ ID NOs: 54 and 55, and any one of SEQ ID NOs: 57, 58, and a sequence differing by no more than 5, 4, 3, 2, or 1 nucleotide(s) from any one or more of SEQ ID NOs: 57 and 58;
(11) or any one or more of SEQ ID NOs: 5, 29, 11, 37, and a sequence differing by no more than 5, 4, 3, 2, or 1 nucleotide(s) from any one or more of SEQ ID NOs: 5, 29, 11, and 37, any one or more of SEQ ID NOs: 3, 27, 4, 28, 12, 38, and a sequence differing by no more than 5, 4, 3, 2, or 1 nucleotide(s) from any one or more of SEQ ID NOs: 3, 27, 4, 28, 12, and 38, any one or more of SEQ ID NOs: 2, 26, 6, 32, 7, 33, 8, 34, 9, 35, 10, 36, and a sequence differing by no more than 5, 4, 3, 2, or 1 nucleotide(s) from any one or more of SEQ ID NOs: 2, 26, 6, 32, 7, 33, 8, 34, 9, 35, 10, and 36, any one of SEQ ID NOs: 54, 55, and a sequence differing by no more than 5, 4, 3, 2, or 1 nucleotide(s) from any one or more of SEQ ID NOs: 54 and 55, any one of SEQ ID NOs: 57, 58, and a sequence differing by no more than 5, 4, 3, 2, or 1 nucleotide(s) from any one or more of SEQ ID NOs: 57 and 58, and any one of SEQ ID NOs: 60, 61 and a sequence differing by no more than 5, 4, 3, 2, or 1 nucleotide(s) from any one or more of SEQ ID NOs: 60 and 61.

Disclosed herein is a gene editing system comprising one or more sgRNA and an endonuclease or a nucleic acid encoding an endonuclease, in which the one or more sgRNA comprises a guide sequence that binds to a target sequence in a target gene. In some embodiments, the target sequence comprises:
(1) any one or more of SEQ ID NOs: 17, 23, 15, 16, and 24;
(2) any one or more of SEQ ID NOs: 14, 18, 19, 20, 21, and 22;
(3) SEQ ID NO: 62;
(4) SEQ ID NO: 56;
(5) SEQ ID NO: 59;
(6) any one or more of SEQ ID NOs: 17, 23, 15, 16, and 24 and any one or more of SEQ ID NOs: 14, 18, 19, 20, 21, and 22;
(7) any one or more of SEQ ID NOs: 17, 23, 15, 16, and 24, any one or more of SEQ ID NOs: 14, 18, 19, 20, 21, and 22, and SEQ ID NO: 62;
(8) any one or more of SEQ ID NOs: 17, 23, 15, 16, and 24, any one or more of SEQ ID NOs: 14, 18, 19, 20, 21, and 22, SEQ ID NO: 56, and SEQ ID NO: 59; or
(9) any one or more of SEQ ID NOs: 17, 23, 15, 16, and 24, any one or more of SEQ ID NOs: 14, 18, 19, 20, 21, and 22, SEQ ID NO: 62, SEQ ID NO: 56, and SEQ ID NO: 59.

In some embodiments, the target sequence comprises:
(1) any one or more of SEQ ID NOs: 17, 23, 15, 16, 24, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 17, 23, 15, 16, and 24;
(2) any one or more of SEQ ID NOs: 14, 18, 19, 20, 21, 22, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 14, 18, 19, 20, 21, and 22;
(3) any one or more of SEQ ID NO: 62 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 62;
(4) any one or more of SEQ ID NO: 56 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 56;
(5) any one or more of SEQ ID NO: 59 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 59;
(6) any one or more of SEQ ID NOs: 17, 23, 15, 16, 24, and a nucleotide sequence having at least 70% (e.g., at least 70%, 75%, 80%, 85%, 90%, 91%, 92%, 93%, 94%, 95%, 96%, 97%, 98%, or 99%) identity to any one or more of SEQ ID NOs: 17, 23, 15, 16, and 24 and any one or more of SEQ ID NOs: 14, 18, 19, 20, 21, 22, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 14, 18, 19, 20, 21, and 22;
(7) any one or more of SEQ ID NOs: 17, 23, 15, 16, 24, and a nucleotide sequence having at least 70% (e.g., at least 70%, 75%, 80%, 85%, 90%, 91%, 92%, 93%, 94%, 95%, 96%, 97%, 98%, or 99%) identity to any one or more of SEQ ID NOs: 17, 23, 15, 16, and 24, any one or more of SEQ ID NOs: 14, 18, 19, 20, 21, 22, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 14, 18, 19, 20, 21, and 22, and any one or more of SEQ ID NO: 62 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 62;
(8) any one or more of SEQ ID NOs: 17, 23, 15, 16, 24, and a nucleotide sequence having at least 70% (e.g., at least 70%, 75%, 80%, 85%, 90%, 91%, 92%, 93%, 94%, 95%, 96%, 97%, 98%, or 99%) identity to any one or more of SEQ ID NOs: 17, 23, 15, 16, and 24, any one or more of SEQ ID NOs: 14, 18, 19, 20, 21, 22, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 14, 18, 19, 20, 21, and 22, any one or more of SEQ ID NO: 56 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 56, and any one or more of SEQ ID NO: 59 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 59;
(9) any one or more of SEQ ID NOs: 17, 23, 15, 16, 24, and a nucleotide sequence having at least 70% (e.g., at least 70%, 75%, 80%, 85%, 90%, 91%, 92%, 93%, 94%, 95%, 96%, 97%, 98%, or 99%) identity to any one or more of SEQ ID NOs: 17, 23, 15, 16, and 24, any one or more of SEQ ID NOs: 14, 18, 19, 20, 21, 22, and a nucleotide sequence having at least 70% (e.g., at least 70%, 75%, 80%, 85%, 90%, 91%, 92%, 93%, 94%, 95%, 96%, 97%, 98%, 99%, or 100%) identity to any one or more of SEQ ID NOs: 14, 18, 19, 20, 21, and 22, any one or more of SEQ ID NO: 56 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 56, any one or more of SEQ ID NO: 59 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 59, and any one or more of SEQ ID NO: 62 and a nucleotide sequence having at least 70% (e.g., at least 70%, 75%, 80%, 85%, 90%, 91%, 92%, 93%, 94%, 95%, 96%, 97%, 98%, 99%, or 100%) identity to SEQ ID NO: 62.

Suitable endonucleases for use in the disclosed gene editing systems may include endonucleases known in the art, including e.g., a Cas endonuclease. In some embodiments, the Cas endonuclease is a Cas9, Cas12i2, Cas12a, Cas12b, Cas12c, Cas13a (C2c2), Cas13b, or MAD7.

In some embodiments, the endonuclease used in the CRISPR gene editing system is a MAD7 endonuclease; and the target sequence of the guide RNA of the CRISPR gene editing system comprises SEQ ID NO: 22 and/or the guide RNA comprises a sequence differing by no more than 5, 4, 3, 2, or 1 nucleotide(s) from SEQ ID NO: 10. In some embodiments, the endonuclease used in the CRISPR gene editing system is a MAD7 endonuclease; and the target sequence of the guide RNA of the CRISPR gene editing system comprises SEQ ID NO: 23 and/or the guide RNA comprises a sequence differing by no more than 5, 4, 3, 2, or 1 nucleotide(s) from SEQ ID NO: 11. In some embodiments, the endonuclease used in the CRISPR gene editing system is a MAD7 endonuclease; and, the target sequence of the guide RNA of the CRISPR gene editing system comprises SEQ ID NO: 24 and/or the guide RNA comprises a sequence differing by no more than 5, 4, 3, 2, or 1 nucleotide(s) from SEQ ID NO: 12.

In some embodiments, the endonuclease used in the CRISPR gene editing system is a Cas9 endonuclease; and the target sequence of the guide RNA of the CRISPR gene editing system comprises any one of SEQ ID NO: 14, 18, 19, 20, and 21, and/or the guide RNA comprises a sequence differing by no more than 5, 4, 3, 2, or 1 nucleotide(s) from any one of SEQ ID NOs: 2, 6, 7, 8, and 9. In some embodiments, the endonuclease used in the CRISPR gene editing system is a Cas9 endonuclease; and the target sequence of the guide RNA of the CRISPR gene editing system comprises SEQ ID NO: 17 and/or the guide RNA comprises a sequence differing by no more than 5, 4, 3, 2, or 1 nucleotide(s) from SEQ ID NO: 5. In some embodiments, the endonuclease used in the CRISPR gene editing system is a Cas9 endonuclease; and, the target sequence of the guide RNA of the CRISPR gene editing system comprises any one of SEQ ID NOs: 15 or 16 and/or the guide RNA comprises a sequence differing by no more than 5, 4, 3, 2, or 1 nucleotide(s) from SEQ ID NOs: 3 or 4. In some embodiments, the endonuclease used in the CRISPR gene editing system is a Cas9 endonuclease; and, the target sequence of the guide RNA of the CRISPR gene editing system comprises SEQ ID NOs: 62 and/or the guide RNA comprises a sequence differing by no more than 5, 4, 3, 2, or 1 nucleotide(s) from SEQ ID NO: 61. In some embodiments, the endonuclease used in the CRISPR gene editing system is a Cas9 endonuclease; and, the target sequence of the guide RNA of the CRISPR gene editing system comprises SEQ ID NOs: 56 and/or the guide RNA comprises a sequence differing by no more than 5, 4, 3, 2, or 1 nucleotide(s) from SEQ ID NO: 55. In some embodiments, the endonuclease used in the CRISPR gene editing system is a Cas9 endonuclease; and, the target sequence of the guide RNA of the CRISPR gene editing system comprises SEQ ID NOs: 59 and/or the guide RNA comprises a sequence differing by no more than 5, 4, 3, 2, or 1 nucleotide(s) from SEQ ID NO: 58.

In some embodiments, the guide RNA comprises SEQ ID NO: 5 (SOCS1), SEQ ID NO: 29 (SOCS1), SEQ ID NO: 11 (SOCS1), SEQ ID NO: 37 (SOCS1), SEQ ID NO: 27 (CISH), SEQ ID NO: 28 (CISH), SEQ ID NO: 12 (CISH), SEQ ID NO: 38 (CISH), SEQ ID NO: 2 (BIM), SEQ ID NO: 26 (BIM), SEQ ID NO: 6 (BIM), SEQ ID NO: 32 (BIM), SEQ ID NO: 7 (BIM), SEQ ID NO: 33 (BIM), SEQ ID NO: 8 (BIM), SEQ ID NO: 34 (BIM), SEQ ID NO: 9 (BIM), SEQ ID NO: 35 (BIM) SEQ ID NO: 10 (BIM), SEQ ID NO: 36 (BIM), SEQ ID NO: 61 (FAS), SEQ ID NO: 60 (FAS), SEQ ID NO: 55 (B2M), SEQ ID NO: 54 (B2M), SEQ ID NO: 58 (CIITA), or SEQ ID NO: 57 (CIITA).

In some embodiments, the guide RNA comprises a CRISPR RNA (crRNA) sequence, in which the crRNA sequence comprises a 2′-O-Methyl modification at each of the first three positions of the 5′ end, and three 2′-O-Methyl modifications at the 3′ end of the crRNA, but not the very last base of the crRNA (e.g., for a 56 nucleotide crRNA, at base 53, 54, 55 of the crRNA, but not at base 56; for a 60 nucleotide crRNA, at base 57, 58, and 59, but not at base 60), and a phosphorothioate linkage between each base in the first three positions of the 5′ end, and a phosphorothioate linkage between each base of the last 3 positions of the crRNA sequence at the 3′ end. In some embodiments, the last base is modified to facilitate use with MAD7. In some embodiments, the guide RNA binds to MAD7. In some embodiments, the methyl modifications at the 5′ end and the phosphorothioate linkages at the 3′ end are to increase the crRNA stability.

In some embodiments, the crRNA is about 56 bases long. In some embodiments, the crRNA sequence is 56 bases long, and comprises a 2′-O-Methyl modification at each of positions 53, 54, and 55 of the crRNA sequence on the 3′ end. In some embodiments, the crRNA sequence is 56 bases long, and comprises a 2′-O-Methyl modification at each of positions 53, 54, and 55 of the crRNA sequence on the 3′ end, and a 2′-O-Methyl modification at each of positions 1, 2, and 3 of the crRNA sequence at the 5′ end. In some embodiments, the crRNA sequence is 56 bases long, and comprises a 2′-O-Methyl modification at each of positions 53, 54, and 55 of the crRNA sequence on the 3′ end, and a 2′-O-Methyl modification at each of positions 1, 2, and 3 of the crRNA sequence at the 5′ end, and a phosphorothioate linkage between each base in the first three positions of the 5′ end, and a phosphorothioate linkage between each base of the last 3 positions of the crRNA sequence at the 3′ end. In some embodiments, the crRNA comprises SEQ ID NO: 36, SEQ ID NO: 37, or SEQ ID NO: 38, which include the above-described modification pattern. In some embodiments, the last nucleotide at the 3′ end of the crRNA is modified with a nucleotide that anchors the crRNA binding to MAD7. In some embodiments, the last nucleotide at the 3′ end of the crRNA is modified with a pseudoknot that anchors the crRNA binding to MAD7.

### B. Programmable Addition Via Site-Specific Target Elements (PASTE)

In some embodiments, the gene editing system used to modify the cells herein (e.g., the iγδT cell, iPSC, iHSC, iCLP cell, or immature iT cell) is a Programmable Addition via Site-Specific Target Elements (PASTE) (see, e.g., Yarnall M T N, Ioannidi E I, Schmitt-Ulms C, Krajeski R N, Lim J, Villiger L, Zhou W, Jiang K, Garushyants S K, Roberts N, Zhang L, Vakulskas C A, Walker J A 2nd, Kadina A P, Zepeda A E, Holden K, Ma H, Xie J, Gao G, Foquet L, Bial G, Donnelly S K, Miyata Y, Radiloff D R, Henderson J M, Ujita A, Abudayyeh O O, Gootenberg J S. Drag-and-drop genome insertion of large sequences without double-strand DNA cleavage using CRISPR-directed integrases. Nat Biotechnol. 2023 April; 41(4):500-512.). The PASTE gene editing system comprises a guide RNA, e.g., an attachment site-containing guide RNA (atgRNA) that incorporates a prime-editing guide RNA (pegRNA) sequence, which both specifies the target site and encodes the desired genomic disruption, and an attachment site for serine integrases. PASTE further comprises a CRISPR-Cas9 nickase fused to a reverse transcriptase and a serine integrase. In some embodiments, the pegRNA targets SOCS1. In some embodiments, the pegRNA targets CISH. In some embodiments, the pegRNA targets BIM. In some embodiments, the pegRNA targets B2M. In some embodiments, the pegRNA targets CIITA. In some embodiments, the pegRNA targets FAS. In some embodiments, the PASTE gene is transfected into γδT-cells, e.g., iPSC-derived γδT-cells, and their precursors (e.g., iPSC (e.g., γδT-iPSC), or an intermediate cell differentiated from an iPSC (e.g., γδT-iHSC, γδT-iCLP, and immature T-iγδT cells)) that is used to generate a modified T-iγδT cell.

### C. Prime Editing

In some embodiments, the gene editing system used to modify the cells herein (e.g., the iγδT cell, iPSC, iHSC, iCLP cell, or immature iT cell) is a Prime Editing (see Anzalone A V, Randolph P B, Davis J R, Sousa A A, Koblan L W, Levy J M, Chen P J, Wilson C, Newby G A, Raguram A, Liu D R. Search-and-replace genome editing without double-strand breaks or donor DNA. Nature. 2019 December; 576(7785):149-157.). The Prime Editing gene editing system comprises a guide RNA, including e.g., a pegRNA, which both specifies the target site and encodes the desired genomic disruption, and a CRISPR-Cas9 endonuclease fused to a reverse transcriptase. In some embodiments, the pegRNA targets SOCS1. In some embodiments, the pegRNA targets CISH. In some embodiments, the pegRNA targets BIM. In some embodiments, the pegRNA targets B2M. In some embodiments, the pegRNA targets CIITA. In some embodiments, the pegRNA targets FAS. In some embodiments, the Prime Editing gene editing system is transfected into γδT-cells, e.g., iPSC-derived γδT-cells, and their precursors (e.g., iPSC (e.g., γδT-iPSC), or an intermediate cell differentiated from an iPSC (e.g., γδT-iHSC, γδT-iCLP, and immature T-iγδT cells)) that is used to generate a modified T-iγδT cell.

### D. Base Editing

In some embodiments, the gene editing system used to modify the cells herein (e.g., the iγδT cell, iPSC, iHSC, iCLP cell, or immature iT cell) is base editing (see, e.g., Gaudelli N M, Komor A C, Rees H A, Packer M S, Badran A H, Bryson D I, Liu D R. Programmable base editing of A⋅T to G⋅C in genomic DNA without DNA cleavage. Nature. 2017 Nov. 23; 551(7681):464-471.). Base editing comprises a base editor, including e.g., a cytosine base editor or an adenine base editor, and a guide sequence used to target the base editor to a specific nucleotide sequence.

The base editor comprises a Cas9 nickase and a nucleoside deaminase, including e.g., a cytosine deaminase or an adenine deaminase. The Cas9 nickase cuts one strand of DNA at a specific location determined by the guide sequence, and the nucleoside deaminase removes an amino group from a specific type of nucleoside (e.g., cytosine or adenine). In some embodiments, the guide sequence targets SOCS1. In some embodiments, the guide sequence targets CISH. In some embodiments, the guide sequence targets BIM. In some embodiments, the sequence targets B2M. In some embodiments, the guide sequence targets CIITA. In some embodiments, the guide sequence targets FAS. In some embodiments, the base editing gene editing system is transfected into γδT-cells, e.g., iPSC-derived γδT-cells, and their precursors (e.g., iPSC (e.g., γδT-iPSC), or an intermediate cell differentiated from an iPSC (e.g., γδT-iHSC, γδT-iCLP, and immature T-iγδT cells)) that is used to generate a modified T-iγδT cell.

### E. Transcription Activator-Like Effector Nucleases (Talens)

In some embodiments, the gene editing system used to modify the cells herein (e.g., the iγδT cell, iPSC, iHSC, iCLP cell, or immature iT cell) is a transcription activator-like effector nuclease (TALEN) (see, e.g., Christian M, Cermark T, Doyle E L, Schmidt C, Zhang F, Hummel A, Bogdanove A J, Voytas D F. Targeting DNA double-strand breaks with TAL effector nucleases. Genetics. 2010; 186:757-761. Further, see Miller J C, Tan S, Qiao G, Barlow K A, et al. A TALE nuclease architecture for efficient genome editing. Nat Biotechnol. 2011; 29:143-148. Also, see Mussolino C, Morbitzer R, Lutge F, Dannemann N, Lahaye T, Cathomen T. A novel TALE nuclease scaffold enables high genome editing activity in combination with low toxicity. Nucl Acids Res. 2011; 39:9283-9293.). The TALEN gene editing system comprises the TALEN, which is a restriction endonuclease, e.g., Fokl, fused to a transcription activator-like effector (TALE) that is engineered to bind to a desired DNA sequence to promote DNA cleavage at a specific location. In some embodiments, two distinct TALENs each target distinct regions of SOCS1. In some embodiments, two distinct TALENs each target distinct regions of CISH. In some embodiments, two distinct TALENs each target distinct regions of BIM. In some embodiments, two distinct TALENs each target distinct regions of B2M. In some embodiments, two distinct TALENs each target distinct regions of CIITA. In some embodiments, two distinct TALENs each target distinct regions of FAS. In some embodiments, the TALENs are transfected into γδT-cells, e.g., iPSC-derived γδT-cells, and their precursors (e.g., iPSC (e.g., γδT-iPSC), or an intermediate cell differentiated from an iPSC (e.g., γδT-iHSC, γδT-iCLP, and immature T-iγδT cells)) that is used to generate a modified T-iγδT cell.

### F. Zinc-Finger Nucleases (Zfns)

In some embodiments, the gene editing system used to modify the cells herein (e.g., the iγδT cell, iPSC, iHSC, iCLP cell, or immature iT cell) is a zinc-finger nuclease (ZFN) (see, e.g., Kim Y-G, Cha J, Chandrasegaran S. Hybrid restriction enzymes: Zinc finger fusions to FokI cleavage domain. Proc NatlAcad Sci USA. 1996; 93:1156-1160.). In some embodiments, the ZFN comprises an endonuclease, including, e.g., Fokl, fused to zinc finger, in which each zinc finger domain of a zinc finger targets a sequence. In some embodiments, two distinct ZFNs each target distinct regions of SOCS1. In some embodiments, two distinct ZFNs each target distinct regions of CISH. In some embodiments, two distinct ZFNs each target distinct regions of BIM. In some embodiments, two distinct ZFNs each target distinct regions of B2M. In some embodiments, two distinct ZFNs each target distinct regions of CIITA. In some embodiments, two distinct ZFNs each target distinct regions of FAS. In some embodiments, ZFNs are transfected into γδT-cells, e.g., iPSC-derived γδT-cells, and their precursors (e.g., iPSC (e.g., γδT-iPSC), or an intermediate cell differentiated from an iPSC (e.g., γδT-iHSC, γδT-iCLP, and immature T-iγδT cells)) that is used to generate a modified T-iγδT cell.

### G. Endonucleases

Described herein are restriction endonuclease for use in the methods described herein, and also as components of the gene editing systems. For example, homing endonucleases and meganucleases are provided. In some embodiments, meganucleases, restriction endonucleases, and/or homing endonucleases are engineered to confer a genomic disruption or inactivating nucleic acid mutation in any one or more of BIM, SOCS, and CISH; SOCS1 and CISH, and, optionally, BIM, B2M, CIITA, and/or FAS in the cells herein (e.g., the iγδT cell, iPSC, iHSC, iCLP cell, or immature iT cell). In some embodiments, one meganuclease targets SOCS1 and a second meganuclease targets CISH. In some embodiments, a third meganuclease targets BIM. In some embodiments, a fourth meganuclease targets B2M. In some embodiments, a fifth meganuclease targets CIITA. In some embodiments, a sixth meganuclease targets FAS. In some embodiments, one restriction endonuclease targets SOCS1 and a second restriction endonuclease targets CISH. In some embodiments, a third restriction endonuclease targets BIM. In some embodiments, a fourth restriction endonuclease targets B2M. In some embodiments, a fifth restriction endonuclease targets CIITA. In some embodiments, a sixth restriction endonuclease targets FAS. In some embodiments, one homing endonuclease targets SOCS1 and a second homing endonuclease targets CISH. In some embodiments, a third homing endonuclease targets BIM. In some embodiments, a fourth homing endonuclease targets B2M. In some embodiments, a fifth homing endonuclease targets CIITA. In some embodiments, a sixth homing endonuclease targets FAS. In some embodiments, the restriction endonuclease, homing endonucleases, and/or meganucleases are transfected into γδT-cells, e.g., iPSC-derived γδT-cells, and their precursors (e.g., iPSC (e.g., γδT-iPSC), or an intermediate cell differentiated from an iPSC (e.g., γδT-iHSC, γδT-iCLP, and immature T-iγδT cells)) that is used to generate a modified T-iγδT cell.

### H. Transfection

The present disclosure provides methods of modifying the cell used herein (e.g., the T-iγδT cell, the γδT-iPSC, T-iHSC, T-iCLP, or immature T-iγδT cell) by transfection, including e.g., electroporation (e.g., Nucleofection® (Lonza Cologne GmbH, Cologne Germany)), physical transfection (e.g., microinjection, particle bombardment, or phototransfection), lipid-mediated transfection, diethylaminoethyl (DEAE)-dextran transfection, calcium phosphate precipitation, cationic polymer transfection, or viral transfection.

To generate a genomic disruption or inactivating nucleic acid mutation, the cell can be transfected (e.g., nucleofected) with a gene editing system. In some embodiments, the gene editing system comprises an endonuclease, including, e.g., Cas9 or MAD7, and an sgRNA that targets SOCS1, CISH, BIM, B2M, CIITA, or FAS. In some embodiments, the cell (e.g., T-iγδT cell, γδT-iPSC, T-iHSC, T-iCLP, or immature T-iγδT cell) undergoes a sequential transfection of the gene editing system. For example, the cell (e.g., T-iγδT cell, γδT-iPSC, T-iHSC, T-iCLP, or immature T-iγδT cell) is transfected with a first endonuclease and an sgRNA that targets SOCS1; then, the cell (e.g., T-iγδT cell, γδT-iPSC, T-iHSC, T-iCLP, or immature T-iγδT cell) is transfected with an sgRNA that targets CISH and, optionally, a second endonuclease or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets CISH; then, the cell (e.g., T-iγδT cell, γδT-iPSC, T-iHSC, T-iCLP, or immature T-iγδT cell) is transfected with an sgRNA that targets SOCS1 and, optionally, a second endonuclease or the first endonuclease.

In some embodiments, the cell (e.g., T-iγδT cell, γδT-iPSC, T-iHSC, T-iCLP, or immature T-iγδT cell) is transfected with a first endonuclease and an sgRNA that targets SOCS1; then, the cell (e.g., T-iγδT cell, γδT-iPSC, T-iHSC, T-iCLP, or immature T-iγδT cell) is transfected with an sgRNA that targets CISH and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets BIM and, optionally, a third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets CISH; then, the cell (e.g., T-iγδT cell, γδT-iPSC, T-iHSC, T-iCLP, or immature T-iγδT cell) is transfected with an sgRNA that targets SOCS1 and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets BIM and, optionally, a third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets SOCS1; then, the cell (e.g., T-iγδT cell, γδT-iPSC, T-iHSC, T-iCLP, or immature T-iγδT cell) is transfected with an sgRNA that targets BIM and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets CISH and, optionally, a third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets CISH; then, the cell (e.g., T-iγδT cell, γδT-iPSC, T-iHSC, T-iCLP, or immature T-iγδT cell) is transfected with an sgRNA that targets BIM and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets SOCS1 and, optionally, a third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets BIM; then, the cell (e.g., T-iγδT cell, γδT-iPSC, T-iHSC, T-iCLP, or immature T-iγδT cell) is transfected with an sgRNA that targets SOCS1 and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets CISH and, optionally, a third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets BIM; then, the cell (e.g., T-iγδT cell, γδT-iPSC, T-iHSC, T-iCLP, or immature T-iγδT cell) is transfected with an sgRNA that targets CISH and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets SOCS1 and, optionally, a third endonuclease, the second endonuclease, and/or the first endonuclease.

In some embodiments, the cell (e.g., T-iγδT cell, γδT-iPSC, T-iHSC, T-iCLP, or immature T-iγδT cell) is transfected with a first endonuclease and an sgRNA that targets SOCS1; then, the cell (e.g., T-iγδT cell, γδT-iPSC, T-iHSC, T-iCLP, or immature T-iγδT cell) is transfected with an sgRNA that targets CISH and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets FAS and, optionally, a third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets CISH; then, the cell (e.g., T-iγδT cell, γδT-iPSC, T-iHSC, T-iCLP, or immature T-iγδT cell) is transfected with an sgRNA that targets SOCS1 and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets FAS and, optionally, a third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets SOCS1; then, the cell (e.g., T-iγδT cell, γδT-iPSC, T-iHSC, T-iCLP, or immature T-iγδT cell) is transfected with an sgRNA that targets FAS and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets CISH and, optionally, a third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets CISH; then, the cell (e.g., T-iγδT cell, γδT-iPSC, T-iHSC, T-iCLP, or immature T-iγδT cell) is transfected with an sgRNA that targets FAS and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets SOCS1 and, optionally, a third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets FAS; then, the cell (e.g., T-iγδT cell, γδT-iPSC, T-iHSC, T-iCLP, or immature T-iγδT cell) is transfected with an sgRNA that targets SOCS1 and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets CISH and, optionally, a third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with a first endonuclease and an sgRNA that targets FAS; then, the cell (e.g., T-iγδT cell, γδT-iPSC, T-iHSC, T-iCLP, or immature T-iγδT cell) is transfected with an sgRNA that targets CISH and, optionally, a second endonuclease or the first endonuclease; then, the cell (e.g., T-iγδT cell, γδT-iPSC, T-iHSC, T-iCLP, or immature T-iγδT cell) is transfected with an sgRNA that targets SOCS1 and, optionally, a third endonuclease, the second endonuclease, and/or the first endonuclease.

In some embodiments, the cell (e.g., T-iγδT cell, γδT-iPSC, T-iHSC, T-iCLP, or immature T-iγδT cell) is transfected with a first endonuclease and an sgRNA that targets SOCS1; and, the cell (e.g., T-iγδT cell, γδT-iPSC, T-iHSC, T-iCLP, or immature T-iγδT cell) is transfected with an sgRNA that targets CISH and, optionally, a second endonuclease or the first endonuclease; and, the cell (e.g., T-iγδT cell, γδT-iPSC, T-iHSC, T-iCLP, or immature T-iγδT cell) is transfected with an sgRNA that targets BIM and, optionally, a third endonuclease, the second endonuclease, or the first endonuclease; and, the cell (e.g., T-iγδT cell, γδT-iPSC, T-iHSC, T-iCLP, or immature T-iγδT cell) is transfected with an sgRNA that targets B2M and, optionally, a fourth endonuclease, the third endonuclease, the second endonuclease, or the first endonuclease; and, the cell (e.g., T-iγδT cell, γδT-iPSC, T-iHSC, T-iCLP, or immature T-iγδT cell) is transfected with an sgRNA that targets CIITA; and, optionally, a fifth endonuclease; the fourth endonuclease, the third endonuclease, the second endonuclease, and/or the first endonuclease. In some embodiments, the cell (e.g., T-iTST cell, γδT-iPSC, T-iHSC, T-iCLP, or immature T-iγδT cell) is transfected with the sgRNA that targets SOCS1, the sgRNA that targets CISH, the sgRNA that targets BIM, and the sgRNA that targets B2M, the sgRNA that targets CIITA in any order. In some embodiments, the cell (e.g., T-iγδT cell, γδT-iPSC, T-iHSC, T-iCLP, or immature T-iγδT cell) is transfected with the first endonuclease, the second endonuclease, the third endonuclease, the fourth endonuclease, the fifth endonuclease, or any combination thereof in any order.

In some embodiments, the cell (e.g., T-iγδT cell, γδT-iPSC, T-iHSC, T-iCLP, or immature T-iγδT cell) is transfected with a first endonuclease and an sgRNA that targets SOCS1; and, the cell (e.g., T-iγδT cell, γδT-iPSC, T-iHSC, T-iCLP, or immature T-iγδT cell) is transfected with an sgRNA that targets CISH and, optionally, a second endonuclease or the first endonuclease; and, the cell (e.g., T-iγδT cell, γδT-iPSC, T-iHSC, T-iCLP, or immature T-iγδT cell) is transfected with an sgRNA that targets BIM and, optionally, a third endonuclease, the second endonuclease, or the first endonuclease; and, the cell (e.g., T-iγδT cell, γδT-iPSC, T-iHSC, T-iCLP, or immature T-iγδT cell) is transfected with an sgRNA that targets B2M and, optionally, a fourth endonuclease, the third endonuclease, the second endonuclease, or the first endonuclease; and, the cell (e.g., T-iγδT cell, γδT-iPSC, T-iHSC, T-iCLP, or immature T-iγδT cell) is transfected with an sgRNA that targets CIITA; and, optionally, a fifth endonuclease, the fourth endonuclease, the third endonuclease, the second endonuclease, and/or the first endonuclease; and, the cell (e.g., T-iγδT cell, γδT-iPSC, T-iHSC, T-iCLP, or immature T-iγδT cell) is transfected with an sgRNA that targets FAS; and, optionally, a sixth endonuclease, the fifth endonuclease, the fourth endonuclease, the third endonuclease, the second endonuclease, and/or the first endonuclease. In some embodiments, the cell (e.g., T-iγδT cell, γδT-iPSC, T-iHSC, T-iCLP, or immature T-iγδT cell) is transfected with the sgRNA that targets SOCS1, the sgRNA that targets CISH, the sgRNA that targets BIM, the sgRNA that targets B2M, the sgRNA that targets CIITA, the sgRNA that targets B2M, and the sgRNA that targets FAS in any order. In some embodiments, the cell (e.g., T-iγδT cell, γδT-iPSC, T-iHSC, T-iCLP, or immature T-iγδT cell) is transfected with the first endonuclease, the second endonuclease, the third endonuclease, the fourth endonuclease, the fifth endonuclease, the sixth endonuclease or any combination thereof in any order.

### VI. Methods of Generating the Modified Cells

In some embodiments, methods of inhibiting expression of any one or more of a SOCS1 gene, a CISH gene and a BIM gene in γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) are provided.

In some embodiments, methods of inhibiting expression of a SOCS1 gene and a CISH gene in γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iTST cells))) are provided. In some embodiments, methods of inhibiting expression of a SOCS1 gene, a CISH gene, and a BIM gene in γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) are provided. In some embodiments, methods of inhibiting expression of a SOCS1 gene, a CISH gene, a BIM gene, and a FAS gene in γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) are provided. In some embodiments, methods of inhibiting expression of a SOCS1 gene, a CISH gene, a BIM gene, a B2M gene, and a CIITA gene in γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) are provided. In some embodiments, methods of inhibiting expression of a SOCS1 gene, a CISH gene, a BIM gene, a B2M gene, a CIITA gene, and a FAS gene in γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) are provided. In some embodiments, the method suppresses or eliminates expression of SOCS1 and CISH. In some embodiments, the method suppresses or eliminates expression of SOCS1, CISH, and BIM. In some embodiments, the method suppresses or eliminates expression of SOCS1, CISH, BIM, and FAS. In some embodiments, the method suppresses or eliminates expression of SOCS1, CISH, BIM, B2M, and CIITA. In some embodiments, the method suppresses or eliminates expression of SOCS1, CISH, BIM, B2M, CIITA, and FAS. In some instances, the expression of BIM_{EL} and BIM_{L} splice variants are reduced in the modified cell relative to that in an unmodified control (e.g., an unmodified T-iγδT-cell). In some embodiments, the expression of BIM_{S} splice variant is retained in the modified cell.

Described herein is a method of inhibiting expression of SOCS1 and/or CISH genes in γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)) that is used to generate a modified T-iγδT cell. The method comprises using a gene editing system (e.g., gene editing system described in Section V) to modify the γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)). For example, the cells (e.g., the T-iγδT cell, the γδT-iPSC, T-iHSC, T-iCLP, or immature T-iγδT cell) can be contacted with guide RNAs and an endonuclease or a nucleic acid encoding an endonuclease, wherein the contacting results in a genomic disruption in an endogenous SOCS1 gene that suppresses or eliminates expression of SOCS1 and/or a genomic disruption in an endogenous CISH gene that suppresses or eliminates expression of CISH. In some embodiments, the genomic disruption within SOCS1 and/or CISH is made by administering guide RNAs to the cells herein (e.g., the iγδT cell, iPSC, iHSC, iCLP cell, or immature iT cell). In some embodiments, the guide RNAs are sgRNAs. In some embodiments, the guide RNAs comprise any one or more of SEQ ID NOs: 5, 29, 11, 37 and/or any one or more of SEQ ID NOs: 3, 27, 4, 28, 12, or 38. In some embodiments, the endonuclease that is administered to the cell is a Cas endonuclease. In some embodiments, the Cas endonuclease that is administered to the cell is MAD7 endonuclease. The MAD7 endonuclease is an engineered class 2 type V-A CRISPR-Cas (Cas12a/Cpf1) system.

Described herein is a method of inhibiting expression of BIM gene in γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)) that is used to generate a modified T-iγδT cell. The method comprises using a gene editing system (e.g., gene editing system described in Section V) to modify the γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)). For example, the cells (e.g., the T-iγδT cell, the γδT-iPSC, T-iHSC, T-iCLP, or immature T-iγδT cell) can be contacted with guide RNAs and an endonuclease or a nucleic acid encoding an endonuclease, wherein the contacting results in a genomic disruption in an endogenous BIM gene that suppresses or eliminates expression of BIM, wherein the genomic disruption is in an exon 2C of an endogenous BIM gene, and the expression and/or function of BIM_{EL} and BIM_{L} splice variants are reduced in the modified cell relative to that in an unmodified control and the expression and/or function of BIM_{S} splice variant is retained in the modified cell. In some embodiments, the genomic disruption within BIM is made by administering guide RNAs to the cells herein (e.g., the iγδT cell, iPSC, iHSC, iCLP cell, or immature iT cell). In some embodiments, the guide RNAs are sgRNAs. In some embodiments, the guide RNAs comprise any one or more of SEQ ID NOs: 2, 26, 8, 34, 9, 35, 10 and 36. In some embodiments, the endonuclease that is administered to the cell is a Cas endonuclease. In some embodiments, the Cas endonuclease that is administered to the cell is MAD7 endonuclease. The MAD7 endonuclease is an engineered class 2 type V-A CRISPR-Cas (Cas12a/Cpf1) system.

Described herein is a method of inhibiting expression of a SOCS1, CISH, and BIM genes by causing a genomic disruption in the SOCS1, CISH, and BIM genes in γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)). The method comprises using a gene editing system (e.g., gene editing system described in Section V) to modify γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)). For example, the cells herein (e.g., the iγδT cell, iPSC, iHSC, iCLP cell, or immature iT cell) can be contacted with guide RNAs and an endonuclease or a nucleic acid encoding an endonuclease, wherein the contacting results in a genomic disruption in an endogenous SOCS1 gene that suppresses or eliminates expression of SOCS1, a genomic disruption in an endogenous CISH gene that suppresses or eliminates expression of CISH, and a genomic disruption in an endogenous BIM gene that suppresses or eliminates expression of BIM. In some embodiments, the expression and/or function of BIM_{EL} and BIM_{L} splice variants are reduced in the modified γδT-cell relative to that in an unmodified γδT-cell or relative to the γδT-cell (e.g., T-iγδT-cell) before modification. In some embodiments, the expression and/or function of BIM_{S} splice variant is retained in the modified γδT-cell.

Described herein is a method of inhibiting expression of a SOCS1, CISH, BIM, and FAS genes by causing a genomic disruption in the SOCS1, CISH, BIM, and FAS genes in γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)). The method comprises using a gene editing system (e.g., gene editing system described in Section V) to modify γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)). For example, the cells herein (e.g., the iγδT cell, iPSC, iHSC, iCLP cell, or immature iT cell) can be contacted with guide RNAs and an endonuclease or a nucleic acid encoding an endonuclease, wherein the contacting results in a genomic disruption in an endogenous SOCS1 gene that suppresses or eliminates expression of SOCS1, a genomic disruption in an endogenous CISH gene that suppresses or eliminates expression of CISH, a genomic disruption in an endogenous BIM gene that suppresses or eliminates expression of BIM, and a genomic disruption in an endogenous FAS gene that suppresses or eliminates expression of FAS. In some embodiments, the expression and/or function of BIM_{EL} and BIM_{L} splice variants are reduced in the modified γδT-cell relative to that in an unmodified γδT-cell or relative to the γδT-cell (e.g., T-iγδT-cell) before modification. In some embodiments, the expression and/or function of BIM_{S} splice variant is retained in the modified γδT-cell.

Described herein is a method of inhibiting expression of a SOCS1, CISH, BIM, B2M, and CIITA genes by causing a genomic disruption in the SOCS1, CISH, BIM, B2M and CIITA genes in γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)). The method comprises using a gene editing system to modify the γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)). For example, the cells herein (e.g., the iγδT cell, iPSC, iHSC, iCLP cell, or immature iT cell) can be contacted with guide RNAs and an endonuclease or a nucleic acid encoding an endonuclease, wherein the contacting results in a genomic disruption in an endogenous SOCS1 gene that suppresses or eliminates expression of SOCS1, a genomic disruption in an endogenous CISH gene that suppresses or eliminates expression of CISH, a genomic disruption in an endogenous BIM gene that suppresses or eliminates expression of BIM, a genomic disruption in an endogenous B2M gene that suppresses or eliminates expression of B2M, and a genomic disruption in an endogenous CIITA gene that suppresses or eliminates expression of CIITA. In some embodiments, the expression and/or function of BIM_{EL} and BIM_{L} splice variants are reduced in the modified γδT-cell relative to that in an unmodified γδT-cell or relative to the γδT-cell (e.g., T-iγδT-cell) before modification. In some embodiments, the expression and/or function of BIM_{S} splice variant is retained in the modified γδT-cell.

Described herein is a method of inhibiting expression of a SOCS1, CISH, BIM, B2M, CIITA, and FAS genes by causing a genomic disruption in the SOCS1, CISH, BIM, B2M, CIITA, and FAS genes in γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)). The method comprises using a gene editing system to modify the γδT-cells, e.g., iPSC-derived γδT-cells, and their precursors (e.g., an iPSC (e.g., γδT-iPSC), or an intermediate cell differentiated from an iPSC (e.g., γδT-iHSC, γδT-iCLP, and immature T-iγδT cells)). For example, the cells herein (e.g., the iγδT cell, iPSC, iHSC, iCLP cell, or immature iT cell) can be contacted with guide RNAs and an endonuclease or a nucleic acid encoding an endonuclease, wherein the contacting results in a genomic disruption in an endogenous SOCS1 gene that suppresses or eliminates expression of SOCS1, a genomic disruption in an endogenous CISH gene that suppresses or eliminates expression of CISH, a genomic disruption in an endogenous BIM gene that suppresses or eliminates expression of BIM, a genomic disruption in an endogenous B2M gene that suppresses or eliminates expression of B2M, a genomic disruption in an endogenous CIITA gene that suppresses or eliminates expression of CIITA, and a genomic disruption in an endogenous FAS gene that suppresses or eliminates expression of FAS. In some embodiments, the expression and/or function of BIM_{EL} and BIM_{L} splice variants are reduced in the modified γδT-cell relative to that in an unmodified γδT-cell or relative to the γδT-cell (e.g., T-iγδT-cell) before modification. In some embodiments, the expression and/or function of BIM_{S} splice variant is retained in the modified γδT-cell.

In some embodiments, the genomic disruption and/or inactivating mutation of the SOCS1 and BIM genes; SOCS1, CISH, and BIM genes; SOCS1, CISH, BIM, and FAS genes; SOCS1, CISH, BIM, B2M, and CIITA genes; or, SOCS1, CISH, BIM, B2M, CIITA, and FAS genes is made by administering guide RNAs to a cell (e.g., a T-iγδT cell, the γδT-iPSC, T-iHSC, T-iCLP, or immature T-iγδT cell). In some embodiments, the guide RNAs are sgRNAs. In some embodiments, the guide RNAs that target the SOCS1 gene comprise any one or more of SEQ ID NOs: 5, 29, 11, 37; the guide RNAs that target the CISH gene comprise any one or more of SEQ ID NOs: 3, 27, 4, 28, 12, or 38; the guide RNAs that target the BIM gene comprise any one or more of SEQ ID NOs: 2, 26, 6, 32, 7, 33, 8, 34, 9, 35, 10, and 36; the guide RNAs that target the FAS gene comprise any one or more of SEQ ID NOs: 60 and 61; the guide RNAs that target the B2M gene comprise any one or more of SEQ ID NOs: 54 and 55; and the guide RNAs that target the CIITA gene comprise any one or more of SEQ ID NOs: 57 and 58. In some embodiments, a nuclease is administered to the cell (e.g., a T-iγδT cell, the γδT-iPSC, T-iHSC, T-iCLP, or immature T-iγδT cell) with the guide RNA. In some embodiments, the nuclease is an endonuclease. In some embodiments, the endonuclease that is administered to the cell is a Cas endonuclease. In some embodiments, the Cas endonuclease that is administered to a cell is a Cas9 endonuclease or a MAD7 endonuclease.

In some embodiments, the genomic disruption of the endogenous SOCS1 gene suppresses or eliminates expression of the SOCS1 gene in the modified γδT-cell (e.g., T-iγδT-cell) relative to an unmodified γδT-cell (e.g., an unmodified T-iγδT-cell) or relative to the γδT-cell (e.g., T-iγδT-cell) before modification. In some embodiments, the genomic disruption in the SOCS1 gene is in exon 2 of the SOCS1 gene. In some embodiments, the genomic disruption in the SOCS1 gene is an inactivating nucleic acid mutation. In some embodiments, the genomic disruption in the SOCS1 gene is an inactivating nucleic acid mutation in exon 2 of the SOCS1 gene. In some embodiments, a genomic disruption or inactivating nucleic acid mutation in the SOCS1 gene is an insertion, deletion, substitution, or any combination thereof. In some embodiments, the genomic disruption is within a target sequence in SOCS1 that comprises the nucleotide sequence of any one of SEQ ID NO: 17, SEQ ID NO: 23, or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, the genomic disruption of the endogenous CISH gene suppresses or eliminates expression of the CISH gene in the modified γδT-cell (e.g., T-iγδT-cell) relative to an unmodified γδT-cell (e.g., an unmodified T-iγδT-cell) or relative to the γδT-cell (e.g., T-iγδT-cell) before modification. In some embodiments, the genomic disruption in the CISH gene is in exon 3, exon 4, or a combination thereof of the CISH gene. In some embodiments, the genomic disruption in the CISH gene is an inactivating nucleic acid mutation.

In some embodiments, the genomic disruption in the CISH gene is an inactivating nucleic acid mutation in exon 3, exon 4, or a combination thereof of the CISH gene. In some embodiments, a genomic disruption or inactivating nucleic acid mutation in the CISH gene is an insertion, deletion, substitution, or any combination thereof. In some embodiments, the genomic disruption is within a target sequence in CISH that comprises the nucleotide sequence of any one of SEQ ID NO: 15, SEQ ID NO: 16 or SEQ ID NO: 24 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, the genomic disruption of the endogenous BIM gene in the modified γδT-cell reduces the expression and/or function of a splice variant of BIM, BIM_{EL}, relative to an unmodified γδT-cell generated from a γδT-iPSC (e.g., an unmodified T-iγδT-cell) or relative to the γδT-cell (e.g., T-iγδT-cell) before modification. In some embodiments, the genomic disruption of the endogenous BIM gene reduces the expression and/or function of a splice variant of BIM, BIM_{L}, relative to an unmodified γδT-cell generated from a γδT-iPSC (e.g., an unmodified T-iγδT-cell) or relative to the γδT-cell (e.g., T-iγδT-cell) before modification. In some embodiments, the genomic disruption of the endogenous BIM gene in the modified γδT-cell reduces the expression and/or function of a splice variants BIM_{EL} and BIM_{L} relative to an unmodified γδT-cell generated from a γδT-iPSC (e.g., an unmodified T-iγδT-cell) or relative to the γδT-cell (e.g., T-iγδT-cell) before modification. In some embodiments, the genomic disruption of the endogenous BIM gene in the modified γδT-cell retains the expression and/or function a splice variant of BIM, BIM_{S}, relative to an unmodified γδT-cell generated from a γδT-iPSC (e.g., an unmodified T-iγδT-cell) or relative to the γδT-cell (e.g., T-iγδT-cell) before modification. In some embodiments, the genomic disruption of the BIM gene is in exon 2C of an endogenous BIM gene. In some embodiments, the genomic disruption of the BIM gene is an inactivating nucleic acid mutation in exon 2C of an endogenous BIM gene. In some embodiments, the inactivating mutation is an insertion, deletion, or substitution. In some embodiments, the genomic disruption is within a target sequence in BIM that comprises the nucleotide sequence of any one of SEQ ID NO: 14, SEQ ID NO: 18, SEQ ID NO: 19, SEQ ID NO: 20, SEQ ID NO: 21, or SEQ ID NO: 22 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, the genomic disruption of the endogenous FAS gene suppresses or eliminates expression of the FAS gene in the modified γδT-cell (e.g., T-iγδT-cell) relative to an unmodified γδT-cell (e.g., an unmodified T-iγδT-cell) or relative to the γδT-cell (e.g., T-iγδT-cell) before modification. In some embodiments, the genomic disruption in the FAS gene is in exon 1 of the FAS gene. In some embodiments, the genomic disruption in the FAS gene is an inactivating nucleic acid mutation. In some embodiments, the genomic disruption in the FAS gene is an inactivating nucleic acid mutation in exon 1 of the FAS gene. In some embodiments, a genomic disruption or inactivating nucleic acid mutation in the FAS gene is an insertion, deletion, substitution, or any combination thereof. In some embodiments, the genomic disruption is within a target sequence in FAS that comprises the nucleotide sequence of SEQ ID NO: 62 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, the genomic disruption of the endogenous B2M gene suppresses or eliminates expression of the B2M gene in the modified γδT-cell (e.g., T-iγδT-cell) relative to an unmodified γδT-cell (e.g., an unmodified T-iγδT-cell) or relative to the γδT-cell (e.g., T-iγδT-cell) before modification. In some embodiments, the genomic disruption in the B2M gene is in exon 2 of the B2M gene. In some embodiments, the genomic disruption in the B2M gene is an inactivating nucleic acid mutation. In some embodiments, the genomic disruption in the B2M gene is an inactivating nucleic acid mutation in exon 2 of the B2M gene. In some embodiments, a genomic disruption or inactivating nucleic acid mutation in the B2M gene is an insertion, deletion, substitution, or any combination thereof. In some embodiments, the genomic disruption is within a target sequence in B2M that comprises the nucleotide sequence of SEQ ID NO: 56 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, the genomic disruption of the endogenous CIITA gene suppresses or eliminates expression of the CIITA gene in the modified γδT-cell (e.g., T-iγδT-cell) relative to an unmodified γδT-cell (e.g., an unmodified T-iγδT-cell) or relative to the γδT-cell (e.g., T-iγδT-cell) before modification. In some embodiments, the genomic disruption in the CIITA gene is in exon 3 of the CIITA gene. In some embodiments, the genomic disruption in the CIITA gene is an inactivating nucleic acid mutation. In some embodiments, the genomic disruption in the CIITA gene is an inactivating nucleic acid mutation in exon 3 of the CIITA gene. In some embodiments, a genomic disruption or inactivating nucleic acid mutation in the CIITA gene is an insertion, deletion, substitution, or any combination thereof. In some embodiments, the genomic disruption is within a target sequence in CIITA that comprises the nucleotide sequence of SEQ ID NO: 59 or a nucleotide sequence having at least 70% (e.g., at least 70%, 75%, 80%, 85%, 90%, 91%, 92%, 93%, 94%, 95%, 96%, 97%, 98%, 99%, or 100%) identity thereto.

In some embodiments, a genomic disruption or inactivating nucleic acid mutation in the SOCS1, CISH, BIM, B2M, CIITA, and/or FAS gene is an insertion, deletion, substitution, or any combination thereof.

In some embodiments, the method comprises contacting the cell with guide RNAs and an endonuclease or a nucleic acid encoding an endonuclease, wherein the contacting results in a genomic disruption in an endogenous SOCS1 gene that suppresses or eliminates expression of SOCS1 and a genomic disruption in an endogenous CISH gene that suppresses or eliminates expression of CISH. If BIM is also being disrupted, then a guide RNA is used that results in inhibiting expression of an endogenous BIM gene. If B2M is also being disrupted, then a guide RNA is used that results in inhibiting expression of an endogenous B2M gene. If CIITA is also being disrupted, then a guide RNA is used that results in inhibiting expression of an endogenous CIITA gene. If FAS is also being disrupted, then a guide RNA is used that results in inhibiting expression of an endogenous FAS gene.

Further information on the guide RNA and endonuclease could be found in other sections of the present disclosure, e.g., Section V: Gene editing systems.

Further information on the cells used in the methods of inhibiting could be found in other sections of the present disclosure, e.g., Section II: Modified cells and Section III: Modified cells comprising CAR, e.g., anti-CD19 CAR.

In some embodiment, methods of introducing any one or more exogenous polynucleotides encoding an IFNγ signal converter in γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) are provided.

In some embodiment, methods of introducing any one or more of the exogenous polynucleotides encoding anti-CD19 CAR, B2M-HLA-E, and IFNγ signal converter in γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) are provided.

In some embodiments, introducing any one or more exogenous polynucleotides to γδT-cells, e.g., iPSC-derived γδT-cells (and their precursors (e.g., an iPSC (e.g., γδT-iPSC), or an intermediate cell differentiated from an iPSC (e.g., γδT-iHSC, γδT-iCLP, and immature T-iγδT cells))) are achieved by knock-in. In some embodiments, the knock-in is a targeted knock-in obtainable by a CRISPR-Cas system (e.g., Cas9, Cas12 or MAD7) targeting a genomic locus of interest, in combination with a donor template comprising the exogenous polynucleotide for integration into the genomic locus of interest by homologous-directed repair.

In some embodiments, the present disclosure provides the modified cells generated from the method of inhibiting expression and/or method of introducing exogenous polynucleotides described herein.

The present disclosure provides a method of generating the modified γδT cells, comprising (i) generating the modified iPSC cells described herein (e.g., Section II and III), and (ii) differentiating the modified iPSC cells into the modified γδT cells.

### VII. Pharmaceutical Compositions

Pharmaceutical compositions comprising any of the modified cells or populations of cells (e.g., modified γδT) are encompassed. In some embodiments, pharmaceutical composition comprises the modified cell or population of cells (e.g., modified γδT) and a pharmaceutically acceptable carrier.

### VIII. Methods of Treatment

Described herein are methods of treating diseases and disorders using the modified cells, population of cells, or pharmaceutical compositions described herein.

In some embodiments, the present disclosure provides a method of killing B cells which are CD19 positive and have abnormal B cell function, comprising administering to a subject in need thereof a therapeutically effective amount of the modified γδT cell described herein or the pharmaceutical composition described herein. In some embodiments, the B cells are the CD19 positive B cells in diffuse large B-cell lymphoma (DLBCL) or systemic lupus erythematosus (SLE) patients.

In some embodiments, the present disclosure also provides a method of treating DLBCL or SLE, comprising administering to a subject in need thereof a therapeutically effective amount of the modified γδT cell described herein or the pharmaceutical composition described herein.

In some embodiments, the present disclosure also provides the modified γδT cell described herein or the pharmaceutical composition described herein for use in therapy.

In some embodiments, the present disclosure also provides the modified γδT described herein or the pharmaceutical composition described herein for use in treatment of DLBCL or SLE.

In some embodiments, the present disclosure also provides use of the modified cell, (e.g., the modified γδT cell, the modified iPSC cells described herein) or the pharmaceutical composition described herein in the manufacturing of a medicament for treatment of DLBCL or SLE.

The abbreviation, “e.g.,” is derived from the Latin exempli gratia, and is used herein to indicate a non-limiting example. Thus, the abbreviation “e.g.,” is synonymous with the term “for example.” The abbreviation, “i.e.,” is derived from the Latin id est, and is used herein to indicate a non-limiting rewording or clarification. Thus, the abbreviation “i.e.,” is synonymous with the term “that is.”

Section headings, materials, methods, and examples are illustrative only and not intended to be limiting.

### EXAMPLES

### Example 1. Generation of CISH^{-/-}SOCS1^{-/-}BIM^{-/-} Triple Knockout (TKO) TiPSC-2dT Cells

### Example 1.1 Methods

### Nucleofection

To generate target gene knockout in T-igdT cells from a Vd2 γδT-derived iPSC (iPSC-derived γδT, wherein the iPSC is generated from a primary Vd2 γδTcell), 5×10⁶ of T-igdT cells were sequentially nucleofected with 200 pmol of Alt-R™ S.p. HiFi Cas9 Nuclease V3 (Integrated DNA Technologies (Coralville, IA); Cat: 1081060) and 1,000 pmol of synthetic single guide RNA (sgRNAs) by Lonza P3 Primary Cell 4D-Nucleofector® X Kit L (Lonza (Basel, Switzerland), Cat: V4XP-3024) and Lonza 4D Nucleofector® Core and X Unit (Lonza, Cat: AAF-1003X) per Nucleocuvette vessel. To create a double knockout (DKO) or triple knockout (TKO) T-igdT cell, T-igdT cells were sequentially nucleofected to ensure the knockout efficiency of each target gene. Nucleofected T-igdT cells were subcultured with culture medium (STEMdiff APEL2 Medium (STEMCELL; Cat: 05275) supplemented with Penicillin-Streptomycin (100 U/mL; Gibco, Cat: 15140-122), AA2P (50 ng/mL; Cayman, Cat: 16457), EliteMu™ human platelet lysate (HIPL) (10%; EliteCell, Cat: EPI-500)) supplemented with recombinant human IL-7 (10 ng/mL; PeproTech (Cranbury, NJ), Cat: 200-07), and recombinant human IL-15 (10 ng/mL; PeproTech, Cat: 200-15) at cell density of 1×10⁶ cells/mL every 2 days prior to next round of nucleofection.

### Example 1.2 Nucleofection with Cas9 Nuclease and sgRNAs in T-igdT Cells

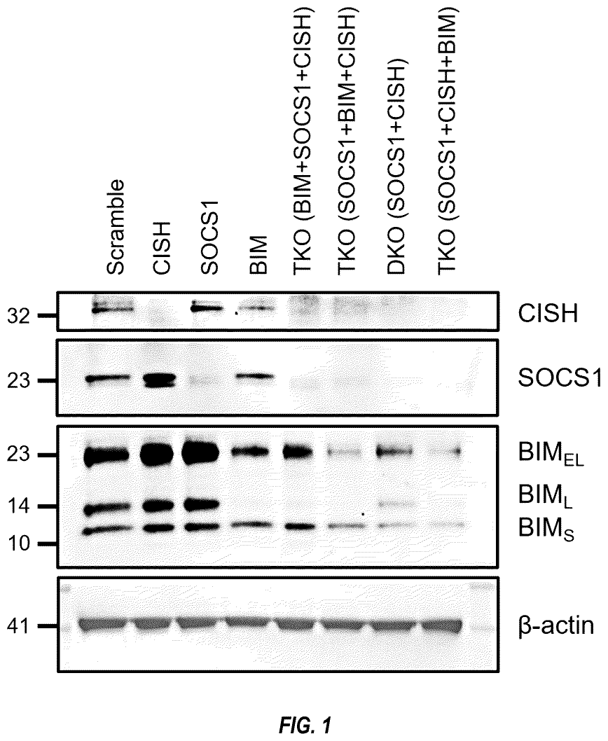

To generate the single, double or triple target gene knockout T-igdT cells, 5×10⁶ T-igdT cells were nucleofected with 200 pmol of Alt-R™ S.p. HiFi Cas9 Nuclease V3 (Integrated DNA Technologies; Cat: 1081060) and 1,000 pmol of synthetic single guide RNA (sgRNAs) as listed in Table 1 by Lonza P3 Primary Cell 4D-Nucleofector® X Kit L (Lonza, Cat: V4XP-3024) and Lonza 4D Nucleofector® Core and X Unit (Lonza, Cat: AAF-1003X) per Nucleocuvette vessel. Each target gene was knocked out by one synthetic sgRNA except CISH, which was knocked out by simultaneously using two sgRNAs (CISH #3 and #4). To ensure the knockout efficiency ofeach target gene, T-igdT cells were sequentially nucleofected. Since the cytotoxic efficacy and survival of T-igdT cells would be affected by nucleofection, all groups were nucleofected for a total of three rounds. For the single knockout group, T-igdT cells were nucleofected with target gene sgRNA(s) in a first round of nucleofection followed with scramble sgRNA for second and third round of nucleofection. For TKO/DKO, the T-igdT cell were sequentially nucleofected in the order as shown in the parentheses of FIG. 1 and reproduced here: TKO (BIN4+SOCS1+CISH) has the T-igdT cell nucleofected with sgRNA targeting BIN4, then sgRNAtargeting SOCS, and finally, with sgRNAtargeting CISH; TKO (SOC51+BIM+CISH) has the T-igdT cell nucleofected with sgRNA targeting SOC51, then sgRNA targeting BIM, and finally, with sgRNA targeting CISH; DKO (SOCS1+CISH) has the T-igdT cell nucleofected with sgRNA targeting SOCS1, and, then, with sgRNA targeting CISH; TKO (SOCS1+CISH+BIM) has the T-igdT cell nucleofected with sgRNA targeting SOCS1, then sgRNA targeting CISH, and finally, with sgRNA targeting BIN4. Nucleofected T-igdT cells were subcultured with culture medium supplemented with recombinant human IL-7 (10 ng/mL; PeproTech, Cat: 200-07), and recombinant human IL-15 (10 ng/mL; PeproTech, Cat: 200-15) at cell density of 1×10⁶ cells/mL every 2 days prior to the next round of nucleofection.

TABLE 1
sgRNA sequence of candidate genes used for nucleofection with Cas9 nuclease

| | Name | | Targeting/ | Target | |
|---|---|---|---|---|---|
| Target | of | | guide sequence | sequence in | Target |
| Gene | sgRNA | Synthetic sgRNA sequence | in sgRNA | target gene | exon |
| / | Scramble | mG*mC*mA*rCrUrCrArCrArUrCrGr | GCACTCACATC | TGATGTAGCG | / |
| | sgRNA | CrUrArCrArUrCrArGrUrUrUrUrArGr | GCTACATCA | ATGTGAGTG | |
| | (Scramble | ArGrCrUrArGrArArArUrArGrCrArAr | (SEQ ID NO: 1) | C | |
| | #2) | GrUrUrArArArArUrArArGrGrCrUrAr | | (SEQ ID NO: | |
| | | GrUrCrCrGrUrUrArUrCrArArCrUrUr | | 13) | |
| | | GrArArArArArGrUrGrGrCrArCrCrGr | | | |
| | | ArGrUrCrGrGrUrGrCmU*mU*mU*r | | | |
| | | U (SEQ ID NO: 25) | | | |
| BIM | BIM | mU*mA*mC*rCrCrArUrUrGrCrArCr | TACCCATTGCA | CTATCTCAGT | Exon |
| (BCL2L11) | sgRNA | UrGrArGrArUrArGrGrUrUrUrUrArGr | CTGAGATAG | GCAATGGGT | 2C |
| | (BIM #5) | ArGrCrUrArGrArArArUrArGrCrArAr | (SEQ ID NO: 2) | A | |
| | | GrUrUrArArArArUrArArGrGrCrUrAr | | (SEQ ID NO: | |
| | | GrUrCrCrGrUrUrArUrCrArArCrUrUr | | 14) | |
| | | GrArArArArArGrUrGrGrCrArCrCrGr | | | |
| | | ArGrUrCrGrGrUrGrCmU*mU*mU*r | | | |
| | | U (SEQ ID NO: 26) | | | |
| CIS | CISH | mA*mG*mG*rCrCrArCrArUrArGrUr | AGGCCACATAG | TGTGCAGCA | Exon 4 |
| (CISH) | sgRNA | GrCrUrGrCrArCrArGrUrUrUrUrArGr | TGCTGCACA | CTATGTGGCC | |
| | (CISH | ArGrCrUrArGrArArArUrArGrCrArAr | (SEQ ID NO: 3) | T | |
| | #3) | GrUrUrArArArArUrArArGrGrCrUrAr | | (SEQ ID NO: | |
| | | GrUrCrCrGrUrUrArUrCrArArCrUrUr | | 15) | |
| | | GrArArArArArGrUrGrGrCrArCrCrGr | | | |
| | | ArGrUrCrGrGrUrGrCmU*mU*mU*r | | | |
| | | U (SEQ ID NO: 27) | | | |
| | CISH | mU*mG*mU*rArCrArGrCrArGrUrGr | TGTACAGCAGT | CCACCAGCC | Exon 4 |
| | sgRNA | GrCrUrGrGrUrGrGrGrUrUrUrUrArGr | GGCTGGTGG | ACTGCTGTAC | |
| | (CISH | ArGrCrUrArGrArArArUrArGrCrArAr | (SEQ ID NO: 4) | A | |
| | #4) | GrUrUrArArArArUrArArGrGrCrUrAr | | (SEQ ID NO: | |
| | | GrUrCrCrGrUrUrArUrCrArArCrUrUr | | 16) | |
| | | GrArArArArArGrUrGrGrCrArCrCrGr | | | |
| | | ArGrUrCrGrGrUrGrCmU*mU*mU*r | | | |
| | | U (SEQ ID NO: 28) | | | |
| SOCS1 | SOCS1 | mA*mG*mU*rArGrArArUrCrCrGrCr | AGTAGAATCCG | GGACGCCTG | Exon 2 |
| | sgRNA | ArGrGrCrGrUrCrCrGrUrUrUrUrArGr | CAGGCGTCC | CGGATTCTAC | |
| | | ArGrCrUrArGrArArArUrArGrCrArAr | (SEQ ID NO: 5) | T | |
| | | GrUrUrArArArArUrArArGrGrCrUrAr | | (SEQ ID NO: | |
| | | GrUrCrCrGrUrUrArUrCrArArCrUrUr | | 17) | |
| | | GrArArArArArGrUrGrGrCrArCrCrGr | | | |
| | | ArGrUrCrGrGrUrGrCmU*mU*mU*r | | | |
| | | U (SEQ ID NO: 29) | | | |

means the phosphorothioate linkage between each base, and “m″”mean 2′-O-Methyl modification.

### Western Blotting

To validate the knockout efficiency of target genes in T-igdT cells post nucleofection, western blotting was performed to examine the protein expression level of various genes. Briefly, T-igdT cells were harvested, and cell pellets were lysed in radioimmunoprecipitation (RIPA) buffer (Millipore (Burlington, MA), Cat: 20-188) containing protease inhibitor cocktail (TargetMol (Boston, MA), Cat: C0001). The mixture was pipetted gently and put on ice for 30 minutes and then centrifuged at 12,000×g for 15 minutes at 4° C. The supernatants were transferred to a new 1.6 mL microcentrifuge tube. The protein concentration of the cell lysate was determined with Protein Assay Dye Reagent (Bio-Rad (Hercules, CA), Cat: 5000006). A total of 20 mg of protein in each lane was separated with mPAGE 4-2O₀% Bis-Tris Precast Gel (Millipore, Cat: M1P42G12). The separated proteins were subsequently transferred to Power Blotter Select Transfer polyvinylidene fluoride (PVDF) membrane (Invitrogen (Waltham, MA), Cat: PB5340). The gel and the PVDF membrane were carefully removed from the stack. The blot was rinsed with 1×TBST (Tris buffered saline and polysorbate 20). The transferred PVDF membrane was blocked with 500 milk in TBST at room temperature for 1 hour. The blot was rinsed three times for 10 minutes with TBST and then incubated with a BIN4-targeting antibody (1:1,000; Cell Signaling Technology (Danvers, MA), Cat: 2933S), a CISH-targeting antibody (1:1,000; Cell Signaling Technology, Cat: 8731S) and a SOCS1-targeting antibody (1:1,000; Cell Signaling Technology, Cat: 6863IS) in 1% milk/TBST overnight at 4° C. The blot was rinsed three times for 10 minutes with TBST and incubated with Peroxidase conjugated goat anti-rabbit IgG antibody (1:5,000; Sigma-Aldrich (St. Louis, MO), Cat: AP132P) in 1% milk/TBST at room temperature for 1 hour. The blot was rinsed three times for 10 minutes with TBST. The SuperSignal™ West Pico PLUS Chemiluminescent Substrate (ThermoFisher Scientific, Cat: 34580) was applied to the blot according to the manufacturer's recommendation. The chemiluminescent signals were captured using the ChemiDoc Imaging System (Bio-Rad). Beta-actin (1:4,000; Santa Cruz Biotechnology (Dallas, TX), Cat: sc-47778 HRP) was used as an internal reference. Western blotting result confirmed the efficient knockout of target genes by nucleofection with Cas9 nuclease and synthetic sgRNAs (FIG. 1).

As shown in FIG. 1, Western blotting results showed that CISH sgRNAs (CISH #3 and #4) and SOCS1 sgRNA could efficiently knock out their protein expression. Human BIM gene, also called BCL2L11 (Gene ID: 10018), is transcribed as three splice variants, BIM_{EL}(NM_138621.5; NP_619527.1, containing coding exons E1, E2A, E2B, E2C, E4 and E5), BIM_{L} (NM_006538.5; NP_006529.1, containing coding exons E1, E2A, E2C, E4 and E5) and BIMs (NM_001204106.1; NP_001191035.1, containing coding exons E1, E2A, E4 and E5). The expression level of BIM_{EL} was higher than BIM_{L} and BIM_{S} splice variants in wild-type T-igdT cells. For BIM knockout in T-igdT cells, nucleofection was performed with BIM sgRNA #5, which targets BIM exon E2C, hence the knockout of BIM_{EL} and BIM_{L} splice variants while preserving the BIM_{S} splice variant in T-igdT cells. Western blotting results showed BIM_{L} was a complete knockout, and BIM_{EL} was a substantial and efficient knockout with trace amounts shown in western blotting. For example, the percentages of BIM_{EL} in BIM single KO and TKO groups range from about 11.3% to about 54.7% relative to the scramble group. Overall, BIM sgRNA #5 could efficiently knock out the BIM_{EL} and BIM_{L} splice variants, while preserving the BIM_{S} splice variant.

### Indel and Knockout Score by ICE Analysis

2×10⁵ of genome-edited T-igdT cells were harvested, and the genomic DNA was extracted with QuickExtract™ DNA Extraction Solution 1.0 (LGC, Teddington, England, Cat: SS000035-D2). Target gene-specific DNA fragment was amplified by KAPA HiFi HotStart PCR Kit ((Roche, Basel, Switzerland), Cat: KR0369) with specific primer pairs as listed in Table 2 and following amplification program:
Step 1, 95° C. for 3 minutes
Step 2, 98° C. for 20 seconds
Step 3, Melting temperature (Tm° C.) for 15 seconds
Step 4, 72° C. for 60 seconds
Repeat Step 2-4 for 30 cycles
Step 5, 72° C. for 60 seconds

The PCR products were sequenced by MISSION BIOTECH (Taipei City, Taiwan). The primer sequences used for sequencing were the same as the PCR primer sequences. The Indel percentages were analyzed by Synthego (Redwood City, CA) Inference of CRISPR Edits (ICE) Analysis. Briefly, the sequencing results of target gene knockout group and the scramble control were imported into the Synthego ICE analysis tool. The indel percentage and knockout score were acquired after the Synthego ICE analysis (Table 3). The results of the indel percentage and knockout score showed an efficient knockout of each target gene by sequential nucleofection with Cas9 nuclease and sgRNAs.

TABLE 2
Primer sequence of candidate genes for ICE analysis

| Primer | Sequence (5′→3′) | Tm (° C.) |
|---|---|---|
| BIM forward | TTTGCTACCAGATCCCCGCT (SEQ ID | 65° C. |
| BIM reverse | CCAAACCCTCTGAGCAATAAGCA | |
| | (SEQ ID NO: 40) | |
| CISH forward | GCACTGCCTTGACTTGCACT (SEQ ID | 67° C. |
| | NO: 41) | |
| CISH reverse | GGGGGCATTTACCTCCAAGT (SEQ ID | |
| | NO: 42) | |
| SOCS1 forward | TCCACTGAGGCTGAACGGAT (SEQ ID | 65° C. |
| | NO: 43) | |
| SOCS1 reverse | CTGAAAGTGCACGCGGATG (SEQ ID | |
| | NO: 44) | |

TABLE 3
Indel percentage and knockout (KO) score of candidate genes

| | CISH | CISH KO | SOCS1 | SOCS1 | BIM | BIM KO |
|---|---|---|---|---|---|---|
| Group | Indel | score | Indel | KO score | Indel | score |
| Scramble | — | — | — | — | — | — |
| CISH | 98% | 93 | — | — | — | — |
| SOCS1 | — | — | 78% | 73 | — | — |
| BIM | — | — | — | — | 92% | 84 |
| SOCS1 + CISH | 99% | 98 | 76% | 71 | — | — |
| SOCS1 + BIM + CISH | 91% | 81 | 76% | 71 | 91% | 77 |

### Example 2. CISH^{-/-}SOCS1^{-/-}DKO and CISH^{-/-}SOCS1^{-/-}BIM^{-/-} (More specifically. BIM_{EL} and BIM_{L} deficiency while preserving BIM_{S}) TKO Enhance the Tumor Killing by T-iγδT Cells and Persistence of T-iγδT Cells

### Serial Killing Assay of T-iγδT Cells Against HepG2 Cells (2D Platform)

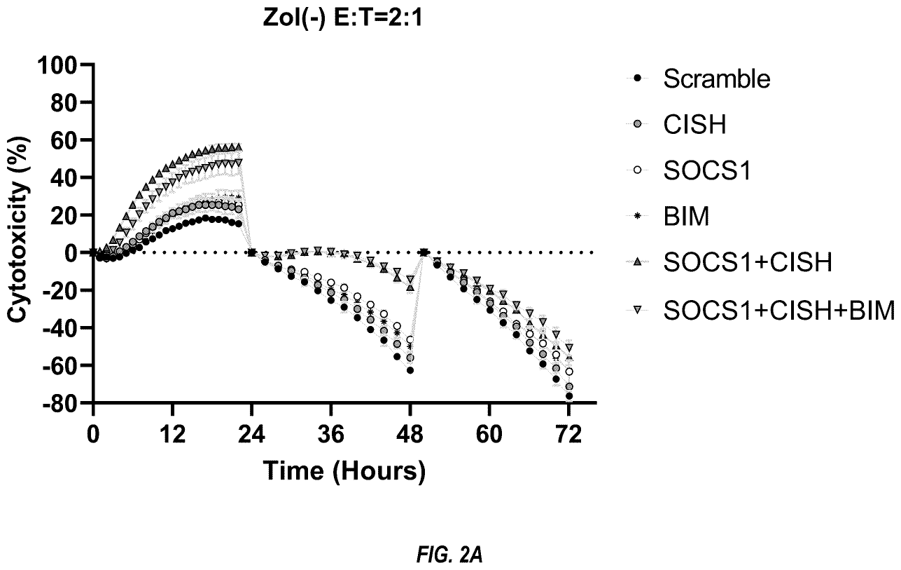

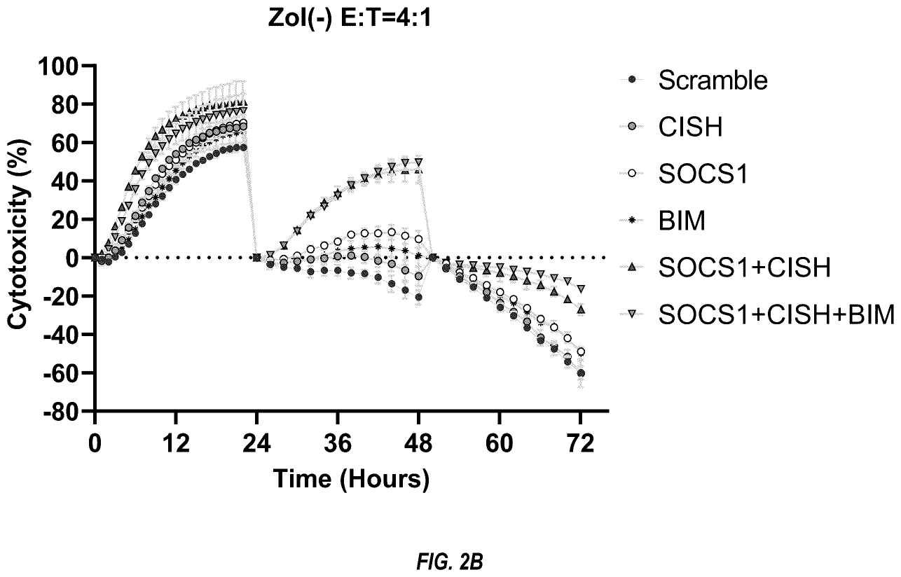

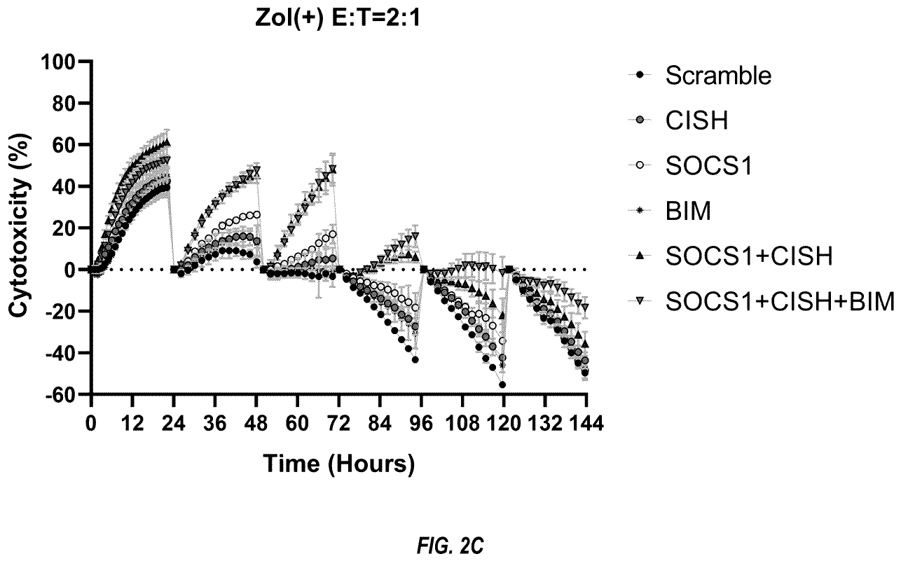

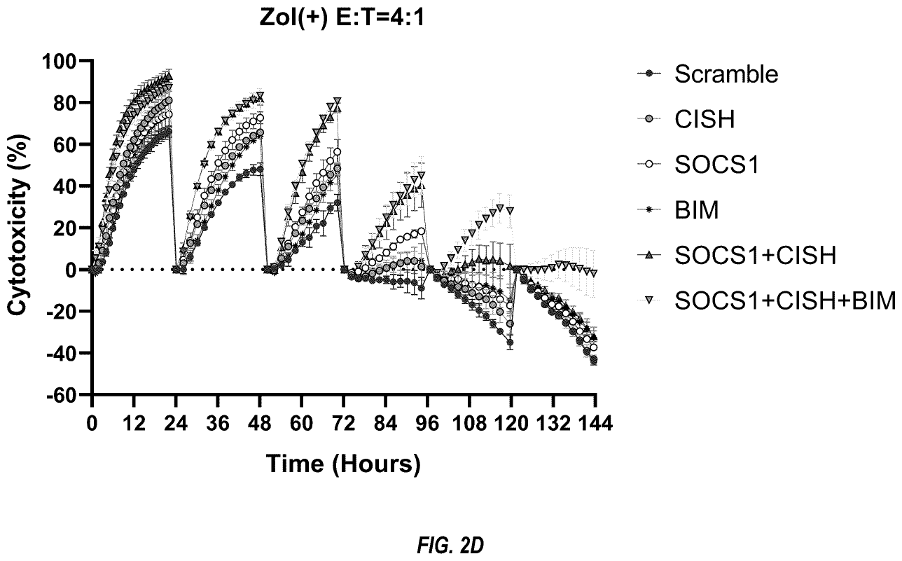

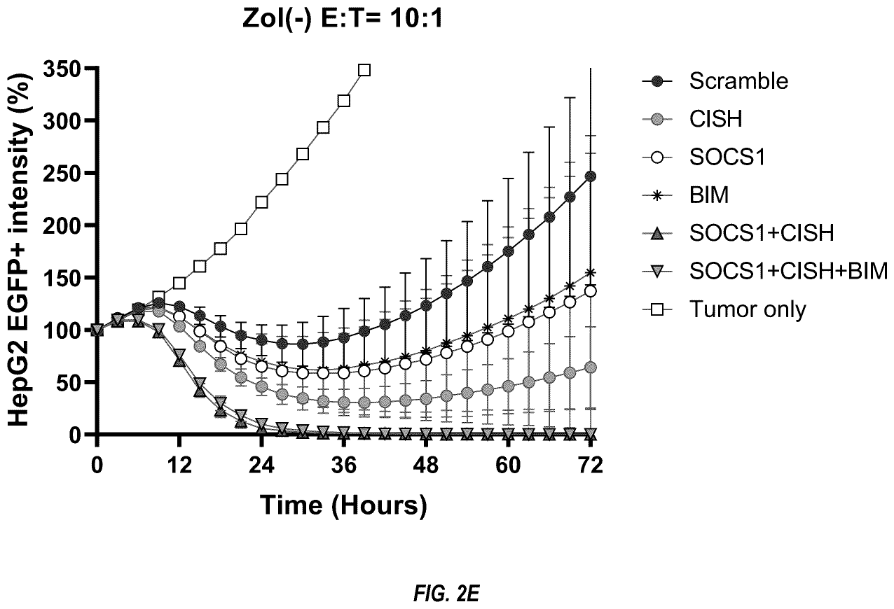

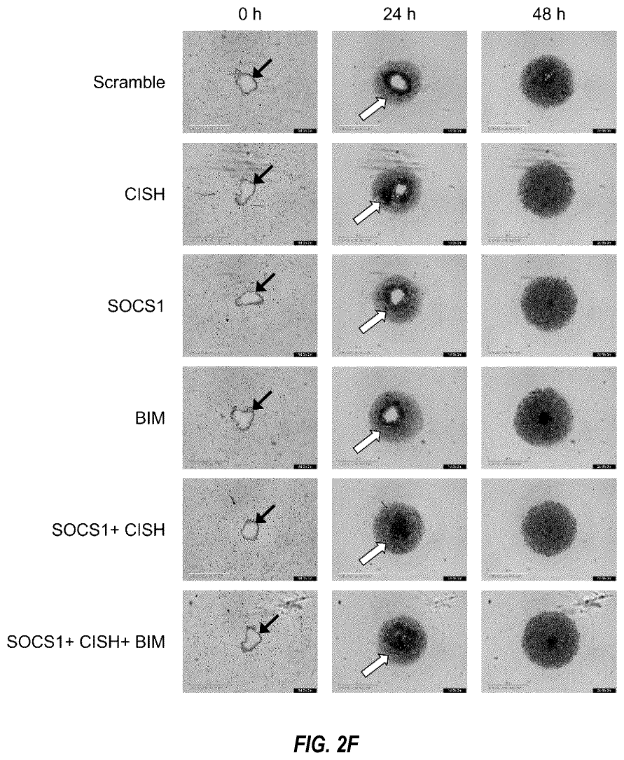

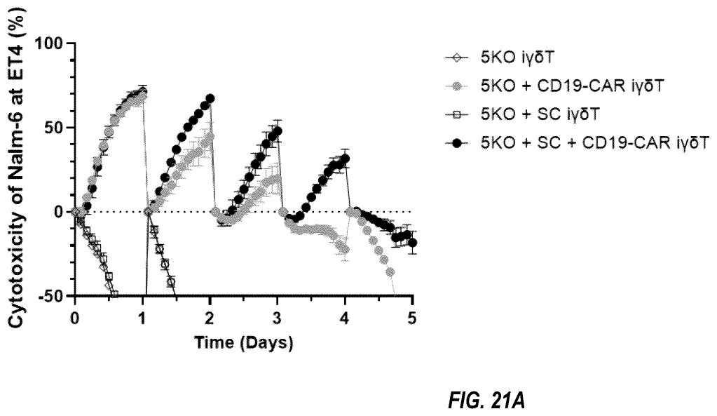

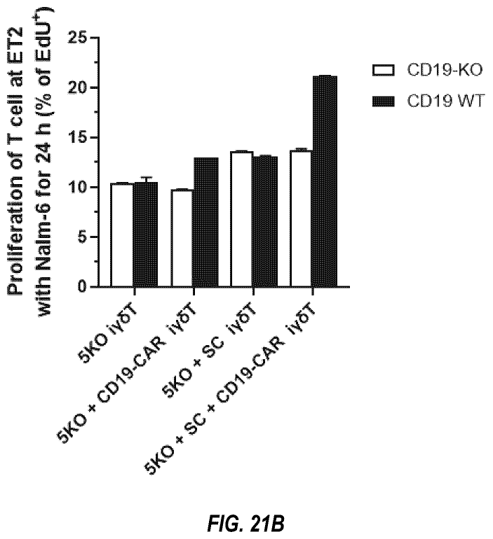

5×10³ of EGFP-HepG2 cells (ATCC (Manassas, VA)), Cat: HB1-8065, a cell line exhibiting epithelial-like morphology that was isolated from a hepatocellular carcinoma) were pre-seeded in a 96-well cell culture plate (Corning, Cat: 3599) and treated without (FIGS. 2A and 21B) or with (FIGS. 2C and 2D) bisphosphonate zoledronic acid (Zol) (5 mM; Sigma-Aldrich, Cat: 1724827) overnight. After Zol treatment, the culture supernatant was aspirated. The effector T-igdT cells with target gene deficiencies, generated from Example 1, were added into the Zol-treated 96-well cell culture plate or the 96-well cell culture plate without a Zol treatment in 200 mL of a culture medium without cytokines at a 4:1 or 2:1 effector to target cell ratio. The cytotoxicity of T-igdT cells was monitored using live-cell imaging and analysis, using the Incucyte® SX5 Live-Cell Analysis System (Sartorius, Gottingen, Germany) through a 10× objective and scheduled 24-hour repeated scanning. Hereafter, the effector T-igdT cells with target gene deficiencies were transferred to another Zol-treated GFP-HepG2 pre-seeded 96-well cell culture plate or Zol-untreated GFP-HepG2 pre-seeded 96-well cell culture plate after 24 hours of tumor killing for a further round of killing. The cytotoxicity percentage was calculated by the formula listed below:

$$Cytotoxicity percentage = [ 1 - ( Total green fluorescence area at indicated timepoint / Total green fluorescence area at starting timepoint of each round of serial killing ) ] \times 100 \% \qquad a$$

Human gdT cells can be activated by phosphoantigen such as Zoledronic acid (Zol), which is expressed on the cell surface of various tumor cells. All single gene knockout T-igdT cells could not execute a second round of killing in the absence of Zol at 2:1 and 4:1 E:T ratios (FIGS. 2A and 2B). In the presence of Zol (FIG. 2D), scramble sgRNA-nucleofected T-igdT cells could exhibit 3 rounds of killing against the EGFP-HepG2 cells compared with one round of killing by T-igdT cells in the absence of Zol at a 4:1 E:T ratio (FIG. 2B).

As shown FIG. 2A-2D, CISH^{-/-}SOCS1^{-/-} double knockout (DKO) T-igdT cells exhibited synergistic effects in promoting T-igdT persistence and/or killing over CISH or SOCS1 single knockout T-igdT cells with or without Zol at a 4:1 or 2:1 E:T ratio (FIGS. 2A-2D). More specifically, as shown in FIG. 2A, CISH^{-/-}SOCS1^{-/-}DKO T-igdT cells showed synergistic effects in killing cancer cells in the first round of killing, compared with CISH or SOCS1 single knockout. As shown in FIG. 2B, CISH^{-/-}SOCS1^{-/-}DKO T-igdT cells showed synergistic effects in promoting gdT persistence and killing, especially in the second round of killing, compared with CISH or SOCS1 single knockout T-igdT cells. As shown in FIG. 2C, CISH^{-/-}SOCS1^{-/-} DKO T-igdT cells showed synergistic effects in promoting gdT cell persistence and killing, especially in the second round, third round and fourth round of killing, compared with CISH or SOCS1 single knockout T-igdT cells. As shown in FIG. 2D, CISH^{-/-}SOCS1^{-/-} DKO T-igdT cells showed synergistic effects in promoting gdT cell persistence and killing, especially in the third round, fourth and fifth round of killing, compared with CISH or SOCS1 single knockout T-igdT cells.

Interestingly, CISH^{-/-}SOCS1^{-/-}BIM^{-/-} (more specifically, BIM_{EL} and BIM_{L} deficiency while preserving BIM_{S}) TKO T-igdT cells could execute one more round (the sixth round) of killing in the presence of Zol at a 4:1 E:T ratio compared with CISH^{-/-}SOCS1^{-/-}DKO T-igdT cells (FIG. 2D).

Overall, CISH^{-/-}SOCS1^{-/-} DKO T-igdT cells or CISH^{-/-}SOCS1^{-/-}BIM^{-/-} (more specifically, BIM_{EL} and BIM_{L} deficiency while preserving BIM_{S}) TKO T-igdT cells exhibited synergistic effects in enhanced cytotoxicity and persistence against HepG2 cells compared with all the other single gene knockout T-igdT cells in the 2D serial killing assay platform (FIGS. 2A-2D).

### Killing Assay of T-igdT Cells Against HepG2 Spheroid (3D Platform)

1×10³ EGFP-HepG2 cells (ATCC, Cat: HB-8065) were seeded in a 96-well Ultra-Low Attachment Microplate (Corning, Cat: 7007). The culture plate was centrifugated at 125×g for 10 minutes and incubated at 37° C. in 5% CO₂ for 3 days. The effector T-igdT cells with a target gene deficiency were added into the above plate in 100 mL of culture medium without cytokines at a 10:1 or 20:1 effector to target cell ratio. The cytotoxicity of T-igdT cells was monitored using live-cell imaging and analysis, using the Incucyte® SX5 Live-Cell Analysis System (Sartorius) through a 4× objective and scheduled, repeated scanning every 3-6 hours for up to 7 days. The cytotoxicity was quantified by total green fluorescence intensity at indicated timepoints normalized to the green fluorescence intensity at the 0 hour timepoint×100%.

CISH single knockout T-igdT cells exhibit about 50% cytotoxicity against HepG2 spheroid and exhibited the among all single gene knockout groups (FIG. 2E). However, all single gene knockout T-igdT cells could not eliminate HepG2 spheroids 24 hours post coculture (FIGS. 2E and 2F). In the left and middle panel of FIG. 2F, EGFP positive HepG2 cells were indicated with a black arrow (bright area), and, in the middle panel of FIG. 2F, surrounded T-igdT cells were indicated with a white arrow. CISH^{-/-}SOCS1^{-/-} double knockout (DKO) T-igdT cells showed synergistic effects in cytotoxicity against HepG2 spheroids over CISH or SOCS1 single knockout T-igdT cells. Overall, CISH^{-/-}SOCS^{-/-}DKO and CISH^{-/-}SOCS1^{-/-}BIM^{-/-} (more specifically, BIM_{EL} and BIM_{L} deficiency while preserving BIM_{S}) TKO T-igdT cells exhibited synergistic effects in enhanced cytotoxicity against HepG2 spheroids and eliminated HepG2 spheroids 24 hours post coculture compared with all the other single gene knockout T-igdT cell groups in the 3D killing assay platform (FIGS. 2E and 2F).

### Example 3. Prolonged Survival of CISH^{-/-}SOCS1^{-/-}BIM^{-/-}Triple Knockout (TKO) T-igdT Cells Post IL-15 Deprivation for 12 Days

### Example 3.1 Methods

Survival Assay with IL-15 Deprivation

T-igdT cells with target gene deficiency were harvested on day 4 post the final round of nucleofection and washed with 1 mL of cytokine-free culture medium. 3×10⁴ of the harvested T-igdT cells were resuspended in 100 mL of pre-warmed cytokine-free culture medium. The two-fold culture medium was supplemented with titration of IL-15 (0, 0.1, 0.6 and 20 ng/mL). 100 mL of the T-igdT cell suspension (3×10⁴ cells) was mixed with 100 mL of the two-fold culture medium and were seeded in a 96-well cell culture plate (Falcon (Corning, Corning, NY), Cat: 353072) at total volume of 200 mL/well. 240 mL of PBS was added to the outer 36 wells to prevent evaporation of the medium. Seeded T-igdT cells were incubated at 37° C. with 5% CO₂ for 2, 4, 6, 8, 10, or 12 days. After IL-15 deprivation, T-igdT cells were harvested and transferred into a fresh 1.6 mL microcentrifuge tube every two days. Cell viability was determined with FITC-Annexin V (1:200; BioLegend (San Diego, CA), Cat: 640905) and Propidium Iodide (1:2,500; BioLegend, Cat: 421301) staining according to the manufacturer's instructions and analyzed by Attune™ NxT Flow Cytometer (ThermoFisher Scientific (Waltham, MA)). A live T-igdT cells was defined as a Annexin V negative−PI negative population analyzed with FlowJo™ software (BD Biosciences (San Jose, CA)). The relative viability at the indicated timepoint was calculated by normalization to the viability at 0 h timepoint.

### Example 3.2 Results

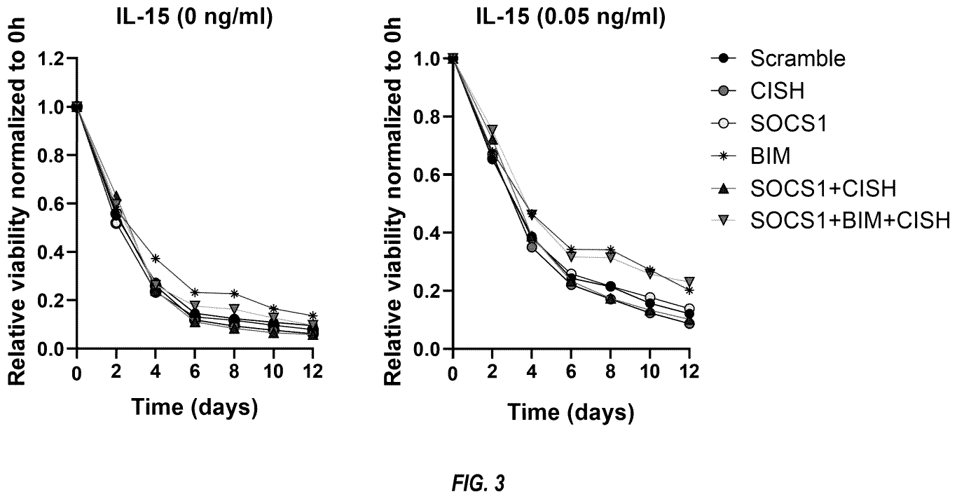

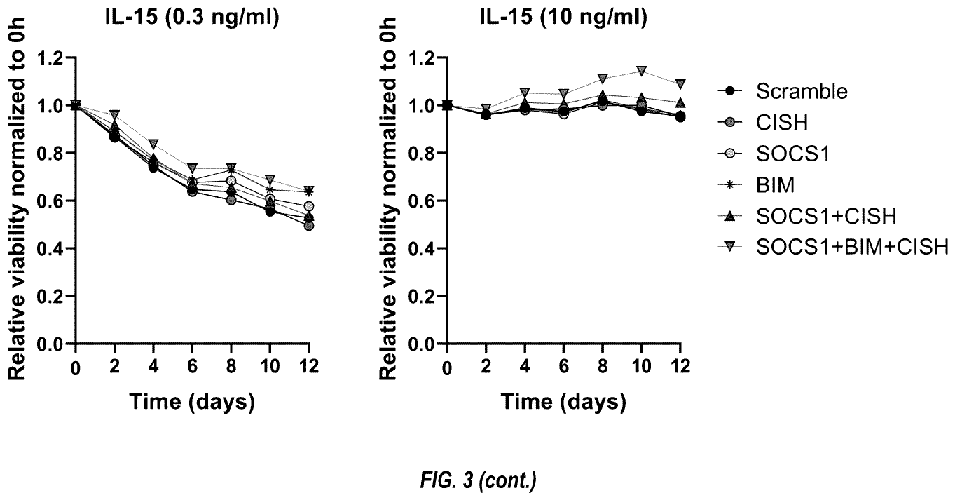

T-igdT cells with a target gene deficiency (generated according to Example 1) were harvested on day 4 post a final round of nucleofection and washed with 1 mL of cytokine-free culture medium. 3×10⁴ of T-igdT cells were resuspended in 100 mL of pre-warmed cytokine-free culture medium. The culture medium supplemented with a two-fold concentration of IL-15 (0, 0.1, 0.6 and 20 ng/mL) was prepared and 100 mL of the T-igdT cell suspension was mixed with 100 mL of a prepared culture medium to reach a final IL-15 concentration of 0, 0.05, 0.3 and 10 ng/mL. T-igdT cells were seeded in a 96-well cell culture plate (Falcon, Cat: 353072) at a total volume of 200 mL/well, and 240 mL of PBS were added to the outer 36 wells to prevent evaporation of the medium. T-igdT cells were incubated at 37° C. with 5% CO₂ for 2-12 days (e.g., 2, 4, 6, 8, 10, or 12 days). After IL-15 deprivation, T-igdT cells were harvested and transferred into a fresh 1.6 mL microcentrifuge tube every two days. Cell viability was determined with FITC-Annexin V (1:200; BioLegend, Cat: 640905) and Propidium Iodide (1:2,500; BioLegend, Cat: 421301) staining according to the manufacturer's instructions and analyzed by Attune™ NxT Flow Cytometer (ThermoFisher Scientific). Live T-igdT cells were defined as an Annexin V negative−PI negative population analyzed with FlowJo™ software (BD Biosciences). The relative viability of indicated timepoints was calculated by normalization to the viability of the 0 h timepoint. T-igdT cells with a BIM_{EL} and BIM_{L} deficiency while preserving BIM_{S} (knockout by BIM sgRNA #5) or CISH-SOCS1-BIM/(more specifically, BIM_{EL} and BIM_{L} deficiency while preserving BIM_{S}) TKO T-igdT cells exhibited better survival post IL-15 deprivation for 2-12 days (e.g., 2, 4, 6, 8, 10, or 12 days) (FIG. 3).

### Example 4. BIM_{S} Splice Variant is Necessary for Prolonging T-igdT Cell Survival Post IL-15 Deprivation

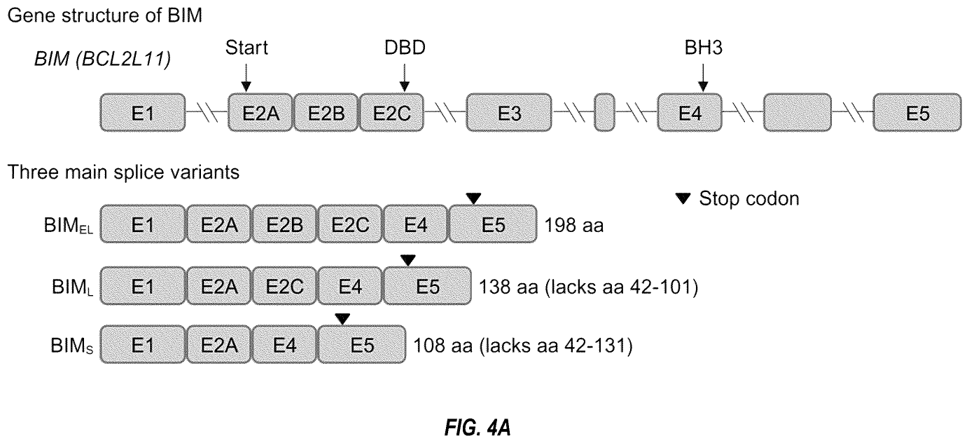

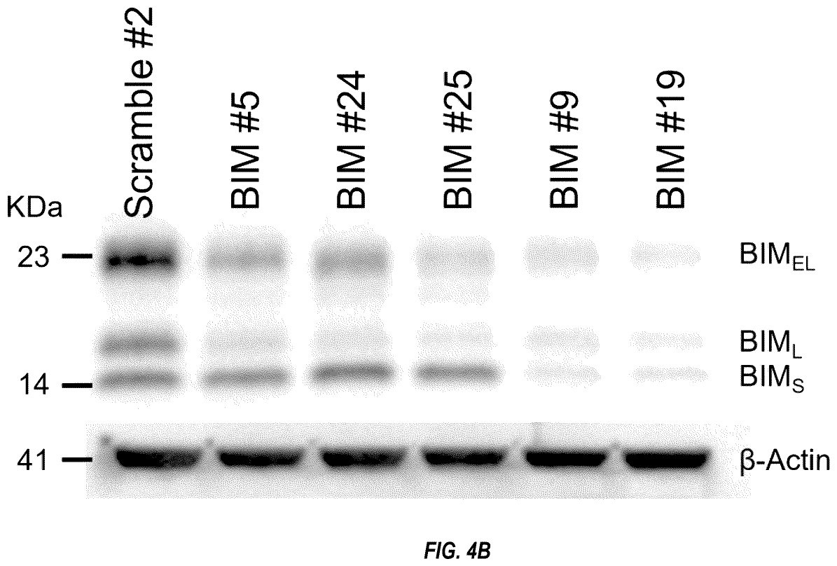

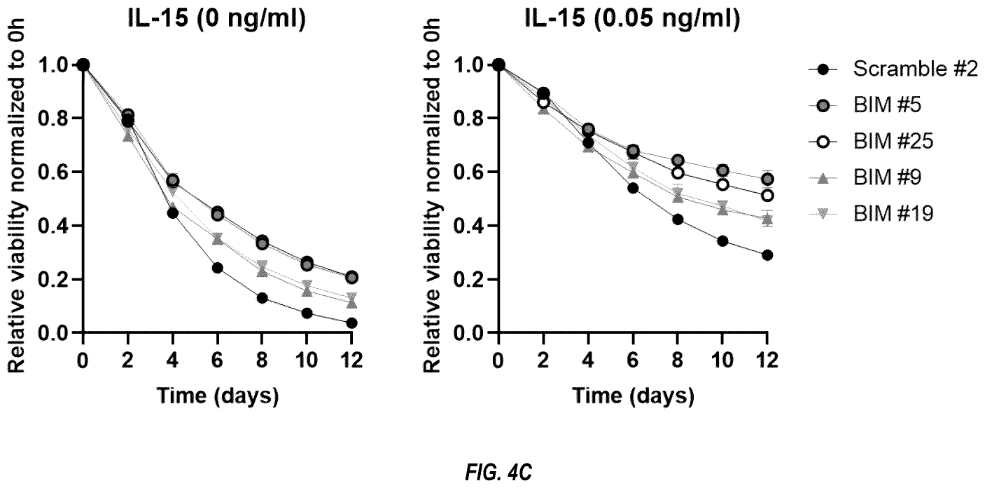

Human BIM gene, also called BCL2L11 (Gene ID: 10018), is transcribed as three splice variants, BIM_{EL} (NM_138621.5; NP_619527.1, containing coding exons E1, E2A, E2B, E2C, E4 and E5), BIM_{L} (NM_006538.5; NP_006529.1, containing coding exons E1, E2A, E2C, E4 and E5) and BIM_{S} (NM_001204106.1; NP_001191035.1, containing coding exons E1, E2A, E4 and E5). FIG. 4A shows gene structure of BIM and three main BIM splice variants expressed in T-igdT cells. T-igdT cells were nucleofected with 200 pmol of Alt-R™ S.p. HiFi Cas9 Nuclease V3 (Integrated DNA Technologies; Cat: 1081060) and 1,000 pmol of synthetic single guide RNA (sgRNAs). We had three synthetic sgRNAs that target BIM exon E2C (BIM #5 (SEQ ID NO: 26), #24 (SEQ ID NO: 34) and #25 (SEQ ID NO: 35)) and two synthetic sgRNAs that target BIM exon E2A (BIM #9 (SEQ ID NO: 32) and #19 (SEQ ID NO: 33)). Scramble #2 (SEQ ID NO: 30; a negative control for gene knockout), BIM #5 (SEQ ID NO: 26; targeting BIM_{EL} and BIM_{L} only), BIM #24 (SEQ ID NO: 34; targeting BIM_{EL} and BIM_{L} only), BIM #25 (SEQ ID NO: 35; targeting BIM_{EL} and BIM_{L} only), BIM #9 (SEQ ID NO: 32; targeting all three BIM splice variants) or BIM #19 (SEQ ID NO: 33; targeting all three BIM splice variants), as listed in Table 4, were nucleofected by Lonza P3 Primary Cell 4D-Nucleofector® X Kit L (Lonza, Cat: V4XP-3024) and Lonza 4D Nucleofector® Core and X Unit (Lonza, Cat: AAF-1003X). Western blotting results showed that BIM_{EL} and BIM_{L} was knocked out in BIM #5-, BIM #24- and BIM #25-nucleofected T-igdT cells and all three BIM splice variants were knockout in BIM #9- and BIM #19-nucleofected T-igdT cells. There were some background bands for BIM_{EL} because the nucleofection efficiency was not 100%, and the western blot samples were from pool populations (FIG. 4B).

T-igdT cells were harvested on day 4 post nucleofection and washed with 1 mL of cytokine-free culture medium. 3×10⁴ of T-igdT cells were resuspended in 100 mL of pre-warmed cytokine-free culture medium. The culture medium supplemented with a two-fold concentration of IL-15 (0 and 0.1 ng/mL) was prepared, and 100 mL of the T-igdT cell suspension was mixed with 100 mL of a prepared culture medium to reach a final IL-15 concentration of 0 and 0.05 ng/mL. T-igdT cells were seeded in a 96-well cell culture plate (Falcon, Cat: 353072) at a total volume of 200 mL/well, and 240 mL of PBS were added to the outer 36 wells to prevent evaporation of the medium. T-igdT cells were incubated at 37° C. with 5% CO₂ for 2-12 days (2, 4, 6, 8, 10 or 12 days). After IL-15 deprivation, T-igdT cells were harvested and transferred into a fresh 1.6 mL microcentrifuge tube every two days. Cell viability was determined with FITC-Annexin V (1:200; BioLegend, Cat: 640905) and Propidium Iodide (1:2,500; BioLegend, Cat: 421301) staining according to the manufacturer's instructions and analyzed by Attune™ NxT Flow Cytometer (ThermoFisher Scientific). Live T-igdT cells were defined as Annexin V negative−PI negative population analyzed with FlowJo™ software (BD Biosciences). The relative viability of indicated timepoints was calculated by normalization to the viability of the 0 h timepoint.

Overall, T-igdT cells that lack BIM_{EL} and BIM_{L} splice variants but retain the BIM_{S} splice variant (BIM #5 or BIM #25) exhibited better survival compared with T-igdT cells lacking all three BIM splice variants (BIM #9 or BIM #19) post IL-15 deprivation for 12 days (FIG. 4C).

TABLE 4
sgRNA sequence for targeting BIM splice variants with Cas9 nuclease

| | Name | | Targeting/guide | Target | |
|---|---|---|---|---|---|
| Target | of | | sequence in | sequence in | Target BIM |
| Gene | sgRNA | Synthetic sgRNA sequence | sgRNA | target gene | isoform |
| — | Scramble | mG*mC*mA*rCrUrCrArCrArUr | GCACTCACATC | TGATGTAGC | / |
| | #2 | CrGrCrUrArCrArUrCrArGrUrUr | GCTACATCA | GATGTGAGT | |
| | | UrUrArGrArGrCrUrArGrArArA | (SEQ ID NO: 1) | GC | |
| | | rUrArGrCrArArGrUrUrArArAr | | (SEQ ID NO: | |
| | | ArUrArArGrGrCrUrArGrUrCrC | | 13) | |
| | | rGrUrUrArUrCrArArCrUrUrGr | | | |
| | | ArArArArArGrUrGrGrCrArCrC | | | |
| | | rGrArGrUrCrGrGrUrGrCmU*m | | | |
| | | U*mU*rU (SEQ ID NO: 30) | | | |
| BIM | BIM #5 | mU*mA*mC*rCrCrArUrUrGrCr | TACCCATTGCA | CTATCTCAG | Target |
| (BCL2L11) | | ArCrUrGrArGrArUrArGrGrUrU | CTGAGATAG | TGCAATGGG | sequence is in |
| | | rUrUrArGrArGrCrUrArGrArAr | (SEQ ID NO: 2) | TA | exon 2C, |
| | | ArUrArGrCrArArGrUrUrArArA | | (SEQ ID NO: | resulting in |
| | | rArUrArArGrGrCrUrArGrUrCr | | 14) | knockout of |
| | | CrGrUrUrArUrCrArArCrUrUrG | | | BIM_{EL} and |
| | | rArArArArArGrUrGrGrCrArCr | | | BIM_{L} |
| | | CrGrArGrUrCrGrGrUrGrCmU* | | | |
| | | mU*mU*rU (SEQ ID NO: 26) | | | |
| | BIM #9 | mA*mG*mU*rUrCrUrGrArGrU | AGTTCTGAGTG | TCTCGGTCA | Target |
| | | rGrUrGrArCrCrGrArGrArGrUr | TGACCGAGA | CACTCAGA | sequence is in |
| | | UrUrUrArGrArGrCrUrArGrArA | (SEQ ID NO: 6) | ACT | exon 2A, |
| | | rArUrArGrCrArArGrUrUrArAr | | (SEQ ID NO: | resulting in |
| | | ArArUrArArGrGrCrUrArGrUrC | | 18) | knockout of |
| | | rCrGrUrUrArUrCrArArCrUrUr | | | BIM_{EL}, BIM_{L} |
| | | GrArArArArArGrUrGrGrCrArC | | | and BIM_{S} |
| | | rCrGrArGrUrCrGrGrUrGrCmU* | | | |
| | | mU*mU*rU (SEQ ID NO: 32) | | | |
| | BIM | mG*mC*mC*rUrCrCrCrCrArGr | GCCTCCCCAGC | CAGGTCTGA | Target |
| | #19 | CrUrCrArGrArCrCrUrGrGrUrUr | TCAGACCTG | GCTGGGGA | sequence is in |
| | | UrUrArGrArGrCrUrArGrArArA | (SEQ ID NO: 7) | GGC | exon 2A, |
| | | rUrArGrCrArArGrUrUrArArAr | | (SEQ ID NO: | resulting in |
| | | ArUrArArGrGrCrUrArGrUrCrC | | 19) | knockout of |
| | | rGrUrUrArUrCrArArCrUrUrGr | | | BIM_{EL}, BIM_{L} |
| | | ArArArArArGrUrGrGrCrArCrC | | | and BIM_{S} |
| | | rGrArGrUrCrGrGrUrGrCmU*m | | | |
| | | U*mU*rU (SEQ ID NO: 33) | | | |
| | BIM | mG*mG*mC*rCrUrGrGrCrArA | GGCCTGGCAAG | CAAGTCCTC | Target |
| | #24 | rGrGrArGrGrArCrUrUrGrGrUr | GAGGACTTG | CTTGCCAGG | sequence is in |
| | | UrUrUrArGrArGrCrUrArGrArA | (SEQ ID NO: 8) | CC | exon 2C, |
| | | rArUrArGrCrArArGrUrUrArAr | | (SEQ ID NO: | resulting in |
| | | ArArUrArArGrGrCrUrArGrUrC | | 20) | knockout of |
| | | rCrGrUrUrArUrCrArArCrUrUr | | | BIM_{EL} and |
| | | GrArArArArArGrUrGrGrCrArC | | | BIM_{L} |
| | | rCrGrArGrUrCrGrGrUrGrCmU* | | | |
| | | mU*mU*rU (SEQ ID NO: 34) | | | |
| | BIM | mU*mG*mG*rUrUrGrArArGrG | TGGTTGAAGGC | CCTTGCCAG | Target |
| | #25 | rCrCrUrGrGrCrArArGrGrGrUr | CTGGCAAGG | GCCTTCAAC | sequence is in |
| | | UrUrUrArGrArGrCrUrArGrArA | (SEQ ID NO: 9) | CA | exon 2C, |
| | | rArUrArGrCrArArGrUrUrArAr | | (SEQ ID NO: | resulting in |
| | | ArArUrArArGrGrCrUrArGrUrC | | 21) | knockout of |
| | | rCrGrUrUrArUrCrArArCrUrUr | | | BIM_{EL} and |
| | | GrArArArArArGrUrGrGrCrArC | | | BIM_{L} |
| | | rCrGrArGrUrCrGrGrUrGrCmU* | | | |
| | | mU*mU*rU (SEQ ID NO: 35) | | | |

### Example 5. Determining the Editing Efficiency of 3 Different crRNAs Through CRISPR/MAD7-Mediated Gene Editing

Electroporation of MAD7 Ribonucleoproteins (RNP) in iPSCs

Electroporation was done using Lonza P3 Primary Cell 4D-Nucleofector® X Kit S (Lonza, V4XP-3032) and Lonza 4D-Nucleofector® System (Lonza, AAF-1003X) according to the manufacture's protocol. For editing of each individual gene, corresponding MAD7 ribonucleoprotein (RNP) was prepared by incubating 30 pmol of MAD7 (GenScript; Piscataway, NJ) and 150 pmol of crRNA targeting the BIM (BCL2L11) (SEQ ID NO: 10), SOCS1 (SEQ ID NO: 11), or CISH (SEQ ID NO: 12)(customized by IDT; see Table 5) in 10 μL of Lonza P3 Primary Nucleofector® solution for 15-20 min at room temperature. For each electroporation reaction, 3×10⁵ to 5×10⁵ iPSCs were first resuspended in the 10 μL of Lonza P3 Primary Nucleofector® solution, then combined with 10 μL of the incubated MAD7 RNP to a final reaction volume of 20 μL. Resuspended cells with RNP were then transferred into Nucleocuvette Strips and electroporation was performed using the CA-137 program. After completion, cells were seeded into vitronectin (VTN)-coated culture plates containing StemFlex™ medium (ThermoFisher, A3349401) with RevitaCell™ (ThermoFisher, A2644501). The second-round of electroporation was performed 48 hours later, and cells were collected for analysis 2 days after the second electroporation. Single clone selection was then performed on the CISH, SOCS1, or BIM edited cells.

TABLE 5
Full-length crRNA sequences and their corresponding target sequences for MAD7
endonuclease at 3 indicated genomic loci.

| | Name | | Targeting/ | Target | |
|---|---|---|---|---|---|
| Target | of | | guide sequence | sequence in | Target |
| gene | crRNA | Full crRNA sequence | in crRNA | target gene | exon |
| BIM | BIM | mG*U*mC*rArArArArGrArCrCrUr | ACACAGACAG | GTGCTGGGCT | Exon 2C |
| (BCL2L11) | crRNA | UrUrGrGrArArUrUrUrCrUrArCrUrCr | GAGCCCAGCA | CCTGTCTGTGT | (designed to |
| | | UrUrGrUrArGrArUrArCrArCrArGrA | C | (SEQ ID | retain BIMs) |
| | | rCrArGrGrArGrCrCrCrAmG*mC*m | (SEQ ID | NO: 22) | |
| | | A*rC (SEQ ID NO: 36) | NO: 10) | | |
| SOCS1 | SOCS1 | mG*mU*mC*rArArArArGrArCrCrUr | ACCTGGATGG | TCTCGCGGCT | Exon 2 |
| | crRNA | UrUrGrGrArArUrUrUrCrUrArCrUrCr | CAGCCGCGAG | GCCATCCAGG | |
| | | UrUrGrUrArGrArUrArCrCrUrGrGrA | A (SEQ ID | T | |
| | | rUrGrGrCrArGrCrCrGrCmG*mA*m | NO: 11) | (SEQ ID | |
| | | G*rA (SEQ ID NO: 37) | | NO: 23) | |
| CISH | CISH | mG*mU*mC*rArArArArGrArCrCrUr | GGTGTACAGC | CACCAGCCAC | Exon 3 |
| | crRNA | UrUrGrGrArArUrUrUrCrUrArCrUrCr | AGTGGCTGGT | TGCTGTACAC | |
| | | UrUrGrUrArGrArUrGrGrUrGrUrArC | G (SEQ ID | C | |
| | | ArGrCrArGrUrGrGrCrUmG*mG*m | NO: 12) | (SEQ ID | |
| | | U*rG (SEQ ID NO: 38) | | NO: 24) | |

### Editing Efficiency Determination by ICE Analysis

Genomic DNA of edited cells was obtained by QuickExtract DNA Extraction Solution (LGC, SS000035-D2) ir QIAmp DNA Mini Kit (Qiagen (Hilden, German), QIA-51306) as per manfacture's protocol. Target locus-specific DNA fragments were amplified using KAPA HiFi HotStart PCR Kit (Roche, KR0369) with specific primer sets (See Table 6). The PCR products were then sequenced using Sanger sequencing by MISSION BIOTECH. Sequencing primers used for each locus are listed in Table 6. The editing efficiency was determined by analyzing the indel formation based on the sequencing results of amplified DNA fragments using the Synthego ICE Analysis tool (Synthego)

TABLE 6
PCR and Sanger sequencing primers used in analyzing editing efficiency of target
loci.

| | PCR primer-Forward | PCR primer-Reverse | Sequencing primer |
|---|---|---|---|
| BIM | ACCGAGAAGGTAGACAATT | AGTTTGACACATCCTCCATT | AATCACGGAGGTGAAGGGG |
| | GCAGC (SEQ ID NO: 45) | CCCA (SEQ ID NO: 46) | A (SEQ ID NO: 47) |
| SOCS1 | GCCCCTTCTGTAGGATGGTA | GGGGAAGGAGCTCAGGTAG | AGCACACAACCAGGTGGCA |
| | GC (SEQ ID NO: 48) | TCG (SEQ ID NO: 49) | G (SEQ ID NO: 50) |
| CISH | GGTTCCATTACGGCCAGCGA | GGCCAGCTGCCAGAGGTAT | CGCACCCCAGCTACCTGTTC |
| | (SEQ ID NO: 51) | G (SEQ ID NO: 52) | (SEQ ID NO: 53) |

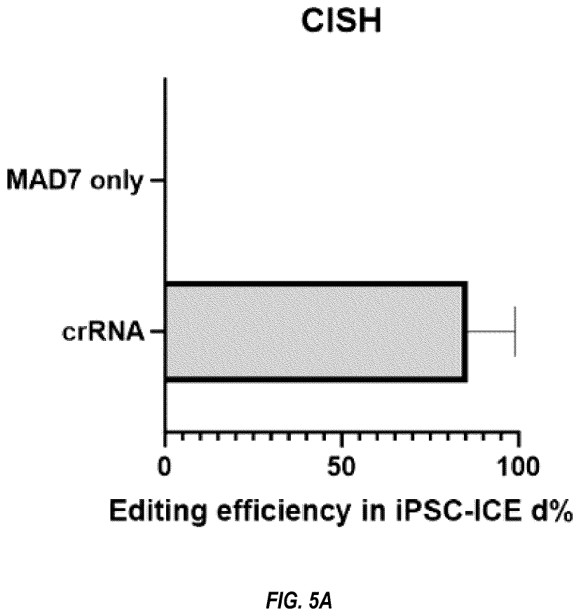

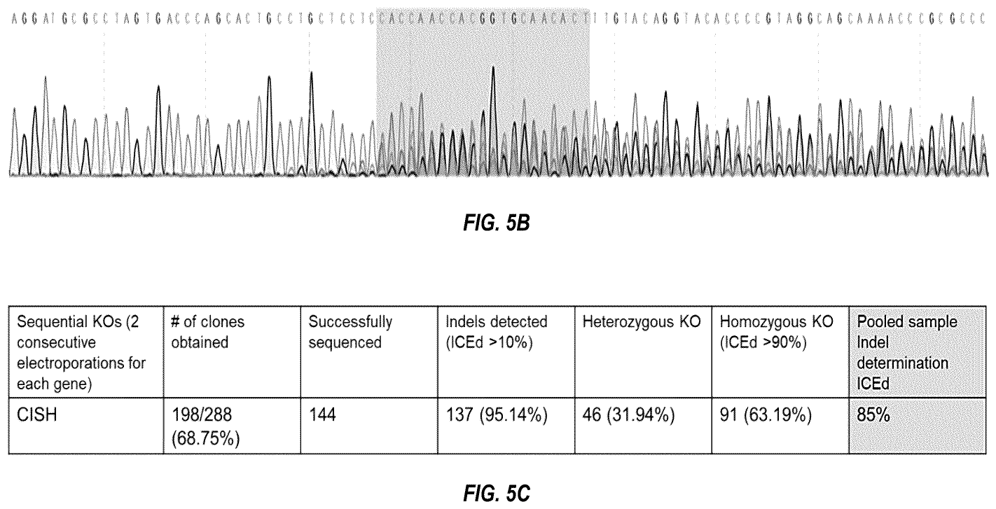

As shown in FIG. 5A, successful CRISPR/MAD7-mediated CISH crRNA gene editing was observed with 85%0 indel (small insertion/deletion resulted from DNA cleavage and repair) formation at the CISH locus; in contrast, there was no indel observed in the MAD7 protein only control. The indel can be visualized in a Sanger sequencing chromatogram as patterns of mixed peaks beginning around the targeting site (shaded area, FIG. 5B). As summarized in FIG. 5C, single clone selection was performed on pools of CISH crRNA edited cells to obtain clones arising from a single cell. Out of all obtained clones, 144 clones were successfully sequenced. Among the 144 clones, CISH editing was observed in 137 (95%), which was significantly higher than observed in the pooled population. Further analysis suggested that 46 of the 137 clones were heterozygous knock outs (with one allele of CISH edited), and the remaining 96 were homozygous knock outs (both alleles edited, complete knock out), again indicating successful CRISPR/MAD7-mediated editing with CISH crRNA.

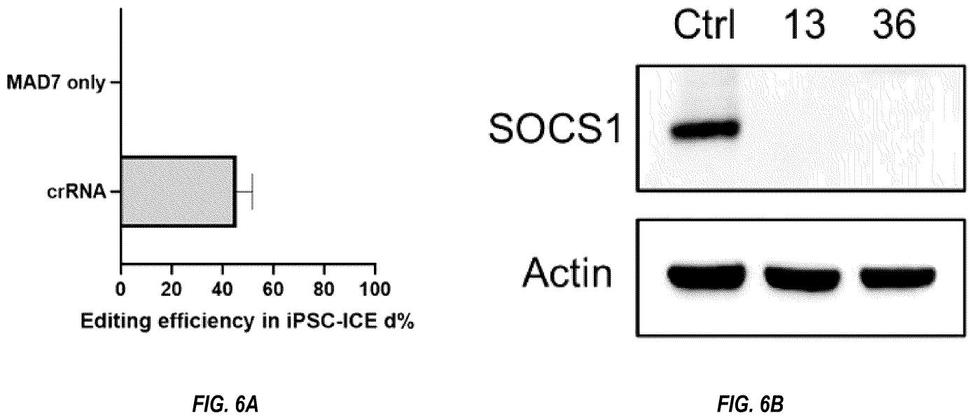

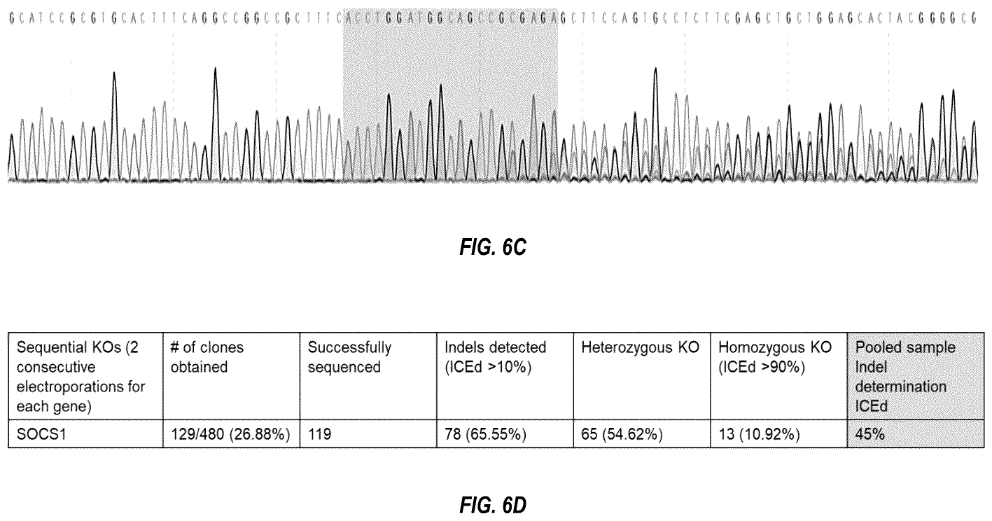

As shown in FIG. 6A, successful CRISPR/MAD7-mediated SOCS1 crRNA editing was observed with 45% indel formation at the SOCS1 locus; in contrast, there was no indel observed in the MAD7 protein only control. The indel can be visualized in a Sanger sequencing chromatogram as patterns of mixed peaks beginning around the targeting site (shaded area, FIG. 6C). As summarized in FIG. 6D, single clone selection was performed on pools of SOCS1 edited cells to obtain clones arising from a single cell. Out of all obtained clones, 119 clones were successfully sequenced. Among the 119 clones, SOCS1 editing was observed in 78 clones (65.55%). Further analysis suggested that 65 out of 78 clones were heterozygous knock outs (with one allele of SOCS1 being edited) and the remaining 13 were homozygous knock outs (both alleles edited, complete knock out). These results again demonstrate successful CRISPR/MAD7-mediated editing with SOCS1 crRNA. To determine the disruption of SOCS1 gene product after editing, western blot analysis was performed on two of the homozygous knockout clones (clone numbers 13 and 36) obtained during the single clone selection process. FIG. 6B shows the complete depletion of SOCS1 protein in both clones, suggesting the translation of successful gene editing/knockout at the protein level.

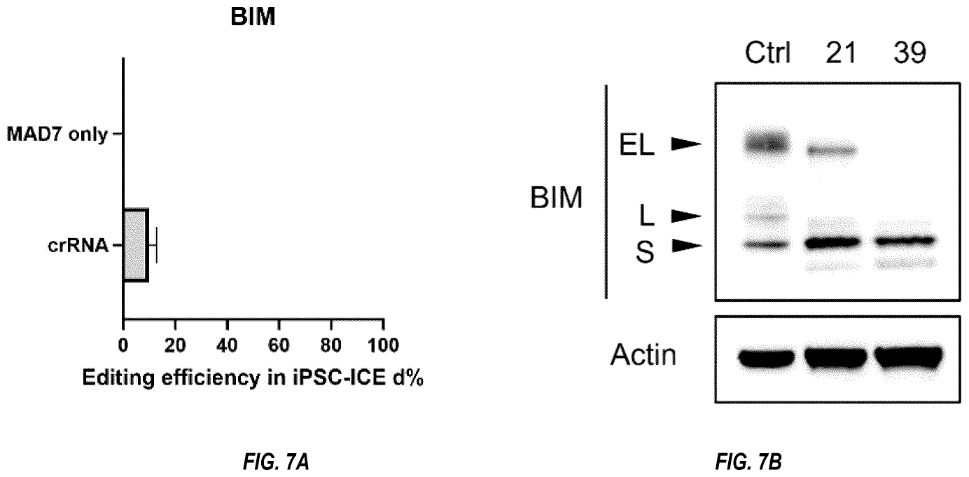

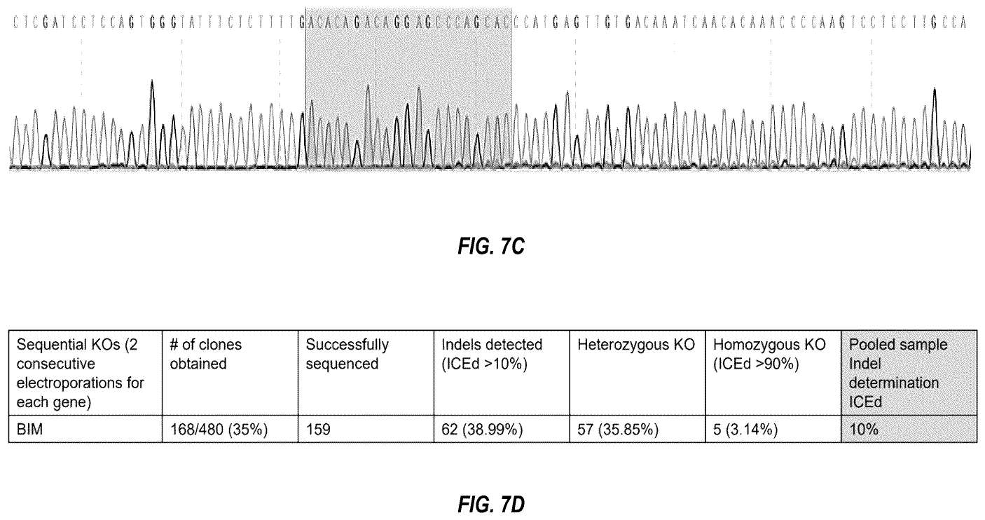

As shown in FIG. 7A, successful editing was observed with BIM crRNA in CRISPR/MAD7-mediated gene editing, evidenced by 10% indel formation at the BIMlocus; in contrast, there was no indel observed in the MAD7 protein only control. The indel can be visualized in a Sanger sequencing chromatogram as patterns of mixed peaks beginning around the targeting site (shaded area, FIG. 7C). As summarized in FIG. 7D, single clone selection was performed on pools of BIM edited cells to obtain clones arising from a single cell. Out of all obtained clones, 159 clones were successfully sequenced, among which, BIMediting was observed in 62 clones (38.99%). Further analysis suggested that 57 of 62 clones were heterozygous knock outs (with one allele ofBIMbeing edited) and the remaining 5 clones were homozygous knock outs (both alleles edited, complete knock out). This again demonstrates the successful CRISPR/MAD7-mediated editing with BIM crRNA. To determine the disruption of the BIM gene product after editing, a western blot analysis was performed on one heterozygous knockout clone (clone number 21) and one homozygous knockout clone (clone number 39) obtained during the single clone selection process. As shown in FIG. 7B, a partial depletion of the BIM_{EL} and BIM_{L} protein was observed in clone 21, whereas clone 39 obtained a complete depletion of both BIM_{EL} and BIM_{L} while retaining BIM_{S}, establishing the effects of successful gene editing/knockout at the protein level. Note that BIM_{S} remained in both clones, as the BIM crRNA was designed to deplete BIM_{EL} and BIM_{L} splice forms but to retain BIM_{S}.

### Single Clone Selection

48 h after gene editing, CISH, SOCS1, or BIM edited iPSCs were single-cell seeded into VTN-coated 96-well plates either by limiting dilution or single cell sorting. Limiting dilution was done by resuspending cells to a density of 5 cells/mL in StemFlex™ medium (ThermoFisher, A3349401) with RevitaCell™ (ThermoFisher, A2644501) and seeding 100 μL of cell suspension into each well of the 96-well plates. Seeding at this low density of 0.5 cells/well ensures the isolation of single cells in individual wells, thus establishing single clones. Alternatively, single cell sorting was performed using a BD FACSAria™ III Sorter (BD Biosciences). Cells were left undisturbed for 48 h to 72 h after seeding to increase cell viability. Regular medium changes were performed every 2-3 days thereafter for 2 weeks or until cell colonies' diameter was larger than 800 μm for collection or further expansion.

### Western Blot

A whole cell lysate was obtained from control, SOCS1, or BIM edited clones for examining the depletion of targeted gene products. Cells were lysed in RIPA buffer (Millipore, 20-188) on ice for 20 min, then centrifuged at 20,000×g for 15 min at 4° C. to remove insoluble debris. Next, the protein concentration was determined using a Protein Assay Dye Reagent (Bio-Rad, 5000006). Equal amounts of protein from each sample were subjected to SDS-PAGE using mPAGE 4-20% Bis-Tris Precast Gel (Millipore, MP42G12). Subsequently, the proteins in the gels were transferred to a Power Blotter Select Transfer PVDF membrane (Invitrogen, PB5340).

After blocking the membrane in 5% milk in TBST for 1 h at room temperature, primary antibodies (BIM, 1:1,000, Cell Signaling Technology, 2933S; SOCS1, 1:1,000, Cell Signaling Technology, 68631S; Beta-actin, 1:5,000, Santa Cruz Biotechnology, sc-47778 HRP) were applied to corresponding blots and incubated O/overnight at 4° C. The membranes were then washed with TBST before incubating with peroxidase conjugated goat anti-rabbit IgG antibody (1:5,000, Sigma-Aldrich, AP132P) for 1 h at room temperature. The blots were finally washed with TBST, and a chemiluminescent signal was developed using SuperSignal™ West Pico PLUS Chemiluminescent Substrate (ThermoFisher Scientific, 34580).

TABLE 7
Summary of targeting/guide sequences in sgRNA/crRNA and target sequences in
target gene

| SEQ ID NO | Name | SEQUENCE |
|---|---|---|
| SEQ ID NO: 1 | Targeting sequence in | GCACTCACATCGCTACATCA |
| | Scramble #2 | |
| SEQ ID NO: 2 | Targeting sequence in | TACCCATTGCACTGAGATAG |
| | BIM #5 | |
| SEQ ID NO: 3 | Targeting sequence in | AGGCCACATAGTGCTGCACA |
| | CISH #3 | |
| SEQ ID NO: 4 | Targeting sequence in | TGTACAGCAGTGGCTGGTGG |
| | CISH #4 | |
| SEQ ID NO: 5 | Targeting sequence in | AGTAGAATCCGCAGGCGTCC |
| | SOCS1 sgRNA | |
| SEQ ID NO: 6 | Targeting sequence in | AGTTCTGAGTGTGACCGAGA |
| | BIM #9 | |
| SEQ ID NO: 7 | Targeting sequence in | GCCTCCCCAGCTCAGACCTG |
| | BIM #19 | |
| SEQ ID NO: 8 | Targeting sequence in | GGCCTGGCAAGGAGGACTTG |
| | BIM #24 | |
| SEQ ID NO: 9 | Targeting sequence in | TGGTTGAAGGCCTGGCAAGG |
| | BIM #25 | |
| SEQ ID NO: 10 | Targeting sequence in | ACACAGACAGGAGCCCAGCAC |
| | BIM crRNA (MAD7) | |
| SEQ ID NO: 11 | Targeting sequence in | ACCTGGATGGCAGCCGCGAGA |
| | SOCS1 crRNA (MAD7) | |
| SEQ ID NO: 12 | Targeting sequence in | GGTGTACAGCAGTGGCTGGTG |
| | CISH crRNA (MAD7) | |
| SEQ ID NO: 13 | Target sequence by | TGATGTAGCGATGTGAGTGC |
| | Scramble #2 sgRNA | |
| SEQ ID NO: 14 | Target sequence in BIM | CTATCTCAGTGCAATGGGTA |
| | by BIM #5 sgRNA | |
| SEQ ID NO: 15 | Target sequence in CISH | TGTGCAGCACTATGTGGCCT |
| | by CISH #3 sgRNA | |
| SEQ ID NO: 16 | Target sequence in CISH | CCACCAGCCACTGCTGTACA |
| | by CISH #4 sgRNA | |
| SEQ ID NO: 17 | Target sequence in | GGACGCCTGCGGATTCTACT |
| | SOCS1 by SOCS1 | |
| | sgRNA | |
| SEQ ID NO: 18 | Target sequence in BIM | TCTCGGTCACACTCAGAACT |
| | by BIM #9 sgRNA | |
| SEQ ID NO: 19 | Target sequence in BIM | CAGGTCTGAGCTGGGGAGGC |
| | by BIM #19 sgRNA | |
| SEQ ID NO: 20 | Target sequence in BIM | CAAGTCCTCCTTGCCAGGCC |
| | by BIM #24 sgRNA | |
| SEQ ID NO: 21 | Target sequence in BIM | CCTTGCCAGGCCTTCAACCA |
| | by BIM #25 sgRNA | |
| SEQ ID NO: 22 | Target sequence in BIM | GTGCTGGGCTCCTGTCTGTGT |
| | by BIM crRNA (MAD7) | |
| SEQ ID NO: 23 | Target sequence in | TCTCGCGGCTGCCATCCAGGT |
| | SOCS1 by SOCS1 | |
| | crRNA (MAD7) | |
| SEQ ID NO: 24 | Target sequence in CISH | CACCAGCCACTGCTGTACACC |
| | by CISH crRNA (MAD7) | |

### Example 6. FAS Deficiency Protects Functionally Enhanced 5KO T-iγδT Cells from FASL-Induced Apoptosis

Generation of Functionally Enhanced 5KO (B2M^{-/-}/CIITA^{-/-}/CISH^{-/-}/SOCS1^{-/-}/BIM^{-/-}) T-iγδT Cells

To generate functional, enhanced quintuple target gene knockout (5KO) T-igdT cells, Vd2 gdT-derived iPSC clones were sequentially nucleofected with Alt-R™ S.p. HiFi Cas9 Nuclease V3 (Integrated DNA Technologies; Cat: 1081060) and synthetic single guide RNAs (sgRNAs) targeting B2M, CIITA, CISH, SOCS1 and BIM, as listed in Table 1 and Table 8, using a Lonza P3 Primary Cell 4D-Nucleofector® X Kit L (Lonza, Cat: V4XP-3024) and a Lonza 4D Nucleofector® Core and X Unit (Lonza, Cat: AAF-1003X) per Nucleocuvette vessel. After the final round of nucleofection, B2M^{-/-}CIITA^{-/-} double knockout iPSCs were purified by single cell sorting. Sorted B2M^{-/-}CIITA^{-/-} iPSC clones were further screened for CISH, SOCS1 and BIM knockouts by sequencing. Thereafter, selected iPSC clones with each of B2M, CIITA, CISH, SOCS1 and BIM knocked out were then differentiated into mature T-igdT cells.

To generate 5KO iγδT cells from 5KO iPSC, stepwise differentiation methods were applied to sequentially induce hematopoietic stem cells (HSCs), common lymphoid progenitors (CLPs), pre-T cells, and γδT cells. For HSC differentiation, single cell suspension of iPSCs was seeded into an AGGREWELL™800 PLATE (STEMCELL Technologies, #34811) for EB formation in iPSC culture medium Essential 8 (Thermo Fisher Scientific, #A1517001) with 1 μM ROCK inhibitor Y-27632 (Selleckchem, #S1049, Houston, TX) and 7. μM CHIR99021 (Selleckchem #S1263). After an overnight incubation, the medium was changed to EB Medium (StemPro™-34 (Thermo Fisher Scientific #10639011), L-alanyl-L-glutamine (Glutamax® (Thermo Fisher Scientific #35050061)), 50 μg/mL ascorbic acid-2-phosphate (AA2P, Cayman Chemical #16457, Ann Arbor, MI), 0.4 mM monothioglycerol (MTG, Merck #M6145, Darmstadt, Germany), and 1×Insulin-Transferrin-Selenium solution (ITS-G, Thermo Fisher Scientific #41400045)). Patterning factors including BMP4, VEGF, bFGF were supplemented in EB Medium. On day 2 of differentiation, SB431542 (Selleckchem #S1067) was added in the culture medium.

On day 4 of differentiation, the medium was replaced with an EB Medium supplemented with VEGF, bFGF, and SCF (PeproTech, Thermo Fisher Scientific, #300-07). On day 7 of differentiation, EBs were transferred to gelatin (Merck G1393-100) coated dishes in EB Medium supplemented with VEGF, bFGF, SCF, TPO (PeproTech, Thermo Fisher Scientific, #300-18), and 10 ng/mL FLT3L (PeproTech, Thermo Fisher Scientific, #300-19). The medium was refreshed every 2 to 3 days with floating cells retained in the same well.

On day 14, the floating HSC were collected for pre-T differentiation by seeding on a culture dish coated with DLL4-Fc, VCAM-1, and RetroNectin® and were cultured in STEMdiff™ APEL™ 2 medium with 0.5% BSA, 250 μg/mL AA2P, 3 μM CHIR99021, 3.1 ng/mL IL-7, 3.1 ng/mL FLT3L, 1.6 ng/mL SCF, 5 ng/mL SDF1-α, and 15 μM SB203580. On day 18 of differentiation, the medium was refreshed. On day 21 of differentiation, the cells were detached from the coated dish using a treatment of Accutase® (Innovative Cell AT 104-500) and cultured in a new, coated dish with STEMdiff™ APEL™ 2 medium containing 0.5% BSA, 250 μg/mL AA2P, 3.1 ng/mL IL-7, 3.1 ng/mL FLT3L, 5 ng/mL SDF1-α, and 15 μM SB203580. On day 25 of differentiation, the medium was refreshed.

On differentiation day 28, cells (composed of pre-T and few γδT cells) were subjected to treatment of 0.3 μg/mL anti-CD3 antibody (BioLegend (Revvity) 317302), 2 ng/mL IL-7, 4 ng/mL IL-21 (PeproTech, Thermo Fisher Scientific, 200-21) in STEMdiff™ APEL™ 2 medium with 10% HPL and 250 μg/mL AA2P. After 3 days, the medium was replenished with a STEMdiff™ APEL™ 2 medium comprising 2 ng/mL IL-7, 4 ng/mL IL-21, 10% HPL, and 250 μg/mL AA2P.

On differentiation day 35, an expansion cycle of T-iγδT cells was initiated by stimulating the T-iγδT cells in STEMdiff™ APEL™ 2 medium with 10% HPL, 250 μg/mL AA2P, Dynabeads™ Human T-Activator CD3/CD28 (Thermo Fisher Scientific 11132D), 0.3 mg/mL human CD30/TNFRSF8 antibody (R&D MAB2291), 50 ng/mL IL-12 (PeproTech, Thermo Fisher Scientific, 200-12), 50 ng/mL IL-18 (MBL B001-5), 50 ng/mL TL-1A (PeproTech, Thermo Fisher Scientific, 310-23), 20 ng/mL IL-21, 1 ng/mL IL-7, 1 ng/mL IL-15 (PeproTech, Thermo Fisher Scientific, 200-15) and 1 μM Z-VAD (Cayman 14463). Three days after stimulation, the Dynabeads™ Human T-Activator CD3/CD28 were removed, and the medium was replaced by STEMdiff™ APEL™ 2 medium with 10% HPL, 250 μg/mL AA2P, 1 ng/mL IL-7 and 1 ng/mL IL-15. The iPSC derived γδT were expanded in this condition for 2 weeks with medium changed every 2 to 3 days and maintained in a density of about 1 to about 2×10⁶ cells/mL.

Generation of FAS-Deficient and Functionally Enhanced 6KO (B2M^{-/-}/CIITA^{-/-}/CISH^{-/-}/SOCS1^{-/-}/BIM^{-/-}/FAS^{-/-}) T-iγδT Cells

To generate the FAS-deficient and functionally enhanced sextuplet target gene knockout (6KO) T-igdT cells, a selected iPSC single clone with a quintuple target gene knockout (5KO; B2M^{-/-}/CIITA^{-/-}/CISH^{-/-}/SOCS1^{-/-}/BIM^{-/-}) was nucleofected with Alt-R™ S.p. HiFi Cas9 Nuclease V3 (Integrated DNA Technologies; Cat: 1081060) and FAS synthetic single guide RNA (sgRNA), as listed in Table 8, using a Lonza P3 Primary Cell 4D-Nucleofector® X Kit L (Lonza, Cat: V4XP-3024) and a Lonza 4D Nucleofector® Core and X Unit (Lonza, Cat: AAF-1003X) per Nucleocuvette vessel. After nucleofection, the FAS-deficient and functionally enhanced 6 Kg iPSC clone was purified by bulk FAS⁻ cell sorting and then differentiated into mature T-igdT cells, by a similar process described above. The mature 5KO (B2M^{-/-}/CIITA^{-/-}/CISH^{-/-}/SOCS1^{-/-}/BIM^{-/-}) and 6KO (B2M^{-/-}/CIITA^{-/-}/CISH^{-/-}/SOCS1^{-/-}/BIM^{-/-}/FAS^{-/-}) T-igdT cells were activated using Dynabeads™ Human T-Activator CD3/CD28 (ThermoFisher 11132D) and cytokine cocktails for 3 days, followed by culturing in STEMdiff™ APEL™ 2 medium with 1000 HIPL, 250 μg/mL AA2P, 1 ng/mL IL-7 and 1 ng/mL IL-15 for 11 days.

TABLE 8
sgRNA sequence of candidate genes used for nucleofection with Cas9 nuclease

| | Name | | Targeting/ | Target | |
|---|---|---|---|---|---|
| Target | of | | guide sequence | sequence in | Target |
| Gene | sgRNA | Synthetic sgRNA sequence | in sgRNA | target gene | exon |
| B2M | B2M | mC*mG*mU*rGrArGrUrArArArCr | CGTGAGTAAAC | AAGATTCAG | Exon 2 |
| | sgRNA | CrUrGrArArUrCrUrUrGrUrUrUrUr | CTGAATCTT | GTTTACTCAC | |
| | (B2M | ArGrArGrCrUrArGrArArArUrArGr | (SEQ ID NO: 55) | G | |
| | #1) | CrArArGrUrUrArArArArUrArArGr | | (SEQ ID NO: | |
| | | GrCrUrArGrUrCrCrGrUrUrArUrCr | | 56) | |
| | | ArArCrUrUrGrArArArArArGrUrGr | | | |
| | | GrCrArCrCrGrArGrUrCrGrGrUrGr | | | |
| | | CmU*mU*mU*rU (SEQ ID NO: 54) | | | |
| CIITA | CIITA | mG*mA*mU*rArUrUrGrGrCrArUr | GATATTGGCAT | GGGAGGCTT | Exon 3 |
| | sgRNA | ArArGrCrCrUrCrCrCrGrUrUrUrUr | AAGCCTCCC | ATGCCAATAT | |
| | (CIITA | ArGrArGrCrUrArGrArArArUrArGr | (SEQ ID NO: 58) | C | |
| | #1) | CrArArGrUrUrArArArArUrArArGr | | (SEQ ID NO: | |
| | | GrCrUrArGrUrCrCrGrUrUrArUrCr | | 59) | |
| | | ArArCrUrUrGrArArArArArGrUrGr | | | |
| | | GrCrArCrCrGrArGrUrCrGrGrUrGr | | | |
| | | CmU*mU*mU*rU (SEQ ID NO: 57) | | | |
| FAS | FAS | mG*mA*mU*rUrGrCrUrCrArArCrAr | GATTGCTCAAC | AGCATGGTT | Exon 1 |
| | sgRNA | ArCrCrArUrGrCrUrGrUrUrUrUrArGr | AACCATGCT | GTTGAGCAA | |
| | (FAS | ArGrCrUrArGrArArArUrArGrCrArAr | (SEQ ID NO: 61) | TC | |
| | #1) | GrUrUrArArArArUrArArGrGrCrUrAr | | (SEQ ID NO: | |
| | | GrUrCrCrGrUrUrArUrCrArArCrUrUr | | 62) | |
| | | GrArArArArArGrUrGrGrCrArCrCrGr | | | |
| | | ArGrUrCrGrGrUrGrCmU*mU*mU*r | | | |
| | | U (SEQ ID NO: 60) | | | |

Survival Assay with IL-15 Deprivation

3×10⁴ of 5KO (B2M^{-/-}/CIITA^{-/-}/CISH^{-/-}/SOCS1^{-/-}/BIM^{-/-}) or 6KO (B2M^{-/-}/CIITA^{-/-}/CISH^{-/-}/SOCS1^{-/-}BIM^{-/-}/FAS^{-/-}) T-iγδT cells were resuspended in 200 μL of pre-warmed cytokine-free culture medium. Both 5KO and 6KO T-iγδT cells were seeded separately in a 96-well cell culture plate (Falcon, Cat: 353072), coated with FAS Ligand (Peprotech, Cat: 310-03H) at 5 mg/mL or 20 mg/mL overnight, and 240 μL of PBS was added to the outer 36 wells to prevent evaporation of the medium. To deprive the T-iγδT cells of IL-15, the T-iγδT cells were incubated at 37° C. with 5% CO₂ for 2-12 days (e.g., 2, 4, 6, 8, 10, or 12 days). After IL-15 deprivation, T-iγδT cells were harvested and transferred into a fresh 1.6 mL microcentrifuge tube every two days for the 2 to 12-day period. Cell viability was determined with FITC-Annexin V (1:200; BioLegend, Cat: 640905) and Propidium Iodide (1:2,500; BioLegend, Cat: 421301) staining according to the manufacturer's instructions and analyzed using an Attune™ NxT Flow Cytometer (ThermoFisher Scientific). Live T-iγδT cells were defined as an Annexin V negative −PI negative population analyzed with FlowJo™ software (BD Biosciences). The relative viability of the indicated timepoints of 2 days, 4 days, 6 days, 8 days, 10 days, and 12 days was calculated by normalization to the viability of the 0 day timepoint.

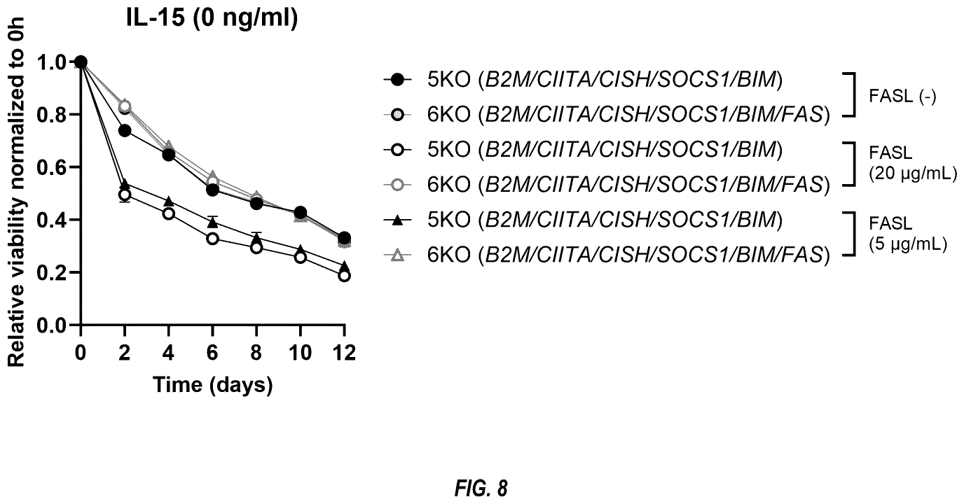

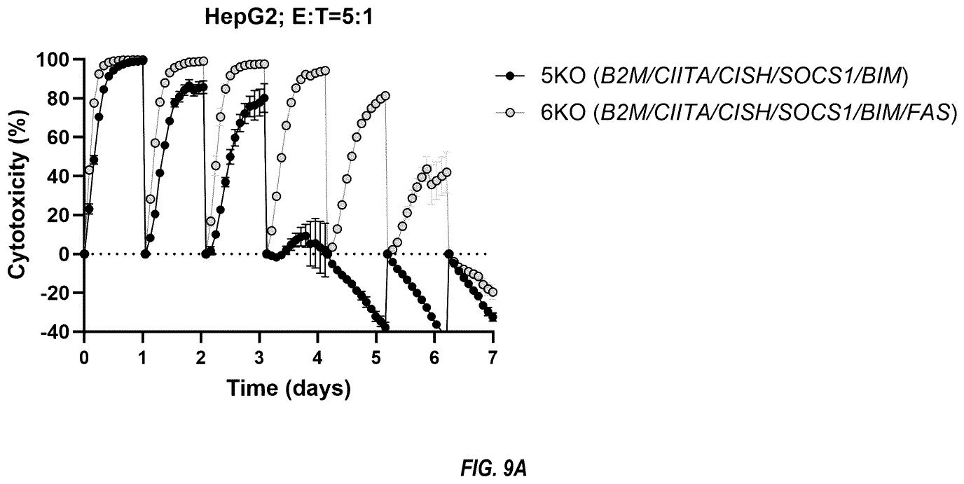

As shown in FIG. 8, there was a comparable survival between 5KO (B2M^{-/-}/CIITA^{-/-}/CISH^{-/-}/SOCS1^{-/-}/BIM^{-/-}) and 6KO (B2M^{-/-}/CIITA^{-/-}/CISH^{-/-}/SOCS1^{-/-}/BIM^{-/-}/FAS^{-/-}) T-iγδT cells post IL-15 deprivation in the absence of FASL for 2-12 days. In the presence of FASL (5 mg/mL or 20 mg/mL), there was a further decrease of survival of 5KO (B2M^{-/-}/CIITA^{-/-}/CISH^{-/-}/SOCS1^{-/-}/BIM^{-/-}T-iγδT cells post IL-15 deprivation for 2-12 days, when compared to the survival of 6KO T-iγδT cells. By contrast, there was a comparable survival of 6KO (B2M^{-/-}/CIITA^{-/-}/CISH^{-/-}/SOCS1^{-/-}/BIM^{-/-}/FAS^{-/-}) regardless of FASL stimulation post IL-15 deprivation for 2-12 days (FIG. 8).

Overall, FAS-deficient and functionally enhanced 6KO (B2M^{-/-}/CIITA^{-/-}/CISH^{-/-}/SOCS1^{-/-}/BIM^{-/-}/FAS^{-/-}) T-iγδT cells exhibited prolonged survival post FASL-induced apoptosis in the absence of IL-15 for 2-12 days (e.g., 2, 4, 6, 8, 10, or 12 days) (FIG. 8).

### Example 7. FAS Deficiency Enhances Durable Tumor Killing in Functionally Enhanced 5KO T-iγδT Cells

### Serial Killing Assay of T-iγδT Cells Against Adherent HepG2 Cells (2D Platform)

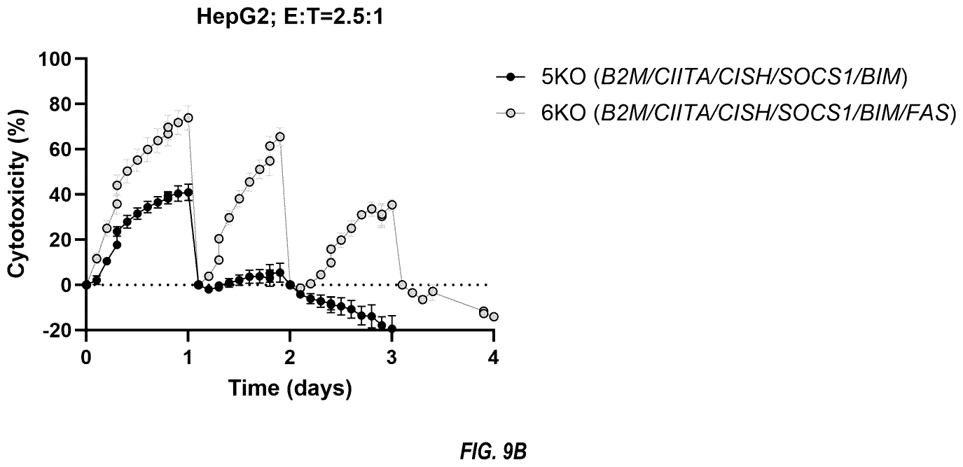

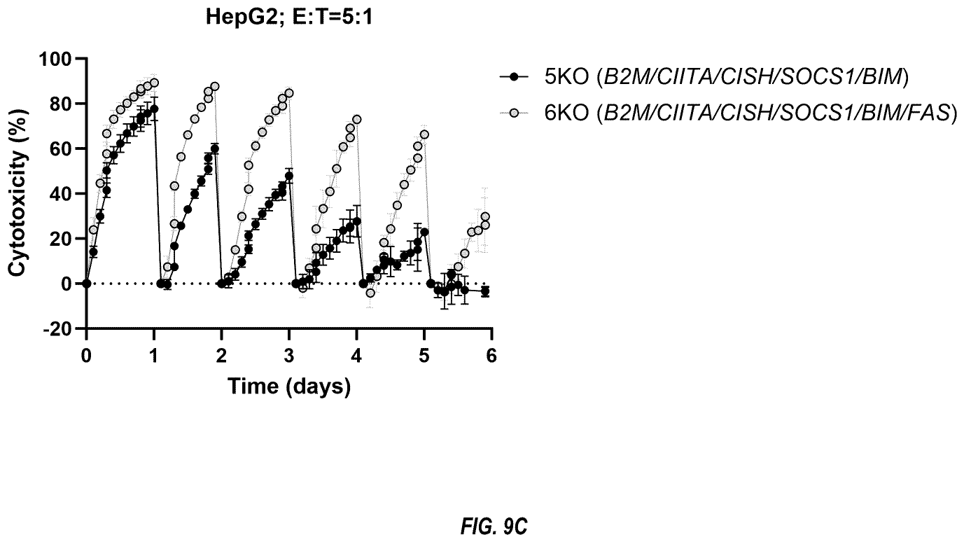

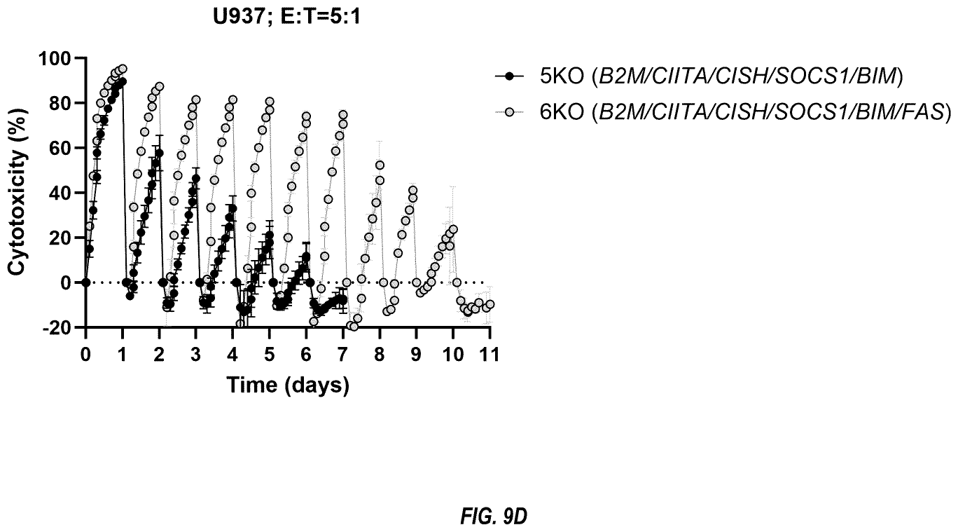

5×10³ of EGFP-HepG2 cells (ATCC (Manassas, VA), Cat: HB-8065, a cell line exhibiting epithelial-like morphology that was isolated from a hepatocellular carcinoma) were pre-seeded in a 96-well cell culture plate (Corning, Cat: 3599) overnight (i.e., about 12 hours to about 20 hours) (FIGS. 9A-9C). After seeding overnight, the culture supernatant was aspirated. The effector 5KO or 6KO T-igdT cells with target gene deficiencies, after a first round of activation using Dynabeads™ Human T-Activator CD3/CD28 (ThermoFisher 11132D) during the differentiation, were added into the 96-well cell culture plate in 200 mL of a culture medium without cytokines at a 5:1 effector to target cell ratio (FIG. 9A). The effector 5KO or 6KO T-igdT cells with target gene deficiencies, after a second around of activation using Dynabeads™ Human T-Activator CD3/CD28 (ThermoFisher 11132D) during the differentiation, were added into the 96-well cell culture plate in 200 mL of a culture medium without cytokines at a 2.5:1 and 5:1 effector to target cell ratio (FIG. 9B-9C). The cytotoxicity of T-igdT cells was monitored using live-cell imaging and analysis, using the Incucyte® SX5 Live-Cell Analysis System (Sartorius, Gottingen, Germany) through a 4× objective with scanning repeated every 24 hours. Hereafter, the effector T-igdT cells with target gene deficiencies were transferred to another GFP-HepG2 pre-seeded 96-well cell culture plate after 24 hours of tumor killing for a further round of killing. The cytotoxicity percentage was calculated using the formula listed below:

$$Cytotoxicity percentage = [ 1 - ( Total green fluorescence area at indicated timepoint / Total green fluorescence area at starting timepoint of each round of serial killing ) ] \times 100 \% \qquad a$$

### Serial Killing Assay of T-igdT Cells Against Suspended U937 Cells (2D Platform)

1×10⁴ of EGFP-U937 cells (ATCC (Manassas, VA), Cat: CRL-1593.2, a cell line exhibiting monocyte-like morphology that was isolated from a histiocytic lymphoma) were co-cultured with the effector 5KO or 6KO T-igdT cells with target gene deficiencies (after a second round of activation using Dynabeads™ Human T-Activator CD3/CD28 (ThermoFisher 11132D) during the differentiation) in 200 mL of a culture medium without cytokines at a 5:1 effector to target cell ratio in a 96-well cell culture plate (Corning, Cat: 3599) (FIG. 9D). The cytotoxicity of T-igdT cells was monitored using live-cell imaging and analysis, using the Incucyte® SX5 Live-Cell Analysis System (Sartorius, Gottingen, Germany) through a 4× objective scanning repeated every 24 hours. Hereafter, the 50 mL of culture supernatants were aspirated and 1×10⁴ of EGFP-U937 cells in 50 mL of culture medium were added into each well of 96-well cell culture plate after 24 hours of tumor killing for a further round of killing. The cytotoxicity percentage was calculated using the formula listed below:

$$Cytotoxicity percentage = [ 1 - ( Total green fluorescence area at indicated timepoint / Total green fluorescence area at starting timepoint of each round of serial killing ) ] \times 100 \% \qquad a$$

As shown in FIG. 9A, 5KO (B2M^{-/-}/CIITA^{-/-}/CISH^{-/-}/SOCS1^{-/-}/BIM^{-/-}) T-igdT cells could exhibit 3 rounds of killing against the EGFP-HepG2 cells at a 5:1 E:T ratio post the first round of activation. By contrast, 6KO (B2M^{-/-}/CIITA^{-/-}/CISH^{-/-}/SOCS1^{-/-}/BIM^{-/-}/FAS^{-/-}) T-igdT exhibited enhanced durable tumor killing (6 rounds) at a 5:1 E:T ratio post the first round of activation (FIG. 9A).

As shown in FIGS. 9B and 9C, 5KO (B2M^{-/-}/CIITA^{-/-}/CISH^{-/-}/SOCS1^{-/-}/BIM^{-/-}) T-igdT cells exhibit 1 and 3 rounds of killing against the EGFP-HepG2 cells at 2.5:1 and 5:1 ratios post the 2^{nd} round of activation, respectively. By contrast, 6KO (B2M^{-/-}/CIITA^{-/-}/CISH^{-/-}/SOCS1^{-/-}/BIM^{-/-}/FAS^{-/-}) T-igdT exhibited superior durable tumor killing against the EGFP-HepG2 cells at 2.5:1 and 5:1 E:T ratios post the 2^{nd} round of activation (FIGS. 9B and 9C).

Using a more sensitive tumor cell line as target cell of T-igdT cells, 5KO (B2M^{-/-}/CIITA^{-/-}/CISH^{-/-}/SOCS1^{-/-}/BIM^{-/-}) T-igdT cells could exhibit 4 rounds of killing against the EGFP-U937 cells at a 5:1 E:T ratio post the 2^{nd} round of activation. By contrast, 6KO (B2M^{-/-}/CIITA^{-/-}/CISH^{-/-}/SOCS1^{-/-}/BIM^{-/-}/FAS^{-/-}) T-igdT exhibited enhanced durable tumor killing (10 rounds) at a 5:1 E:T ratio post the 2^{nd} round of activation (FIG. 9D).

Overall, the FAS deficiency of the 6KO (B2M^{-/-}/CIITA^{-/-}/CISH^{-/-}/SOCS1^{-/-}/BIM^{-/-}/FAS^{-/-}) T-igdT cells enhanced cytotoxicity and persistence against HepG2 and U937 cells compared with the 5KO (B2M^{-/-}/CIITA^{-/-}/CISH^{-/-}/SOCS1^{-/-}/BIM^{-/-}) T-igdT cells in the 2D serial killing assay platform (FIGS. 9A-9D).

### Example 8. FAS Deficiency Increases Effector Molecules Post Serial Killing Against HepG2 Cells

The effector 5KO (B2M^{-/-}/CIITA^{-/-}/CISH^{-/-}/SOCS1^{-/-}/BIM^{-/-}) or 6KO (B2M^{-/-}/CIITA^{-/-}/CISH^{-/-}/SOCS1^{-/-}/BIM^{-/-}/FAS^{-/-}) T-igdT cells were co-cultured with 5×10³ of HepG2 cells (ATCC (Manassas, VA), Cat: HB-8065, a cell line exhibiting epithelial-like morphology that was isolated from a hepatocellular carcinoma) in 200 mL of a culture medium without cytokines at a 5:1 effector to target cell ratio in a 96-well cell culture plate (Corning, Cat: 3599) for 24 hours. After 24 hours of co-culturing, 50 ml of culture supernatant was harvested and stored at −80° C. Hereafter, 50 ml of fresh culture medium was added into each well and the effector T-igdT cells with target gene deficiencies were transferred to another HepG2 pre-seeded 96-well cell culture plate after 24 hours of tumor killing for a further round of killing. Similarly, 50 ml of culture supernatant was harvested after a further round of killing for 24 hours, until six rounds of killing (R1, R2, R3, R4, R5, R6) were performed. The effector molecules in the culture supernatants were quantified using a bead-based multiplex assay, LEGENDplex™ Human CD8/NK Panel (13-plex) w/filter plate V02 Assay Kit (BioLegend, Cat: 741186) according to the manufacturer's instructions. The data was acquired using a flow cytometer, Attune™ NxT Flow Cytometer (ThermoFisher Scientific), and analyzed using Qognit (San Carlos, CA) cloud-based program.

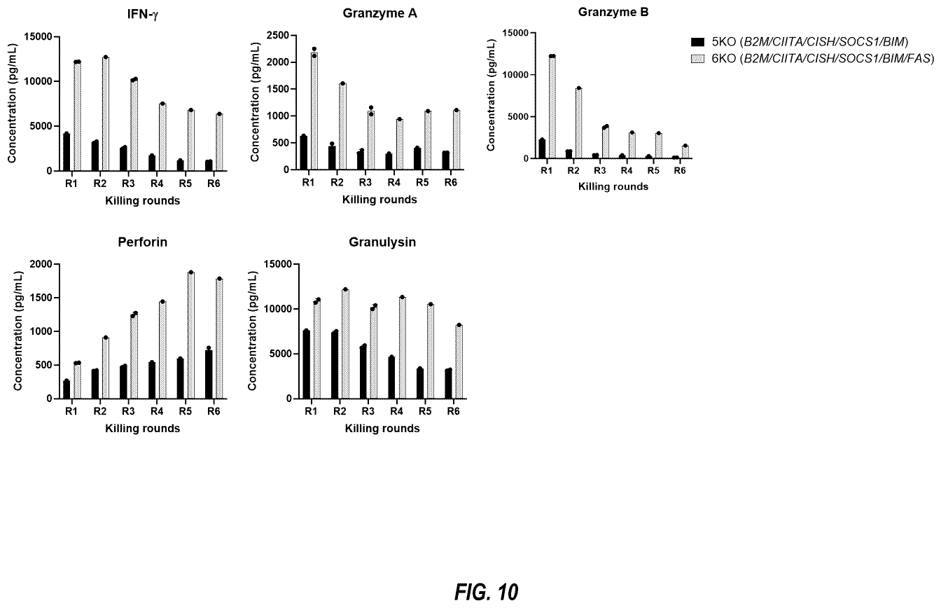

As shown is FIG. 10, the 6KO (B2M^{-/-}/CIITA^{-/-}/CISH^{-/-}/SOCS1^{-/-}/BIM^{-/-}/FAS^{-/-}) T-igdT cells secreted higher amounts of effector molecules (IFN-g, Granzyme A, Granzyme B, Perforin and Granulysin) into the culture supernatant compared with 5KO (B2M^{-/-}/CIITA^{-/-}/CISH^{-/-}/SOCS1^{-/-}/BIM^{-/-}) T-igdT cells post co-culturing with HepG2 cells at a 5:1 E:T ratio and post each round of killing (R1-R6) (FIG. 10).

Overall, the FAS deficiency of the 6KO (B2M^{-/-}/CIITA^{-/-}/CISH^{-/-}/SOCS1^{-/-}/BIM^{-/-}/FAS^{-/-}) T-igdT cells increased effector molecules (IFN-g, Granzyme A, Granzyme B, Perforin and Granulysin) and enhanced cytotoxicity compared with the 5KO (B2M^{-/-}/CIITA^{-/-}/CISH^{-/-}/SOCS1^{-/-}/BIM^{-/-}) T-igdT cells.

### Example 9. FAS Deficiency Increased IFN-γ-Producing T-iγδT Cells

The effector 5KO (B2M^{-/-}/CIITA^{-/-}/CISH^{-/-}/SOCS1^{-/-}/BIM^{-/-}) or 6KO (B2M^{-/-}/CIITA^{-/-}/CISH^{-/-}/SOCS1^{-/-}/BIM^{-/-}/FAS^{-/-}) T-igdT cells were co-cultured with 6×10⁴ of HepG2 cells (ATCC (Manassas, VA), Cat: HB-8065, a cell line exhibiting epithelial-like morphology that was isolated from a hepatocellular carcinoma) in 2 mL of a culture medium without cytokines at a 5:1 effector to target cell ratio in a 12-well cell culture plate (Falcon, Cat: 353043) for 24 hours. The Brefeldin A solution (BioLegend, Cat: 420601) was added into culture well for intracellular IFN-g staining 6 hours before the end of the co-culturing. The T-igdT cell suspension was harvested 24 hours post co-culturing and centrifuged at 300×g for 5 minutes. The supernatant was aspirated, and the cell pellet was washed once with a FACS staining buffer (autoMACS® Rinsing solution (Miltenyi Biotec, Bergisch Gladbach, North Rhine-Westphalia, Germany; Cat:130-091-222) with 2% FBS (Gibco, Cat: 10437-028)). The cell suspension was centrifuged at 300×g for 5 minutes and supernatant was aspirated. The T-igdT cells were stained with Brilliant Violet™ 605 anti-human TCR Vd2 antibody (BioLegend, Cat: 331430) and Brilliant Violet™ 711 anti-human CD95 (FAS) antibody (BioLegend, Cat: 305644) at 4° C. for 30 minutes. After cell surface marker staining, the cells were washed twice with a FACS staining buffer and fixed with an intracellular (IC) Fixation Buffer (eBioscience; ThermoFisher Scientific, Cat: 00-8222-49) at room temperature for 20 minutes. The fixed T-igdT cells were washed twice with 2 mL of 1× Permeabilization Buffer (eBioscience; ThermoFisher Scientific, Cat: 00-8333-56) and centrifuged at 400×g for 5 minutes at room temperature. The supernatant was aspirated, and the cell pellet was resuspended in 100 mL of 1× Permeabilization Buffer containing PE anti-human IFN-g antibody (BioLegend, Cat: 502509) for 20 minutes at room temperature. The stained T-igdT cells were washed twice with 2 mL of 1× Permeabilization Buffer and centrifuged at 400×g for 5 minutes at room temperature. The cell pellet was resuspended with FACS staining buffer. The FACS data for individual cell was acquired using a flow cytometer, Attune™ NxT Flow Cytometer (ThermoFisher Scientific) and analyzed using FlowJo™ software (BD Biosciences).

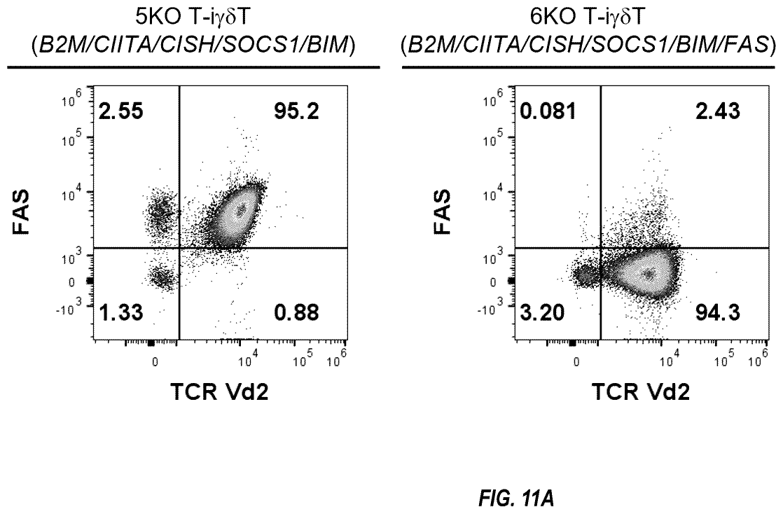

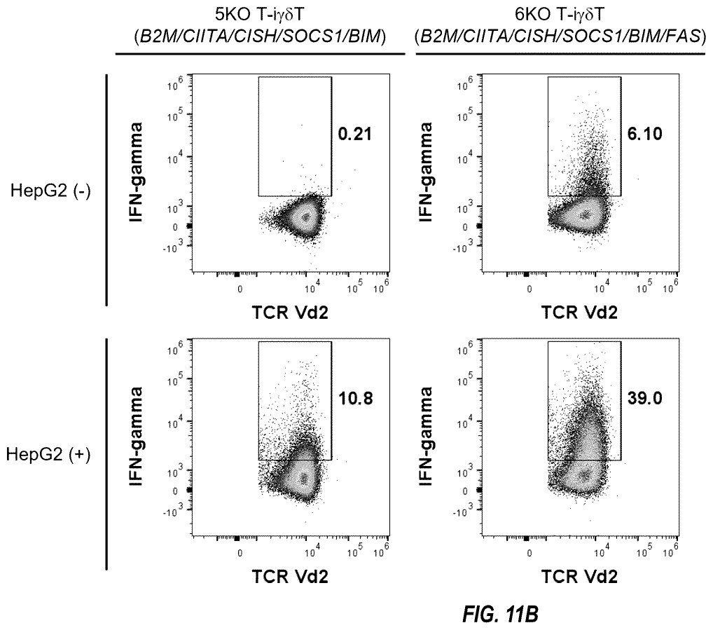

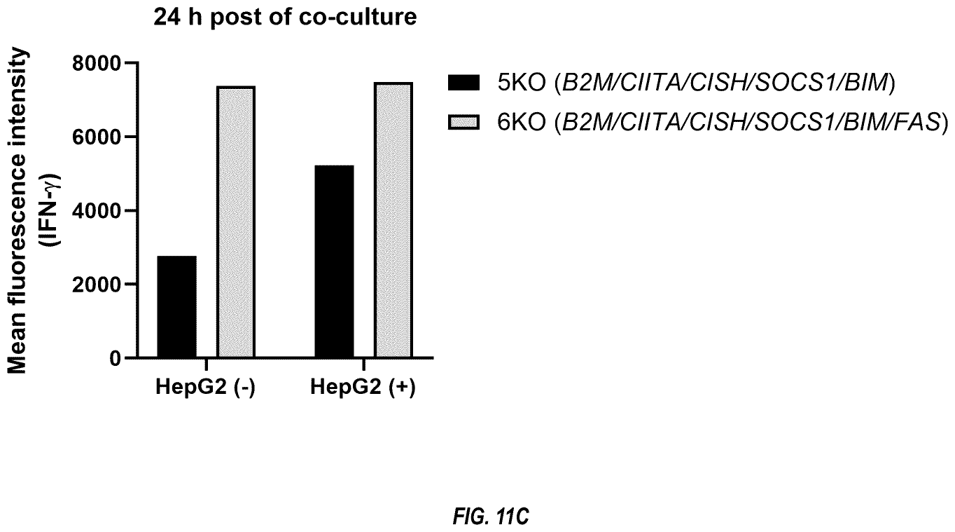

As shown in FIG. 11A, both 5KO (B2M^{-/-}/CIITA^{-/-}/CISH^{-/-}/SOCS1^{-/-}/BIM^{-/-}) and 6KO (B2M^{-/-}/CIITA^{-/-}/CISH^{-/-}/SOCS1^{-/-}/BIM^{-/-}/FAS^{-/-}) T-igdT cells were primarily TCR Vd2⁺ cells (˜96%). The FAS⁺population of the 5KO T-igdT cells was 97.75% compared with only 2.5% of the 6KO T-igdT cells (FIG. 11A).

As shown in FIG. 11B, there was a higher percentage of IFN-g-producing cells in 6KO (B2M^{-/-}/CIITA^{-/-}/CISH^{-/-}/SOCS1^{-/-}/BIM^{-/-}/FAS^{-/-}) T-igdT cells compared with 5KO (B2M^{-/-}/CIITA^{-/-}/CISH^{-/-}/SOCS1^{-/-}/BIM^{-/-}) T-igdT cells (6.1% vs 0.21%, respectively) in the absence of HepG2 cells. The percentage of IFN-g-producing cells was increased in both 5KO and 6KO T-igdT cells after co-culturing with HepG2 cells for 24 hours. There was a higher percentage of IFN-g-producing cells in 6KO (B2M^{-/-}/CIITA^{-/-}/CISH^{-/-}/SOCS1^{-/-}/BIM^{-/-}/FAS^{-/-}) T-igdT cells compared with 5KO (B2M^{-/-}/CIITA^{-/-}/CISH^{-/-}/SOCS1^{-/-}/BIM^{-/-}) T-igdT cells (39% vs 10.8%, respectively) in the presence of HepG2 cells (FIG. 11B).

As shown in FIG. 11C, The IFN-g-producing ability of each T-igdT cell, as indicated by MFI (mean fluorescence intensity), was also higher in 6KO T-igdT cells compared with 5KO T-igdT cells both in the absence of HepG2 cells (IFN-g⁺MFI: 7,384 vs 2,770, respectively) and with co-culturing with HepG2 cells for 24 hours (IFN-g⁺MFI: 7,493 vs 5,223, respectively) (FIG. 11C).

Overall, the FAS deficiency of the 6KO (B2M^{-/-}/CIITA^{-/-}/CISH^{-/-}/SOCS1^{-/-}/BIM^{-/-}/FAS^{-/-}) T-igdT cells increased not only the percentage of IFN-g-producing cells but also the ability of IFN-g production compared with the 5KO (B2M^{-/-}/CIITA^{-/-}/CISH^{-/-}/SOCS1^{-/-}/BIM^{-/-}) T-igdT cells (FIGS. 11A-11C).

### Example 10. IFNγR-IL2/IL15R Signal Converter can Induce STAT Signaling, Increase Proliferation, Enhance Tumor Serial Killing and Cytokines Secretion in Mature iγδT 5KO Cells

### Example 10.1 Methods

### Virus Infection and Sorting

Effector cells iγδT 5KO (B2M^{-/-}/CIITA^{-/-}/CISH^{-/-}/SOCS1^{-/-}/BIM^{-/-}) cells were co-transduced with IFNγ signal converter-encoding viruses in the presence of Vectofusin-1 (Miltenyi Biotec (Bergisch Gladbach, North Rhine-Westphalia, Germany), Cat: 130-111-163) by spin infection at 400×g for 2 hours. After incubation for another 24 hours, the virus was replaced with a fresh complete medium. The resulting cells containing the IFNγ signal converter were sorted 3 days post transduction. To ensure the surface expression levels of IFNγR-IL2/IL15R signal converter, sorted cells were stained with phycoerythrin (PE) anti-human IFNγR1 (1:100; BioLegend (Revvity, Waltham, MA), Cat: 308606) and APC anti-human IFNγR2 (1:100; R&D systems, Cat: FAB773A) according to the manufacturer's instructions and analyzed by a flow cytometer, e.g. the Attune NxT Flow Cytometer (ThermoFisher Scientific (Waltham, MA)). The percentage of cells with IFNγR-IL2/IL15R signal converter was analyzed using the FlowJo software (BD Biosciences (San Jose, CA)).

### Western Blotting

Effector cells iγδT 5KO cells, without (Control cells) or with expressing IFNγR-IL2/IL15R signal converter (signal converter transduced cells) were subjected to cytokines starvation overnight. Then, effector cells were treated with 5 ng/mL IFN-γ in a time-dependent manner (15, 60, and 120 minutes), effector cells without IFN-γ treatment but with cytokines supplementation (iγδT 5KO cells were maintained in IL7 plus IL15 culture conditions) were set as positive control for both Control cells and signal converter transduced cells. Treated cells were harvested, and cell pellets were lysed in a radioimmunoprecipitation assay (RIPA) buffer (Millipore (Burlington, MA), Cat: 20-188) containing a protease inhibitor cocktail (TargetMol (Boston, MA), Cat: C0001) and phosphatase inhibitor cocktail I & II (Omics Bio, Cat: C0002 & C0003). The mixture was vortexed for 1 minute and put on ice for 30 minutes and then centrifuged at 12,000×g for 15 minutes at 4° C. The supernatants were transferred to a new 1.6 mL microcentrifuge tube. The protein concentration of the cell lysate was determined by adding an acidic dye to a solution containing a protein, e.g., the Protein Assay Dye Reagent (Bio-Rad (Hercules, CA), Cat: 5000006). A total of 20 mg of protein in each lane was separated using a polyacrylamide gel, e.g. 4-15% Mini-PROTEAN® TGX™ Precast Protein Gels (Bio-Rad, Cat: 4561086). The separated proteins were subsequently transferred to a polyvinylidene fluoride (PVDF) membrane, e.g., a Power Blotter Select Transfer PVDF membrane (Invitrogen (Waltham, MA), Cat: PB5340). The transferred PVDF membrane was rinsed with 1×TBST (Tris buffered saline and 0.05% Tween 20) and blocked with 5% non-fat milk in TBST at room temperature for 1 hour. The blot was rinsed once for 10 minutes with TBST and then incubated with an anti-phospho-STAT1 (Tyr701) antibody (Cell Signaling Technology (Danvers, MA), Cat: 9167), an anti-phospho-STAT3 (Tyr705) antibody (Cell Signaling Technology, Cat: 9145), and an anti-phospho-STAT5 (Tyr694) antibody (Cell Signaling Technology, Cat: 9359) in 1% non-fat milk/TBST overnight at 4° C. The blot was washed three times for 10 minutes with TBST and incubated with Peroxidase conjugated goat anti-rabbit IgG antibody (1:5,000; Sigma-Aldrich (St. Louis, MO), Cat: AP132P) in 1% non-fat milk/TBST at room temperature for 1 hour. The blot was washed three times for 10 minutes with TBST. A chemiluminescent (ECL) horseradish peroxidase (HRP) substrate, e.g. SuperSignal West Pico PLUS Chemiluminescent Substrate (ThermoFisher Scientific, Cat: 34580), was applied to the blot according to the manufacturer's recommendation. The chemiluminescent signals were captured using an imaging system, e.g. the ChemiDoc™ Imaging System (Bio-Rad). The blots were further stripped by Restore™ PLUS Western Blot Stripping Buffer (ThermoFisher Scientific, Cat: 46430) and re-probed with an anti-STAT1 antibody (Cell Signaling Technology, Cat: 14994), an anti-STAT3 antibody (Cell Signaling Technology, Cat: 9139), and an anti-STAT5 antibody (Cell Signaling Technology, Cat: 94205) in 1% non-fat milk/TBST overnight at 4° C. Beta-actin (1:4,000; Santa Cruz Biotechnology (Dallas, TX), Cat: sc-47778 RP) was used as an internal control to ensure almost equal amount of sample loading for each lane.

Proliferation Assay with IFN-γ Treatment

3×10⁶ of control effector cells (iγδT 5KO cells) and IFNgR-IL2/IL15R expressing effector cells (i.e., IFNgR-IL2/IL15R expressing iγδT 5KO cells), were harvested and stained with a cell dye, e.g., Tag-it Violet™ working solution (1:1,000 in PBS; BioLegend, Cat: 425101) for 20 minutes at 37° C. in the dark. The staining was quenched by adding 5 times the original staining volume of cell culture medium containing 10% FBS. Cells were then centrifuged at 300×g for 5 minutes and resuspended in pre-warmed cell culture medium and incubated for 10 minutes. Next, Tag-it Vilolet™ (VL1, Biolegend, Cat #425101 Lot #345776) labeled effector cells are ready for further incubation in cytokines starvation (negative control), IL7 (1 ng/mL)+IL15 (1 ng/mL) supplemented (for iγδT 5KO cells) (positive control), or IFN-γ (5 ng/mL) supplemented culture medium. The unstained and Tag-it labeled effector cells were analyzed using a flow cytometer, e.g. Attune NxT Flow Cytometer (ThermoFisher Scientific), at day 0 for the gating setting. After 6 days of incubation, the cells were collected and analyzed by flow cytometry following the gating setting at day 0.

### Serial Killing Assay for Adhesion Target Cells

5×10³ of EGFP-SW620 HLA-A11 or EGFP-HepG2 cells were pre-seeded in a 96-well cell culture plate (Corning, Cat: 3599) overnight. The culture supernatant was aspirated. The effector cells, i.e., iγδT 5KO cells, were added into the 96-well cell culture plate in 200 mL of a culture medium without cytokines at a 2:1 or 5:1 effector to target cell ratio. The cytotoxicity of effector cells was monitored with live cell imaging, for example, the Incucyte SX5 Live-Cell Analysis System (Sartorius, Gottingen, Germany). For a further round of killing, the effector cells were transferred to another EGFP-SW620 HLA-A11 or EGFP-HepG2 pre-seeded 96-well cell culture plate after 48 or 24 hours of tumor killing. The cytotoxicity percentage was calculated by the formula listed below:

Cytotoxicity percentage=[1−(Total iRFP713 fluorescence area at indicated timepoint/Total green fluorescence area at starting timepoint of each round of serial killing)]×100%

### Cytokines Secretion

Following the serial killing assay described previously, at the end of each round (e.g., 24, 48, 72, and 96 hours) of killing, the supernatant was collected and cytokine secretion was evaluated using, e.g. LEGENDplex™ Human CD8/NK Panel (BioLegend, Cat: 741186), according to the manufacturer's instructions and analyzed using a flow cytometer, e.g., Attune NxT Flow Cytometer (ThermoFisher Scientific (Waltham, MA)).

### Example 10.2 IFN-γ Signal Converters Design

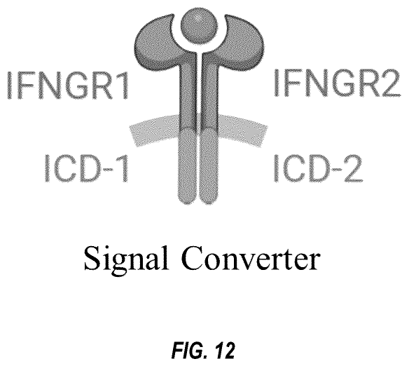

The IFN-γ signal converter (FIG. 12) contains a pair of subunits: one has an IFNGR1 extracellular domain, while the other has an IFNGR2 extracellular domain. IFNGR1 extracellular domain is fused sequentially with membrane-proximal region, transmembrane domain, and intracellular domain of common γ chain (γc) of IL2R (i.e., IL2RG, CD132). IFNGR2 extracellular domain is fused sequentially with membrane-proximal region, transmembrane domain, and intracellular domain of IL2RB. The structures and amino acid sequences of each IFN-γ signal converter subunit are provided in Table 9.

TABLE 9
Design and sequence table of IFN-γ signal converter

| Description | Sequence | SEQ ID NO: |
|---|---|---|
| ECD OF | EMGTADLGPSSVPTPTNVTIESYNMNPIVYWEYQIMPQVPVFTVEVKN | 63 |
| IFNGR1 | YGVKNSEWIDACINISHHYCNISDHVGDPSNSLWVRVKARVGQKESAY | |
| | AKSEEFAVCRDGKIGPPKLDIRKEEKQIMIDIFHPSVFVNGDEQEVDYDP | |
| | ETTCYIRVYNVYVRMNGSEIQYKILTQKEDDCDEIQCQLAIPVSSLNSQ | |
| | YCVSAEGVLHVWGVTTEKSKEVCITIFNSSIKG | |
| ECD of | APPDPLSQLPAPQHPKIRLYNAEQVLSWEPVALSNSTRPVVYQVQFKYT | 64 |
| IFNGR2 | DSKWFTADIMSIGVNCTQITATECDFTAASPSAGFPMDFNVTLRLRAEL | |
| | GALHSAWVTMPWFQHYRNVTVGPPENIEVTPGEGSLIIRFSSPFDIADT | |
| | STAFFCYYVHYWEKGGIQQVKGPFRSNSISLDNLKPSRVYCLQVQAQL | |
| | LWNKSNIFRVGHLSNISCYETMADASTELQQ | |
| Membrane- | FALEA | 65 |
| proximal region | | |
| of IL2RG | | |
| Membrane- | LGKDT | 66 |
| proximal region | | |
| of IL2RB | | |
| Transmembrane | VVISVGSMGLIISLLCVYFWL | 67 |
| domain of | | |
| IL2RG | | |
| Transmembrane | IPWLGHLLVGLSGAFGFIILVYLLI | 68 |
| domain of | | |
| IL2RB | | |
| Intracellular | ERTMPRIPTLKNLEDLVTEYHGNFSAWSGVSKGLAESLQPDYSERLCLV | 69 |
| domain of | SEIPPKGGALGEGPGASPCNQHSPYWAPPCYTLKPET | |
| IL2RG | | |
| Intracellular | NCRNTGPWLKKVLKCNTPDPSKFFSQLSSEHGGDVQKWLSSPFPSSSFS | 70 |
| domain of | PGGLAPEISPLEVLERDKVTQLLLQQDKVPEPASLSSNHSLTSCFTNQGY | |
| IL2RB | FFFHLPDALEIEACQVYFTYDPYSEEDPDEGVAGAPTGSSPQPLQPLSGE | |
| | DDAYCTFPSRDDLLLFSPSLLGGPSPPSTAPGGSGAGEERMPPSLQERVP | |
| | RDWDPQPLGPPTPGVPDLVDFQPPPELVLREAGEEVPDAGPREGVSFP | |
| | WSRPPGQGEFRALNARLPLNTDAYLSLQELQGQDPTHLV | |
| IFNGR1 | EMGTADLGPSSVPTPTNVTIESYNMNPIVYWEYQIMPQVPVFTVEVKN | 71 |
| Subunit: | YGVKNSEWIDACINISHHYCNISDHVGDPSNSLWVRVKARVGQKESAY | |
| IFNGR1- | AKSEEFAVCRDGKIGPPKLDIRKEEKQIMIDIFHPSVFVNGDEQEVDYDP | |
| IL2RG without | ETTCYIRVYNVYVRMNGSEIQYKILTQKEDDCDEIQCQLAIPVSSLNSQ | |
| signal peptide | YCVSAEGVLHVWGVTTEKSKEVCITIFNSSIKGFALEAVVISVGSMGLII | |
| | SLLCVYFWLERTMPRIPTLKNLEDLVTEYHGNFSAWSGVSKGLAESLQ | |
| | PDYSERLCLVSEIPPKGGALGEGPGASPCNQHSPYWAPPCYTLKPET | |
| IFNGR2 | APPDPLSQLPAPQHPKIRLYNAEQVLSWEPVALSNSTRPVVYQVQFKYT | 72 |
| Subunit: | DSKWFTADIMSIGVNCTQITATECDFTAASPSAGFPMDFNVTLRLRAEL | |
| IFNGR2- | GALHSAWVTMPWFQHYRNVTVGPPENIEVTPGEGSLIIRFSSPFDIADT | |
| IL2RB without | STAFFCYYVHYWEKGGIQQVKGPFRSNSISLDNLKPSRVYCLQVQAQL | |
| signal peptide | LWNKSNIFRVGHLSNISCYETMADASTELQQLGKDTIPWLGHLLVGLS | |
| | GAFGFIILVYLLINCRNTGPWLKKVLKCNTPDPSKFFSQLSSEHGGDVQ | |
| | KWLSSPFPSSSFSPGGLAPEISPLEVLERDKVTQLLLQQDKVPEPASLSS | |
| | NHSLTSCFTNQGYFFFHLPDALEIEACQVYFTYDPYSEEDPDEGVAGAP | |
| | TGSSPQPLQPLSGEDDAYCTFPSRDDLLLFSPSLLGGPSPPSTAPGGSGA | |
| | GEERMPPSLQERVPRDWDPQPLGPPTPGVPDLVDFQPPPELVLREAGEE | |
| | VPDAGPREGVSFPWSRPPGQGEFRALNARLPLNTDAYLSLQELQGQDP | |
| | THLV | |
| Signal peptide | MALLFLLPLVMQGVSRA | 79 |
| of IFNGR1 | | |
| Signal peptide | MRPTLLWSLLLLLGVFAAAAA | 80 |
| of IFNGR2 | | |
| IFNGR1 | MALLFLLPLVMQGVSRAEMGTADLGPSSVPTPTNVTIESYNMNPIVYW | 81 |
| Subunit: | EYQIMPQVPVFTVEVKNYGVKNSEWIDACINISHHYCNISDHVGDPSN | |
| IFNGR1- | SLWVRVKARVGQKESAYAKSEEFAVCRDGKIGPPKLDIRKEEKQIMIDIF | |
| IL2RG with | HPSVFVNGDEQEVDYDPETTCYIRVYNVYVRMNGSEIQYKILTQKEDD | |
| signal peptide | CDEIQCQLAIPVSSLNSQYCVSAEGVLHVWGVTTEKSKEVCITIFNSSIK | |
| | GFALEAVVISVGSMGLIISLLCVYFWLERTMPRIPTLKNLEDLVTEYHGN | |
| | FSAWSGVSKGLAESLQPDYSERLCLVSEIPPKGGALGEGPGASPCNQHS | |
| | PYWAPPCYTLKPET | |
| IFNGR2 | MRPTLLWSLLLLLGVFAAAAAAPPDPLSQLPAPQHPKIRLYNAEQVLS | 82 |
| Subunit: | WEPVALSNSTRPVVYQVQFKYTDSKWFTADIMSIGVNCTQITATECDFT | |
| IFNGR2- | AASPSAGFPMDFNVTLRLRAELGALHSAWVTMPWFQHYRNVTVGPPE | |
| IL2RB with | NIEVTPGEGSLIIRFSSPFDIADTSTAFFCYYVHYWEKGGIQQVKGPFRS | |
| signal peptide | NSISLDNLKPSRVYCLQVQAQLLWNKSNIFRVGHLSNISCYETMADAST | |
| | ELQQLGKDTIPWLGHLLVGLSGAFGFIILVYLLINCRNTGPWLKKVLKC | |
| | NTPDPSKFFSQLSSEHGGDVQKWLSSPFPSSSFSPGGLAPEISPLEVLER | |
| | DKVTQLLLQQDKVPEPASLSSNHSLTSCFTNQGYFFFHLPDALEIEACQ | |
| | VYFTYDPYSEEDPDEGVAGAPTGSSPQPLQPLSGEDDAYCTFPSRDDLL | |
| | LFSPSLLGGPSPPSTAPGGSGAGEERMPPSLQERVPRDWDPQPLGPPTPG | |
| | VPDLVDFQPPPELVLREAGEEVPDAGPREGVSFPWSRPPGQGEFRALNA | |
| | RLPLNTDAYLSLQELQGQDPTHLV | |

### Example 10.3 Validation of the Functions of the IFNγR-IL2/IL15R Signal Converter in iPSC-Derived Mature iγδT 5KO Cells

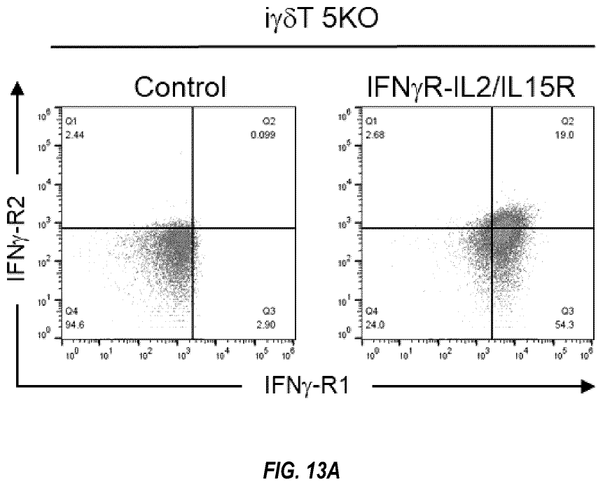

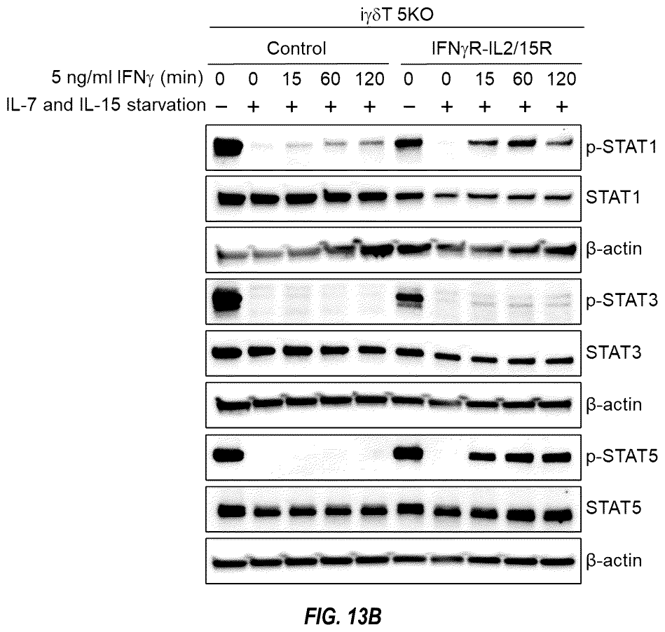

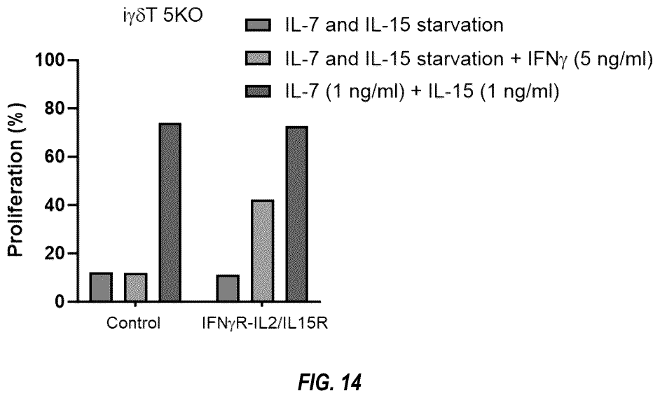

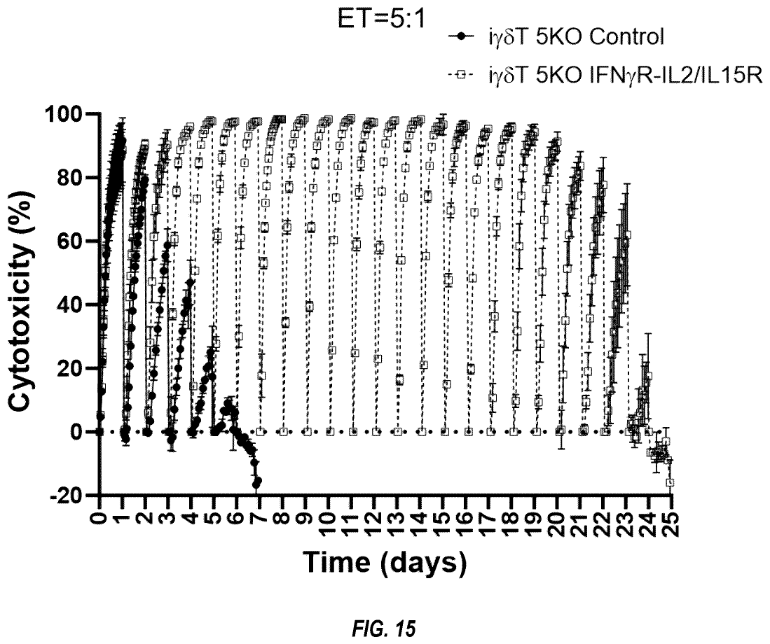

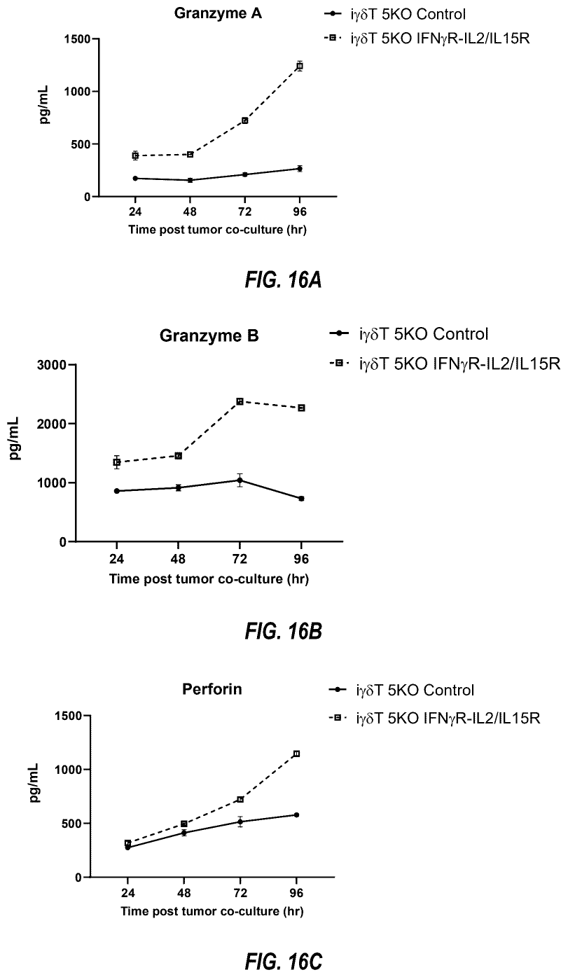

To validate the functions of the IFNγR-IL2/IL,15R signal converter in iPSC-derived mature iγδT 5KO cells, we transfected nucleic acids encoding the IFNγR-IL2/IL15R signal converter by virus infection and sorting according to the protocol described previously. As shown in FIG. 13A, the percentage of cells with the IFNγR-IL2/IL15R signal converter in mature iγδT 5KO cells is about 19%. Phosphorylation of STAT1 and STAT5 were increased in mature iγδT 5KO cells with the IFNγR-IL2/IL15R signal converter under IFN-γ treatment (FIG. 13B). Next, we also performed Tag-it vilolet (VL1, Biolegend, Cat #425101 Lot #345776) cell tracking staining, to analyze the cell proliferation of mature iγδT 5KO cells with the IFNγR-IL2/IL15R signal converter. The results show that the IFNγR-IL2/IL15R signal converter increases cell proliferation of mature iγδT 5KO cells by approximately 40% for 6 days under IFN-γ treatment compared to control iγδT 5KO cells, which lack the IFNγR-IL2/IL15R signal converter (FIG. 14). Additionally, the IFNγR-IL2/IL15R signal converter greatly enhanced the serial tumor killing effect of mature iγδT 5KO cells against HepG2 cells and maintained the serial tumor killing effect for 24 rounds (FIG. 15). The secretion of cytokines, including Granzyme A (FIG. 16A), Granzyme B (FIG. 16B), and Perforin (FIG. 16C), from mature iγδT 5KO cells with the IFNγR-IL2/IL15R signal converter was increased when co-cultured with HepG2 cells in serial killing assays.

Overall, these results indicate that overexpression of the IFNgR-IL2/IL15R signal converter in mature iγδT 5KO cells derived from an iPSC can induce STAT1 and STAT5 activation, promote cell proliferation, greatly improve multi-rounds tumor killing, and further increase cytokine production.

### Example 11. In Vitro Cell Data of iγδT Harboring Anti-CD19 CAR

### Example 11.1 Methods

### Antigen-Specific Recognition and Killing

For luciferase-based killing, luciferase-expressing target cells (Nalm6-Luc or Raji-Luc) were seeded at 1×10⁴ cells per well in a 96-well Flat-bottom Cell Culture Microplate (Greiner, Cat No. 655083) and co-cultured with either control or modified CD19-CAR iγδT cells for 24 hours. Target cell viability was assessed 24 hours post co-culturing using the ONE-Glo™ Luciferase Assay System (Promega, Cat No. E6120) according to the manufacturer's instructions. Basal luminescence from the culture medium alone was measured and subtracted from the luminescence values of each sample well to account for media effects. The percent lysis was established using the following formula: [1−the ratio of the luminescence signal of target cells in the presence of effector cells to the maximum luminescence signal from target cells only]×100.

For FACS-based killing assays involving tumor or human primary normal cells, 1×10⁴ normal cells (fibroblast, smooth muscle, and endothelial cells, purchased from ScienCell Research Laboratories) were seeded in a Falcon® 96-well Flat-Bottom microplate (Corning, Cat No. 353072) and allowed to adhere and spread for 4 hours prior to co-culture with effector cells. Effector cells were pre-labeled with 5 μM Tag-it Violet™ Cell Tracking dye (BioLegend, Cat No. 425101) and co-cultured with target cells (tumor or normal) at specified E:T ratios for 24 hours. After incubation, mixed samples were harvested and stained with DARQ-7 Cell Viability Dye (BioLegend, Cat No. 424001) at a 1:1000 dilution. Flow cytometric analysis was performed immediately after staining. Total live cell counts (DRAQ-7 negative) were quantified using Precision Count Beads (BioLegend, Cat No. 424902), following the manufacturer's protocols. The percent cytotoxicity was calculated as the percentage of dead target cells in the presence of effector cells minus the percentage of spontaneous death in the absence of the effector cells.

### Facs-Based In Vitro PBMCs Cytotoxicity

Healthy-donor PBMC and patient derived (from NHL and SLE patients, three donors each) PBMCs were obtained from Lonza Bioscience and Discovery Life Sciences, respectively. For in vitro cytotoxicity assay, control or modified CD19-CAR iγδT cells were labeled with 5 μM Tag-it Violet™ Cell Tracking dye (1:1000; BioLegend, Cat No. 425101) in accordance with the manufacturer's instructions. Labeled effector cells were co-cultured with 4×10⁴ PBMCs at indicated E:T ratios in a 96-well U-bottom microplate (TPP, Cat No. 92097) and incubated for 24 hours in a humidified 37 degrees with 5% CO₂. Single cultures of target cells were included to evaluate spontaneous death. At the assay endpoint, non-specific Fc-mediated interactions were blocked with human Fc receptor blocking solution (BioLegend, Cat No. 422301) for 10 minutes at room temperature. Afterwards, surface markers were stained with the following antibodies: anti-human CD3 (1:200; MACS, Cat No. 130-113-141), anti-human CD19 (1:100; BioLegend, Cat No. 982402), and anti-human CD20 (1:100; BioLegend, Cat No. 302311) for 20 minutes at room temperature. Viability was determined using a fixable live/dead dye at (1:1000; Invitrogen, Cat No. L34968). Compensation for the full panel was conducted using the stained cells described above, with an unstained group serving as the control. FACS data were acquired on Attune Flow Cytometer (Thermo Fisher Scientific) and were analyzed using FlowJo v10.10.0 (FlowJo LLC). The cytolytic rate was calculated as previously described in the method for Antigen-specific recognition and killing—FACS-based killing assays.

### Serial Killing

To assess tumor clearance, the imaged time-lapse was performed and analyzed frame-by-frame at two-hour intervals using the Incucyte® SX5 live-cell analysis system (Sartorius). Nalm-6 cells expressing the fluorescent reporter iRFP713 (near-infrared fluorescent protein, Nalm-6-iRFP713) were seeded at 5,000 cells per well and co-cultured with modified CD19-CAR iγδT cells at the specified E:T ratios in a Falcon® 96-well Flat-Bottom microplate (Corning, Cat No 353072). Fluorescent signals were followed over time to evaluate the tumor clearance rate. At the endpoint of each assay round, one-fifth of the culture medium in a single well was removed, and additional of Nalm-6-iRFP713 cells was added. Cytotoxicity was calculated using the equation: [1−(fluorescence signal of target cells in the presence of effector cells at each timepoint over maximum fluorescent signals at the initial point of target cell addition)]×100.

### Cell Proliferation Monitoring

Nalm-6-iRFP713 (5,000 cells per well) were seeded into Falcon® 96-well Flat-Bottom microplate (Corning, Cat No 353072) and co-cultured with modified CD19-CAR iγδT cells at indicated E:T ratios. Cells were harvested from a single well and stained in DPBS with anti-human CD3 (1:200; MACS, Cat No. 130-113-141) and fixable live/dead dye (1:1000; Invitrogen™, Cat No. L34968) for 20 minutes at room temperature. Cell proliferation was quantified every two days by determining the total viable absolute number of CD3⁺ cells using the Attune Flow Cytometer and Precision Count Beads (BioLegend, Cat No. 424902) following the manufacturer's instructions.

### Cytokine Release Assay (Bead-Based Multiplex Analysis)

At the endpoint of the tumor killing assay, half of the co-culture supernatant from the tumor-effector mixture was collected to measure cytokine production. Cytokine release assays were performed using the bead-based LEGENDplex™ system (BioLegend, Cat No. 741186) according to the manufacturer's protocols.

### Example 11.2 CD19-CAR Sequence Design

A second-generation anti-CD19-chimeric antigen receptor (CD19-CAR) contain anti-CD19 single-chain variable fragment derived from a murine FMC63 monoclonal antibody (CD19 scFv) leaded by CD8α signal peptide and fused to the hinge region of CD8α (CD8α loop), the transmembrane domain of CD8α (CD8α TM), one intracellular costimulatory domain of 4-1BB (41BB ICD), and a CD3ζ signaling domain (CD3z AD). The structures and amino acid sequences of each domain are provided in Table 10.

TABLE 10
Design and sequence table of CD19-CAR and B2M-HLA-E

| | | |
|---|---|---|
| SEQ ID | CD8α signal | MALPVTALLLPLALLLHAARP |
| NO: 73 | peptide | |
| SEQ ID | CD19 scFv | DIQMTQTTSSLSASLGDRVTISCRASQDISKYLNWYQQKPDGTVKLLIYHTSR |
| NO: 74 | | LHSGVPSRFSGSGSGTDYSLTISNLEQEDIATYFCQQGNTLPYTFGGGTKLEIT |
| | | GGGGSGGGGSGGGGSEVKLQESGPGLVAPSQSLSVTCTVSGVSLPDYGVSWI |
| | | RQPPRKGLEWLGVIWGSETTYYNSALKSRLTIIKDNSKSQVFLKMNSLQTDD |
| | | TAIYYCAKHYYYGGSYAMDYWGQGTSVTVSS |
| SEQ ID | CD8α loop | TTTPAPRPPTPAPTIASQPLSLRPEACRPAAGGAVHTRGLDFACD |
| NO: 75 | | |
| SEQ ID | CD8α TM | IYIWAPLAGTCGVLLLSLVITLYC |
| NO: 76 | | |
| SEQ ID | 41BB ICD | KRGRKKLLYIFKQPFMRPVQTTQEEDGCSCRFPEEEEGGCEL |
| NO: 77 | | |
| SEQ ID | CD3z AD | RVKFSRSADAPAYKQGQNQLYNELNLGRREEYDVLDKRRGRDPEMGGKPRR |
| NO: 78 | | KNPQEGLYNELQKDKMAEAYSEIGMKGERRRGKGHDGLYQGLSTATKDTYD |
| | | ALHMQALPPR |
| SEQ ID | CD19-CAR | DIQMTQTTSSLSASLGDRVTISCRASQDISKYLNWYQQKPDGTVKLLIYHTSR |
| NO: 83 | without | LHSGVPSRFSGSGSGTDYSLTISNLEQEDIATYFCQQGNTLPYTFGGGTKLEIT |
| | signal | GGGGSGGGGSGGGGSEVKLQESGPGLVAPSQSLSVTCTVSGVSLPDYGVSWI |
| | peptide | RQPPRKGLEWLGVIWGSETTYYNSALKSRLTIIKDNSKSQVFLKMNSLQTDD |
| | | TAIYYCAKHYYYGGSYAMDYWGQGTSVTVSSTTTPAPRPPTPAPTIASQPLSL |
| | | RPEACRPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAYKQGQ |
| | | NQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| SEQ ID | CD19-CAR | MALPVTALLLPLALLLHAARPDIQMTQTTSSLSASLGDRVTISCRASQDISKYL |
| NO: 84 | with signal | NWYQQKPDGTVKLLIYHTSRLHSGVPSRFSGSGSGTDYSLTISNLEQEDIATY |
| | peptide | FCQQGNTLPYTFGGGTKLEITGGGGSGGGGSGGGGSEVKLQESGPGLVAPSQ |
| | | SLSVTCTVSGVSLPDYGVSWIRQPPRKGLEWLGVIWGSETTYYNSALKSRLTI |
| | | IKDNSKSQVFLKMNSLQTDDTAIYYCAKHYYYGGSYAMDYWGQGTSVTVSS |
| | | TTTPAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEEGG |
| | | CELRVKFSRSADAPAYKQGQNQLYNELNLGRREEYDVLDKRRGRDPEMGGK |
| | | PRRKNPQEGLYNELQKDKMAEAYSEIGMKGERRRGKGHDGLYQGLSTATKD |
| | | TYDALHMQALPPR |
| SEQ ID | B2M-HLA-E | MSRSVALAVLALLSLSGLEAVMAPRTLILGGGGSGGGGSGGGGSIQRTPKIQV |
| NO: 85 | fusion | YSRHPAENGKSNFLNCYVSGFHPSDIEVDLLKNGERIEKVEHSDLSFSKDWSF |
| | protein | YLLYYTEFTPTEKDEYACRVNHVTLSQPKIVKWDRDMGGGGSGGGGSGGGG |
| | | SGGGGSGSHSLKYFHTSVSRPGRGEPRFISVGYVDDTQFVRFDNDAASPRMV |
| | | PRAPWMEQEGSEYWDRETRSARDTAQIFRVNLRTLRGYYNQSEAGSHTLQW |
| | | MHGCELGPDGRFLRGYEQFAYDGKDYLTLNEDLRSWTAVDTAAQISEQKSN |
| | | DASEAEHQRAYLEDTCVEWLHKYLEKGKETLLHLEPPKTHVTHHPISDHEAT |
| | | LRCWALGFYPAEITLTWQQDGEGHTQDTELVETRPAGDGTFQKWAAVVVPS |
| | | GEEQRYTCHVQHEGLPEPVTLRWKPASQPTIPIVGIIAGLVLLGSVVSGAVVAA |
| | | VIWRKKSSGGKGGSYSKAEWSDSAQGSESHSL* |

### Example 11.3 Generation of Functional Enhanced iγδT with 6KO, CD19-CAR KI, B2M-HLA-E KI and IFNγR-IL2/IL15R Signal Converter (IFNγSC) KI (Modified CD19-CAR iγδT Cells)

To generate the functional enhanced 6KO iPSC with CD19-CAR KI, B2M-HLA-E KI and IFNγR-IL2/IL15R signal converter (also referred to as IFNγSC) KI (also referred to as modified CD19-CAR iPSC cells), the 5KO iPSC clone as described in Example 6 was nucleofected with a MAD7 RNP containing crRNAs targeting to B32M and a DNA template of B2M-HLA-E fusion sequence (SEQ TD NO: 85) flanking by B32M targeting sequence. After 2 passages, nucleofected iPSCs were stained with PE/Cyanine7 anti-human TILA-E Antibody (Biolegend 342608) and FACS purified to enrich HLA-E expressing iPSCs. Next, the sorted iPSC (5KO and HLA-E KI) was nucleofected with Alt-R™ S.p. HiFi Cas9 Nuclease V3 (Integrated DNA Technologies; Cat: 1081060) and crRNAs targeting to FAS and then FACS purified for FAS negative population. Next, the sorted iPSC (6KO and B2M-HLA-E KI) was nucleofected with Alt-R™ S.p. HiFi Cas9 Nuclease V3 (Integrated DNA Technologies; Cat: 1081060), crRNAs targeting to AAVS1 and a DNA template of CD19-CAR. Nucleofected iPSCs were then stained with anti-FMC63 antibody (ACROBliosystems, #FM3-HIPY53) and subjected to single cell sorting for clone selection. The selected clone with stable CAR expression (6KO, HLA-E KI and CD19-CAR KI) was further nucleofected with a MAD7 RNP containing crRNAs targeting to ROSA26 and a DNA template of IFNGR2 subunit (shown in Table 10). Bulk sorted IFNGR2 subunit expression iPSC was then nucleofected with a MAD7 RNP containing crRNAs targeting to SOCS1 and a DNA template of IFNGR1 subunit (shown in Table 10). The cells were stained with PE anti-human CD119 (IFN-γ R α chain) Antibody (Biolegend 308606) and Human IFN-gamma R2 APC-conjugated Antibody (R&D FAB773A), followed by single-cell sorting and clone selection. A final clone of modified CD19-CAR iPSC cells (6KO, B2M-HLA-E KI, CD19-CAR KI and IFNγSC KI) was selected for differentiation into modified CD19-CAR iγδT.

### Example 11.4 In Vitro Functional Characterization of the Modified CD19-CAR iγδT

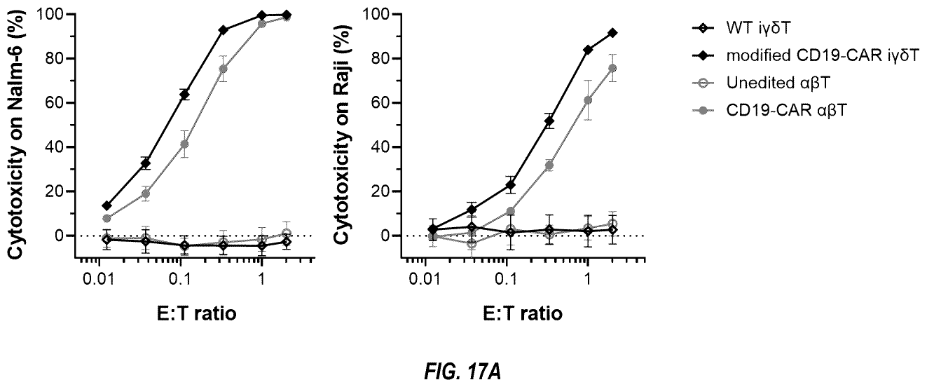

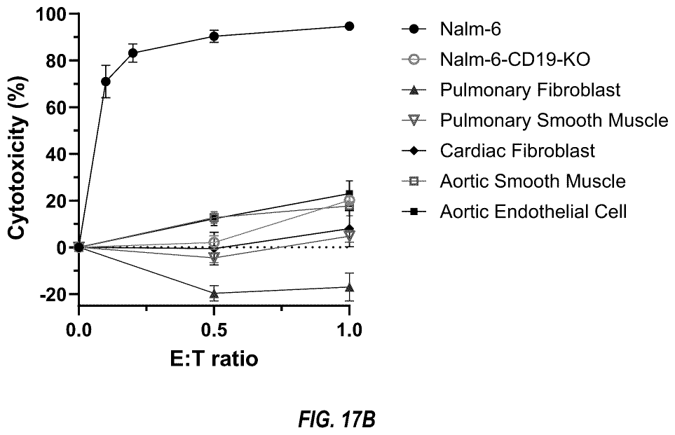

To illustrate the specific potency of the modified CD19-CAR iγδT, antigen specific recognition and killing assay were conducted against CD19-positive tumor cells, including human B-cell acute lymphoblastic leukemia (Nalm-6) and human Burkitt's lymphoma (Raji) as well as CD19-negative Nalm-6 (Nalm-6-CD19-KO, CD19 knock-out on Nalm-6) and human primary cells from normal tissues including pulmonary fibroblast, pulmonary smooth muscle, cardiac fibroblast, aortic smooth muscle, and aortic endothelial cells. As shown in FIG. 17A, wild-type (WT, unedited) iγδT, modified CD19-CAR iγδT, donor-derived unedited aPT (unedited αβT, human primary T cell isolated and activated from healthy donors' PBMC), donor-derived CD19-CAR αβT cells (human primary T cell transduced with lentivirus-derived CD19-CAR expression) were co-cultured with luciferase stable-expressing tumor cell lines Nalm-6-Luc and Raji-Luc at defined Effector:Target (E:T) ratio (0.01, 0.03, 0.1, 0.3, 1 or 2) for 24 hours. The modified CD19-CAR iγδT showed dose-dependent tumor killing potency and substantial tumor elimination at E:T ratio of 1 or 2. The cytotoxicity of modified CD19-CAR iγδT against CD19-positive tumor cells was comparable to or even better than that of donor-derived CD19-CAR αβT cells. As shown in FIG. 17B, the modified CD19-CAR iγδT cells were co-culture with Nalm-6, CD19-negative Nalm-6 and several normal human primary cells at E:T ratio of 0, 0.5 or 1 for 24 hours. The cytotoxicity assay was performed by FACS-based analysis through fluorescence dye (Tag-it Violet™ Cell Tracking dye) labeling on effector T cells and counting the viable target cells number by absolute counting beads after co-culture. The results showed minimal activity of modified CD19-CAR iγδT against CD19-negative Nalm-6 and normal primary cells. Overall, FIG. 17 demonstrate dose-dependent and antigen-specific cytotoxicity of the modified CD19-CAR iγδT cells.

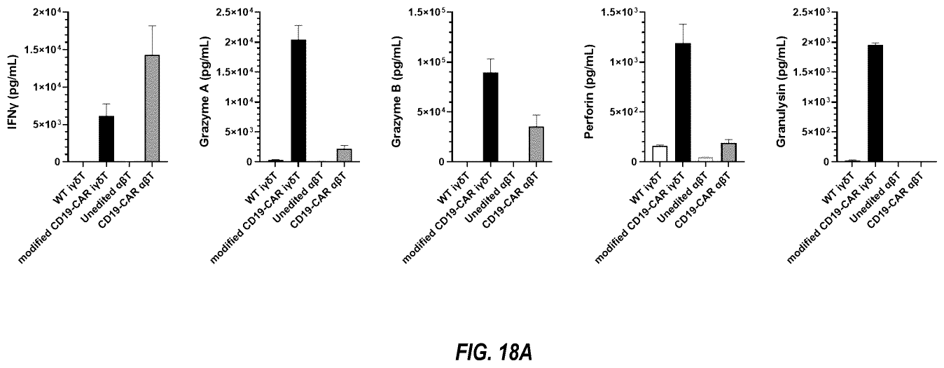

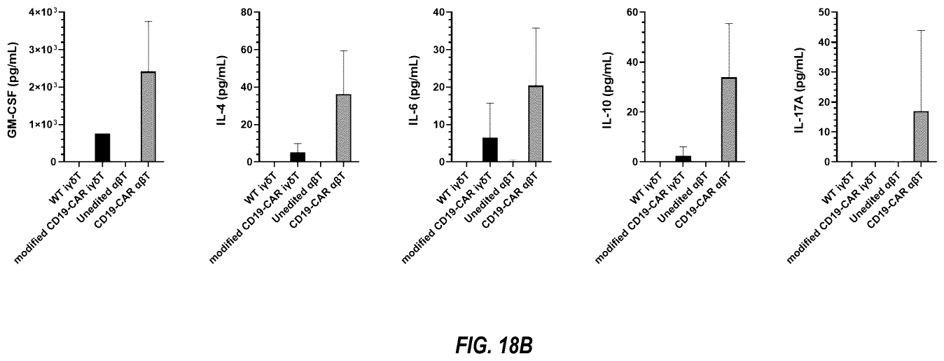

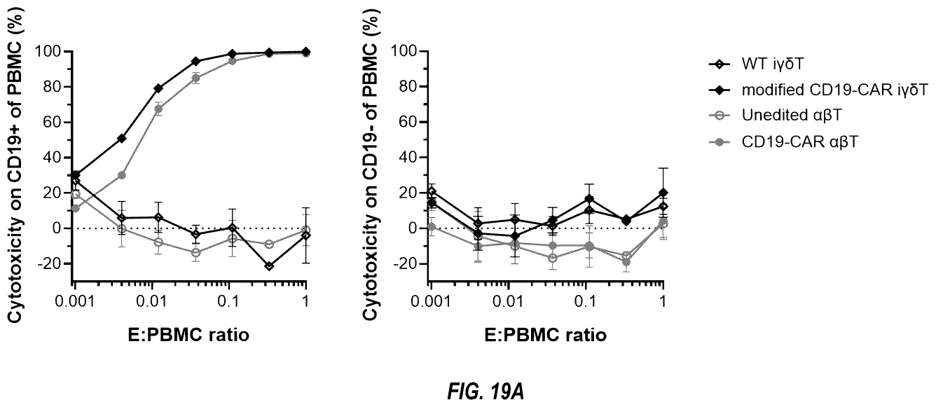

Furthermore, to evaluate the cytokine-releasing capability of modified CD19-CAR iγδT cells, a potency assay of WT iγδT, modified CD19-CAR iγδT, unedited αβT and CD19-CAR αβT cells against CD19-positive Nalm-6 cells were conducted at E:T ratio of 2 for 24 hours and conditioned co-culture medium were harvested to determine cytokine secretion using the bead-based LEGENDplex™ Human CD8/NK Panel analysis. As shown in FIG. 18A, the modified CD19-CAR iγδT cells produced significantly increased amounts of granzyme A, granzyme B, perforin, and granulysin upon co-culture with CD19-positive Nalm-6 cells, compared with CD19-CAR αβT cells. As shown in FIG. 18B, the modified CD19-CAR iγδT cells produced relatively lower amount of inflammatory (IL-4, IL-6, and IL-17a) and immune-modulatory (GM-CSF and IL-10) cytokines by comparison with CD19-CAR αβT cells.

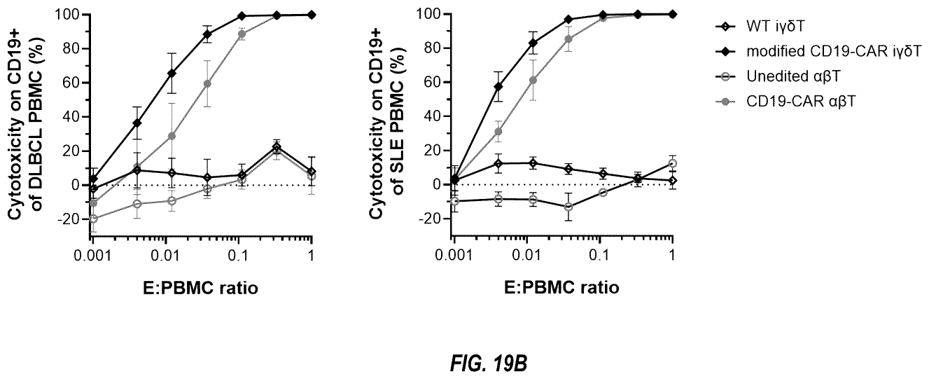

To demonstrate that the modified CD19-CAR iγδT exhibits the potency against the primary B cell in human, the FACS-based killing assay were performed by co-culture WT iγδT, modified CD19-CAR iγδT, unedited αβT and CD19-CAR αβT cells with primary human PBMCs from healthy donors (FIG. 19A) or from diffuse large B-cell lymphoma (DLBCL) and systemic lupus erythematosus (SLE) patients (FIG. 19B) at multiple E:T ratios (0.001, 0.003, 0.01, 0.03, 0.1, 0.3, 1) for 24 hours. The cytotoxicity was determined by fluorescence dye (Tag-it Violet™ Cell Tracking dye) labeling on effector T cells and calculated the viable CD19-positive or -negative cells number of PBMC by absolute counting beads after co-culture. As shown in FIG. 19A, the modified CD19-CAR iγδT cells showed dose-dependent and comparable killing potency with CD19-CAR αβT cells on CD19-positive B cells of healthy BPMCs. Moreover, the modified CD19-CAR iγδT cells showed minimal activity against CD19-negative cells of PMMCs. As shown in FIG. 19B, the modified CD19-CAR iγδT cells showed robust, consistent antigen-dependent cytotoxicity against PBMCs derived from patients with diffuse large B-cell lymphoma (DLBCL) or systemic lupus erythematosus (SLE). The cytotoxicity of the modified CD19-CAR iγδT cells against CD19-positive B cells in patients' PBMC was comparable to or even better than that of donor-derived CD19-CAR αβT cells. Overall, the modified CD19-CAR iγδT exhibits the excellent efficacy and antigen specificity against CD19-expressing cells within both healthy-donor and patient-derived PBMCs.

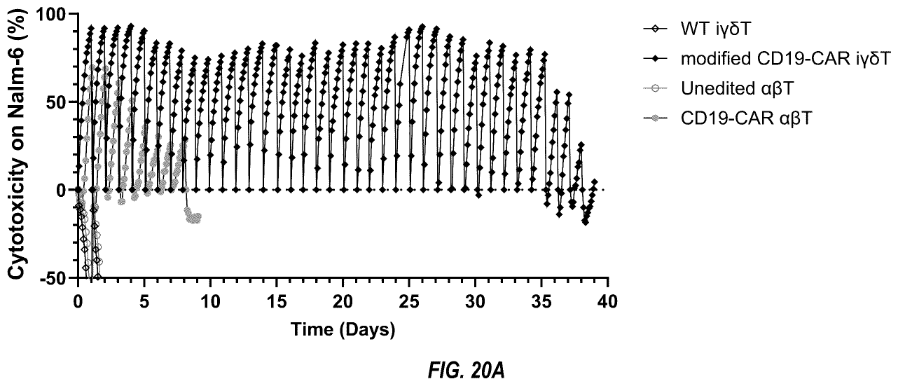

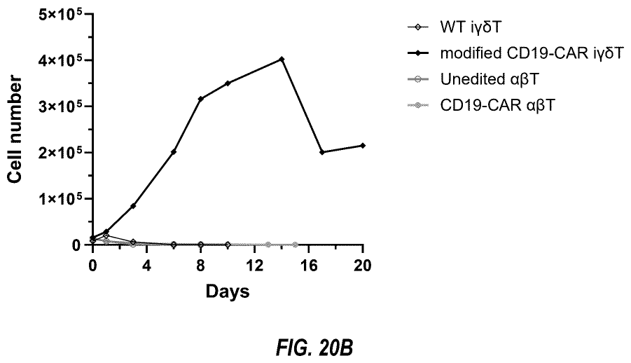

To evaluate the persistence of the modified CD19-CAR iγδT, a serial killing assay was conducted by tumor cell rechallenge experiment using the IncuCyte live-cell imaging system, wherein the target cell death is monitored over time. As shown in FIG. 20A, the modified CD19-CAR iγδT displayed persistent cytotoxicity against Nalm-6-iRFP713 for up to 40 rounds of reintroduction of Nalm-6-iRFP713 every day, without exogenous cytokine support. In addition, As shown in FIG. 20B, the cell number of the effector T cells during this serial killing assay by flow cytometric analysis suggested that the modified CD19-CAR iγδ T cells could further expand after Nalm-6-iRFP713 rechallenge, while the donor-derived CD19-CAR αβ T cells could not. Overall, the modified CD19-CAR iγδT shows the excellent durable cytotoxicity and the cell proliferation capability against antigen-specific tumor cells.

### Example 12. Synergistics Effect of IFNγSC and Anti-CD19 CAR in iγδT

### Example 12.1 Methods

### Serial Killing

To assess tumor clearance, the imaged time-lapse was performed and analyzed frame-by-frame at two-hour intervals using the Incucyte® SX5 live-cell analysis system (Sartorius). Nalm-6 cells expressing the fluorescent reporter iRFP713 (near-infrared fluorescent protein, Nalm-6-iRFP713) were seeded at 5,000 cells per well and co-cultured with modified iγδT cells at the specified E:T ratios in a Corning® 96-well Flat-Bottom microplate (Cat No 3599). Fluorescent signals were followed over time to evaluate the tumor clearance rate. At the endpoint of each assay round, one-fifth of the culture medium in a single well was removed, and additional of Nalm-6-iRFP713 cells was added. Cytotoxicity was calculated using the equation: [1−(fluorescence signal of target cells in the presence of effector cells at each timepoint over maximum fluorescent signals at the initial point of target cell addition)]×100.

### Cell Proliferation Assay by EdU Incorporation

The Click-iT™ Plus EdU Pacific Blue™ Flow Cytometry Assay Kit (Invitrogen, C10636) was performed to assess proliferate activity of iγδT cells following the manufacturer's instructions. Nalm-6 CD19-KO or WT (10,000 cells per well) were seeded into Falcon® 96-well Flat-Bottom microplate (Cat No 353072) and co-cultured with effector iγδT cells at indicated E:T ratios for 24 hours. 4 μM EdU was added to each well for 4 hours incubation before cell harvested. At the assay endpoint, cells were stained with anti-human CD3 (1:200; MACS, Cat No. 130-113-141), and fixable live/dead dye (1:1000; Invitrogen, Cat No. L34968). FACS data were acquired on Attune Flow Cytometer (Thermo Fisher Scientific) and were analyzed using FlowJo v10.10.0 (FlowJo LLC). The percentage of S-phase cells in the population was determined by EdU+percentage of viable CD3+ cells.

### Example 12.2 Generation of Functional Enhanced 5KO iγδT with IFNγSC and/or Anti-CD19 CAR

To generate the functional enhanced 5KO iPSC with IFNγR-IL2/IL15R signal converter (also referred to as IFNγSC), the 5KO iPSC clone as described in Example 6 was co-transduced with IFNγ signal converter-encoding viruses in the presence of protamine sulfate (8 μg/mL). After incubation for 24 hours, the virus was replaced with a fresh complete medium. The resulting cells containing the IFNγSC were sorted 3 days post-transduction. To ensure the surface expression levels of IFNγR-IL2/IL15R signal converter, sorted cells were stained with phycoerythrin (PE) anti-human IFNγR1 (1:100; BioLegend, Cat: 308606) and APC anti-human IFNγR2 (1:100; R&D systems, Cat: FAB773A) according to the manufacturer's instructions. The sorted IFNγSC iPSC cells (5KO with IFNγSC overexpression) were differentiated into IFNγSC 5KO iγδT.

Next, the differentiated 5KO and IFNγSC 5KO iγδT cells were transduced with anti-CD19 CAR-encoding viruses respectively in the presence of Vectofusin-1 (Miltenyi Biotec, Cat: 130-111-163) by spin infection at 400×g for 2 hours. After incubation for another 24 hours, the virus was replaced with a fresh complete medium. To ensure the surface expression levels of anti-CD19 CAR, modified CD19-CAR 5KO iγδT cells were then stained with anti-FMC63 antibody (ACROBiosystems, Cat. FM3-HPY53) and performed functional assay post-transduction for 7 days.

### Example 12.3 In Vitro Functional Assessment of Synergistic Effect in IFNγSC and Anti-CD19 CAR iγδT

To evaluate the synergistic effect of IFNγSC and anti-CD19 CAR in iγδT cells, a serial killing assay was conducted by tumor cell rechallenge experiment using the IncuCyte® live-cell imaging system, wherein the target cell death is monitored over time. As shown in FIG. 21A, the 5KO, IFNγSC 5KO, CD19-CAR 5KO, and IFNγSC+CD19-CAR 5KO iγδT cells were performed against Nalm-6-iRFP713 for repeated tumor challenge every day at ET4, without exogenous cytokine support. The results illustrate the prolong durability of IFNγSC+CD19-CAR 5KO iγδT than CD19-CAR 5KO iγδT cells.

The synergistic effect of IFNγSC and anti-CD19 CAR on cell proliferate activity in iγδT cells was shown in FIG. 21B. The 5KO, IFNγSC 5KO, CD19-CAR 5KO, and IFNγSC+CD19-CAR 5KO iγδT cells were coculture with CD19-KO or CD19 wild-type (WT) Nalm-6 tumor cells for 24 hours at ET2. The results demonstrate that IFNγSC+CD19-CAR 5KO iγδT cells exhibited higher proliferate ability than single effect induced by IFNγSC or CD19-CAR on 5KO iγδT cells. Overall, the IFNγSC and CD19-CAR showed synergistic effect on modified iTST cells.

1. A modified iPSC, γδT cell, or intermediate cell differentiated from an iPSC, comprising 1) an exogenous polynucleotide encodes a CAR which specifically binds human CD19, 2) a genomic disruption in an endogenous suppressor of cytokine signaling 1 (SOCS1) gene that suppresses or eliminates expression of a functional SOCS1 protein, 3) a genomic disruption in an endogenous cytokine-inducible SH2-containing protein (CISH) gene that suppresses or eliminates expression of a functional CISH protein, and 4) a genomic disruption in an exon 2C of an endogenous BIM gene, wherein the expression and/or function of BIM_{EL} and BIM_{L} splice variants are reduced in the modified cell relative to that in an unmodified control and the expression and/or function of BIM_{S} splice variant is retained in the modified cell.

2. The modified cell of claim 1, wherein the genomic disruption is in exon 2 of the SOCS1 gene; and/or wherein the genomic disruption is in exon 3 of the CISH gene.

3. The modified cell of claim 1, wherein the genomic disruption is within a target sequence in BIM that comprises SEQ ID NO: 22; wherein the genomic disruption is within a target sequence in SOCS1 that comprises SEQ ID NO: 23; and/or wherein the genomic disruption is within a target sequence in CISH that comprises SEQ ID NO: 24.

4. The modified cell of claim 1, wherein the genomic disruption is made by administering a guide RNA to said cell, wherein the guide RNA is a single-guide RNA (sgRNA) or crRNA, and wherein an endonuclease or a nucleic acid encoding the endonuclease is also administered to said cell.

5. The modified cell of claim 4, wherein the guide RNA comprises SEQ ID NO: 36 or comprises the guide sequence of SEQ ID NO: 10, and the endonuclease is a MAD7 endonuclease; wherein the guide RNA comprises SEQ ID NO: 37 or comprises the guide sequence of SEQ ID NO: 11, and the endonuclease is a MAD7 endonuclease; and/or wherein the guide RNA comprises SEQ ID NO: 38 or comprises the guide sequence of SEQ ID NO: 12, and the endonuclease is a MAD7 endonuclease.

6. The modified cell of claim 1, wherein the exogenous polynucleotide encoding the CAR is inserted into an AAVS1 locus.

7. The modified cell of claim 1, wherein the CAR which specifically binds human CD19 comprises the amino acid of SEQ ID NO: 83 or SEQ ID NO: 84.

8. The modified cell of claim 1, further comprising a genomic disruption in an endogenous FAS cell surface death receptor (FAS) gene that suppresses or eliminates expression of a functional FAS protein.

9. The modified cell of claim 1, further comprising one or more exogenous polynucleotides encoding an IFNγ signal converter, wherein the IFNγ signal converter comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB).

10. The modified cell of claim 9, wherein a first exogenous polynucleotide encoding the first IFNγ fusion protein subunit of the IFNγ signal converter is inserted into a SOCS1 locus; and/or wherein a second exogenous polynucleotide encoding the second IFNγ fusion protein subunit of the IFNγ signal converter is inserted into a ROSA26 locus; and/or wherein the first IFNγ fusion protein subunit of the IFNγ signal converter comprises an amino acid sequence of SEQ ID NO: 71, and the second IFNγ fusion protein subunit of the IFNγ signal converter comprises an amino acid sequence of SEQ ID NO: 72.

11. A pharmaceutical composition comprising the modified γδT cell of claim 1 and a pharmaceutically acceptable carrier.

12. A method of killing B cells which are CD19 positive and have abnormal B cell function, comprising administering to a subject in need thereof a therapeutically effective amount of the modified γδT cell of claim 11.

13. A modified iPSC, γδT cell, or intermediate cell differentiated from an iPSC, comprising a genomic disruption in an endogenous suppressor of cytokine signaling 1 (SOCS1) gene that suppresses or eliminates expression of a functional SOCS1 protein and a genomic disruption in an endogenous cytokine-inducible SH2-containing protein (CISH) gene that suppresses or eliminates expression of a functional CISH protein.

14. A modified iPSC, γδT cell, or intermediate cell differentiated from an iPSC, comprising a genomic disruption in an exon 2C of an endogenous BIM gene, wherein the expression and/or function of BIM_{EL} and BIM_{L} splice variants are reduced in the modified cell relative to that in an unmodified control, and the expression and/or function of BIM_{S} splice variant is retained in the modified cell.

15. A modified iPSC, γδT cell, or intermediate cell differentiated from an iPSC, comprising one or more exogenous polynucleotides encoding an IFNγ signal converter, wherein the IFNγ signal converter comprises a first IFNγ fusion protein subunit and a second IFNγ fusion protein subunit, wherein the first IFNγ fusion protein subunit comprises an extracellular domain (ECD) of interferon gamma receptor 1 (IFNGR1) and an intracellular domain of interleukin-2 receptor subunit gamma (IL2RG), and wherein the second IFNγ fusion protein subunit comprises an ECD of interferon gamma receptor 2 (IFNGR2) and an intracellular domain of interleukin-2 receptor subunit beta (IL2RB); optionally further comprising an exogenous polynucleotide encoding a chimeric antigen receptor (CAR), optionally wherein the exogenous polynucleotide encoding the CAR is inserted into an AAVS1 locus, optionally wherein the exogenous polynucleotide encodes the CAR which specifically binds human CD19, optionally wherein the CAR which specifically binds human CD19 comprises the amino acid of SEQ ID NO: 83 or SEQ ID NO: 84.

16. A gene editing system comprising one or more guide RNA and an endonuclease or a nucleic acid encoding the endonuclease, wherein the one or more guide RNAs comprises one or more single guide RNA (sgRNA) or CRISPR RNA (crRNA) that comprises:
a. a guide sequence that binds to a target sequence comprising any one of SEQ ID NOs: 17, 23, 15, 16, and 24;
b. a guide sequence that binds to a target sequence comprising any one of SEQ ID NOs: 14, 18, 19, 20, 21, and 22;
c. a first guide sequence that binds to a target sequence comprising any one of SEQ ID NOs: 17 and 23; and a second guide sequence that binds to a target sequence comprising any one of SEQ ID NOs: 15, 16, and 24;
d. a first guide sequence that binds to a target sequence comprising any one of SEQ ID NOs: 17 and 23; a second guide sequence that binds to a target sequence comprising any one of SEQ ID NOs: 15, 16, and 24; and a third guide sequence that binds to a target sequence comprising any one of SEQ ID NOs: 14, 18, 19, 20, 21, and 22;
e. a first guide sequence that binds to a target sequence comprising any one of SEQ ID NOs: 17 and 23; a second guide sequence that binds to a target sequence comprising any one of SEQ ID NOs: 15, 16, and 24; a third guide sequence that binds to a target sequence comprising any one of SEQ ID NOs: 14, 18, 19, 20, 21, and 22; a fourth guide sequence that binds to a target sequence comprising SEQ ID NO: 56; and, a fifth guide sequence that binds to a target sequence comprising SEQ ID NO: 59;
f. a first guide sequence that binds to a target sequence comprising any one of SEQ ID NOs: 17 and 23; a second guide sequence that binds to a target sequence comprising any one of SEQ ID NOs: 15, 16, and 24; a third guide sequence that binds to a target sequence comprising any one of SEQ ID NOs: 14, 18, 19, 20, 21, and 22; a sixth guide sequence that binds to a target sequence comprising SEQ ID NO: 62; a fourth guide sequence that binds to a target sequence comprising SEQ ID NO: 56; and a fifth guide sequence that binds to a target sequence comprising SEQ ID NO: 59;
g. a guide sequence that comprises any one of SEQ ID NOs: 5 and 11;
h. a sgRNA or crRNA sequence that comprises SEQ ID NO: 29 and 37;
i. a guide sequence that comprises any one of SEQ ID NOs: 3, 4, and 12;
j. a sgRNA or crRNA sequence that comprises any one of SEQ ID NOs: 27, 28, and 38;
k. a guide sequence that comprises any one of SEQ ID NOs: 2, 6, 7, 8, 9, 10;
l. a sgRNA or crRNA sequence that comprises any one of SEQ ID NOs: 26, 32, 33, 34, 35, and 36;
m. a first guide sequence that comprises any one of SEQ ID NOs: 5 and 11, and a second guide sequence that comprises any one of SEQ ID NOs: 3, 4, and 12;
n. a first sgRNA or crRNA sequence that comprises any one of SEQ ID NOs: 29 and 37, and a second sgRNA or crRNA sequence that comprises any one of SEQ ID NOs: 27, 28, and 38;
o. a first guide sequence that comprises any one of SEQ ID NOs: 5 and 11; a second guide sequence that comprises any one of SEQ ID NOs: 3, 4, and 12; and a third guide sequence that comprises any one of SEQ ID NOs: 2, 6, 7, 8, 9, and 10;
p. a first sgRNA or crRNA sequence that comprises any one of SEQ ID NOs: 29 and 37; a second sgRNA or crRNA sequence that comprises any one of SEQ ID NOs: 27, 28, and 38; and a third sgRNA or crRNA sequence that comprises any one of SEQ ID NOs: 26, 32, 33, 34, 35, and 36;
q. a first guide sequence that comprises any one of SEQ ID NOs: 5, and 11; a second guide sequence that comprises any one of SEQ ID NOs: 3, 4, and 12; a third guide sequence that comprises any one of SEQ ID NOs: 2, 6, 7, 8, 9, and 10; a fourth guide sequence that comprises SEQ ID NO: 55; and, a fifth guide sequence that comprises SEQ ID NO: 58;
r. a first sgRNA or crRNA sequence that comprises any one of SEQ ID NOs: 29 and 37; a second sgRNA or crRNA sequence that comprises any one of SEQ ID NOs: 27, 28, and 38; a third sgRNA or crRNA sequence that comprises any one of SEQ ID NOs: 26, 32, 33, 34, 35, and 36; a fourth sgRNA sequence that comprises SEQ ID NO: 54; and, a fifth sgRNA sequence that comprises SEQ ID NO: 57;
s. a first guide sequence that comprises any one of SEQ ID NOs: 5, and 11; a second guide sequence that comprises any one of SEQ ID NOs: 3, 4, and 12; a third guide sequence that comprises any one of SEQ ID NOs: 2, 6, 7, 8, 9, and 10; a sixth guide sequence that comprises SEQ ID NO: 61; a fourth guide sequence that comprises SEQ ID NO: 55; and, a fifth guide sequence that comprises SEQ ID NO: 58; or
t. a first sgRNA or crRNA sequence that comprises any one of SEQ ID NOs: 29 and 37; a second sgRNA or crRNA sequence that comprises any one of SEQ ID NOs: 27, 28, and 38; a third sgRNA or crRNA sequence that comprises any one of SEQ ID NOs: 26, 32, 33, 34, 35, and 36; a sixth sgRNA sequence that comprises SEQ ID NO: 60; a fourth sgRNA sequence comprises SEQ ID NO: 54;
and, a fifth sgRNA sequence that comprises SEQ ID NO: 57.

17. A guide RNA comprising SEQ ID NO: 5 (SOCS1), SEQ ID NO: 29 (SOCS1), SEQ ID NO: 11 (SOCS1), SEQ ID NO: 37 (SOCS1), SEQ ID NO: 27 (CISH), SEQ ID NO: 28 (CISH), SEQ ID NO: 12 (CISH), SEQ ID NO: 38 (CISH), SEQ ID NO: 6 (BIM), SEQ ID NO: 32 (BIM), SEQ ID NO: 7 (BIM), SEQ ID NO: 33 (BIM), SEQ ID NO: 8 (BIM), SEQ ID NO: 34 (BIM), SEQ ID NO: 9 (BIM), SEQ ID NO: 35 (BIM), SEQ ID NO: 10 (BIM), or SEQ ID NO: 36 (BIM).

18. A modified iPSC, γδT cell, or intermediate cell differentiated from an iPSC, comprising a genomic disruption in an exon 2C of an endogenous BIM gene, wherein the expression and/or function of BIM_{EL} and BIM_{L} splice variants are reduced in the modified cell relative to that in an unmodified control, and the expression and/or function of BIM_{S} splice variant is retained in the modified cell, wherein the genomic disruption is within a target sequence in BIM that comprises any one of SEQ ID NOs: 14, 20, 21, and 22.

19. A modified iPSC, γδT cell, or intermediate cell differentiated from an iPSC, comprising a genomic disruption in a target sequence of an endogenous SOCS1 gene that suppresses or eliminates expression of a functional SOCS1 protein, wherein the target sequence comprises SEQ ID NO: 17 or SEQ ID NO: 23.

20. A modified iPSC, γδT cell, or intermediate cell differentiated from an iPSC, comprising a genomic disruption in a target sequence of an endogenous CISH gene that suppresses or eliminates expression of CISH, wherein the target sequence comprises SEQ ID NO: 24.


SEQUENCE LISTING (85 sequences)

SEQ ID NO: 1 - RNA, 20 nt, synthetic construct
  source 1..20: mol_type = other RNA; note = Targeting sequence in Scramble #2
gcactcacatcgctacatca

SEQ ID NO: 2 - RNA, 20 nt, synthetic construct
  source 1..20: mol_type = other RNA; note = Targeting sequence in BIM # 5
tacccattgcactgagatag

SEQ ID NO: 3 - RNA, 20 nt, synthetic construct
  source 1..20: mol_type = other RNA; note = Targeting sequence in CISH #3
aggccacatagtgctgcaca

SEQ ID NO: 4 - RNA, 20 nt, synthetic construct
  source 1..20: mol_type = other RNA; note = Targeting sequence in CISH #4
tgtacagcagtggctggtgg

SEQ ID NO: 5 - RNA, 20 nt, synthetic construct
  source 1..20: mol_type = other RNA; note = Targeting sequence in SOCS1 sgRNA
agtagaatccgcaggcgtcc

SEQ ID NO: 6 - RNA, 20 nt, synthetic construct
  source 1..20: mol_type = other RNA; note = Targeting sequence in BIM #9
agttctgagtgtgaccgaga

SEQ ID NO: 7 - RNA, 20 nt, synthetic construct
  source 1..20: mol_type = other RNA; note = Targeting sequence in BIM #19
gcctccccagctcagacctg

SEQ ID NO: 8 - RNA, 20 nt, synthetic construct
  source 1..20: mol_type = other RNA; note = Targeting sequence in BIM #24
ggcctggcaaggaggacttg

SEQ ID NO: 9 - RNA, 20 nt, synthetic construct
  source 1..20: mol_type = other RNA; note = Targeting sequence in BIM #25
tggttgaaggcctggcaagg

SEQ ID NO: 10 - RNA, 21 nt, synthetic construct
  source 1..21: mol_type = other RNA; note = Targeting sequence in BIM crRNA (MAD7)
acacagacaggagcccagcac

SEQ ID NO: 11 - RNA, 21 nt, synthetic construct
  source 1..21: mol_type = other RNA; note = Targeting sequence in SOCS1 crRNA (MAD7)
acctggatggcagccgcgaga

SEQ ID NO: 12 - RNA, 21 nt, synthetic construct
  source 1..21: mol_type = other RNA; note = Targeting sequence in CISH crRNA (MAD7)
ggtgtacagcagtggctggtg

SEQ ID NO: 13 - RNA, 20 nt, synthetic construct
  source 1..20: mol_type = other RNA; note = Target sequence by Scramble #2 sgRNA
tgatgtagcgatgtgagtgc

SEQ ID NO: 14 - RNA, 20 nt, synthetic construct
  source 1..20: mol_type = other RNA; note = Target sequence in BIM by BIM # 5 sgRNA
ctatctcagtgcaatgggta

SEQ ID NO: 15 - RNA, 20 nt, synthetic construct
  source 1..20: mol_type = other RNA; note = Target sequence in CISH by CISH #3 sgRNA
tgtgcagcactatgtggcct

SEQ ID NO: 16 - RNA, 20 nt, synthetic construct
  source 1..20: mol_type = other RNA; note = Target sequence in CISH by CISH #4 sgRNA
ccaccagccactgctgtaca

SEQ ID NO: 17 - RNA, 20 nt, synthetic construct
  source 1..20: mol_type = other RNA; note = Target sequence in SOCS1 by SOCS1 sgRNA
ggacgcctgcggattctact

SEQ ID NO: 18 - RNA, 20 nt, synthetic construct
  source 1..20: mol_type = other RNA; note = Target sequence in BIM by BIM #9 sgRNA
tctcggtcacactcagaact

SEQ ID NO: 19 - RNA, 20 nt, synthetic construct
  source 1..20: mol_type = other RNA; note = Target sequence in BIM by BIM #19 sgRNA
caggtctgagctggggaggc

SEQ ID NO: 20 - RNA, 20 nt, synthetic construct
  source 1..20: mol_type = other RNA; note = Target sequence in BIM by BIM #24 sgRNA
caagtcctccttgccaggcc

SEQ ID NO: 21 - RNA, 20 nt, synthetic construct
  source 1..20: mol_type = other RNA; note = Target sequence in BIM by BIM #25 sgRNA
ccttgccaggccttcaacca

SEQ ID NO: 22 - RNA, 21 nt, synthetic construct
  source 1..21: mol_type = other RNA; note = Target sequence in BIM by BIM crRNA (MAD7)
gtgctgggctcctgtctgtgt

SEQ ID NO: 23 - RNA, 21 nt, synthetic construct
  source 1..21: mol_type = other RNA; note = Target sequence in SOCS1 by SOCS1 crRNA (MAD7)
tctcgcggctgccatccaggt

SEQ ID NO: 24 - RNA, 21 nt, synthetic construct
  source 1..21: mol_type = other RNA; note = Target sequence in CISH by CISH crRNA (MAD7)
caccagccactgctgtacacc

SEQ ID NO: 25 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
gcactcacatcgctacatcagttttagagctagaaatagcaagttaaaataaggctagtccgttatcaacttgaaaaagtggcaccgagtcggtgctttt

SEQ ID NO: 26 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
tacccattgcactgagataggttttagagctagaaatagcaagttaaaataaggctagtccgttatcaacttgaaaaagtggcaccgagtcggtgctttt

SEQ ID NO: 27 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
aggccacatagtgctgcacagttttagagctagaaatagcaagttaaaataaggctagtccgttatcaacttgaaaaagtggcaccgagtcggtgctttt

SEQ ID NO: 28 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
tgtacagcagtggctggtgggttttagagctagaaatagcaagttaaaataaggctagtccgttatcaacttgaaaaagtggcaccgagtcggtgctttt

SEQ ID NO: 29 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
agtagaatccgcaggcgtccgttttagagctagaaatagcaagttaaaataaggctagtccgttatcaacttgaaaaagtggcaccgagtcggtgctttt

SEQ ID NO: 30 - RNA, 100 nt, synthetic construct; identical to SEQ ID NO: 25
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage

SEQ ID NO: 31 - skipped

SEQ ID NO: 32 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
agttctgagtgtgaccgagagttttagagctagaaatagcaagttaaaataaggctagtccgttatcaacttgaaaaagtggcaccgagtcggtgctttt

SEQ ID NO: 33 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
gcctccccagctcagacctggttttagagctagaaatagcaagttaaaataaggctagtccgttatcaacttgaaaaagtggcaccgagtcggtgctttt

SEQ ID NO: 34 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
ggcctggcaaggaggacttggttttagagctagaaatagcaagttaaaataaggctagtccgttatcaacttgaaaaagtggcaccgagtcggtgctttt

SEQ ID NO: 35 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
tggttgaaggcctggcaagggttttagagctagaaatagcaagttaaaataaggctagtccgttatcaacttgaaaaagtggcaccgagtcggtgctttt

SEQ ID NO: 36 - RNA, 56 nt, synthetic construct
  source 1..56: mol_type = other RNA
  modified_base 1..3: mod_base = OTHER; note = 2-O-methyl modifications
  modified_base 53..55: mod_base = OTHER; note = 2-O-methyl modifications
  modified_base 1..4: mod_base = OTHER; note = phosphorothioate linkage
  modified_base 53..56: mod_base = OTHER; note = phosphorothioate linkage
gtcaaaagacctttggaatttctactcttgtagatacacagacaggagcccagcac

SEQ ID NO: 37 - RNA, 56 nt, synthetic construct
  source 1..56: mol_type = other RNA
  modified_base 1..3: mod_base = OTHER; note = 2-O-methyl modifications
  modified_base 53..55: mod_base = OTHER; note = 2-O-methyl modifications
  modified_base 1..4: mod_base = OTHER; note = phosphorothioate linkage
  modified_base 53..56: mod_base = OTHER; note = phosphorothioate linkage
gtcaaaagacctttggaatttctactcttgtagatacctggatggcagccgcgaga

SEQ ID NO: 38 - RNA, 56 nt, synthetic construct
  source 1..56: mol_type = other RNA
  modified_base 1..3: mod_base = OTHER; note = 2-O-methyl modifications
  modified_base 53..55: mod_base = OTHER; note = 2-O-methyl modifications
  modified_base 1..4: mod_base = OTHER; note = phosphorothioate linkage
  modified_base 53..56: mod_base = OTHER; note = phosphorothioate linkage
gtcaaaagacctttggaatttctactcttgtagatggtgtacagcagtggctggtg

SEQ ID NO: 39 - RNA, 20 nt, synthetic construct
  source 1..20: mol_type = other RNA; note = BIM forward
tttgctaccagatccccgct

SEQ ID NO: 40 - RNA, 23 nt, synthetic construct
  source 1..23: mol_type = other RNA; note = BIM reverse
ccaaaccctctgagcaataagca

SEQ ID NO: 41 - RNA, 20 nt, synthetic construct
  source 1..20: mol_type = other RNA; note = CISH forward
gcactgccttgacttgcact

SEQ ID NO: 42 - RNA, 20 nt, synthetic construct
  source 1..20: mol_type = other RNA; note = CISH reverse
gggggcatttacctccaagt

SEQ ID NO: 43 - RNA, 20 nt, synthetic construct
  source 1..20: mol_type = other RNA; note = SOCS1 forward
tccactgaggctgaacggat

SEQ ID NO: 44 - RNA, 19 nt, synthetic construct
  source 1..19: mol_type = other RNA; note = SOCS1 reverse
ctgaaagtgcacgcggatg

SEQ ID NO: 45 - RNA, 24 nt, synthetic construct
  source 1..24: mol_type = other RNA; note = PCR primer - Forward BIM
accgagaaggtagacaattgcagc

SEQ ID NO: 46 - RNA, 24 nt, synthetic construct
  source 1..24: mol_type = other RNA; note = PCR primer - Reverse BIM
agtttgacacatcctccattccca

SEQ ID NO: 47 - RNA, 20 nt, synthetic construct
  source 1..20: mol_type = other RNA; note = Sequencing primer BIM
aatcacggaggtgaagggga

SEQ ID NO: 48 - RNA, 22 nt, synthetic construct
  source 1..22: mol_type = other RNA; note = PCR primer - Forward SOCS1
gccccttctgtaggatggtagc

SEQ ID NO: 49 - RNA, 22 nt, synthetic construct
  source 1..22: mol_type = other RNA; note = PCR primer - Reverse SOCS1
ggggaaggagctcaggtagtcg

SEQ ID NO: 50 - RNA, 20 nt, synthetic construct
  source 1..20: mol_type = other RNA; note = Sequencing primer SOCS1
agcacacaaccaggtggcag

SEQ ID NO: 51 - RNA, 20 nt, synthetic construct
  source 1..20: mol_type = other RNA; note = PCR primer - Forward CISH
ggttccattacggccagcga

SEQ ID NO: 52 - RNA, 20 nt, synthetic construct
  source 1..20: mol_type = other RNA; note = PCR primer - Reverse CISH
ggccagctgccagaggtatg

SEQ ID NO: 53 - RNA, 20 nt, synthetic construct
  source 1..20: mol_type = other RNA; note = Sequencing primer CISH
cgcaccccagctacctgttc

SEQ ID NO: 54 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
cgtgagtaaacctgaatcttgttttagagctagaaatagcaagttaaaataaggctagtccgttatcaacttgaaaaagtggcaccgagtcggtgctttt

SEQ ID NO: 55 - RNA, 20 nt, synthetic construct
  source 1..20: mol_type = other RNA; note = B2M sgRNA (B2M #1)
cgtgagtaaacctgaatctt

SEQ ID NO: 56 - RNA, 20 nt, synthetic construct
  source 1..20: mol_type = other RNA; note = B2M sgRNA (B2M #1)
aagattcaggtttactcacg

SEQ ID NO: 57 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
gatattggcataagcctcccgttttagagctagaaatagcaagttaaaataaggctagtccgttatcaacttgaaaaagtggcaccgagtcggtgctttt

SEQ ID NO: 58 - RNA, 20 nt, synthetic construct
  source 1..20: mol_type = other RNA; note = CIITA sgRNA (CIITA #1)
gatattggcataagcctccc

SEQ ID NO: 59 - RNA, 20 nt, synthetic construct
  source 1..20: mol_type = other RNA; note = CIITA sgRNA (CIITA #1)
gggaggcttatgccaatatc

SEQ ID NO: 60 - RNA, 100 nt, synthetic construct
  source 1..100: mol_type = other RNA
  modified_base 1..3: mod_base = OTHER; note = 2-O-methyl modifications
  modified_base 97..99: mod_base = OTHER; note = 2-O-methyl modifications
  modified_base 1..4: mod_base = OTHER; note = phosphorothioate linkage
  modified_base 97..100: mod_base = OTHER; note = phosphorothioate linkage
gattgctcaacaaccatgctgttttagagctagaaatagcaagttaaaataaggctagtccgttatcaacttgaaaaagtggcaccgagtcggtgctttt

SEQ ID NO: 61 - RNA, 20 nt, synthetic construct
  source 1..20: mol_type = other RNA; note = FAS sgRNA (FAS # 1)
gattgctcaacaaccatgct

SEQ ID NO: 62 - RNA, 20 nt, synthetic construct
  source 1..20: mol_type = other RNA; note = FAS sgRNA (FAS # 1)
agcatggttgttgagcaatc

SEQ ID NO: 63 - protein, 228 aa, synthetic construct [sequence omitted]

SEQ ID NO: 64 - protein, 226 aa, synthetic construct
  source 1..226: note = ECD of IFNGR2
APPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQ

SEQ ID NO: 65 - protein, 5 aa, synthetic construct
  source 1..5: note = Membrane-proximal region of IL2RG
FALEA

SEQ ID NO: 66 - protein, 5 aa, synthetic construct
  source 1..5: note = Membrane-proximal region of IL2RB
LGKDT

SEQ ID NO: 67 - protein, 21 aa, synthetic construct
  source 1..21: note = Transmembrane domain of IL2RG
VVISVGSMGLIISLLCVYFWL

SEQ ID NO: 68 - protein, 25 aa, synthetic construct
  source 1..25: note = Transmembrane domain of IL2RB
IPWLGHLLVGLSGAFGFIILVYLLI

SEQ ID NO: 69 - protein, 86 aa, synthetic construct
  source 1..86: note = Intracellular domain of IL2RG
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 70 - protein, 286 aa, synthetic construct
  source 1..286: note = Intracellular domain of IL2RB
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 71 - protein, 340 aa, synthetic construct
  source 1..340: note = IFNGR1 Subunit: IFNGR1-IL2RG without signal peptide
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 72 - protein, 542 aa, synthetic construct
  source 1..542: note = IFNGR2 Subunit: IFNGR2-IL2RB without signal peptide
APPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQLGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 73 - protein, 21 aa, synthetic construct
  source 1..21: note = CD8 signal peptide
MALPVTALLLPLALLLHAARP

SEQ ID NO: 74 - protein, 242 aa, synthetic construct
  source 1..242: note = CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 75 - protein, 45 aa, synthetic construct
  source 1..45: note = CD8 loop
TTTPAPRPPTPAPTIASQPLSLRPEACRPAAGGAVHTRGLDFACD

SEQ ID NO: 76 - protein, 24 aa, synthetic construct
  source 1..24: note = CD8 TM
IYIWAPLAGTCGVLLLSLVITLYC

SEQ ID NO: 77 - protein, 42 aa, synthetic construct
  source 1..42: note = 41BB ICD
KRGRKKLLYIFKQPFMRPVQTTQEEDGCSCRFPEEEEGGCEL

SEQ ID NO: 78 - protein, 112 aa, synthetic construct
  source 1..112: note = CD3z AD
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 17 aa, synthetic construct
  source 1..17: note = Signal peptide of IFNGR1
MALLFLLPLVMQGVSRA

SEQ ID NO: 80 - protein, 21 aa, synthetic construct
  source 1..21: note = Signal peptide of IFNGR2
MRPTLLWSLLLLLGVFAAAAA

SEQ ID NO: 81 - protein, 357 aa, synthetic construct
  source 1..357: note = IFNGR1 Subunit: IFNGR1-IL2RG with signal peptide
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 82 - protein, 563 aa, synthetic construct
  source 1..563: note = IFNGR2 Subunit: IFNGR2-IL2RB with signal peptide
MRPTLLWSLLLLLGVFAAAAAAPPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQLGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 83 - protein, 465 aa, synthetic construct
  source 1..465: note = CD19-CAR without signal peptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 486 aa, synthetic construct
  source 1..486: note = CD19-CAR with signal peptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 500 aa, synthetic construct
  source 1..500: note = B2M-HLA-E fusion protein
MSRSVALAVLALLSLSGLEAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL